(19) United States
(12) Patent Application Publication
(10) Pub. No.: US 2026/0284213 A1
(43) Pub. Date: Sep. 24, 2026

(54) LINKERS FOR DRUG CONJUGATES AND ANTIBODY DRUG CONJUGATES

(71) Applicant: BIOATLA, INC., San Diego, CA (US)

(72) Inventors: Samapika SINHA, Basel (CH); Sylvain BLANC, Basel (CH); Martin BUESCHLEB, Basel (CH); Gerhard FREY, San Diego, CA (US)

(73) Assignee: BIOATLA, INC., San Diego, CA (US)

(21) Appl. No.: 19/474,932

(22) PCT Filed: Apr. 24, 2024
(86) PCT No.: PCT/US24/25939

§ 371 Date: Oct. 13, 2025

Related U.S. Application Data
(60) Provisional application No. 63/498,810, filed on Apr. 27, 2023.

(51) Int. Cl. A61K 47/68; A61K 47/54; A61P 35/00

(52) U.S. Cl. CPC A61K 47/68031; A61K 47/549; A61K 47/6889; A61P 35/00

(57) ABSTRACT

The present disclosure provides novel and advantageous synthetic linkers and conjugates capable of covalently coupling to one or more isolated polypeptide or one or more antibody or antibody fragment. The isolated polypeptide, antibody, or antibody fragment having a heavy chain variable region and/or light chain variable region that specifically binds to Nectin-4 protein. The linker-coupled polypeptides, antibodies or antibody fragments and pharmaceutical compositions and kits containing the same are also provided, as well as methods of making the synthetic linker and conjugates, and methods of therapeutically using the same.

### INCORPORATION OF MATERIAL OF XML SEQUENCE LISTING BY REFERENCE

The sequence listing submitted herewith as an XML file named “BIAT-1036WOSequenceListingXML” created on Apr. 24, 2024, which is 81 kilobytes in size, is hereby incorporated by reference in its entirety.

### FIELD OF THE DISCLOSURE

This disclosure relates to synthetic linkers capable of covalently coupling to an isolated polypeptide, an antibody, or an antibody fragment. The synthetic linkers may be coupled to an isolated polypeptide, antibody or an antibody fragment, and compositions and/or kits containing the same are provided, as well as methods of producing and using the covalently coupled polypeptide, antibody, or antibody fragment for therapy. The improved stability of the linker-coupled polypeptide, antibody or antibody fragment of the present disclosure provides the significant advantage of reducing adverse events associated with payload release often encountered with use of other types of linkers, which enhances the therapeutic efficacy of the linker-coupled polypeptide, antibody or antibody fragment in treatments such as immunoconjugate therapies. The isolated polypeptides, antibodies, or antibody fragments may be conditionally active or non-conditionally active. In particular, the conditionally or non-conditionally active isolated polypeptides, antibodies, or antibody fragments may be anti-Nectin-4 polypeptides, anti-Nectin-4 antibodies, anti-Nectin-4 antibody fragments, or anti-Nectin-4 multi-specific antibodies.

### BACKGROUND OF THE DISCLOSURE

Antibody-drug conjugates (ADCs) are complex molecules composed of an antibody (such as a monoclonal antibody) connected to a biologically active, highly cytotoxic drug moiety via a cleavable (such as acid labile linkers, protease linkers, and disulfide linkers) or a non-cleavable linker. The conjugation of the cytotoxic drug moiety to an antibody allows for targeted delivery of the drug payloads to disease tissue surfaces (such as a tumor surface), minimizing toxic effects to healthy tissue and hence, improves the therapeutic window of ADCs in treatment therapies such as cancer treatment.

A “linker” is a bifunctional or multifunctional moiety that can be used to link one or more drug (D) moieties to a polypeptide such as an antibody (Ab) to form an immunoconjugate or an antibody-drug conjugate (ADC). In this respect, linkers play a significant role in the stability of ADCs and the drug payload release profile important for their efficacy. One of the challenges in developing safe and effective ADCs is the generation of suitable chemical linkers between cytotoxic drugs and antibodies (such as monoclonal antibodies). Biopharma PEG, What are ADC linkers: Cleavable vs. Non-Cleavable Linkers; biochempeg.com/article/87.html; Dec. 19, 2019 release date.

Ideally, a linker should be stable in the circulatory system and release the cytotoxic payload specifically to a target, such as a tumor. Su Z. et al., Acta Pharmaceutica Sinica B., 11(12): 3889-3907 (2021). However, existing linkers suffer a defect that restricts the development of ADCs in that they release payloads nonspecifically, which leads to off-target toxicity. Su Z., et al. (2021).

Nectin-4 is a surface molecule that belongs to the nectin family of proteins, which comprises four members. Nectins are cell adhesion molecules that play a key role in various biological processes during development and adult life such as polarity, proliferation, differentiation and migration of epithelial, endothelial, immune and neuronal cells. Nectins are involved in several pathological processes in humans. Nectins are the main receptors for polio, herpes simplex and measles viruses. Mutations in the genes encoding Nectin-1 (PVRL1) and Nectin-4 (PVRL4) cause ectodermal dysplasia syndromes that are associated with several abnormalities. Nectin-4 is expressed during fetal development. In adult tissues its expression is more restricted than that of other members of the family.

Nectin-4 is a tumor-associated antigen in 30%, 49%, and 86% of breast, ovarian and lung carcinomas, respectively, and is frequently associated with aggressive tumors. In breast tumors, Nectin-4 is expressed mainly in triple-negative carcinomas. In the serum of patients with these cancers, the detection of soluble forms of Nectin-4 is associated with a poor prognosis. Levels of serum Nectin-4 increase during metastatic progression and decrease after treatment. These results suggest that Nectin-4 could be a reliable target for the treatment of cancer.

Accordingly, several anti-Nectin-4 antibodies have been described in the prior art. In particular, Enfortumab Vedotin (ASG-22ME) is an antibody-drug conjugate (ADC) targeting Nectin-4 and is currently in clinical investigation for the treatment of patients suffering from solid tumors.

The present disclosure aims to provide antibody drug conjugates including anti-Nectin-4 antibodies or antibody fragments and novel linkers, which conjugates are suitable for therapeutic and diagnostic use, especially for diagnosis and treatment of cancers, having reduced or minimal side effects. Some of the anti-Nectin-4 antibodies or antibody fragments may have a higher binding activity or binding affinity to Nectin-4 in a tumor microenvironment in comparison with the binding activity or binding affinity to Nectin-4 in a non-tumor microenvironment. The anti-Nectin-4 antibodies or antibody fragments of the present disclosure typically have at least comparable efficacy to known anti-Nectin-4 antibodies. In addition, the present anti-Nectin-4 antibodies or antibody fragments may exhibit reduced side effects in comparison with monoclonal anti-Nectin-4 antibodies known in the art for having a relatively low binding affinity to Nectin-4 in normal tissues such as non-tumor environments and microenvironments. These advantages may provide a more selective targeting of the Nectin-4 expressed in a tumor and may permit use of higher dosages of the present anti-Nectin-4 antibodies or antibody fragments as a result of their increased binding activity to Nectin-4 present in a tumor microenvironment, whereby more effective therapeutic treatments can be realized without a corresponding increase in undesirable side effects.

### SUMMARY OF THE DISCLOSURE

In one aspect, the present disclosure provides a conjugate of Formula I,

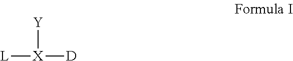

in which L is represented by:

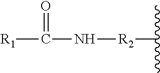

where R₁ is a nucleophilic functional group capable of coupling to an antibody, said functional group being selected from the group consisting of a C₁-C₁₀ alkyl halide, a C₁-C₁₀ alkyl alcohol, a phenol, a C₁-C₁₀ alkyl thiol, and a C₁-C₁₀ alkyl azide, and R₂ is a substituted or unsubstituted C₁-C₂₀ alkylene, or a substituted or unsubstituted (PEG)ₙ moiety represented by:

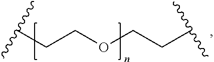

where n is 1 to 20;
X is a substituted nitrophenyl represented by:

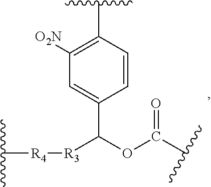

where R₃ is a C₁-C₂₀ alkylene;
R₄ is a triazole ring having a C═C double bond in the ring;
Y is a glycoside or glycoside derivative;
D is a drug; and
the wavy lines indicate a site of covalent attachment.
Y is bonded to X at the site of the wavy line attached to the nitrophenyl ring, D is bonded to X at the site of the wavy line attached to the carbonyl, and R₂ is bonded to R₄.

In the above embodiment, R₁ can be C₁-C₆ alkyl halide with a halide selected from the group consisting of Br, I, Cl, and F, a C₁-C₂ alkyl alcohol, a 4-phenol, or a C₁-C₂ alkyl thiol.

In each of the embodiments of paragraphs [0009]-[0010], R₁ can be —CH₂—Br.

In each of the embodiments of paragraphs [0009]-[0011], R₂ can be an unsubstituted C₁-C₂₀ alkylene.

In an embodiment of paragraph [0012], R₂ can be an unsubstituted C₂-C₅ alkylene.

In each of the previous embodiments of paragraphs [0009]-[0011], R₂ can be an unsubstituted (PEG)ₙ where n can be 8.

In each of the previous embodiments of paragraphs [0009]-[0014], R₃ can be —CH₂—.

In each of the embodiments of paragraphs [0009]-[0015], the glycoside or glycoside derivative Y can be selected from the following structures:

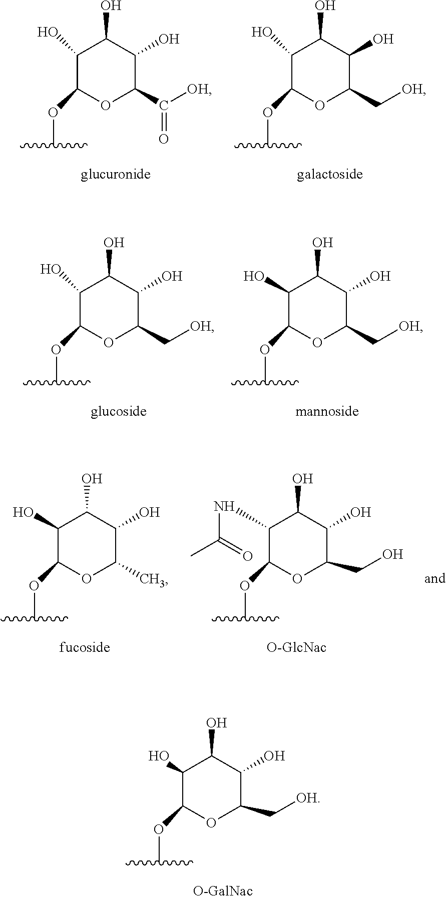

In each of the previous embodiments of paragraphs [0009]-[0016], the glycoside or glycoside derivative Y can be a glucuronide.

In each of the previous embodiments of paragraphs [0009]-[0017], the triazole ring can be formed with a linker intermediate (L^{int}) having a formula represented by:

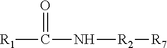

where R₁ can be a nucleophilic functional group capable of coupling to an antibody, said functional group being selected from the group consisting of a C₁-C₁₀ alkyl halide, a C₁-C₁₀ alkyl alcohol, a phenol, a C₁-C₁₀ alkyl thiol, and a C₁-C₁₀ alkyl azide,
R₂ can be a substituted or unsubstituted C₁-C₂₀ alkylene, or a substituted or unsubstituted (PEG)ₙ moiety represented by

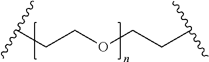

where n can be 1 to 20, and R₇ can be an azide group or an acetylene group, wherein the nitrogen atoms of the azide group or the carbons of the acetylene group can become ring members in the triazole ring. In view of the fact that an acetylene group can react with an azide group in a Click reaction, as described herein below, it is envisioned that the triazole ring of R₄ can be formed by reacting an azide group bonded to R₃ with an acetylene group at R₇ or vice versa, i.e. by reacting an acetylene group bonded to R₃ with an azide group at R₇.

In each of the previous embodiments of paragraphs [0009]-[0018], the triazole ring can be formed with an azide group or an acetylene group bonded to L which can be represented by

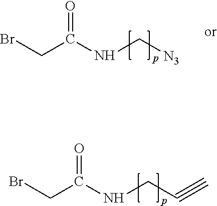

where p can be 2 to 5, or
L can be represented by

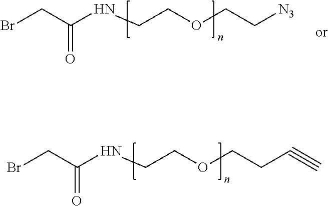

where n can be 8.

In each of the previous embodiments of paragraphs [0009]-[0019], the linker can have a structure represented by

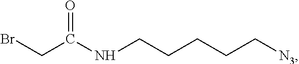 [SMILES: [N-]=[N+]=NCCCCCNC(=O)CBr]

or the linker can have a structure represented by

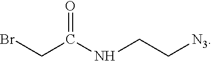

In each of the previous embodiments of paragraphs [0009]-[0020], the conjugate can be SPC07 having the following structure:

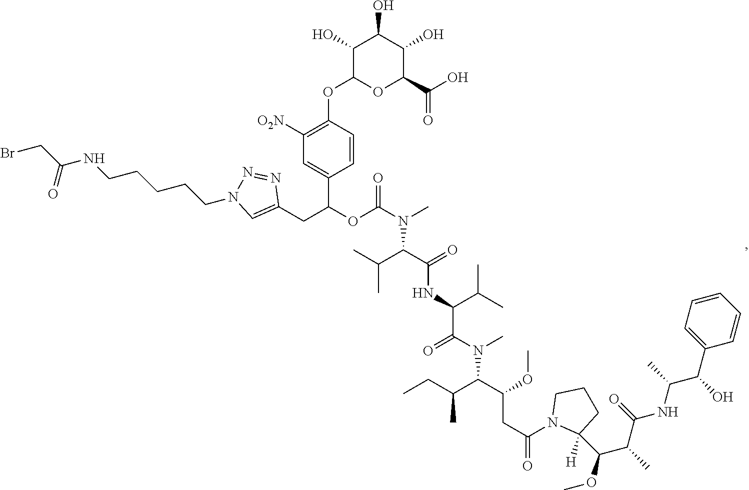 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(Cc1cn(CCCCCNC(=O)CBr)nn1)c1ccc(OC2O[C@H](C(=O)O)[C@@H](O)[C@H](O)[C@H]2O)c([N+](=O)[O-])c1)C(C)C]

or
the conjugate can be SPC19 having the following structure:

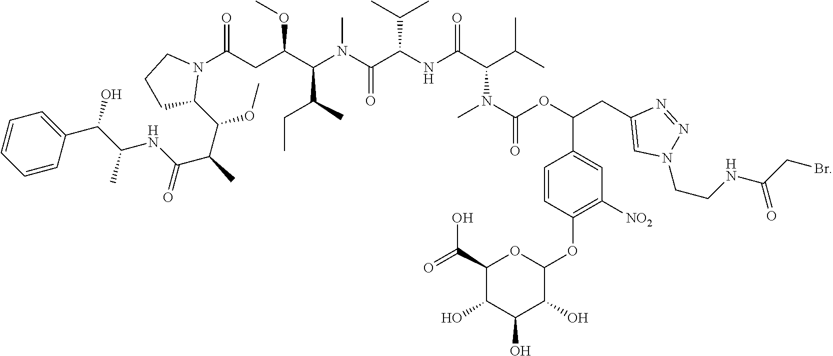 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(Cc1cn(CCNC(=O)CBr)nn1)c1ccc(OC2O[C@H](C(=O)O)[C@@H](O)[C@H](O)[C@H]2O)c([N+](=O)[O-])c1)C(C)C]

In an embodiment of paragraph [0018], Y may not be a galactoside when R₂ is a substituted or unsubstituted (PEG)n.

In each of the embodiments of paragraphs [0009]-[0018], the conjugate can be SPC17 having the following structure:

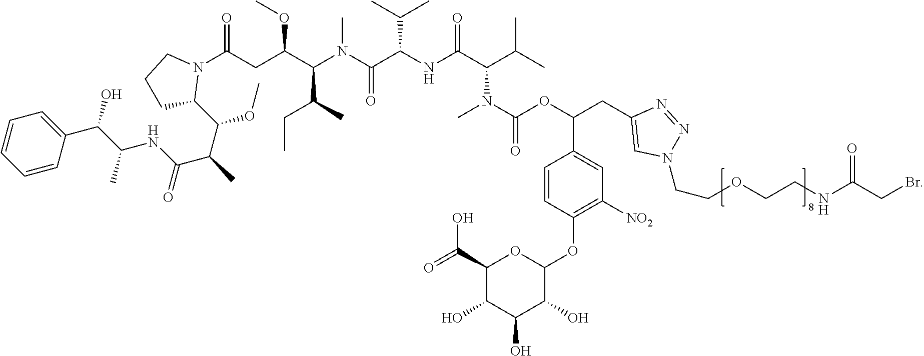

In any one of the previous embodiments of paragraphs [0009]-[0023], the drug D can be selected from the group consisting of an auristatin, a dolastatin, a maytansinoid, a calicheamicin, a pyrrolobenzodiazepine, an anthracycline, a ribonuclease, and a DNA endonuclease.

In a particular embodiment of the above paragraph [0024], the drug D can be an auristatin selected from the group consisting of monomethyl auristatin E (MMAE) and monomethyl auristatin F (MMAF).

In another aspect, the present disclosure provides a linker intermediate (L^{int}) having a formula represented by

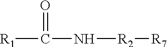

where R₁ is nucleophilic functional group capable of coupling to an antibody selected from the group consisting of a C₁-C₁₀ alkyl halide, a C₁-C₁₀ alkyl alcohol, a phenol, a C₁-C₁₀ alkyl thiol, and a C₁-C₁₀ alkyl azide,
R₂ is a substituted or unsubstituted C₁-C₂₀ alkylene, or a substituted or unsubstituted (PEG)n moiety represented by

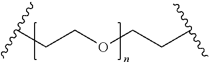

where n is 1 to 20, and
R₇ is an azide group or an acetylene group.

In an embodiment of paragraph [0026], R₁ can be C₁-C₆ alkyl halide with a halide selected from the group consisting of Br, I, Cl, and F, a C₁-C₂ alkyl alcohol, a 4-phenol, or a C₁-C₂ alkyl thiol.

In each of the previous embodiments of paragraphs [0026]-[0027], R₁ can be —CH₂—Br.

In each of the previous embodiments of paragraphs [0026]-[0028], R₂ can be an unsubstituted C₁-C₂₀ alkylene, or R₂ can be an unsubstituted (PEG)n where n can be 8.

In an embodiment of paragraph [0029], R₂ can be an unsubstituted C₂-C₅ alkylene.

In each of the previous embodiments of paragraphs [0026]-[0030], R₇ can be an azide group or an acetylene group.

In an embodiment of the above paragraph [0031], the linker can have a structure represented by

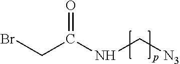

where p can be 2 to 5.

In an embodiment of the above paragraph [0032], the linker can have a structure represented by

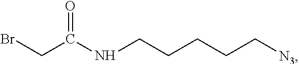 [SMILES: [N-]=[N+]=NCCCCCNC(=O)CBr]

or the linker can have a structure represented by

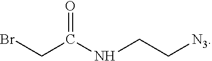

In each of the previous embodiments of paragraphs [0026]-[0028], the linker can have a structure represented by

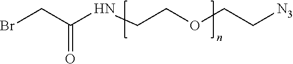

where n can be 8.

In another aspect, the present disclosure provides an immunoconjugate of Formula II,

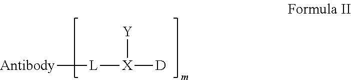

wherein:
L is represented by:

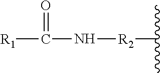

where R₁ is nucleophilic functional group capable of coupling to an antibody selected from the group consisting of a C₁-C₁₀ alkyl halide, a C₁-C₁₀ alkyl alcohol, a phenol, a C₁-C₁₀ alkyl thiol, and a C₁-C₁₀ alkyl azide,
R₂ is a substituted or unsubstituted C₁-C₂₀ alkylene, or a substituted or unsubstituted (PEG)n moiety represented by

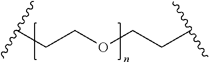

where n is 1 to 20;
X is a substituted nitrophenyl represented by

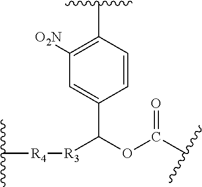

where R₃ is a C₁-C₂₀ alkylene; R₄ is a triazole ring having a C═C double bond in the ring;
Y is a glycoside or glycoside derivative;
D is a drug;
the wavy lines indicate a site of covalent attachment; and
m is 1 to 10,
wherein the term Antibody in Formula II represents an antibody or antibody fragment.

In the above embodiment of paragraph [0035], R₁ can be C₁-C₆ alkyl halide with a halide selected from the group consisting of Br, I, Cl, and F, a C₁-C₂ alkyl alcohol, a 4-phenol, or a C₁-C₂ alkyl thiol.

In each of the previous embodiments of paragraphs [0035]-[0036], R₁ can be —CH₂—Br.

In each of the previous embodiments of paragraphs [0035]-[0037], R₂ can be an unsubstituted C₁-C₂₀ alkylene.

In a particular embodiment of the above paragraph [0038], R₂ can be an unsubstituted C₂-C₅ alkylene.

In each of the previous embodiments of paragraphs [0035]-[0037], R₂ can be an unsubstituted (PEG)n where n is 8.

In each of the previous embodiments of paragraphs [0035]-[0040], R₃ can be —CH₂—.

In each of the previous embodiments of paragraphs [0035]-[0041], the glycoside or glycoside derivative Y can be selected from the following structures

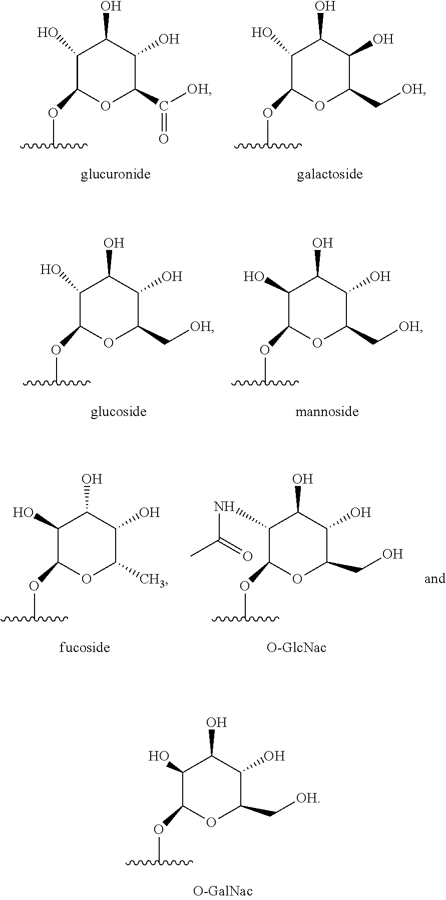

In each of the previous embodiments of paragraphs [0035]-[0042], the glycoside or glycoside derivative Y can be a glucuronide.

In each of the previous embodiments of paragraphs [0035]-[0043], L can be represented by

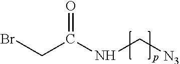

where p can be 2 to 5, or L can be represented by

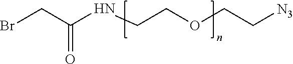

where n can be 8.

In an embodiment of the above paragraph [0044], the linker can have a structure represented by

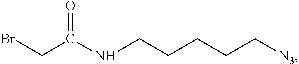 [SMILES: [N-]=[N+]=NCCCCCNC(=O)CBr]

or the linker can have a structure represented by

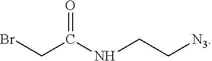

In any one of the previous embodiments of paragraphs [0035]-[0045], the drug D can be selected from the group consisting of an auristatin, a dolastatin, a maytansinoid, a calicheamicin, a pyrrolobenzodiazepine, an anthracycline, a ribonuclease, and a DNA endonuclease.

In a particular embodiment of the above paragraph [0046], the drug D can be an auristatin selected from the group consisting of monomethyl auristatin E (MMAE) and monomethyl auristatin F (MMAF).

In an embodiment of the above of paragraphs [0035]-[0044], the

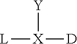

can have a structure selected from

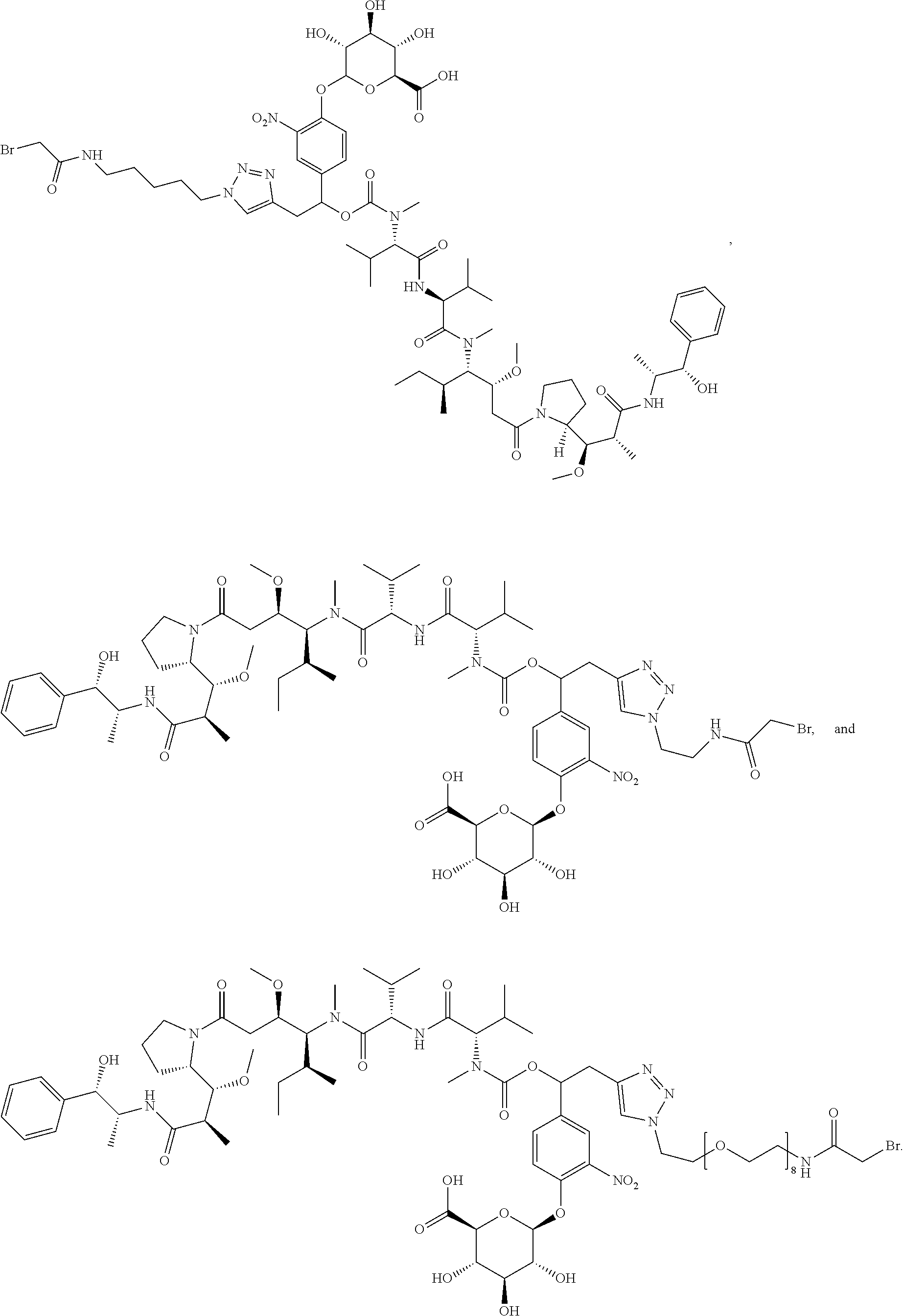

In another embodiment of the above paragraph [0044], Y may not be a galactoside when R₂ is a substituted or unsubstituted (PEG)n.

In each of the previous embodiments of [0035]-[0049], the antibody can be an anti-Nectin-4 antibody or antibody fragment comprising an isolated polypeptide having a heavy chain variable region including three complementarity determining regions (CDRs) having sequences H1, H2, and H3, wherein:
the H1 sequence can be GFTFSSYNX₁N (SEQ ID NO: 1);
the H2 sequence can be YISSSSSTIYYADSVKG (SEQ ID NO: 2); and
the H3 sequence can be AYYYGX₂DX₃ (SEQ ID NO: 3);
   wherein X₁ can be M or D; X₂ can be M or D; X₃ can be V or K, with the proviso that the heavy and light chain variable regions cannot be SEQ ID NOS: 18 and 31 in combination;
   and a light chain variable region including three CDRs having sequences L1, L2, and L3, wherein:
the L1 sequence can be X₄ASQGISGWX₅A (SEQ ID NO: 4);
the L2 sequence can be AASTLQS (SEQ ID NO: 5); and
the L3 sequence can be QQANSX₆PX₇T (SEQ ID NO: 6),
   wherein X₄ can be R or H; X₅ can be L or E; X₆ can be For E; and X₇ can be P or D, and
   with the proviso that X₁, X₂, X₃, X₄, X₅, X₆ and X₇ cannot simultaneously be, M, M, V, R, L, F and P, respectively.

In the previous embodiment of paragraph [0050], the antibody or antibody fragment can comprise a heavy chain variable region and a light chain variable region that specifically binds to Nectin-4 wherein the heavy chain variable region includes three complementarity determining regions having sequences H1, H2, and H3, wherein:
the H1 sequence can be GFTFSSYNX₁N (SEQ ID NO: 1);
the H2 sequence can be YISSSSSTIYYADSVKG (SEQ ID NO: 2); and
the H3 sequence can be AYYYGX₂DX₃ (SEQ ID NO: 3);
   wherein X₁ can be M or D; X₂ can be M or D; X₃ can be V or K; and
   the light chain variable region includes three complementarity determining regions having sequences L1, L2, and L3, wherein:
the L1 sequence can be X₄ASQGISGWX₅A (SEQ ID NO: 4);
the L2 sequence can be AASTLQS (SEQ ID NO: 5); and
the L3 sequence can be QQANSX₆PX₇T (SEQ ID NO: 6),
   wherein X₄ can be R or H; X₅ can be L or E; X₆ can be For E; and X₇ can be P or D; and
   with the proviso that X₁, X₂, X₃, X₄, X₅, X₆ and X₇ cannot simultaneously be, M, M, V, R, L, F and P, respectively, with the proviso that the heavy and light chain variable regions cannot be SEQ ID NOS: 18 and 31 in combination.

In each of the previous embodiments of paragraphs [0050] to [0051] above, the H1 sequence may be selected from GFTFSSYNMN (SEQ ID NO: 7), and GFTFSSYNDN (SEQ ID NO: 8). The H3 sequence may be selected from AYYYGMDV (SEQ ID NO: 9), AYYYGDDV (SEQ ID NO: 10), and AYYYGMDK (SEQ ID NO: 11).

In each of the previous embodiments of paragraphs [0050] to [0052] above, the L1 sequence may be selected from RASQGISGWLA (SEQ ID NO: 12), RASQGISGWEA (SEQ ID NO: 13), and HASQGISGWLA (SEQ ID NO: 14). The L3 sequence may be selected from QQANSFPPT (SEQ ID NO: 15), QQANSEPPT (SEQ ID NO: 16), and QQANSFPDT (SEQ ID NO: 17).

In each of the previous embodiments of paragraphs [0050] to [0053] above, the antibody or antibody fragment may comprise a heavy chain variable region has a sequence selected from SEQ ID NOS: 18-30.

In each of the foregoing embodiments of paragraphs [0050] to [0054] above, the antibody or antibody fragment may comprise a light chain variable region having a sequence selected from SEQ ID NOS: 31-43.

In each of the previous embodiments of paragraphs [0050] to [0055], the antibody or antibody fragment can comprise a heavy chain variable region and a light chain variable region having any one pair of sequences selected from: SEQ ID NOS: 19 and 32, SEQ ID NOS: 20 and 33, SEQ ID NOS: 21 and 34, SEQ ID NOS: 22 and 35, SEQ ID NOS: 23 and 36, SEQ ID NOS: 24 and 37, SEQ ID NOS: 25 and 38, SEQ ID NOS: 26 and 39, SEQ ID NOS: 27 and 40, SEQ ID NOS: 28 and 41 and SEQ ID NOS: 29 and 42.

In each of the previous embodiments of paragraphs [0050] to [0056], the antibody or antibody fragment can comprise a heavy chain variable region and a light chain variable region each region independently having at least 80%, 85%, 90%, 95%, 98% or 99% identity to a combination of amino acid sequences selected from one of SEQ ID NOS: 18-30 in combination with one of SEQ ID NOS: 31-43; with the proviso that the heavy and light chain variable regions cannot be SEQ ID NOS: 18 and 31 in combination; and said antibody or antibody fragment can specifically bind to human Nectin-4 protein.

In each of the previous embodiments of paragraphs [0050] to [0057], the antibody or antibody fragment can comprise a heavy chain variable region and a light chain variable region each region independently having at least 80%, 85%, 90%, 95%, 98% or 99% identity to a pair of amino acid sequences selected from: SEQ ID NOS: 19 and 32, SEQ ID NOS: 20 and 33, SEQ ID NOS: 21 and 34, SEQ ID NOS: 22 and 35, SEQ ID NOS: 23 and 36, SEQ ID NOS: 24 and 37, SEQ ID NOS: 25 and 38, SEQ ID NOS: 26 and 39, SEQ ID NOS: 27 and 40, SEQ ID NOS: 28 and 41 and SEQ ID NOS: 29 and 42, respectively; with the proviso that the heavy and light chain variable regions cannot be SEQ ID NOS: 18 and 31 in combination; and said antibody or antibody fragment can specifically bind to human Nectin-4 protein.

In each of the previous embodiments of paragraphs [0050] to [0058], the antibody or antibody fragment may specifically bind to Nectin-4, or especially human Nectin-4 protein.

In a yet another aspect, the antibody or antibody fragment can be multi-specific and specifically bind to Nectin-4, or especially human Nectin-4 protein and to CD3 and the antibody can comprise a heavy chain variable region and a light chain variable region, wherein the heavy chain variable region includes three complementarity determining regions having sequences H1, H2, and H3, wherein:
the H1 sequence can be selected from SEQ ID NO: 7 and SEQ ID NO: 8,
the H2 sequence can be SEQ ID NO: 2, and
the H3 sequence can be selected from SEQ ID NO: 9, SEQ ID NO: 10, and SEQ ID NO: 11; and
the light chain variable region can include three complementarity determining regions having sequences L1, L2, and L3, wherein:
   the L1 sequence can be X₄ASQGISGWX₅A (SEQ ID NO: 4);
   the L2 sequence can be AASTLQS (SEQ ID NO: 5); and
   the L3 sequence can be QQANSX₆PX₇T (SEQ ID NO: 6),
wherein X₄ can be R or H; X₅ can be L or E; X₆ can be For E; and X₇ can be P or D; and
with the proviso that X₁, X₂, X₃, X₄, X₅, X₆ and X₇ cannot simultaneously be, M, M, V, R, L, F and P, respectively, and
six anti-CD3 complementarity determining regions L4, L5, L6, L7, L8, and L9 wherein:
the L4 sequence can be GFTFNTYAMN (SEQ ID NO: 44),
the L5 sequence can be RIRSKYNNYATYYADSVKD (SEQ ID NO: 45),
the L6 sequence can be HX₁₁NFX₁₂NSX₁₃ VSWFX₁₄Y (SEQ ID NO: 46),
the L7 sequence can be RSSTGAVTTSNYX₁₅N (SEQ ID NO: 47),
the L8 sequence can be GTNKRAP (SEQ ID NO: 48), and
the L9 sequence can be ALWYSNLWV (SEQ ID NO: 49),
wherein X₁₁ can be G or S, X₁₂ can be G or P, X₁₃ can be Y or K, X₁₄ can be A or Q and X₁₅ can be A or D.

In another aspect of the antibody or antibody fragment of paragraph [0060], the L6 sequence can be selected from any one of SEQ ID NOs: 50-53, and the L7 sequence can be selected from SEQ ID NOs: 54 and 55.

In a preferred aspect, the antibody or antibody fragment of paragraphs [0060]-[0061] can comprise a heavy chain variable region that includes three complementarity determining regions, H1, H2, and H3, wherein:
the H1 sequence can be selected from SEQ ID NO: 7 and SEQ ID NO: 8,
the H2 sequence can be SEQ ID NO: 2, and
the H3 sequence can be selected from SEQ ID NO: 9, SEQ ID NO: 10, and SEQ ID NO: 11; and
the light chain variable region includes three complementarity determining regions can have sequences L1, L2, and L3, wherein:
the L1 sequence can be selected from SEQ ID NO: 12, SEQ ID NO: 13, and SEQ ID NO: 14,
the L2 sequence can be SEQ ID NO: 5,
the L3 sequence can be selected from SEQ ID NO: 15, SEQ ID NO: 16, and SEQ ID NO: 17, and six anti-CD3 complementarity determining regions L4, L5, L6, L7, L8, and L9 wherein:
the L4 sequence can be GFTENTYAMN (SEQ ID NO: 44),
the L5 sequence can be RIRSKYNNYATYYADSVKD (SEQ ID NO: 45),
the L6 sequence can be selected from HGNFGNSYVSWFAY (SEQ ID NO: 50),
HSNFGNSKVSWFAY (SEQ ID NO: 51), HGNFPNSKVSWFQY (SEQ ID NO: 52), and
HSNFGNSKVSWFAY (SEQ ID NO: 53),
the L7 sequence can be selected from RSSTGAVTTSNYAN (SEQ ID NO: 54) and RSSTGAVTTSNYDN (SEQ ID NO: 55),
the L8 sequence can be GTNKRAP (SEQ ID NO: 48), and
the L9 sequence can be ALWYSNLWV (SEQ ID NO: 49).

In another preferred aspect, the antibody or antibody fragment of paragraphs [0060]-[0062] can comprise a heavy chain variable region that includes three complementarity determining regions, H1, H2, and H3, wherein:
the H1 sequence can be SEQ ID NO: 7,
the H2 sequence can be SEQ ID NO: 2, and
the H3 sequence can be selected from SEQ ID NO: 9, SEQ ID NO: 10, and SEQ ID NO: 11; and
the light chain variable region can include three complementarity determining regions having sequences L1, L2, and L3, wherein:
the L1 sequence can be selected from SEQ ID NO: 12 and SEQ ID NO: 13,
the L2 sequence can be SEQ ID NO: 5,
the L3 sequence can be SEQ ID NO: 15, and six anti-CD3 complementarity determining regions L4, L5, L6, L7, L8, and L9 wherein:
the L4 sequence can be GFTFNTYAMN (SEQ ID NO: 44),
the L5 sequence can be RIRSKYNNYATYYADSVKD (SEQ ID NO: 45),
the L6 sequence can be selected from HGNFGNSYVSWFAY (SEQ ID NO: 50),
HSNFGNSKVSWFAY (SEQ ID NO: 51), HGNFPNSKVSWFQY (SEQ ID NO: 52), and
HSNFGNSKVSWFAY (SEQ ID NO: 53),
the L7 sequence can be selected from RSSTGAVTTSNYAN (SEQ ID NO: 54) and RSSTGAVTTSNYDN (SEQ ID NO: 55),
the L8 sequence can be GTNKRAP (SEQ ID NO: 48), and
the L9 sequence can be ALWYSNLWV (SEQ ID NO: 49).

In each of the previous embodiments of paragraphs [0060]-[0063], the antibody or antibody fragment may comprise a heavy chain variable region having a sequence selected from SEQ ID NOS: 18, 25, 27, and 29.

In each of the previous embodiments of paragraphs [0060]-[0064], the antibody or antibody fragment may comprise a light chain variable region having a sequence selected from SEQ ID NOS: 56-60, with the proviso that the heavy and light chain variable regions cannot be SEQ ID NOS: 18 and 56 combination.

In each of the previous embodiments of paragraphs [0060]-[0065], the antibody or antibody fragment may comprise a heavy chain variable region having a sequence selected from SEQ ID NOs: 18, 25, 27, and 29, and a light chain variable region having a sequence selected from SEQ ID NOs: 56-60, with the proviso that the heavy and light chain variable regions cannot be SEQ ID NOS: 18 and 56 combination.

In each of the previous embodiments of paragraphs [0060]-[0066], the antibody or antibody fragment may comprise a heavy chain variable region and a light chain variable region having any one pair of sequences selected from: SEQ ID NO: 25 and SEQ ID NO: 57, SEQ ID NO: 27 and SEQ ID NO: 58, SEQ ID NO: 29 and SEQ ID NO: 59, and SEQ ID NO: 29 and SEQ ID NO: 60.

In each of the previous embodiments of paragraphs [0050]-[0063], the the antibody or antibody fragment of the present disclosure may comprise a heavy chain variable region and a light chain variable region, each region independently having at least 80%, 85%, 90%, 95%, 98% or 99% identity to a combination of amino acid sequences selected from one of SEQ ID NOS: 18, 25, 27, and 29 in combination with one of SEQ ID NOs: 56-60, with the proviso that the heavy and light chain variable regions cannot be SEQ ID NOS: 18 and 56 combination; and said isolated polypeptides may specifically bind to human Nectin-4 protein.

In each of the previous embodiments of paragraphs [0050]-[0063], the the antibody or antibody fragment may comprise a heavy chain variable region and a light chain variable region each region independently having at least 80%, 85%, 90%, 95%, 98% or 99% identity to a pair of amino acid sequences selected from: SEQ ID NOS: 25 and 57, SEQ ID NOS: 27 and 58, SEQ ID NOS: 29 and 59, SEQ ID NOS: 29 and 60; and said antibody or antibody fragment may specifically bind to human Nectin-4 protein.

In each of the previous embodiments of paragraphs to [0069], the antibody or antibody fragment may be a multispecific antibody or antibody fragment that specifically binds to Nectin-4, especially human Nectin-4 protein.

In a preferred aspect of paragraphs to [0070], the bispecific antibody or antibody fragment can specifically bind to Nectin-4 and CD3, especially human Nectin-4 protein and CD3.

In each of the previous embodiments of paragraphs to [0071], the antibody or antibody fragment may have a higher binding affinity to Nectin-4 protein, especially human Nectin-4 protein, at a value of a condition in a tumor microenvironment in comparison with a different value of the same condition that occurs in a non-tumor microenvironment. In one embodiment, the condition can be pH.

In each of the previous embodiments of paragraphs to [0072], the antibody or antibody fragment may have at least 70% of the antigen binding activity at pH 6.0 as compared to the same antigen binding activity of the parent antibody or antibody fragment at pH 6.0, and the antibody or antibody fragment may have less than 50%, or less than 40%, or less than 30%, or less than 20% or less than 10% of the antigen binding activity at pH 7.4 as compared to the same antigen binding activity of the parent antibody or antibody fragment at pH 7.4. The antigen binding activity may be binding to Nectin-4 protein.

In each of the foregoing embodiments of paragraphs to [0073], the antibody or antibody fragment may have a higher binding affinity to Nectin-4 protein, especially human Nectin-4 protein, at a pH in a tumor microenvironment in comparison with a pH that occurs in a non-tumor microenvironment. The pH in the tumor microenvironment may range of from 5.0 to 6.8 and the pH in the non-tumor microenvironment may range from 7.0 to 7.6.

In each of the previous embodiments, the antigen binding activity of the antibody or antibody fragment may be measured by an ELISA assay.

In yet another aspect, the present disclosure provides an immunoconjugate that includes any of the antibodies or antibody fragments of the disclosure described above. In the immunoconjugate, the antibody or antibody fragment may be conjugated to an agent selected from a chemotherapeutic agent, a radioactive atom, a cytostatic agent and a cytotoxic agent.

In yet another aspect, the present disclosure provides a pharmaceutical composition that includes any of the antibodies or antibody fragments, or the immunoconjugates of the disclosure described above, together with a pharmaceutically acceptable carrier.

A single dose of the pharmaceutical composition may include an amount of the the antibody, the antibody fragment, or the immunoconjugate of about 135 mg, 235 mg, 335 mg, 435 mg, 535 mg, 635 mg, 735 mg, 835 mg, 935 mg, 1035 mg, 1135 mg, 1235 mg, or 1387 mg.

A single dose of the pharmaceutical composition may include an amount of the the antibody or antibody fragment, or the immunoconjugate in a range of 135-235 mg, 235-335 mg, 335-435 mg, 435-535 mg, 535-635 mg, 635-735 mg, 735-835 mg, 835-935 mg, 935-1035 mg, 1035-1135 mg, 1135-1235 mg, or 1235-1387 mg.

Each of the foregoing pharmaceutical compositions may further include an immune checkpoint inhibitor molecule. The immune checkpoint inhibitor molecule may be an antibody or antibody fragment against an immune checkpoint. The immune checkpoint may be selected from LAG3, TIM3, TIGIT, VISTA, BTLA, OX40, CD40, 4-1BB, CTLA4, PD-1, PD-L1, GITR, B7-H3, B7-H4, KIR, A2aR, CD27, CD70, DR3, and ICOS or the immune checkpoint may be CTLA4, PD-1 or PD-L1.

Each of the foregoing pharmaceutical compositions may further include an antibody or antibody fragment against an antigen selected from PD1, PD-L1, CTLA4, AXL, ROR2, CD3, HER2, B7-H3, ROR1, SFRP4 and a WNT protein. The WNT protein may be selected from WNT1, WNT2, WNT2B, WNT3, WNT4, WNT5A, WNT5B, WNT6, WNT7A, WNT7B, WNT8A, WNT8B, WNT9A, WNT9B, WNT10A, WNT10B, WNT11 and WNT16.

In yet another aspect, the present disclosure provides a kit for diagnosis or treatment including any of the antibodies or antibody fragments, the immunoconjugates, or the pharmaceutical compositions of the present disclosure described above.

In yet another aspect, the present disclosure provides a pharmaceutical composition that includes any of the linker, conjugate, and/or immunoconjugate, of the disclosure described above, together with a pharmaceutically acceptable carrier.

The present disclosure also provides use of a linker, conjugate, and/or immunoconjugate of any one of the embodiments described above, for the treatment of cancer.

The present disclosure further provides a kit for diagnosis or treatment, said kit comprising of a linker, conjugate, and/or immunoconjugate of any one of the embodiments of described above, and instructions for using the a linker, conjugate, and/or immunoconjugate for diagnosis or treatment of cancer.

### BRIEF DESCRIPTION OF THE DRAWINGS

FIG. 1 shows the structure of SPC07, a conjugate comprising a linker of the present disclosure.

FIGS. 2A and 2B show amino acid sequences of the light chain variable regions (FIG. 2A) and the heavy chain variable regions (FIG. 2B) of a conditionally active anti-Nectin-4 antibody of the present disclosure (BA-143-01-VK and BA-143-01-VH) and of the wild type anti-Nectin-4 antibody (BAP143-00-VK and BAP143-00-VH).

FIGS. 3A and 3B show PK analysis with pH affinity ELISA of BA-143-00-MC, BA-143-00-SPC04, BA-143-00-SPC07, BA-143-00-SPC08, BA-143-01-MC, BA-143-01-SPC04, BA-143-01-SPC07, and BA-143-01-SPC08 from 0.17 h to 168 h. The results are summarized in TABLES 3 and 4.

FIGS. 4A and 4B show PK analysis with MMAE affinity ELISA of BA-143-00-MC, BA-143-00-SPC04, BA-143-00-SPC07, BA-143-00-SPC08, BA-143-01-MC, BA-143-01-SPC04, BA-143-01-SPC07, and BA-143-01-SPC08 from 0.17 h to 168 h. The results are summarized in TABLES 5 and 6.

FIG. 5A shows the mean tumor volume of Groups 1 (vehicle), 2 (BA-143-00-MC), 4 (BA-143-00-SPC07), 6 (BA-143-01-MC), 8 (BA-143-01-SPC07), 10 (BA-B12-MC), and 12 (BA-B12-SPC07) of TABLE 10-4.

FIG. 5B shows the mean tumor volume of Group 1 (vehicle), Group 2 (BA-185.01-00502-02-MC), Group 3 (BA-185.01-00502-02-SPC07), Group 4 (BA-185.01-00502-02-SPC17), and Group 5 (BA-185.01-00502-02-SPC19) of Table 7-7.

FIG. 5C shows the mean tumor volume of Group 1 (vehicle), Group 2 (BA-104.01-00523-01-MC), Group 3 (BA-104.01-00523-01-SPC07), Group 4 (BA-104.01-00523-01-SPC17), and Group 5 (BA-104.01-00523-01-SPC19) of Table 7-8.

FIG. 5D shows the mean tumor volume of Group 1 (vehicle), Group 2 (BA-143-00-MC), Group 3 (BA-143-00-SPC07), Group 4 (BA-143-00-SPC17), and Group 5 (BA-143-00-SPC19) of Tables 7-9 and 7-10.

FIG. 5E shows the tumor volume of Group 1 (vehicle), Group 2 (BA-091-00-2E2-MC), Group 3 (BA-091-00-2E2-SPC07), Group 4 (BA-091-00-2E2-SPC17), and Group 5 (BA-091-00-2E2-SPC19) of Table 7-11.

FIG. 6A shows no significant body weight loss (10% or above) in any of the groups.

FIG. 6B shows relative body weight changes of each of the different groups.

FIG. 7 shows the tumor sizes of each of the different groups at time points after treatment.

FIG. 8A shows there was no difference in the DAR in human serum of BA-143-00-MC, BA-143-00-SPC04, BA-143-00-SPC07, and BA-143-00-SPC08.

FIG. 8B shows the difference in % DAR change between BA-143-00-MC and the groups BA-143-00-SPC04, BA-143-00-SPC07, and BA-143-00-SPC08.

FIG. 9A shows the binding activities of BA-143-00-MC, BA-143-00-SPC07, BA-143-01-MC, and BA-143-01-SPC07 to human Nectin-4 as measured by pH affinity ELISA assay.

FIG. 9B shows the binding activities of BA-143-00-MC, BA-143-00-SPC07, BA0143-01-MC, and BA-143-01-SPC07 to cyno Nectin-4 as measured by pH affinity ELISA assay.

FIG. 10A shows BA-143-00-MC, BA-143-00-SPC04, BA-143-00-SPC07, BA-143-00-SPC08, BA-143-01-MC, BA-143-01-SPC04, BA-143-01-SPC07, and BA-143-01-SPC08 specifically bind to Nectin4, and to a similar degree at pH 6.0.

FIG. 10B shows BA-143-00-MC, BA-143-00-SPC04, BA-143-00-SPC07, BA-143-00-SPC08, BA-143-01-MC, BA-143-01-SPC04, BA-143-01-SPC07, and BA-143-01-SPC08 specifically bind to Nectin4, but BA-143-01-MC, BA-143-01-SPC04, BA-143-01-SPC07, and BA-143-01-SPC08 exhibit reduced binding activity to Nectin4 at pH 7.4 in comparison to BA-143-00-MC, BA-143-00-SPC04, BA-143-00-SPC07, and BA-143-00-SPC08.

FIG. 11A shows BA-143-00-MC, BA-143-00-SPC04, BA-143-00-SPC07, BA-143-00-SPC08, BA-143-01-MC, BA-143-01-SPC04, BA-143-01-SPC07, and BA-143-01-SPC08 specifically bind to human Nectin4, and to a similar degree at pH 6.0.

FIG. 11B shows BA-143-00-MC, BA-143-00-SPC04, BA-143-00-SPC07, BA-143-00-SPC08, BA-143-01-MC, BA-143-01-SPC04, BA-143-01-SPC07, and BA-143-01-SPC08 specifically bind to human Nectin4, but BA-143-01-MC, BA-143-01-SPC04, BA-143-01-SPC07, and BA-143-01-SPC08 exhibit reduced binding activity to human Nectin4 at pH 7.4 in comparison to BA-143-00-MC, BA-143-00-SPC04, BA-143-00-SPC07, and BA-143-00-SPC08.

FIGS. 12A and 12B show BA-143-00-MC, BA-143-00-SPC04, BA-143-00-SPC07, BA-143-00-SPC08, BA-143-01-MC, BA-143-01-SPC04, BA-143-01-SPC07, and BA-143-01-SPC08 specifically bind to human Nectin4, and that at 0.1 μl/ml of each of BA-143-01-MC, BA-143-01-SPC04, BA-143-01-SPC07, and BA-143-01-SPC08 exhibited reduced binding activity to human Nectin4 at pH 7.4 in comparison to BA-143-00-MC, BA-143-00-SPC04, BA-143-00-SPC07, and BA-143-00-SPC08.

FIG. 13 shows the inhibitions of different doses of BA-143-00-SPC17 and BA-143-00-SPC19 plotted as concentration-response luminescence signals.

FIG. 14A shows was no difference in the DAR in human serum on Day 0, Day 7, and Day 14, between each of BA-143-00-MC, BA-143-00-SPC17, and BA-143-00-SPC19.

FIG. 14B shows a difference in % DAR change between BA-143-00-MC and the groups BA-143-00-SP17, and BA-143-00-SPC19.

FIG. 15A shows the mean tumor volume of vehicle group (G1), BA-143-00-SPC17 (G6) and BA-143-00-SPC19 (G8).

FIG. 15B shows the relative body weight changes of vehicle group (G1), BA-143-00-SPC17 (G6) and BA-143-00-SPC19 (G8).

FIG. 16A shows the mean body weight of different treatment groups, with no group demonstrating significant body weight loss (10% or above).

FIG. 16B shows the body weight change (%) of different groups.

FIG. 16C shows the mean tumor volume of different groups.

FIG. 17 shows the effect on tumor volumes of xxT47D xenograft mice of treatment with representative CAB ADCs of the present disclosure and the WT ADC of the present disclosure.

FIG. 18 shows protein sequences of the heavy and light chain variable regions of representative conditionally active anti-Nectin-4 antibodies of the present disclosure and the heavy and light chain variable regions of the benchmark wild type antibody.

FIG. 19A shows the higher binding activity of CAB Nectin-4×CAB CD3 affinity in a tumor microenvironment pH in comparison to physiological pH as measured by ELISA.

FIG. 19B shows the differential binding affinity of CAB Nectin-4×CAB CD3 and WT Nectin-4×WT CD3 in the pH range 6.0-7.4.

FIG. 19C shows the in vivo efficacy of CAB Nectin4×CAB CD3 in comparison to Isotype x WT CD3 and WT Nectin-6×WT CD3.

FIGS. 20A-20B show protein sequences of the heavy and light chain variable regions of representative conditionally active Nectin-4×CD3 bispecific antibodies of the present disclosure and the heavy and light chain variable regions of the wild type antibody. The heavy chains (FIG. 20A) are: BA-150-19-01-01-BF1-VH (SEQ ID NO: 18), BA-150-30-33-16-BF11-VH (SEQ ID NO: 25), BA-150-30-33-16-BF19-VH (SEQ ID NO: 27), BA-150-30-03-12-BF11-VH (SEQ ID NO: 29) and BA-150-30-03-12-BF19-VH (SEQ ID NO: 29). The light chains (FIG. 20B) are: BA-150-19-01-01-BF1-LC (SEQ ID NO: 56), BA-150-30-33-16-BF11-LC (SEQ ID NO: 57), BA-150-30-33-16-BF19-LC (SEQ ID NO: 58), BA-150-30-03-12-BF11-LC (SEQ ID NO: 59), and BA-150-30-03-12-BF19-LC (SEQ ID NO: 60).

FIG. 21A shows the structure of SPC17, a conjugate comprising a linker of the present disclosure.

FIG. 21B shows the structure of SPC19, a conjugate comprising a linker of the present disclosure.

FIG. 22A shows the structure of SPC02, 2-(1-(4-((2-bromoacetamido)methyl)benzyl)-1H-1,2,3-triazol-4-yl)-1-(3-nitro-4-(((3R,4S,5R,6R)-3,4,5-trihydroxy-6-(hydroxymethyl)tetrahydro-2H-pyran-2-yl)oxy)phenyl)ethyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl) pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl) carbamate.

FIG. 22B shows the structure of SPC04, 2-(1-(5-(2-bromoacetamido) pentyl)-1H-1,2,3-triazol-4-yl)-1-(3-nitro-4-(((3R,4S,5R,6R)-3,4,5-trihydroxy-6-(hydroxymethyl)tetrahydro-2H-pyran-2-yl)oxy)phenyl)ethyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl) pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl) carbamate.

FIG. 22C shows the structure of SPC05, 2-(1-(1-bromo-2-oxo-6,9,12-trioxa-3-azatetradecan-14-yl)-1H-1,2,3-triazol-4-yl)-1-(3-nitro-4-(((3R,4S,5R,6R)-3,4,5-trihydroxy-6-(hydroxymethyl)tetrahydro-2H-pyran-2-yl)oxy)phenyl)ethyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl) pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl) carbamate.

FIG. 22D shows the structure of SPC06, 2-(1-(10-(2-bromoacetamido) decyl)-1H-1,2,3-triazol-4-yl)-1-(3-nitro-4-(((3R,4S,5R,6R)-3,4,5-trihydroxy-6-(hydroxymethyl)tetrahydro-2H-pyran-2-yl)oxy)phenyl)ethyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl) pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl) carbamate.

FIG. 22E shows the structure of SPC08, 2-(1-(10-(2-bromoacetamido) decyl)-1H-1,2,3-triazol-4-yl)-1-(3-nitro-4-(((3R,4S,5R,6R)-3,4,5-trihydroxy-6-(hydroxymethyl)tetrahydro-2H-pyran-2-yl)oxy)phenyl)ethyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl) pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl) carbamate.

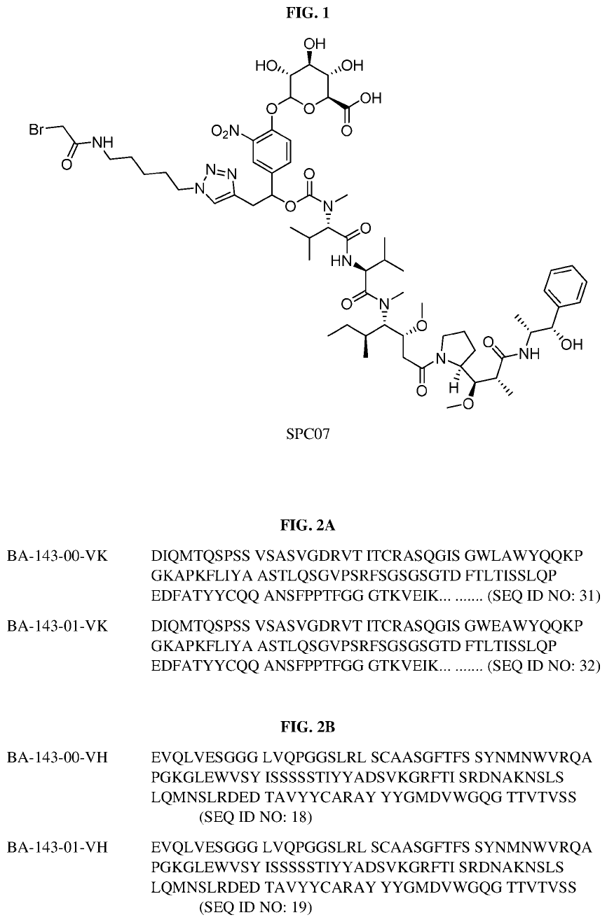

### DEFINITIONS

In order to facilitate understanding of the examples provided herein, certain frequently occurring terms are defined herein.

In connection with a measured quantity, the term “about” as used herein refers to the normal variation in that measured quantity that would be expected by a skilled person making the measurement and exercising a level of care commensurate with the objective of the measurement and the precision of the measuring equipment used. Unless otherwise indicated, “about” refers to a variation of +/−10% of the value provided.

The term “alkyl” as used herein is a branched or unbranched saturated hydrocarbon group of 1 to 20 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C₁ alkyl, C₁-C₂ alkyl, C₁-C₃ alkyl, C₁-C₄ alkyl, C₁-C₈alkyl, C₁-C₆ alkyl, C₁-C₇ alkyl, C₁-C₈alkyl, C₁-C₉ alkyl, C₁-C₁₀ alkyl, and the like up to and including a C₁-C₂₀ alkyl.

The term, “alkylene” as used herein refers to a bivalent saturated aliphatic radical (such as ethylene derived from an alkene by opening of the double bond or from an alkane by removal of two hydrogen atoms from different carbon atoms.

The term “affinity” as used herein refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

The term “affinity matured” antibody as used herein refers to an antibody with one or more alterations in one or more heavy chain or light chain variable regions, compared to a parent antibody which does not possess such alterations, such alterations resulting in an improvement in the affinity of the antibody for antigen.

The term “amino acid” as used herein refers to any organic compound that contains an amino group (—NH2) and a carboxyl group (—COOH); preferably either as free groups or alternatively after condensation as part of peptide bonds. The “twenty naturally encoded polypeptide-forming alpha-amino acids” are understood in the art and refer to: alanine (ala or A), arginine (arg or R), asparagine (asn or N), aspartic acid (asp or D), cysteine (cys or C), gluatamic acid (glu or E), glutamine (gin or Q), glycine (gly or G), histidine (his or H), isoleucine (ile or I), leucine (leu or L), lysine (lys or K), methionine (met or M), phenylalanine (phe or F), proline (pro or P), serine (ser or S), threonine (thr or T), tryptophan (tip or W), tyrosine (tyr or Y), and valine (val or V).

The term “antibody” as used herein refers to intact immunoglobulin molecules, as well as fragments of immunoglobulin molecules, such as Fab, Fab′, (Fab′)2, Fv, and SCA fragments, that are capable of binding to an epitope of an antigen. These antibody fragments, which retain some ability to selectively bind to an antigen (e.g., a polypeptide antigen) of the antibody from which they are derived, can be made using well known methods in the art (see, e.g., Harlow and Lane, supra), and are described further, as follows. Antibodies can be used to isolate preparative quantities of the antigen by immunoaffinity chromatography. Various other uses of such antibodies are to diagnose and/or stage disease (e.g., neoplasia) and for therapeutic application to treat disease, such as for example: neoplasia, autoimmune disease, AIDS, cardiovascular disease, infections, and the like. Chimeric, human-like, humanized or fully human antibodies are particularly useful for administration to human patients.

An Fab fragment consists of a monovalent antigen-binding fragment of an antibody molecule, and can be produced by digestion of a whole antibody molecule with the enzyme papain, to yield a fragment consisting of an intact light chain and a portion of a heavy chain.

An Fab′ fragment of an antibody molecule can be obtained by treating a whole antibody molecule with pepsin, followed by reduction, to yield a molecule consisting of an intact light chain and a portion of a heavy chain. Two Fab′ fragments are obtained per antibody molecule treated in this manner.

An (Fab′) 2 fragment of an antibody can be obtained by treating a whole antibody molecule with the enzyme pepsin, without subsequent reduction. A (Fab′) 2 fragment is a dimer of two Fab′ fragments, held together by two disulfide bonds.

An Fv fragment is defined as a genetically engineered fragment containing the variable region of a light chain and the variable region of a heavy chain expressed as two chains.

The term “antibody fragment” as used herein refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

The terms “anti-Nectin-4 antibody”, “Nectin-4 antibody” and “an antibody that binds to Nectin-4” as used herein refers to an antibody that is capable of binding Nectin-4 with sufficient affinity such that the antibody is useful as a diagnostic and/or therapeutic agent in targeting Nectin-4. In one embodiment, the extent of binding of an anti-Nectin-4 antibody to an unrelated, non-Nectin-4 protein is less than about 10% of the binding of an antibody to Nectin-4 as measured, e.g. by a radioimmunoassay (RIA). In certain embodiments, an antibody that binds to Nectin-4 has a dissociation constant≤1 μM, ≤100 nM, ≤10 nM, ≤1 nM, ≤0.1 nM, ≤0.01 nM, or ≤0.001 nM (e.g. 10⁻⁸M or less, e.g. from 10⁻⁸M to 10⁻¹³ M, e.g., from 10⁻⁹M to 10⁻¹³ M). In certain embodiments, an anti-Nectin-4 antibody binds to an epitope of Nectin-4 that is conserved among Nectin-4 from different species, for example, the extracellular domain of Nectin-4.

The term “Nectin-4” has its general meaning in the art and includes human Nectin-4, in particular the native-sequence polypeptide, isoforms, chimeric polypeptides, all homologs, fragments, and precursors of human Nectin-4. The amino acid sequence for native Nectin-4 includes the NCBI Reference Sequence: NP_112178.2.

The term “binding” as used herein refers to interaction of the variable region or an Fv of an antibody with an antigen, wherein the interaction depends upon the presence of a particular structure (e.g., an antigenic determinant or epitope) within the antigen. As used herein, the term “specifically binding” or “binding specifically” means that an antibody variable region or Fv binds to or associates with a particular antigen (i.e., “its” antigen) more frequently, more rapidly, with greater duration, and/or with greater affinity than it binds to or associates with other proteins. For example, an antibody variable region or Fv specifically binds to its particular antigen with greater affinity, avidity, more readily, and/or with greater duration than it binds to other antigens. For another example, an antibody variable region or Fv binds to a particular cell surface protein (antigen) with a materially greater affinity than it binds to related proteins or other cell surface proteins or to antigens commonly recognized by polyreactive natural antibodies (i.e., by naturally occurring antibodies known to bind to a variety of antigens naturally found in humans). However, “specific binding” does not require “selective binding”, i.e. exclusive binding to a particular antigen with zero or undetectable binding to any other antigen. In one example, “specific binding” of an antibody variable region or Fv (or other binding region) to an antigen means that the antibody variable region or Fv binds to the antigen with a dissociation constant (Kd) of, for example, 100 nM or less, or 50 nM or less, or 20 nM or less, or 15 nM or less, or 10 nM or less, or 5 nM or less, or 2 nM or less, or 1 nM or less.

The terms “cancer” and “cancerous” as used herein refer to or describe the physiological condition in mammals that is typically characterized by unregulated cell growth/proliferation. Examples of cancer include, but are not limited to, carcinoma, lymphoma (e.g., Hodgkin's and non-Hodgkin's lymphoma), blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma, leukemia and other lymphoproliferative disorders, and various types of head and neck cancer.

The terms “cell proliferative disorder” and “proliferative disorder” as used herein refer to disorders that are associated with some degree of abnormal cell proliferation. In one embodiment, the cell proliferative disorder is cancer.

The term “chemotherapeutic agent” as used herein refers to a chemical compound useful in the treatment of cancer. Examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Nicolaou et al., Angew. Chem. Intl. Ed. Engl., 33:183-186 (1994)); CDP323, an oral alpha-4 integrin inhibitor; dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®), liposomal doxorubicin TLC D-99 (MYOCET®), peglylated liposomal doxorubicin (CAELYX®), and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2′-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoid, e.g., paclitaxel (TAXOL®), albumin-engineered nanoparticle formulation of paclitaxel (ABRAXANE™), and docetaxel (TAXOTERE®); chloranbucil; 6-thioguanine; mercaptopurine; methotrexate; platinum agents such as cisplatin, oxaliplatin (e.g., ELOXATIN®), and carboplatin; vincas, which prevent tubulin polymerization from forming microtubules, including vinblastine (VELBAN®), vincristine (ONCOVIN®), vindesine (ELDISINE®, FILDESIN®), and vinorelbine (NAVELBINE®); etoposide (VP-16); ifosfamide; mitoxantrone; leucovorin; novantrone; edatrexate; daunomycin; aminopterin; ibandronate; topoisomerase inhibitor RFS 2000; difluoromethylornithine (DMF®); retinoids such as retinoic acid, including bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); BAY439006 (sorafenib; Bayer); SU-11248 (sunitinib, SUTENT®, Pfizer); perifosine, COX-2 inhibitor (e.g. celecoxib or etoricoxib), proteosome inhibitor (e.g. PS341); bortezomib (VELCADE®); CCI-779; tipifarnib (R11577); orafenib, ABT510; Bcl-2 inhibitor such as oblimersen sodium (GENASENSE®); pixantrone; EGFR inhibitors (see definition below); tyrosine kinase inhibitors (see definition below); serine-threonine kinase inhibitors such as rapamycin (sirolimus, RAPAMUNE®); farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

Chemotherapeutic agents as defined herein include “anti-hormonal agents” or “endocrine therapeutics,” which act to regulate, reduce, block, or inhibit the effects of hormones that can promote the growth of cancer. They may be hormones themselves, including, but not limited to: anti-estrogens with mixed agonist/antagonist profile, including, tamoxifen (NOLVADEX®), 4-hydroxytamoxifen, toremifene (FARESTON®), idoxifene, droloxifene, raloxifene (EVISTA®), trioxifene, keoxifene, and selective estrogen receptor modulators (SERMs) such as SERM3; pure anti-estrogens without agonist properties, such as fulvestrant (FASLODEX®), and EM800 (such agents may block estrogen receptor (ER) dimerization, inhibit DNA binding, increase ER turnover, and/or suppress ER levels); aromatase inhibitors, including steroidal aromatase inhibitors such as formestane and exemestane (AROMASIN®), and nonsteroidal aromatase inhibitors such as anastrazole (ARIMIDEX®), letrozole (FEMARA®) and aminoglutethimide, and other aromatase inhibitors include vorozole (RIVISOR®), megestrol acetate (MEGASE®), fadrozole, and 4(5)-imidazoles; lutenizing hormone-releaseing hormone agonists, including leuprolide (LUPRON® and ELIGARD®), goserelin, buserelin, and tripterelin; sex steroids, including progestines such as megestrol acetate and medroxyprogesterone acetate, estrogens such as diethylstilbestrol and premarin, and androgens/retinoids such as fluoxymesterone, all transretionic acid and fenretinide; onapristone; anti-progesterones; estrogen receptor down-regulators (ERDs); anti-androgens such as flutamide, nilutamide and bicalutamide; and pharmaceutically acceptable salts, acids or derivatives of any of the above; as well as combinations of two or more of the above.

The term “chimeric” antibody as used herein refers to an antibody in which a portion of the heavy and/or light chain is derived from a particular source or species, while the remainder of the heavy and/or light chain is derived from a different source or species.

The term “conditionally active antibody” as used herein refers to an anti-Nectin-4 antibody which is more active under a condition in the tumor microenvironment compared to under a condition in the non-tumor microenvironment. The conditions in the tumor microenvironment include lower pH, higher concentrations of lactate and pyruvate, hypoxia, lower concentration of glucose, and slightly higher temperature in comparison with non-tumor microenvironment. For example, a conditionally active antibody is virtually inactive at normal body temperature but is active at a higher temperature in a tumor microenvironment. In yet another aspect, the conditionally active antibody is less active in normal oxygenated blood, but more active under a less oxygenated environment exists in tumor. In yet another aspect, the conditionally active antibody is less active in normal physiological pH 7.0-7.6, but more active under an acidic pH 5.0-6.8, or 6.0-6.8 that exists in a tumor microenvironment. There are other conditions in the tumor microenvironment know to a person skilled in the field may also be used as the condition in the present disclosure under which the anti-Nectin-4 antibodies to have different binding affinity to Nectin-4.

The term “cytostatic agent” as used herein refers to a compound or composition which arrests growth of a cell either in vitro or in vivo. Thus, a cytostatic agent may be one which significantly reduces the percentage of cells in S phase. Further examples of cytostatic agents include agents that block cell cycle progression by inducing G0/G1 arrest or M-phase arrest. The humanized anti-Her2 antibody trastuzumab (HERCEPTIN®) is an example of a cytostatic agent that induces G0/G1 arrest. Classical M-phase blockers include the vincas (vincristine and vinblastine), taxanes, and topoisomerase II inhibitors such as doxorubicin, epirubicin, daunorubicin, etoposide, and bleomycin. Certain agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in Mendelsohn and Israel, eds., The Molecular Basis of Cancer, Chapter 1, entitled “Cell cycle regulation, oncogenes, and antineoplastic drugs” by Murakami et al. (W. B. Saunders, Philadelphia, 1995), e.g., p. 13. The taxanes (paclitaxel and docetaxel) are anticancer drugs both derived from the yew tree. Docetaxel (TAXOTERE®, Rhone-Poulenc Rorer), derived from the European yew, is a semisynthetic analogue of paclitaxel (TAXOL®, Bristol-Myers Squibb). Paclitaxel and docetaxel promote the assembly of microtubules from tubulin dimers and stabilize microtubules by preventing depolymerization, which results in the inhibition of mitosis in cells.

The term “cytotoxic agent” as used herein refers to a substance that inhibits or prevents a cellular function and/or causes cell death or destruction. Cytotoxic agents include, but are not limited to radioactive isotopes (e.g., At²¹¹, I¹³¹, I¹²⁵, Y⁹⁰, Re¹⁸⁶, Re¹⁸⁸, Sm¹⁵³, Bi²¹², p³², Pb²¹² and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

The term “diabodies” as used herein refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites.

The term “detectably label” as used herein refers to any substance whose detection or measurement, either directly or indirectly, by physical or chemical means, is indicative of the presence of an antigen in a sample. Representative examples of useful detectable labels include, but are not limited to the following: molecules or ions directly or indirectly detectable based on light absorbance, fluorescence, reflectance, light scatter, phosphorescence, or luminescence properties; molecules or ions detectable by their radioactive properties; molecules or ions detectable by their nuclear magnetic resonance or paramagnetic properties. Included among the group of molecules indirectly detectable based on light absorbance or fluorescence, for example, are various enzymes which cause appropriate substrates to convert, e.g., from non-light absorbing to light absorbing molecules, or from non-fluorescent to fluorescent molecules.

The term “diagnostics” as used herein refers to determination of a subject's susceptibility to a disease or disorder, determination as to whether a subject is presently affected by a disease or disorder, prognosis of a subject affected by a disease or disorder (e.g., identification of pre-metastatic or metastatic cancerous states, stages of cancer, or responsiveness of cancer to therapy), and therametrics (e.g., monitoring a subject's condition to provide information as to the effect or efficacy of therapy). In some embodiments, the diagnostic method of this disclosure is particularly useful in detecting early stage cancers.

The term “diagnostic agent” as used herein refers to a molecule which can be directly or indirectly detected and is used for diagnostic purposes. The diagnostic agent may be administered to a subject or a sample. The diagnostic agent can be provided per se or may be conjugated to a vehicle such as a conditionally active antibody.

The term “effector functions” as used herein refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

The term “effective amount” of an agent as used herein, e.g., a pharmaceutical formulation, refers to an amount effective, at dosages and for periods of time necessary, to achieve the desired therapeutic or prophylactic result.

The term “Fc region” as used herein is used to define a C-terminal region of an immunoglobulin heavy chain that contains at least a portion of the constant region. The term includes native sequence (or wildtype) Fc regions and variant (or mutant) Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

The term “framework” or “FR” as used herein refers to variable domain residues other than complementarity determining regions (CDRs or H1-3 in the heavy chain and L1-3 in the light chain) residues. The FR of a variable domain generally consists of four FR domains: FR1, FR2, FR3, and FR4. Accordingly, the CDR and FR sequences generally appear in the following sequence in VH_{H} (or V_{L}): FR1-H1 (L1)-FR2-H2 (L2)-FR3-H3 (L3)-FR4.

The term “full length antibody,” “intact antibody,” or “whole antibody” refers to an antibody which comprises an antigen-binding variable region (V_{H} or V_{L}) as well as a light chain constant domain (CL) and heavy chain constant domains, CH1, CH2 and CH3. The constant domains may be native sequence constant domains (e.g. human native sequence constant domains) or amino acid sequence variants thereof. Depending on the amino acid sequence of the constant domain of their heavy chains, full length antibodies can be assigned to different “classes”. There are five major classes of full length antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called alpha, delta, epsilon, gamma, and mu, respectively. The subunit structures and three-dimensional configurations of different classes of immunoglobulins are well known.

The term “function-conservative variants” as used herein refers a given amino acid residue in a protein or enzyme has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids other than those indicated as conserved may differ in a protein so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A “function-conservative variant” also includes a polypeptide which has at least 60% amino acid identity as determined by BLAST or FASTA algorithms, preferably at least 75%, more preferably at least 85%, still preferably at least 90%, and even more preferably at least 95%, and which has the same or substantially similar properties or functions as the native or parent protein to which it is compared.

The terms “host cell,” “host cell line,” and “host cell culture” as used herein are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

The term “human antibody” as used herein is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

The term “humanized” antibody as used herein refers to a chimeric antibody comprising amino acid residues from non-human CDRs and amino acid residues from human FRs. In certain embodiments, a humanized antibody will comprise substantially all of at least one, and typically two, variable domains, in which all or substantially all of the CDRs correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “immunoconjugate” as used herein is an antibody conjugated to one or more heterologous molecule(s), including but not limited to a cytotoxic agent.

The term “individual” or “subject” as used herein refers to a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

The term “inhibiting cell growth or proliferation” as used herein means decreasing a cell's growth or proliferation by at least 10%, 20%, 30%, 40%, 50%, 60%, 70%, 80%, 90%, 95%, or 100%, and includes inducing cell death.

The term “isolated” antibody as used herein is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase High Performance Liquid Chromatography (HPLC)). For review of methods for assessment of antibody purity, see, e.g., Flatman et al., J. Chromatogr. B, vol. 848, pp. 79-87, 2007.

The term “isolated nucleic acid encoding an anti-Nectin-4 antibody” as used herein refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The term “metastasis” as used herein refers to all Nectin-4-involving processes that support cancer cells to disperse from a primary tumor, penetrate into lymphatic and/or blood vessels, circulate through the bloodstream, and grow in a distant focus (metastasis) in normal tissues elsewhere in the body. In particular, it refers to cellular events of tumor cells such as proliferation, migration, anchorage independence, evasion of apoptosis, or secretion of angiogenic factors, that underlie metastasis and are stimulated or mediated by Nectin-4.

The term “microenvironment” as used herein means any portion or region of a tissue, organ or body that has constant or temporal, physical or chemical differences from other regions of the tissue, organ or regions of the body. For tumors, the term “tumor microenvironment” as used herein refers to the environment in which a tumor exists, which is the non-cellular area within the tumor and the area directly outside the tumorous tissue but does not pertain to the intracellular compartment of the cancer cell itself. The tumor and the tumor microenvironment are closely related and interact constantly. A tumor can change its microenvironment, and the microenvironment can affect how a tumor grows and spreads. Typically, the tumor microenvironment has a low pH in the range of 5.0 to 6.8, or in the range of 5.8 to 6.8, or in the range of 6.2-6.8. On the other hand, a normal physiological pH is in the range of 7.0-7.6. The tumor microenvironment is also known to have lower concentration of glucose and other nutrients, but higher concentration of lactic acid, in comparison with blood plasma. Furthermore, the tumor microenvironment can have a temperature that is 0.3 to 1° C. higher than the normal physiological temperature. The tumor microenvironment has been discussed in Gillies et al., “MRI of the Tumor Microenvironment,” Journal of Magnetic Resonance Imaging, vol. 16, pp. 430-450, 2002, hereby incorporated by reference herein its entirety. The term “non-tumor microenvironment” refers to a microenvironment at a site other than a tumor.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “naked antibody” as used herein refers to an antibody that is not conjugated to a heterologous moiety (e.g., a cytotoxic moiety) or radiolabel. The naked antibody may be present in a pharmaceutical formulation.

The term “package insert” as used herein is used to refer to instructions customarily included in commercial packages of therapeutic products, that contain information about the indications, usage, dosage, administration, combination therapy, contraindications and/or warnings concerning the use of such therapeutic products.

The term “percent (%) amino acid sequence identity” with respect to a reference polypeptide sequence as used herein is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

$$100 times the fraction X / Y$$

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “pharmaceutical formulation” as used herein refers to a preparation which is in such form as to permit the biological activity of an active ingredient contained therein to be effective, and which contains no additional components which are unacceptably toxic to a subject to which the formulation would be administered.

The term “pharmaceutically acceptable carrier” as used herein refers to an ingredient in a pharmaceutical formulation, other than an active ingredient, which is nontoxic to a subject. A pharmaceutically acceptable carrier includes, but is not limited to, a buffer, excipient, stabilizer, or preservative.

The terms “purified” and “isolated” used herein refer to an antibody according to the disclosure or to a nucleotide sequence, that the indicated molecule is present in the substantial absence of other biological macromolecules of the same type. The term “purified” as used herein preferably means at least 75% by weight, more preferably at least 85% by weight, more preferably still at least 95% by weight, and most preferably at least 98% by weight, of biological macromolecules of the same type are present. An “isolated” nucleic acid molecule which encodes a particular polypeptide refers to a nucleic acid molecule which is substantially free of other nucleic acid molecules that do not encode the polypeptide; however, the molecule may include some additional bases or moieties which do not deleteriously affect the basic characteristics of the composition.

The term “recombinant antibody” as used herein refers to an antibody (e.g. a chimeric, humanized, or human antibody or antigen-binding fragment thereof) that is expressed by a recombinant host cell comprising nucleic acid encoding the antibody. Examples of “host cells” for producing recombinant antibodies include: (1) mammalian cells, for example, Chinese Hamster Ovary (CHO), COS, myeloma cells (including Y0 and NS0 cells), baby hamster kidney (BHK), Hela and Vero cells; (2) insect cells, for example, sf9, sf21 and Tn5; (3) plant cells, for example plants belonging to the genus Nicotiana (e.g. Nicotiana tabacum); (4) yeast cells, for example, those belonging to the genus Saccharomyces (e.g. Saccharomyces cerevisiae) or the genus Aspergillus (e.g. Aspergillus niger); (5) bacterial cells, for example Escherichia coli cells or Bacillus subtilis cells, etc.

The term “single chain Fv” (“scFv”) as used herein is a covalently linked V_{H}::V_{L} heterodimer which is usually expressed from a gene fusion including V_{H} and V_{L} encoding genes linked by a peptide-encoding linker. “dsFv” is a V_{H}:V_{L} heterodimer stabilized by a disulfide bond. Divalent and multivalent antibody fragments, such as divalent sc(Fv)₂, can either form spontaneously by non-covalent association of monovalent scFvs, or can be generated by covalent coupling of monovalent scFvs with a peptide linker.

The term “therapeutically effective amount” of the antibody of the disclosure is meant a sufficient amount of the antibody to treat said cancer, at a reasonable benefit/risk ratio applicable to any medical treatment. It will be understood, however, that the total daily usage of the antibodies and compositions of the present disclosure will be decided by the attending physician within the scope of sound medical judgment. The specific therapeutically effective dose level for any particular patient will depend upon a variety of factors including the disorder being treated and the severity of the disorder; activity of the specific antibody employed; the specific composition employed, the age, body weight, general health, sex and diet of the patient; the time of administration, route of administration, and rate of excretion of the specific antibody employed; the duration of the treatment; drugs used in combination or coincidental with the specific antibody employed; and like factors well known in the medical arts. For example, it is well known within the skill of the art to start doses of the compound at levels lower than those required to achieve the desired therapeutic effect and to gradually increase the dosage until the desired effect is achieved.

The term “treatment,” “treat,” or “treating” as used herein refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the disclosure are used to delay development of a disease or to slow the progression of a disease.

The term “tumor” as used herein refers to all neoplastic cell growth and proliferation, whether malignant or benign, and all pre-cancerous and cancerous cells and tissues. The terms “cancer,” “cancerous,” “cell proliferative disorder,” “proliferative disorder” and “tumor” are not mutually exclusive as referred to herein.

The term “variable region” or “variable domain” as used herein refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three complementarity determining regions (CDRs). (See, e.g., Kindt et al. Kuby Immunology, 6th ed., W. H. Freeman and Co., page 91 (2007).) A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol., vol. 150, pp. 880-887, 1993; Clarkson et al., Nature, vol. 352, pp. 624-628, 1991.

The term “vector” as used herein refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

### DETAILED DESCRIPTION

For illustrative purposes, the principles of the present disclosure are described by referencing various exemplary embodiments. Although certain embodiments of the disclosure are specifically described herein, one of ordinary skill in the art will readily recognize that the same principles are equally applicable to, and can be employed in, other systems and methods. Before explaining the disclosed embodiments of the present disclosure in detail, it is to be understood that the disclosure is not limited in its application to the details of any particular embodiment shown. Additionally, the terminology used herein is for the purpose of description and not for limitation. Furthermore, although certain methods are described with reference to steps that are presented herein in a certain order, in many instances, these steps can be performed in any order as may be appreciated by one skilled in the art; the novel method is therefore not limited to the particular arrangement of steps disclosed herein.

It must be noted that as used herein and in the appended claims, the singular forms “a”, “an”, and “the” include plural references unless the context clearly dictates otherwise. Furthermore, the terms “a” (or “an”), “one or more”, and “at least one” can be used interchangeably herein. The terms “comprising”, “including”, “having” and “constructed from” can also be used interchangeably.

Unless otherwise indicated, all numbers expressing quantities of ingredients, properties such as molecular weight, percent, ratio, reaction conditions, and so forth used in the specification and claims are to be understood as being modified in all instances by the term “about,” whether or not the term “about” is present. Accordingly, unless indicated to the contrary, the numerical parameters set forth in the specification and claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

It is to be understood that each component, compound, substituent, or parameter disclosed herein is to be interpreted as being disclosed for use alone or in combination with one or more of each and every other component, compound, substituent, or parameter disclosed herein.

It is also to be understood that each amount/value or range of amounts/values for each component, compound, substituent, or parameter disclosed herein is to be interpreted as also being disclosed in combination with each amount/value or range of amounts/values disclosed for any other component(s), compounds(s), substituent(s), or parameter(s) disclosed herein and that any combination of amounts/values or ranges of amounts/values for two or more component(s), compounds(s), substituent(s), or parameters disclosed herein are thus also disclosed in combination with each other for the purposes of this description.

It is further understood that each lower limit of each range disclosed herein is to be interpreted as disclosed in combination with each upper limit of each range disclosed herein for the same component, compounds, substituent, or parameter. Thus, a disclosure of two ranges is to be interpreted as a disclosure of four ranges derived by combining each lower limit of each range with each upper limit of each range. A disclosure of three ranges is to be interpreted as a disclosure of nine ranges derived by combining each lower limit of each range with each upper limit of each range, etc. Furthermore, specific amounts/values of a component, compound, substituent, or parameter disclosed in the description or an example is to be interpreted as a disclosure of either a lower or an upper limit of a range and thus can be combined with any other lower or upper limit of a range or specific amount/value for the same component, compound, substituent, or parameter disclosed elsewhere in the application to form a range for that component, compound, substituent, or parameter.

### A. Linker

The present disclosure provides a linker (L) having a formula represented by

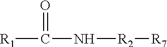

where R₁ is a nucleophilic functional group capable of coupling to an antibody selected from the group consisting of a C₁-C₁₀ alkyl halide, a C₁-C₁₀ alkyl alcohol, a phenol, a C₁-C₁₀ alkyl thiol, C₁-C₁₀ alkyl carboxylic acid, and a C₁-C₁₀ alkyl azide,
R₂ is a substituted or unsubstituted C₁-C₂₀ alkylene, a substituted or unsubstituted C₈-C₁₀ alkylene-phenyl-alkylene, or a substituted or unsubstituted (PEG)n moiety represented by

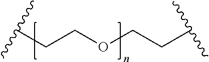

where n is 1 to 20, and
R₇ is an azide group or an acetylene group.

An embodiment of the above linker, in which R₁ can be C₁-C₆ alkyl halide with a halide selected from the group consisting of Br, I, Cl, and F, a C₁-C₂ alkyl alcohol, a 4-phenol, or a C₁-C₂ alkyl thiol.

In the embodiment of the above linker, R₁ can be —CH2-Br.

In any one of the embodiments of the above linker, R₂ can be an unsubstituted C₁-C₂₀ alkylene.

In the above embodiment of the linker, R₂ can be an unsubstituteda C₂-C₅ alkylene.

In any one of the embodiments of the above linker, R₂ can be an unsubstituted (PEG)ₙ where n is 8.

In any one of the embodiments of the above linker, R₇ can be an azide group.

In any one of the above embodiments, the linker can have a structure represented by

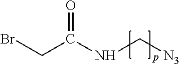

where p can be 1 to 20, preferably p can be 2 to 5.

In the above embodiment, the linker can have a structure represented by

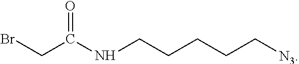 [SMILES: [N-]=[N+]=NCCCCCNC(=O)CBr]

In another of the above embodiment, the linker can have a structure represented by

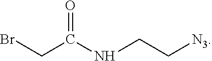

In another of the above embodiments, the linker can have a structure represented by

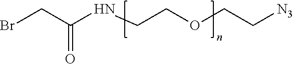

where n is 8.

The linker may comprise a substituted or unsubstituted C₁-C₂₀ alkyl, or a substituted or unsubstituted (PEG)n moiety represented by

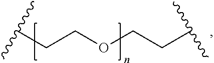

where n is 1 to 20.

The “linker” of the present disclosure may be a bifunctional or multifunctional component or moiety that can be used to link one or more drug (D) moieties (payload) to a polypeptide such as an antibody (Ab) to form a drug conjugate, an immunoconjugate, or an antibody-drug conjugate (ADC). Linkers of the present disclosure play a significant role in the stability of drug conjugates and ADCs and provide a targeted payload release (release of the drug moiety or drug moieties) profile important for the efficacy of ADC drugs. The linker of the present disclosure is stable in the circulatory system, and reduces systemic off-target toxicity by releasing cytotoxic payloads specifically to a target, such as tumor.

It is desirable to generate a linker of the present disclosure capable of covalently coupling one or more drug moieties to an antibody, or an antibody fragment thereof.

Specifically, the linker of the present disclosure is capable of covalently coupling to an antibody (Ab) or antibody fragment thereof to form ADCs without the defect of releasing payloads nonspecifically in order to optimize the therapeutic windows of ADCs. The linker of the present disclosure may be covalently coupled directly or indirectly to drug moeity as described herein.

As described above, the linker of the present disclosure comprises an antibody coupling group or a nucleophilic functional group capable of coupling to an antibody at one end and a reactive group at the other end that is complementary to an acetylene or azide group of a tether group (“X”) as described herein. Preferably, the reactive group is an azide or acetylene at R₇ that may react with the acetylene or azide group attached to R₃ of the tether group by acetylene-azide cycloaddition to provide a conjugate useful for conjugating to an antibody or antibody fragment of the present disclosure. This cycloaddition reaction between an acetylene moiety and an azide moiety is an example of a “click reaction” or “click chemistry,” the technique of which has been described by Kolb H. C. and K. B. Sharpless, Drug Discov. Today, 8:1128-37 (2003), the disclosure of which is incorporated herein in its entirety. The click reaction or click chemistry is chemoselective and may complement other conjugation chemistries such as thiol-maleimide reaction.

The antibody coupling group or nucleophilic functional group capable of coupling to an antibody may include, but is not limited to, a C₁-C₁₀ alkyl halide, a C₁-C₁₀ alkyl alcohol, a phenol, a C₁-C₁₀ alkyl thiol, C₁-C₁₀ alkyl carboxylic acid, and a C₁-C₁₀ alkyl azide. Preferably, the antibody coupling group or nucleophilic functional group capable of coupling to an antibody is a C₁-C₆ alkyl halide, in which the halide is selected from the group consisting of Br, I, Cl, and F, a C₁-C₂ alkyl alcohol, a 4-phenol, or a C₁-C₂ alkyl thiol.

### B. Drug Conjugate

A tether group (“X”) as described herein may comprise an acetylene or azide group useful for conjugating with a linker of the present disclosure. In some instances, and as herein and applicable throughout the present disclosure, a substituent of a substituted alkyl, a substituted aryl, a substituted heteroaryl, a substituted cycloalkyl, or substituted heterocyclyl, may be a substituted or unsubstituted C₁-C₂₀ alkyl, a substituted or unsubstituted C₆-C₂₅ aryl, a substituted or unsubstituted C₂-C₂₅ heteroaryl, a substituted or unsubstituted C₃-C₂₅ cycloalkyl, and a substituted C₂-C₂₅ heterocyclyl, and/or a PEGₙ where n is 1-25.

As described herein and applicable throughout the present disclosure, the substituted or unsubstituted C₁-C₂₀ alkyl may be straight chain or branched. The alkyl group includes from 1 to 20 carbon atoms, such as 1 to 10 carbon atoms, 1 to 8 carbon atoms, 1 to 6 carbon atoms, 1 to 5 carbon atoms, 1 to 4 carbon atoms, 1 to 3 carbon atoms, or 1 to 2 carbon atoms. For example, the substituted or unsubstituted C₁-C₂₀ alkyl may be a substituted or unsubstituted alkylene, such as substituted or unsubstituted C₁-C₁₀ alkylene, or a substituted or unsubstituted C₁-C₈alkylene, substituted or unsubstituted C₁-C₆ alkylene, a substituted or unsubstituted C₁-C₅ alkylene, a substituted or unsubstituted C₁-C₄ alkylene, a substituted or unsubstituted C₁-C₃ alkylene, or a substituted or unsubstituted C₁-C₂ alkylene. In some instances, substituted alkyl may include a substituent such as a cleavable moiety, e.g., an enzymatically cleavable moiety like a glycoside or glycoside derivative.

As described herein and applicable throughout the present disclosure, the substituted or unsubstituted aryl may include a phenyl. In some instances, the substituted aryl is a substituted phenyl, where the substituent includes a cleavable moiety such as an enzymatically cleavable moiety like a glycoside or glycoside derivative.

Likewise, in some instances, the substituent on the substituted heteroaryl, substituted cycloalkyl, or substituted heterocyclyl may include a cleavable moiety such as an enzymatically cleavable moiety like a glycoside or glycoside derivative.

Preferably, the tether group (“X”) is represented by

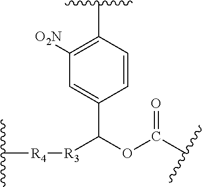

where R₃ can be a C₁-C₂₀ alkylene; R₄ can be a triazole ring having a C═C double bond in the ring. The triazole ring of R₄ may be formed in a Click reaction between an azide group and an acetylene group.

The present disclosure provides a conjugate of Formula I,

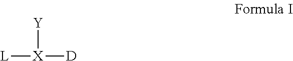

wherein:
L is represented by:

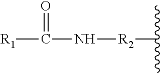

where R₁ can be a nucleophilic functional group capable of coupling to an antibody selected from the group consisting of a C₁-C₁₀ alkyl halide, a C₁-C₁₀ alkyl alcohol, a phenol, a C₁-C₁₀ alkyl thiol, and a C₁-C₁₀ alkyl azide,
R₂ can be a substituted or unsubstituted C₁-C₂₀ alkylene, a substituted or unsubstituted C₈-C₁₀ alkylene-phenyl-alkylene, or a substituted or unsubstituted (PEG)ₙ moiety represented by

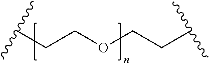

where n can be 1 to 20;
X is a tether group. Preferably, X is represented by

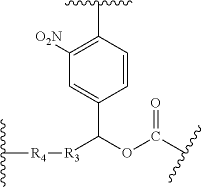

where R₃ can be a C₁-C₂₀ alkylene; R₄ can be a triazole ring having a C═C double bond in the ring;
Y can be a glycoside or glycoside derivative;
D can be a drug; and
the wavy lines indicate a site of covalent attachment.

In an embodiment of the above conjugate, R₁ may be a C₁-C₆ alkyl halide with a halide selected from the group consisting of Br, I, Cl, and F, a C₁-C₂ alkyl alcohol, a 4-phenol, or a C₁-C₂ alkyl thiol.

In any one of the embodiments of the above conjugate, R₁ may be —CH₂—Br.

In any one of the embodiments of the above conjugate, R₂ may be an unsubstituted C₁-C₂₀ alkylene.

In a specific embodiment of the above conjugate, R₂ can be an unsubstituted C₂-C₅ alkylene.

Alternately, in any one of the embodiments of the above conjugate, R₂ can be an unsubstituted (PEG)ₙ where n is 8.

In any one of the embodiments of the above conjugate, R₃ can be —CH₂—.

In any one of the embodiments of the above conjugate, the glycoside Y can be selected from the following structures:

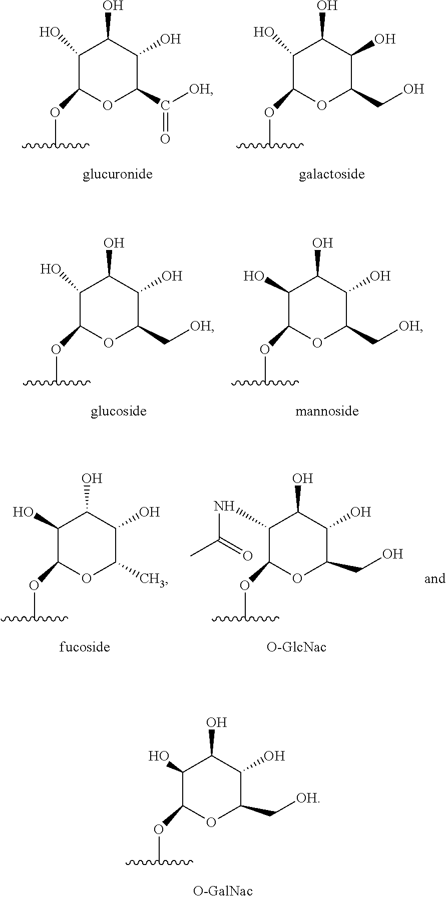

In any one of the embodiments of the above conjugate, the glycoside or glycoside derivative Y can be a glucuronide.

In any one of the embodiments of the above conjugate, the triazole ring of R₄ can be formed with an azide group bonded to L which can be represented by

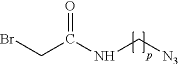

where p is 2 to 5.

Alternately, in any one of the embodiments of the above conjugate, the triazole ring can be formed with an azide group bonded to L which can be represented by

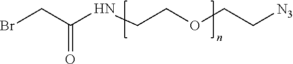

where n is 8.

In any one of the embodiments of the above conjugate, when R₂ is a substituted or unsubstituted (PEG)ₙ, Y is not a galactoside.

In any one of the embodiments of the above conjugate, the drug D can be selected from the group consisting of an auristatin, a dolastatin, a maytansinoid, a calicheamicin, a pyrrolobenzodiazepine, an anthracycline, a ribonuclease, and a DNA endonuclease.

In any one of the embodiments of the above conjugate, the drug D can be an auristatin selected from the group consisting of monomethyl auristatin E (MMAE) and monomethyl auristatin F (MMAF).

Examples of conjugates of the present disclosure include, but are not limited to the structures below.

In any one of the embodiments of the above conjugate, the conjugate may have a structure represented by SPC07

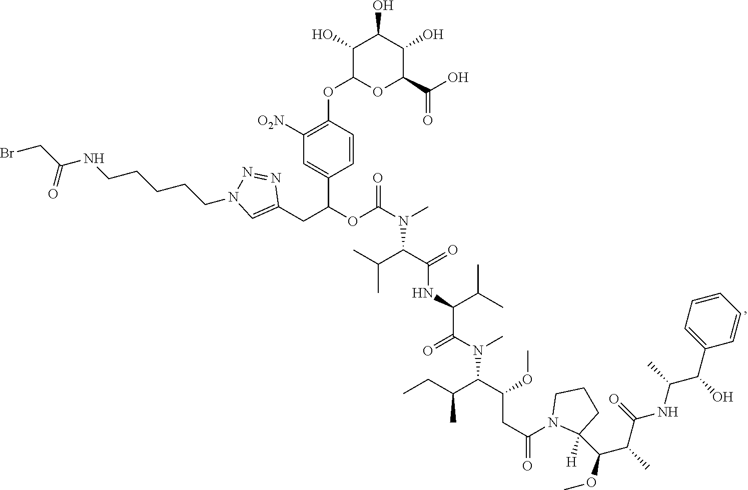 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(Cc1cn(CCCCCNC(=O)CBr)nn1)c1ccc(OC2O[C@H](C(=O)O)[C@@H](O)[C@H](O)[C@H]2O)c([N+](=O)[O-])c1)C(C)C]

or
the conjugate may have a structure represented by SPC19

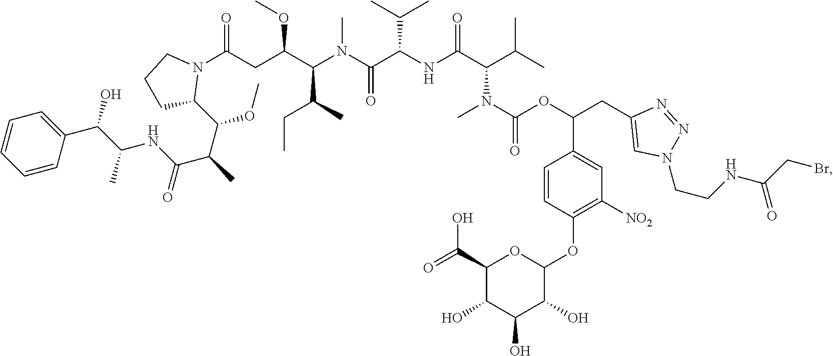 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(Cc1cn(CCNC(=O)CBr)nn1)c1ccc(OC2O[C@H](C(=O)O)[C@@H](O)[C@H](O)[C@H]2O)c([N+](=O)[O-])c1)C(C)C]

or
the conjugate may have a structure represented by SPC17

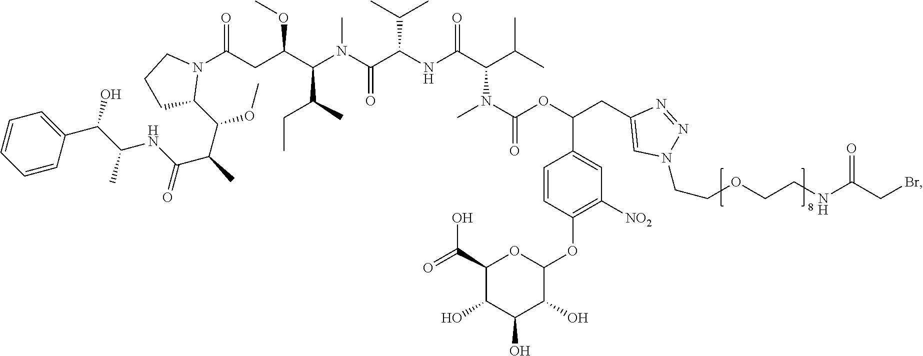

or
the conjugate may have a structure represented by SPC02

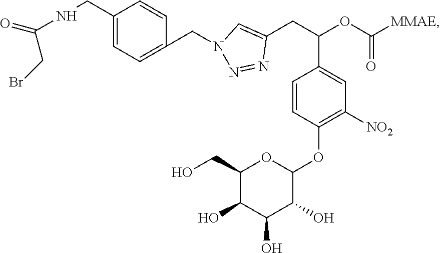

or
the conjugate may have a structure represented by SPC04

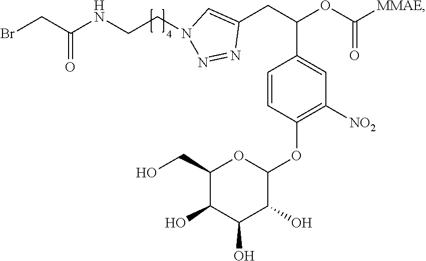

or
the conjugate may have a structure represented by SPC05

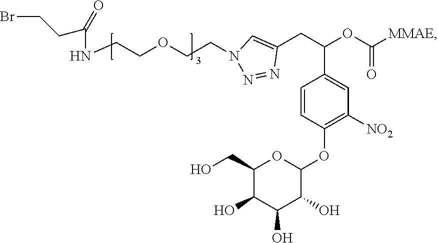

or
the conjugate may have a structure represented by SPC06

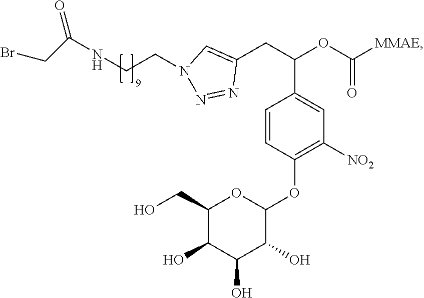

or
the conjugate may have a structure represented by SPC08

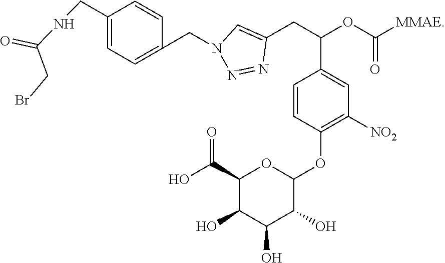

### D. Methods of Preparing Linker and Drug Conjugate

The present disclosure also provides methods of making a linker and drug conjugate of the present disclosure, as exemplified in EXAMPLES 1-6 described below.

In preparation of the linker or drug conjugate of the present disclosure, a compound represented by Formula III is also provided:

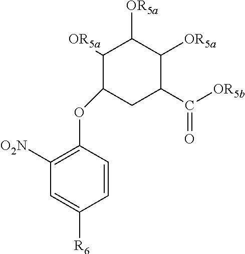

wherein R₅ₐ and R_{5b} are independently or at the same time, a hydrogen, a substituted or unsubstituted C₁-C₆ acyl, or a substituted or unsubstituted C₁-C₆ alkyl; and
R6 is a group selected from the group consisting of:

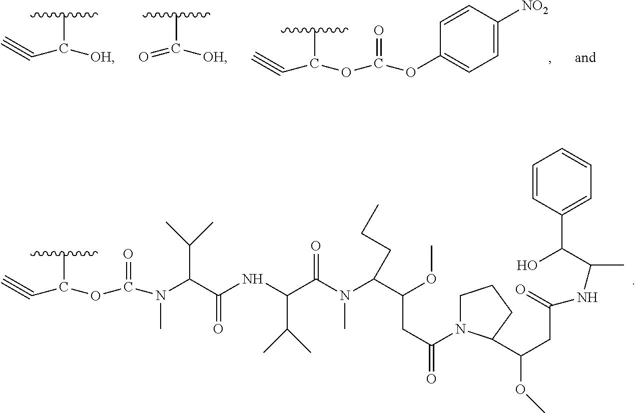

In the above embodiment, R₅ₐ is an acetyl group, and R_{5b} is hydrogen when R₆ is:

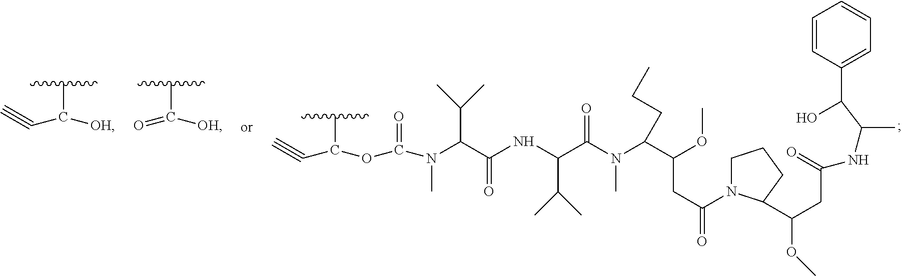

R₅ₐ is an acetyl group and R_{5b} is a methyl group when R₆ is

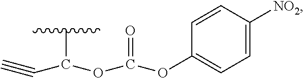 [SMILES: *C(C#C)OC(=O)Oc1ccc([N+](=O)[O-])cc1]

or
R₅ₐ and R_{5b} are each hydrogen when R₆ is

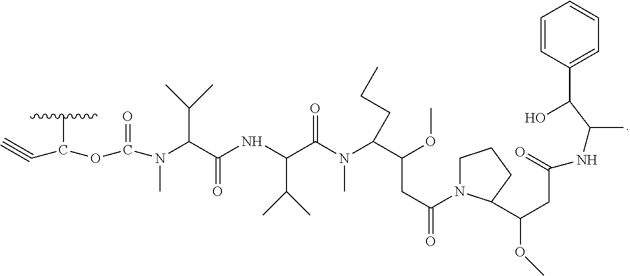 [SMILES: *C(C#C)OC(=O)N(C)C(C(=O)NC(C(=O)N(C)C(CCC)C(CC(=O)N1CCCC1C(CC(=O)NC(C)C(O)c1ccccc1)OC)OC)C(C)C)C(C)C]

### E. Immunoconjugate

The present disclosure provides an immunoconjugate of Formula II,

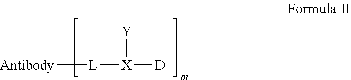

wherein:
L can be represented by:

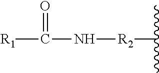

where R₁ can be a nucleophilic functional group capable of coupling to an antibody selected from the group consisting of a C₁-C₁₀ alkyl halide, a C₁-C₁₀ alkyl alcohol, a phenol, a C₁-C₁₀ alkyl thiol, and a C₁-C₁₀ alkyl azide,
R₂ can be a substituted or unsubstituted C₁-C₂₀ alkylene, a substituted or unsubstituted C₈-C₁₀ alkylene-phenyl-alkylene, or a substituted or unsubstituted (PEG)ₙ moiety represented by

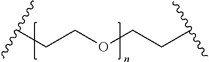

where n can be 1 to 20;
X can be a substituted nitrophenyl represented by

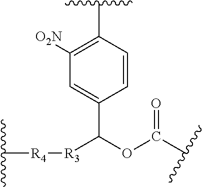

where R₃ can be a C₁-C₂₀ alkylene; R₄ can be a triazole ring having a C═C double bond in the ring;
Y can be a glycoside or glycoside derivative;
D can be a drug;
the wavy lines indicate a site of covalent attachment; and
m can be 1 to 10.

In any one of the embodiments for an immunoconjugate described herein, the antibody may include an antibody as described in Sections G and H below.

In an embodiment of the above immunoconjugate, R₁ may be a C₁-C₆ alkyl halide with a halide selected from the group consisting of Br, I, Cl, and F, a C₁-C₂ alkyl alcohol, a 4-phenol, or a C₁-C₂ alkyl thiol.

In any one of the embodiments of the above immunoconjugate, R₁ may be —CH2-Br.

In any one of the embodiments of the above immunoconjugate, R₂ may be an unsubstituted C₁-C₂₀ alkylene.

In a specific embodiment of the above immunoconjugate, R₂ may be an unsubstituted C₂-C₅ alkylene.

Alternately, in any one of the embodiments of the above immunoconjugate, R₂ may be an unsubstituted (PEG)n where n is 8.

In any one of the embodiments of the above immunoconjugate, R₃ may be —CH₂—.

In any one of the embodiments of the above immunoconjugate, the glycoside or glycoside derivative Y may be selected from the following structures:

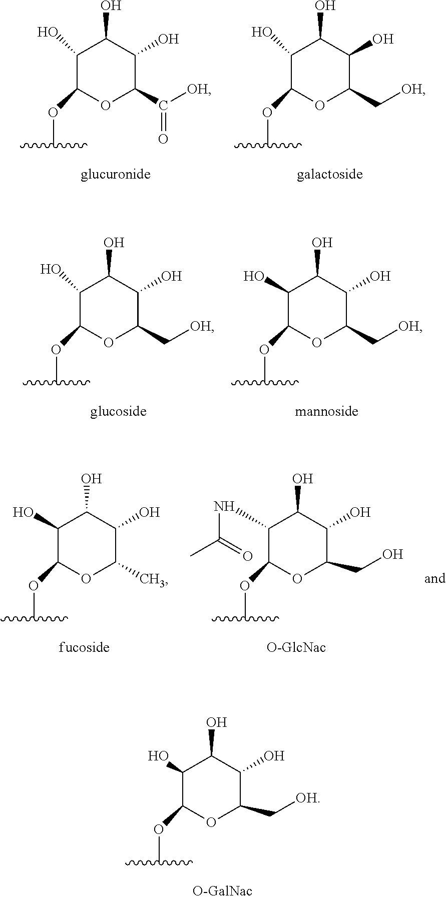

In any one of the embodiments of the above immunoconjugate, the glycoside or glycoside derivative Y may be a glucuronide.

In any one of the embodiments of the above immunoconjugate, the triazole ring at R₄ can be formed with an azide group or an acetylene group bonded to L which is represented by

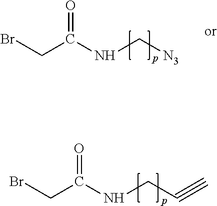

where p is 2 to 5.

Alternately, in any one of the embodiments of the above immunoconjugate, the triazole ring at R₄ can be formed with an azide group or an acetylene group bonded to L which is represented by

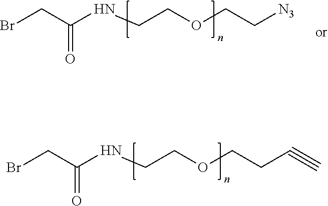

where n is 8.

In any one of the embodiments of the above immunoconjugate, when R₂ is a substituted or unsubstituted (PEG)ₙ, Y is not a galactoside.

In any one of the embodiments of the above immunoconjugate, the drug D can be selected from the group consisting of an auristatin, a dolastatin, a maytansinoid, a calicheamicin, a pyrrolobenzodiazepine, an anthracycline, a ribonuclease, and a DNA endonuclease.

In any one of the embodiments of the above immunoconjugate, the drug D can be an auristatin selected from the group consisting of monomethyl auristatin E (MMAE) and monomethyl auristatin F (MMAF).

In any one of the embodiments of the above immunoconjugate, the conjugate may have the structure of SPC07, represented by:

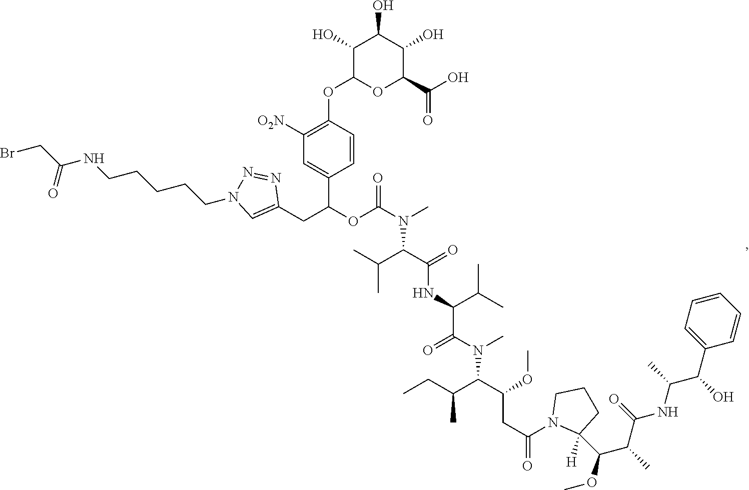 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(Cc1cn(CCCCCNC(=O)CBr)nn1)c1ccc(OC2O[C@H](C(=O)O)[C@@H](O)[C@H](O)[C@H]2O)c([N+](=O)[O-])c1)C(C)C]

or
the conjugate may have the structure of SPC19, represented by:

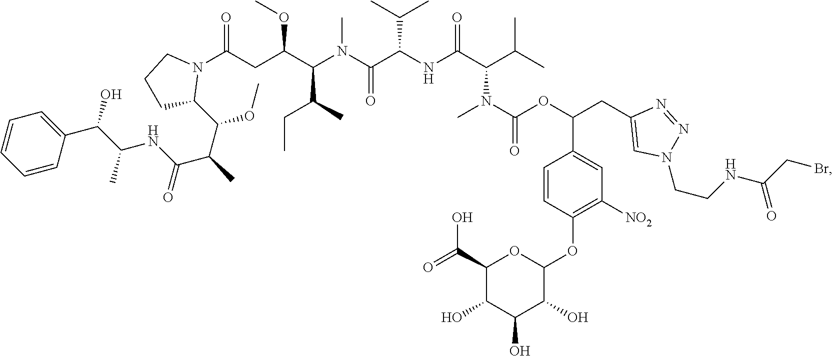 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(Cc1cn(CCNC(=O)CBr)nn1)c1ccc(OC2O[C@H](C(=O)O)[C@@H](O)[C@H](O)[C@H]2O)c([N+](=O)[O-])c1)C(C)C]

or
the conjugate may have the structure of SPC17, represented by:

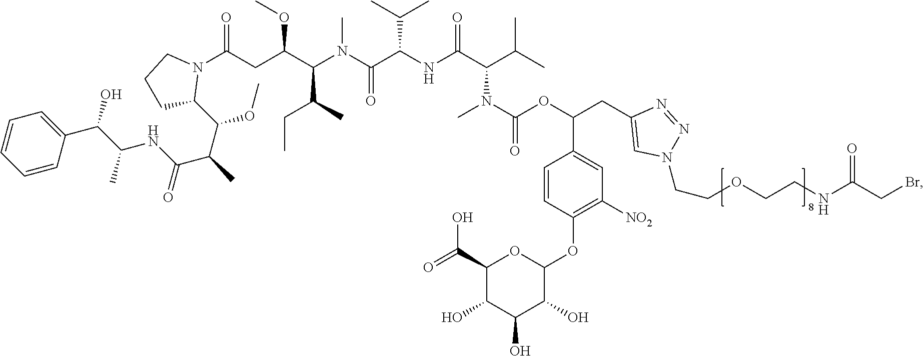

or
the conjugate may have the structure of SPC02, represented by:

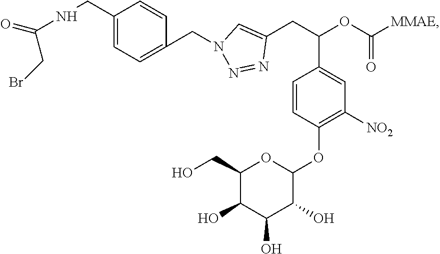

or
the conjugate may have the structure of SPC04, represented by:

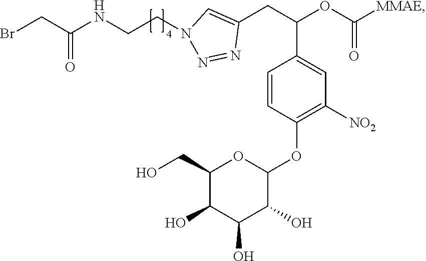

or
the conjugate may have the structure of SPC05, represented by:

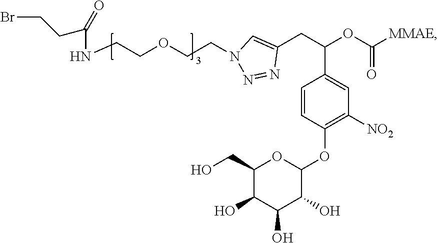

or
the conjugate may have the structure of SPC06, represented by:

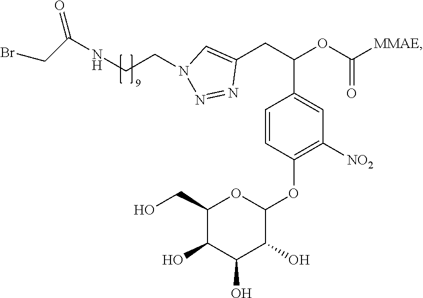

or
the conjugate may have the structure of SPC08, represented by:

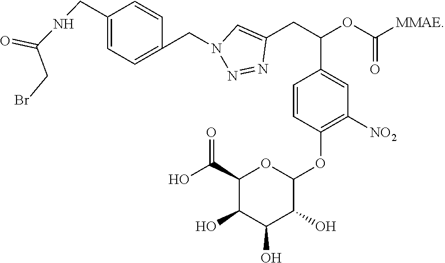

### F. Isolated Anti-Nectin-4 Polypeptides

In one aspect, the present disclosure provides an isolated polypeptide (which in some embodiments described below may be an antibody or antibody fragment) comprising a heavy chain variable region that specifically binds to Nectin-4, or especially human Nectin-4 protein. The heavy chain variable region includes three complementarity determining regions (CDRs) having sequences H1, H2, and H3, wherein:
the H1 sequence is GFTFSSYNX₁N (SEQ ID NO: 1);
the H2 sequence is YISSSSSTIYYADSVKG (SEQ ID NO: 2); and
the H3 sequence is AYYYGX₂DX₃ (SEQ ID NO: 3);
wherein X₁ is M or D; X₂ is M or D; X₃ is V or K, with the proviso that the heavy and light chain variable regions cannot be SEQ ID NOS: 18 and 31 in combination.

The H1 sequence may be selected from GFTFSSYNMN (SEQ ID NO: 7), and GFTFSSYNDN (SEQ ID NO: 8). The H3 sequence may be selected from AYYYGMDV (SEQ ID NO: 9), AYYYGDDV (SEQ ID NO: 10), and AYYYGMDK (SEQ ID NO: 11).

In another aspect, the present disclosure provides an isolated polypeptide comprising a light chain variable region that specifically binds to human Nectin-4. The light chain variable region includes three complementarity determining regions having sequences L1, L2, and L3, wherein:
the L1 sequence is X₄ASQGISGWX₅A (SEQ ID NO: 4);
the L2 sequence is AASTLOS (SEQ ID NO: 5); and
the L3 sequence is QQANSX₆PX₇T (SEQ ID NO: 6),
wherein X₄ is R or H; X₅ is L or E; X₆ is F or E; and X₇ is P or D, with the proviso that the heavy and light chain variable regions cannot be SEQ ID NOS: 18 and 31 in combination.

The L1 sequence may be selected from RASQGISGWLA (SEQ ID NO: 12), RASQGISGWEA (SEQ ID NO: 13), and HASQGISGWLA (SEQ ID NO: 14). The L3 sequence may be selected from QQANSFPPT (SEQ ID NO: 15), QQANSEPPT (SEQ ID NO: 16), and QQANSFPDT (SEQ ID NO: 17).

In another aspect, the present disclosure provides isolated polypeptides that specifically bind to Nectin-4, or especially human Nectin-4 protein comprising a heavy chain variable region and a light chain variable region, wherein the heavy chain variable region includes three complementarity determining regions having sequences H1, H2, and H3, wherein:
the H1 sequence is GFTFSSYNX₁N (SEQ ID NO: 1);
the H2 sequence is YISSSSSTIYYADSVKG (SEQ ID NO: 2); and
the H3 sequence is AYYYGX₂DX₃ (SEQ ID NO: 3);
   wherein X₁ is M or D; X₂ is M or D; X₃ is V or K; and
   the light chain variable region includes three complementarity determining regions having sequences L1, L2, and L3, wherein:
the L1 sequence is X₄ASQGISGWX₅A (SEQ ID NO: 4);
the L2 sequence is AASTLQS (SEQ ID NO: 5); and
the L3 sequence is QQANSX₆PX₇T (SEQ ID NO: 6),
wherein X₄ is R or H; X₅ is L or E; X₆ is F or E; and X₇ is P or D; and with the proviso that X₁, X₂, X₃, X₄, X₅, X₆ and X₇ cannot simultaneously be, M, M, V, R, L, F and P,
   respectively, with the proviso that the heavy and light chain variable regions cannot be SEQ ID NOS: 18 and 31 in combination.

Exemplary Anti-Nectin-4 Isolated Polypeptides

| H1, H2, H3 CDRs | L1, L2, L3 CDRs |
|---|---|
| SEQ ID NOs: 7 + 2 + 9 | SEQ ID NOs: 12 + 5 + 15 |
| SEQ ID NOs: 7 + 2 + 9 | SEQ ID NOs: 12 + 5 + 16 |
| SEQ ID NOs: 7 + 2 + 9 | SEQ ID NOs: 12 + 5 + 17 |
| SEQ ID NOs: 7 + 2 + 9 | SEQ ID NOs: 13 + 5 + 15 |
| SEQ ID NOs: 7 + 2 + 9 | SEQ ID NOs: 13 + 5 + 16 |
| SEQ ID NOs: 7 + 2 + 9 | SEQ ID NOs: 13 + 5 + 17 |
| SEQ ID NOs: 7 + 2 + 9 | SEQ ID NOs: 14 + 5 + 15 |
| SEQ ID NOs: 7 + 2 + 9 | SEQ ID NOs: 14 + 5 + 16 |
| SEQ ID NOs: 7 + 2 + 9 | SEQ ID NOs: 14 + 5 + 17 |
| SEQ ID NOs: 7 + 2 + 10 | SEQ ID NOs: 12 + 5 + 15 |
| SEQ ID NOs: 7 + 2 + 10 | SEQ ID NOs: 12 + 5 + 16 |
| SEQ ID NOs: 7 + 2 + 10 | SEQ ID NOs: 12 + 5 + 17 |
| SEQ ID NOs: 7 + 2 + 10 | SEQ ID NOs: 13 + 5 + 15 |
| SEQ ID NOs: 7 + 2 + 10 | SEQ ID NOs: 13 + 5 + 16 |
| SEQ ID NOs: 7 + 2 + 10 | SEQ ID NOs: 13 + 5 + 17 |
| SEQ ID NOs: 7 + 2 + 10 | SEQ ID NOs: 14 + 5 + 15 |
| SEQ ID NOs: 7 + 2 + 10 | SEQ ID NOs: 14 + 5 + 16 |
| SEQ ID NOs: 7 + 2 + 10 | SEQ ID NOs: 14 + 5 + 17 |
| SEQ ID NOs: 7 + 2 + 11 | SEQ ID NOs: 12 + 5 + 15 |
| SEQ ID NOs: 7 + 2 + 11 | SEQ ID NOs: 12 + 5 + 16 |
| SEQ ID NOs: 7 + 2 + 11 | SEQ ID NOs: 12 + 5 + 17 |
| SEQ ID NOs: 7 + 2 + 11 | SEQ ID NOs: 13 + 5 + 15 |
| SEQ ID NOs: 7 + 2 + 11 | SEQ ID NOs: 13 + 5 + 16 |
| SEQ ID NOs: 7 + 2 + 11 | SEQ ID NOs: 13 + 5 + 17 |
| SEQ ID NOs: 7 + 2 + 11 | SEQ ID NOs: 14 + 5 + 15 |
| SEQ ID NOs: 7 + 2 + 11 | SEQ ID NOs: 14 + 5 + 16 |
| SEQ ID NOs: 7 + 2 + 11 | SEQ ID NOs: 14 + 5 + 17 |
| SEQ ID NOs: 8 + 2 + 9 | SEQ ID NOs: 12 + 5 + 15 |
| SEQ ID NOs: 8 + 2 + 9 | SEQ ID NOs: 12 + 5 + 16 |
| SEQ ID NOs: 8 + 2 + 9 | SEQ ID NOs: 12 + 5 + 17 |
| SEQ ID NOs: 8 + 2 + 9 | SEQ ID NOs: 13 + 5 + 15 |
| SEQ ID NOs: 8 + 2 + 9 | SEQ ID NOs: 13 + 5 + 16 |
| SEQ ID NOs: 8 + 2 + 9 | SEQ ID NOs: 13 + 5 + 17 |
| SEQ ID NOs: 8 + 2 + 9 | SEQ ID NOs: 14 + 5 + 15 |
| SEQ ID NOs: 8 + 2 + 9 | SEQ ID NOs: 14 + 5 + 16 |
| SEQ ID NOs: 8 + 2 + 9 | SEQ ID NOs: 14 + 5 + 17 |
| SEQ ID NOs: 8 + 2 + 10 | SEQ ID NOs: 12 + 5 + 15 |
| SEQ ID NOs: 8 + 2 + 10 | SEQ ID NOs: 12 + 5 + 16 |
| SEQ ID NOs: 8 + 2 + 10 | SEQ ID NOs: 12 + 5 + 17 |
| SEQ ID NOs: 8 + 2 + 10 | SEQ ID NOs: 13 + 5 + 15 |
| SEQ ID NOs: 8 + 2 + 10 | SEQ ID NOs: 13 + 5 + 16 |
| SEQ ID NOs: 8 + 2 + 10 | SEQ ID NOs: 13 + 5 + 17 |
| SEQ ID NOs: 8 + 2 + 10 | SEQ ID NOs: 14 + 5 + 15 |
| SEQ ID NOs: 8 + 2 + 10 | SEQ ID NOs: 14 + 5 + 16 |
| SEQ ID NOs: 8 + 2 + 10 | SEQ ID NOs: 14 + 5 + 17 |
| SEQ ID NOs: 8 + 2 + 11 | SEQ ID NOs: 12 + 5 + 15 |
| SEQ ID NOs: 8 + 2 + 11 | SEQ ID NOs: 12 + 5 + 16 |
| SEQ ID NOs: 8 + 2 + 11 | SEQ ID NOs: 12 + 5 + 17 |
| SEQ ID NOs: 8 + 2 + 11 | SEQ ID NOs: 13 + 5 + 15 |
| SEQ ID NOs: 8 + 2 + 11 | SEQ ID NOs: 13 + 5 + 16 |
| SEQ ID NOs: 8 + 2 + 11 | SEQ ID NOs: 13 + 5 + 17 |
| SEQ ID NOs: 8 + 2 + 11 | SEQ ID NOs: 14 + 5 + 15 |
| SEQ ID NOs: 8 + 2 + 11 | SEQ ID NOs: 14 + 5 + 16 |
| SEQ ID NOs: 8 + 2 + 11 | SEQ ID NOs: 14 + 5 + 17 |

The isolated polypeptide may include a heavy chain variable region having a sequence selected from SEQ ID NOS: 18-30, with the proviso that the heavy and light chain variable regions cannot be SEQ ID NOS: 18 and 31 in combination.

The isolated polypeptide may include a light chain variable region having a sequence selected from SEQ ID NOS: 31-43, with the proviso that the heavy and light chain variable regions cannot be SEQ ID NOS: 18 and 31 in combination.

Exemplary Anti-Nectin-4 Isolated Polypeptides

| | | |
|---|---|---|
| Heavy chain region | + | Light chain region |

| | |
|---|---|
| SEQ ID NO: 18 | SEQ ID NO: 32 |
| SEQ ID NO: 18 | SEQ ID NO: 33 |
| SEQ ID NO: 18 | SEQ ID NO: 34 |
| SEQ ID NO: 18 | SEQ ID NO: 35 |
| SEQ ID NO: 18 | SEQ ID NO: 36 |
| SEQ ID NO: 18 | SEQ ID NO: 37 |
| SEQ ID NO: 18 | SEQ ID NO: 38 |
| SEQ ID NO: 18 | SEQ ID NO: 39 |
| SEQ ID NO: 18 | SEQ ID NO: 40 |
| SEQ ID NO: 18 | SEQ ID NO: 41 |
| SEQ ID NO: 18 | SEQ ID NO: 42 |
| SEQ ID NO: 18 | SEQ ID NO: 43 |
| SEQ ID NO: 19 | SEQ ID NO: 31 |
| SEQ ID NO: 19 | SEQ ID NO: 32 |
| SEQ ID NO: 19 | SEQ ID NO: 33 |
| SEQ ID NO: 19 | SEQ ID NO: 34 |
| SEQ ID NO: 19 | SEQ ID NO: 35 |
| SEQ ID NO: 19 | SEQ ID NO: 36 |
| SEQ ID NO: 19 | SEQ ID NO: 37 |
| SEQ ID NO: 19 | SEQ ID NO: 38 |
| SEQ ID NO: 19 | SEQ ID NO: 39 |
| SEQ ID NO: 19 | SEQ ID NO: 40 |
| SEQ ID NO: 19 | SEQ ID NO: 41 |
| SEQ ID NO: 19 | SEQ ID NO: 42 |
| SEQ ID NO: 19 | SEQ ID NO: 43 |
| SEQ ID NO: 20 | SEQ ID NO: 31 |
| SEQ ID NO: 20 | SEQ ID NO: 32 |
| SEQ ID NO: 20 | SEQ ID NO: 33 |
| SEQ ID NO: 20 | SEQ ID NO: 34 |
| SEQ ID NO: 20 | SEQ ID NO: 35 |
| SEQ ID NO: 20 | SEQ ID NO: 36 |
| SEQ ID NO: 20 | SEQ ID NO: 37 |
| SEQ ID NO: 20 | SEQ ID NO: 38 |
| SEQ ID NO: 20 | SEQ ID NO: 39 |
| SEQ ID NO: 20 | SEQ ID NO: 40 |
| SEQ ID NO: 20 | SEQ ID NO: 41 |
| SEQ ID NO: 20 | SEQ ID NO: 42 |
| SEQ ID NO: 20 | SEQ ID NO: 43 |
| SEQ ID NO: 21 | SEQ ID NO: 31 |
| SEQ ID NO: 21 | SEQ ID NO: 32 |
| SEQ ID NO: 21 | SEQ ID NO: 33 |
| SEQ ID NO: 21 | SEQ ID NO: 34 |
| SEQ ID NO: 21 | SEQ ID NO: 35 |
| SEQ ID NO: 21 | SEQ ID NO: 36 |
| SEQ ID NO: 21 | SEQ ID NO: 37 |
| SEQ ID NO: 21 | SEQ ID NO: 38 |
| SEQ ID NO: 21 | SEQ ID NO: 39 |
| SEQ ID NO: 21 | SEQ ID NO: 40 |
| SEQ ID NO: 21 | SEQ ID NO: 41 |
| SEQ ID NO: 21 | SEQ ID NO: 42 |
| SEQ ID NO: 21 | SEQ ID NO: 43 |
| SEQ ID NO: 22 | SEQ ID NO: 31 |
| SEQ ID NO: 22 | SEQ ID NO: 32 |
| SEQ ID NO: 22 | SEQ ID NO: 33 |
| SEQ ID NO: 22 | SEQ ID NO: 34 |
| SEQ ID NO: 22 | SEQ ID NO: 35 |
| SEQ ID NO: 22 | SEQ ID NO: 36 |
| SEQ ID NO: 22 | SEQ ID NO: 37 |
| SEQ ID NO: 22 | SEQ ID NO: 38 |
| SEQ ID NO: 22 | SEQ ID NO: 39 |
| SEQ ID NO: 22 | SEQ ID NO: 40 |
| SEQ ID NO: 22 | SEQ ID NO: 41 |
| SEQ ID NO: 22 | SEQ ID NO: 42 |
| SEQ ID NO: 22 | SEQ ID NO: 43 |
| SEQ ID NO: 23 | SEQ ID NO: 31 |
| SEQ ID NO: 23 | SEQ ID NO: 32 |
| SEQ ID NO: 23 | SEQ ID NO: 33 |
| SEQ ID NO: 23 | SEQ ID NO: 34 |
| SEQ ID NO: 23 | SEQ ID NO: 35 |
| SEQ ID NO: 23 | SEQ ID NO: 36 |
| SEQ ID NO: 23 | SEQ ID NO: 37 |
| SEQ ID NO: 23 | SEQ ID NO: 38 |
| SEQ ID NO: 23 | SEQ ID NO: 39 |
| SEQ ID NO: 23 | SEQ ID NO: 40 |
| SEQ ID NO: 23 | SEQ ID NO: 41 |
| SEQ ID NO: 23 | SEQ ID NO: 42 |
| SEQ ID NO: 23 | SEQ ID NO: 43 |
| SEQ ID NO: 24 | SEQ ID NO: 31 |
| SEQ ID NO: 24 | SEQ ID NO: 32 |
| SEQ ID NO: 24 | SEQ ID NO: 33 |
| SEQ ID NO: 24 | SEQ ID NO: 34 |
| SEQ ID NO: 24 | SEQ ID NO: 35 |
| SEQ ID NO: 24 | SEQ ID NO: 36 |
| SEQ ID NO: 24 | SEQ ID NO: 37 |
| SEQ ID NO: 24 | SEQ ID NO: 38 |
| SEQ ID NO: 24 | SEQ ID NO: 39 |
| SEQ ID NO: 24 | SEQ ID NO: 40 |
| SEQ ID NO: 24 | SEQ ID NO: 41 |
| SEQ ID NO: 24 | SEQ ID NO: 42 |
| SEQ ID NO: 24 | SEQ ID NO: 43 |
| SEQ ID NO: 25 | SEQ ID NO: 31 |
| SEQ ID NO: 25 | SEQ ID NO: 32 |
| SEQ ID NO: 25 | SEQ ID NO: 33 |
| SEQ ID NO: 25 | SEQ ID NO: 34 |
| SEQ ID NO: 25 | SEQ ID NO: 35 |
| SEQ ID NO: 25 | SEQ ID NO: 36 |
| SEQ ID NO: 25 | SEQ ID NO: 37 |
| SEQ ID NO: 25 | SEQ ID NO: 38 |
| SEQ ID NO: 25 | SEQ ID NO: 39 |
| SEQ ID NO: 25 | SEQ ID NO: 40 |
| SEQ ID NO: 25 | SEQ ID NO: 41 |
| SEQ ID NO: 25 | SEQ ID NO: 42 |
| SEQ ID NO: 25 | SEQ ID NO: 43 |
| SEQ ID NO: 26 | SEQ ID NO: 31 |
| SEQ ID NO: 26 | SEQ ID NO: 32 |
| SEQ ID NO: 26 | SEQ ID NO: 33 |
| SEQ ID NO: 26 | SEQ ID NO: 34 |
| SEQ ID NO: 26 | SEQ ID NO: 35 |
| SEQ ID NO: 26 | SEQ ID NO: 36 |
| SEQ ID NO: 26 | SEQ ID NO: 37 |
| SEQ ID NO: 26 | SEQ ID NO: 38 |
| SEQ ID NO: 26 | SEQ ID NO: 39 |
| SEQ ID NO: 26 | SEQ ID NO: 40 |
| SEQ ID NO: 26 | SEQ ID NO: 41 |
| SEQ ID NO: 26 | SEQ ID NO: 42 |
| SEQ ID NO: 26 | SEQ ID NO: 43 |
| SEQ ID NO: 27 | SEQ ID NO: 31 |
| SEQ ID NO: 27 | SEQ ID NO: 32 |
| SEQ ID NO: 27 | SEQ ID NO: 33 |
| SEQ ID NO: 27 | SEQ ID NO: 34 |
| SEQ ID NO: 27 | SEQ ID NO: 35 |
| SEQ ID NO: 27 | SEQ ID NO: 36 |
| SEQ ID NO: 27 | SEQ ID NO: 37 |
| SEQ ID NO: 27 | SEQ ID NO: 38 |
| SEQ ID NO: 27 | SEQ ID NO: 39 |
| SEQ ID NO: 27 | SEQ ID NO: 40 |
| SEQ ID NO: 27 | SEQ ID NO: 41 |
| SEQ ID NO: 27 | SEQ ID NO: 42 |
| SEQ ID NO: 27 | SEQ ID NO: 43 |
| SEQ ID NO: 28 | SEQ ID NO: 31 |
| SEQ ID NO: 28 | SEQ ID NO: 32 |
| SEQ ID NO: 28 | SEQ ID NO: 33 |
| SEQ ID NO: 28 | SEQ ID NO: 34 |
| SEQ ID NO: 28 | SEQ ID NO: 35 |
| SEQ ID NO: 28 | SEQ ID NO: 36 |
| SEQ ID NO: 28 | SEQ ID NO: 37 |
| SEQ ID NO: 28 | SEQ ID NO: 38 |
| SEQ ID NO: 28 | SEQ ID NO: 39 |
| SEQ ID NO: 28 | SEQ ID NO: 40 |
| SEQ ID NO: 28 | SEQ ID NO: 41 |
| SEQ ID NO: 28 | SEQ ID NO: 42 |
| SEQ ID NO: 28 | SEQ ID NO: 43 |
| SEQ ID NO: 29 | SEQ ID NO: 31 |
| SEQ ID NO: 29 | SEQ ID NO: 32 |
| SEQ ID NO: 29 | SEQ ID NO: 33 |
| SEQ ID NO: 29 | SEQ ID NO: 34 |
| SEQ ID NO: 29 | SEQ ID NO: 35 |
| SEQ ID NO: 29 | SEQ ID NO: 36 |
| SEQ ID NO: 29 | SEQ ID NO: 37 |
| SEQ ID NO: 29 | SEQ ID NO: 38 |
| SEQ ID NO: 29 | SEQ ID NO: 39 |
| SEQ ID NO: 29 | SEQ ID NO: 40 |
| SEQ ID NO: 29 | SEQ ID NO: 41 |
| SEQ ID NO: 29 | SEQ ID NO: 42 |
| SEQ ID NO: 29 | SEQ ID NO: 43 |
| SEQ ID NO: 30 | SEQ ID NO: 31 |
| SEQ ID NO: 30 | SEQ ID NO: 32 |
| SEQ ID NO: 30 | SEQ ID NO: 33 |
| SEQ ID NO: 30 | SEQ ID NO: 34 |
| SEQ ID NO: 30 | SEQ ID NO: 35 |
| SEQ ID NO: 30 | SEQ ID NO: 36 |
| SEQ ID NO: 30 | SEQ ID NO: 37 |
| SEQ ID NO: 30 | SEQ ID NO: 38 |
| SEQ ID NO: 30 | SEQ ID NO: 39 |
| SEQ ID NO: 30 | SEQ ID NO: 40 |
| SEQ ID NO: 30 | SEQ ID NO: 41 |
| SEQ ID NO: 30 | SEQ ID NO: 42 |
| SEQ ID NO: 30 | SEQ ID NO: 43 |

In a preferred embodiment, the isolated polypeptides of the present disclosure comprise a heavy chain variable region and a light chain variable region having any one pair of sequences selected from: SEQ ID NOS: 19 and 32, SEQ ID NOS: 20 and 33, SEQ ID NOS: 21 and 34, SEQ ID NOS: 22 and 35, SEQ ID NOS: 23 and 36, SEQ ID NOS: 24 and 37, SEQ ID NOS: 25 and 38, SEQ ID NOS: 26 and 39, SEQ ID NOS: 27 and 40, SEQ ID NOS: 28 and 41, and SEQ ID NOS: 29 and 42.

In another aspect, the isolated polypeptides of the present disclosure comprise a heavy chain variable region and a light chain variable region each region independently having at least 80%, 85%, 90%, 95%, 98% or 99% identity to a combination of amino acid sequences selected from one of SEQ ID NOS: 18-30 in combination with one of SEQ ID NOS: 31-43; with the proviso that the heavy and light chain variable regions cannot be SEQ ID NOS: 18 and 31 in combination and said isolated polypeptides specifically bind to human Nectin-4 protein.

In yet another aspect, the isolated polypeptides of the present disclosure comprise a heavy chain variable region and a light chain variable region each region independently having at least 80%, 85%, 90%, 95%, 98% or 99% identity to a pair of amino acid sequences selected from:: SEQ ID NOS: 19 and 32, SEQ ID NOS: 20 and 33, SEQ ID NOS: 21 and 34, SEQ ID NOS: 22 and 35, SEQ ID NOS: 23 and 36, SEQ ID NOS: 24 and 37, SEQ ID NOS: 25 and 38, SEQ ID NOS: 26 and 39, SEQ ID NOS: 27 and 40, SEQ ID NOS: 28 and 41, with the proviso that the heavy and light chain variable regions cannot be SEQ ID NOS: 18 and 31 in combination and SEQ ID NOS: 29 and 42, respectively; and said isolated polypeptides specifically bind to human Nectin-4 protein.

In a yet another aspect, the isolated polypeptide of the present disclosure specifically binds to Nectin-4, or especially human Nectin-4 protein and to CD3, and comprises a heavy chain variable region and a light chain variable region, wherein the heavy chain variable region includes three complementarity determining regions having sequences H1, H2, and H3, wherein:
the H1 sequence is selected from SEQ ID NO: 7 and SEQ ID NO: 8,
the H2 sequence is SEQ ID NO: 2, and
the H3 sequence is selected from SEQ ID NO: 9, SEQ ID NO: 10, and SEQ ID NO: 11; and
the light chain variable region includes three complementarity determining regions having sequences L1, L2, and L3, wherein:
the L1 sequence is X₄ASQGISGWX₅A (SEQ ID NO: 4);
the L2 sequence is AASTLOS (SEQ ID NO: 5); and
the L3 sequence is QQANSX₆PX₇T (SEQ ID NO: 6),
wherein X₄ is R or H; X₅ is L or E; X₆ is F or E; and X₇ is P or D; and with the proviso that X₁, X₂, X₃, X₄, X₅, X₆ and X₇ cannot simultaneously be, M, M, V, R, L, F and P, respectively, and
six anti-CD3 complementarity determining regions L4, L5, L6, L7, L8, and L9 wherein:
the L4 sequence is GFTFNTYAMN (SEQ ID NO: 44),
the L5 sequence is RIRSKYNNYATYYADSVKD (SEQ ID NO: 45),
the L6 sequence is HX₁₁NFX₁₂NSX₁₃ VSWFX₁₄Y (SEQ ID NO: 46),
the L7 sequence is RSSTGAVTTSNYX₁₅N (SEQ ID NO: 47),
the L8 sequence is GTNKRAP (SEQ ID NO: 48), and
the L9 sequence is ALWYSNLWV (SEQ ID NO: 49),
wherein X₁₁ is G or S, X₁₂ is G or P, X₁₃ is Y or K, X₁₄ is A or Q and X₁₅ is A or D.

In another aspect of the isolated polypeptide with nine CDRs, the L6 sequence is selected from any one of SEQ ID NOs: 50-53, and the L7 sequence is selected from SEQ ID NOs: 54 and 55.

In a preferred aspect, the isolated polypeptide specifically binds to Nectin-4 and CD3 and comprises a heavy chain variable region and a light chain variable region, wherein the heavy chain variable region includes three complementarity determining regions, H1, H2, and H3, wherein:
the H1 sequence is selected from SEQ ID NO: 7 and SEQ ID NO: 8,
the H2 sequence is selected from SEQ ID NO: 2, and
the H3 sequence is selected from SEQ ID NO: 9, SEQ ID NO: 10, and SEQ ID NO: 11, and
the light chain variable region includes three complementarity determining regions having sequences L1, L2, and L3, wherein:
the L1 sequence is selected from SEQ ID NO: 12, SEQ ID NO: 13, and SEQ ID NO: 14,
the L2 sequence is SEQ ID NO: 5,
the L3 sequence is selected from SEQ ID NO: 15, SEQ ID NO: 16, and SEQ ID NO: 17, and six anti-CD3 complementarity determining regions L4, L5, L6, L7, L8, and L9 wherein:
the L4 sequence is GFTFNTYAMN (SEQ ID NO: 44),
the L5 sequence is RIRSKYNNYATYYADSVKD (SEQ ID NO: 45),
the L6 sequence is selected from HGNFGNSYVSWFAY (SEQ ID NO: 50), HSNFGNSKVSWFAY (SEQ ID NO: 51), HGNFPNSKVSWFQY (SEQ ID NO: 52), and HSNFGNSKVSWFAY (SEQ ID NO: 53),
the L7 sequence is selected from RSSTGAVTTSNYAN (SEQ ID NO: 54) and RSSTGAVTTSNYDN (SEQ ID NO: 55),
the L8 sequence is GTNKRAP (SEQ ID NO: 48), and
the L9 sequence is ALWYSNLWV (SEQ ID NO: 49).

In another preferred aspect, the isolated polypeptide specifically binds to Nectin-4 and CD3 and comprises a heavy chain variable region and a light chain variable region that, wherein the heavy chain variable region includes three complementarity determining regions, H1, H2, and H3, wherein:
the H1 sequence is selected from SEQ ID NO: 7,
the H2 sequence is selected from SEQ ID NO: 2, and
the H3 sequence is selected from SEQ ID NO: 9, SEQ ID NO: 10, and SEQ ID NO: 11, and
the light chain variable region includes three complementarity determining regions L1, L2, and L3, wherein
the L1 sequence is selected from SEQ ID NO: 12, SEQ ID NO: 13, and SEQ ID NO: 14,
the L2 sequence is SEQ ID NO: 5,
the L3 sequence is SEQ ID NO: 15, and six anti-CD3 complementarity determining regions L4, L5, L6, L7, L8, and L9 wherein:
the L4 sequence is GFTFNTYAMN (SEQ ID NO: 44),
the L5 sequence is RIRSKYNNYATYYADSVKD (SEQ ID NO: 45),
the L6 sequence is selected from HGNFGNSYVSWFAY (SEQ ID NO: 50), HSNFGNSKVSWFAY (SEQ ID NO: 51), HGNFPNSKVSWFQY (SEQ ID NO: 52), and HSNFGNSKVSWFAY (SEQ ID NO: 53),
the L7 sequence is selected from RSSTGAVTTSNYAN (SEQ ID NO: 54) and RSSTGAVTTSNYDN (SEQ ID NO: 55),
the L8 sequence is GTNKRAP (SEQ ID NO: 48), and
the L9 sequence is ALWYSNLWV (SEQ ID NO: 49).

Exemplary Anti-Nectin-4 Isolated Polypeptides With Nine CDRs

Each of the “Exemplary Anti-Nectin-4 Isolated Polypeptides” listed above having H1, H2, H3, L1, L2 and L3 sequences, may further include any one of the combinations of L4, L5, L6, L7, L8 and L9 set forth below.

| | L4+ | L5+ | L6+ | L7+ | L8+ | L9 CDRs |
|---|---|---|---|---|---|---|
| SEQ ID NOS: | 44+ | 45+ | 50+ | 54+ | 48+ | 49 |
| SEQ ID NOS: | 44+ | 45+ | 50+ | 55+ | 48+ | 49 |
| SEQ ID NOS: | 44+ | 45+ | 51+ | 54+ | 48+ | 49 |
| SEQ ID NOS: | 44+ | 45+ | 51+ | 55+ | 48+ | 49 |
| SEQ ID NOS: | 44+ | 45+ | 52+ | 54+ | 48+ | 49 |
| SEQ ID NOS: | 44+ | 45+ | 52+ | 55+ | 48+ | 49 |
| SEQ ID NOS: | 44+ | 45+ | 53+ | 54+ | 48+ | 49 |
| SEQ ID NOS: | 44+ | 45+ | 53+ | 55+ | 48+ | 49 |

In each of the previous aspects, the isolated polypeptide with nine CDRs comprises a heavy chain variable region having a sequence selected from SEQ ID NOS: 18, 25, 27, and 29.

In each of the previous aspects, the isolated polypeptide with nine CDRs comprises a light chain variable region having a sequence selected from SEQ ID NOS: 56-60.

In certain aspects, the isolated polypeptide with nine CDRs comprises a heavy chain variable sequence of any one of SEQ ID NOs: 18, 25, 27, and 29, and a light chain variable sequence of any one of SEQ ID NOs: 56-60.

Exemplary Anti-Nectin-4 Isolated Polypeptides

| | | |
|---|---|---|
| Heavy chain region | + | Light chain region |

| | |
|---|---|
| SEQ ID NO: 18 | SEQ ID NO: 57 |
| SEQ ID NO: 18 | SEQ ID NO: 58 |
| SEQ ID NO: 18 | SEQ ID NO: 59 |
| SEQ ID NO: 18 | SEQ ID NO: 60 |
| SEQ ID NO: 25 | SEQ ID NO: 56 |
| SEQ ID NO: 25 | SEQ ID NO: 57 |
| SEQ ID NO: 25 | SEQ ID NO: 58 |
| SEQ ID NO: 25 | SEQ ID NO: 59 |
| SEQ ID NO: 25 | SEQ ID NO: 60 |
| SEQ ID NO: 27 | SEQ ID NO: 56 |
| SEQ ID NO: 27 | SEQ ID NO: 57 |
| SEQ ID NO: 27 | SEQ ID NO: 58 |
| SEQ ID NO: 27 | SEQ ID NO: 59 |
| SEQ ID NO: 27 | SEQ ID NO: 60 |
| SEQ ID NO: 29 | SEQ ID NO: 56 |
| SEQ ID NO: 29 | SEQ ID NO: 57 |
| SEQ ID NO: 29 | SEQ ID NO: 58 |
| SEQ ID NO: 29 | SEQ ID NO: 59 |
| SEQ ID NO: 29 | SEQ ID NO: 60 |

In certain preferred aspects, isolated polypeptides with nine CDRs of the present disclosure comprise a heavy chain variable region and a light chain variable region having any one pair of sequences selected from: SEQ ID NO: 25 and SEQ ID NO: 57, SEQ ID NO: 27 and SEQ ID NO: 58, SEQ ID NO: 29 and SEQ ID NO: 59, and SEQ ID NO: 29 and SEQ ID NO: 60.

In another aspect, the isolated polypeptides with nine CDRs of the present disclosure comprise a heavy chain variable region and a light chain variable region each region independently having at least 80%, 85%, 90%, 95%, 98% or 99% identity to a combination of amino acid sequences selected from one of SEQ ID NOS: 18, 25, 27, and 29 in combination with one of SEQ ID NOs: 56-60; with the proviso that the heavy and light chain variable regions cannot be SEQ ID NOS: 18 and 56 combination; and said isolated polypeptides specifically bind to human Nectin-4 protein.

In another aspect of the present disclosure, the isolated polypeptides with nine CDRs comprise a heavy chain variable region and a light chain variable region each region independently having at least 80%, 85%, 90%, 95%, 98% or 99% identity to a pair of amino acid sequences selected from: SEQ ID NOS: 25 and 57, SEQ ID NOS: 27 and 58, SEQ ID NOS: 29 and 59, SEQ ID NOS: 29 and 60; and said isolated polypeptides specifically bind to human Nectin-4 protein.

In each of the previous aspects, the isolated polypeptide of the present disclosure which specifically binds to Nectin-4, or especially human Nectin-4 protein and to CD3, may also comprise the sequences described hereinabove for specific binding to Nectin-4, and a single-chain fragment variable (scFv) of any known CD3 antibody. In this aspect of the disclosure, the isolated polypeptide binds CD3 independent of the conditionally active Nectin-4 binding. For example, in one embodiment, an isolated polypeptide of the present disclosure which specifically binds to Nectin-4, or especially human Nectin-4 protein and to CD3, comprises a heavy chain variable region and a light chain variable region, wherein the heavy chain variable region includes three complementarity determining regions having sequences H1, H2, and H3, wherein:
the H1 sequence is selected from SEQ ID NO: 7 and SEQ ID NO: 8,
the H2 sequence is SEQ ID NO: 2, and
the H3 sequence is selected from SEQ ID NO: 9, SEQ ID NO: 10, and SEQ ID NO: 11; and
the light chain variable region includes three complementarity determining regions having sequences L1, L2, and L3, wherein:
the L1 sequence is X₄ASQGISGWX₅A (SEQ ID NO: 4);
the L2 sequence is AASTLQS (SEQ ID NO: 5); and
the L3 sequence is QQANSX₆PX₇T (SEQ ID NO: 6),
wherein X₄ is R or H; X₅ is L or E; X₆ is F or E; and X₇ is P or D; and with the proviso that X₁, X₂, X₃, X₄, X₅, X₆ and X₇ cannot simultaneously be, M, M, V, R, L, F and P, respectively, and an scFv comprising six anti-CD3 complementarity determining regions of any known CD3 antibody.

The heavy chain variable regions and the light chain variable regions of the present disclosure were each obtained from a parent antibody using a method disclosed in U.S. Pat. Nos. 8,709,755 and 8,859,467. This method of generating the heavy chain variable regions and the light chain variable regions, as well as the method of generating antibodies and antibody fragments disclosed in U.S. Pat. Nos. 8,709,755 and 8,859,467, are hereby incorporated by reference herein.

### G. Anti-Nectin-4 Antibodies

The isolated polypeptide may be an antibody or antibody fragment. Antibodies and antibody fragments including these heavy chain variable regions and light chain variable regions can specifically bind to Nectin-4, or especially human Nectin-4. Antibodies or antibody fragments comprising a combination of one of these heavy chain variable regions and one of these light chain variable regions have been found to have higher binding to Nectin-4 at a pH in the tumor microenvironment (e.g. pH 5.0-6.8, preferably, pH 6.0-6.8) than at a pH in a non-tumor microenvironment (e.g. pH 7.0-7.6). As a result, the anti-Nectin-4 antibodies or antibody fragments have a higher binding to Nectin-4 in a tumor microenvironment in comparison with their binding to Nectin-4 in a typical normal tissue microenvironment. In one aspect, binding is measured by affinity.

In any of the embodiments of the isolated polypeptides, antibodies and antibody fragments described herein, the conditionally active isolated polypeptide, antibody or antibody fragment may be less active or virtually inactive at a normal physiological condition (such as a non-tumor microenvironment) and more active at an aberrant condition (such as a tumor microenvironment), in comparison to the activity at the normal physiological condition of the parent or wild-type polypeptide, antibody or antibody fragment from which it is derived. As a result, the isolated polypeptides, anti-Nectin-4 antibodies or anti-Nectin-4 antibody fragments of the present disclosure may have a lower binding to Nectin-4 at a normal physiological condition (such as a non-tumor microenvironment) in comparison to the parent or wild-type polypeptide, antibody or antibody fragment from which it is derived. For example, the conditionally active isolated polypeptide, anti-Nectin-4 antibody or anti-Nectin-4 antibody fragment may be less active or virtually inactive at a pH of 7.0-7.6 in comparison to the parent or wild-type polypeptide, antibody or antibody fragment, but is active at a lower pH of 5.0-6.8 in comparison to the parent or wild-type polypeptide, antibody or antibody fragment. In some cases, the conditionally active isolated polypeptide, antibody or antibody fragment is reversibly or irreversibly inactivated at the normal physiological condition (such as a non-tumor microenvironment) in comparison to the parent or wild-type polypeptide, antibody or antibody fragment.

Anti-Nectin-4 antibodies or antibody fragments of the present disclosure are thus expected to exhibit reduced side-effects, relative to non-conditionally active anti-Nectin-4 antibodies, due to their reduced binding to Nectin-4 in the normal tissue microenvironment. Anti-Nectin-4 antibodies or antibody fragments of the present disclosure are also expected to have a comparable efficacy to monoclonal anti-Nectin-4 antibodies known in the art. This combination of features permits use of a higher dosage of these anti-Nectin-4 antibodies or antibody fragments due to the reduced side effects, which may provide a more effective therapy option.

The present disclosure provides an antibody or antibody fragment that specifically binds to Nectin-4, or especially human Nectin-4 protein, comprising a heavy chain variable region that includes three complementarity determining regions (CDRs) having sequences H1, H2, and H3, wherein:
the H1 sequence is GFTFSSYNX₁N (SEQ ID NO: 1);
the H2 sequence is YISSSSSTIYYADSVKG (SEQ ID NO: 2); and
the H3 sequence is AYYYGX₂DX₃ (SEQ ID NO: 3);
wherein X₁ is M or D; X₂ is M or D; X₃ is V or K, with the proviso that the heavy and light chain variable regions cannot be SEQ ID NOS: 18 and 31 in combination.

The H1 sequence may be selected from GFTFSSYNMN (SEQ ID NO: 7), and GFTFSSYNDN (SEQ ID NO: 8). The H3 sequence may be selected from AYYYGMDV (SEQ ID NO: 9), AYYYGDDV (SEQ ID NO: 10), and AYYYGMDK (SEQ ID NO: 11).

In another aspect, the present disclosure provides an antibody or antibody fragment comprising a light chain variable region that specifically binds to human Nectin-4. The light chain variable region includes three complementarity determining regions having sequences L1, L2, and L3, wherein:
the L1 sequence is X₄ASQGISGWX₅A (SEQ ID NO: 4);
the L2 sequence is AASTLQS (SEQ ID NO: 5); and
the L3 sequence is QQANSX₆PX₇T (SEQ ID NO: 6),
wherein X₄ is R or H; X₅ is L or E; X₆ is F or E; and X₇ is P or D, with the proviso that the heavy and light chain variable regions cannot be SEQ ID NOS: 18 and 31 in combination.

The L1 sequence may be selected from RASQGISGWLA (SEQ ID NO: 12), RASQGISGWEA (SEQ ID NO: 13), and HASQGISGWLA (SEQ ID NO: 14). The L3 sequence may be selected from QQANSFPPT (SEQ ID NO: 15), QQANSEPPT (SEQ ID NO: 16), and QQANSFPDT (SEQ ID NO: 17).

In a more specific aspect, the present disclosure provides an antibody or antibody fragment comprising a heavy chain variable region and a light chain variable region, wherein the heavy chain variable region includes three complementarity determining regions having sequences H1, H2, and H3, wherein:
the H1 sequence is GFTFSSYNX₁N (SEQ ID NO: 1);
the H2 sequence is YISSSSSTIYYADSVKG (SEQ ID NO: 2); and
the H3 sequence is AYYYGX₂DX₃ (SEQ ID NO: 3);
   wherein X₁ is M or D; X₂ is M or D; X₃ is V or K; and the light chain variable region includes three complementarity determining regions, having sequences L1, L2, and L3, wherein:
the L1 sequence is X₄ASQGISGWX₅A (SEQ ID NO: 4);
the L2 sequence is AASTLOS (SEQ ID NO: 5); and
the L3 sequence is QQANSX₆PX₇T (SEQ ID NO: 6),
   wherein X₄ is R or H; X₅ is L or E; X₆ is F or E; and X₇ is P or D; and with the proviso that X₁, X₂, X₃, X₄, X₅, X₆ and X₇ cannot simultaneously be, M, M, V, R, L, F and P, respectively, with the proviso that the heavy and light chain variable regions cannot be SEQ ID NOS: 18 and 31 in combination.

The heavy chain variable region may have a sequence selected from SEQ ID NOS: 18-30, with the proviso that the heavy and light chain variable regions cannot be SEQ ID NOS: 18 and 31 in combination.

The light chain variable region may have a sequence selected from SEQ ID NOS: 31-43, with the proviso that the heavy and light chain variable regions cannot be SEQ ID NOS: 18 and 31 in combination.

### Exemplary Anti-Nectin-4 Antibodies

In certain embodiments the anti-Nectin-4 antibodies and antibody fragments of the present disclosure include the combinations of H1, H2, H3, L1, L2, and L3 CDRs or the combinations of heavy variable chain regions (selected from SEQ ID NOS: 18-30) and light chain variable regions (selected from SEQ ID NOS: 31-43) set forth above for the isolated polypeptides. Preferred Nectin-4 antibodies and antibody fragments of the present disclosure are those that include the preferred combinations of these heavy and light chain variable regions set forth above for the isolated polypeptides. For example, preferred, antibody or antibody fragments of the present disclosure comprise a heavy chain variable region and a light chain variable region having any one pair of sequences selected from: SEQ ID NOS: 32 and 19, SEQ ID NOS: 33 and 20, SEQ ID NOS: 34 and 21, SEQ ID NOS: 35 and 22, SEQ ID NOS: 36 and 23, SEQ ID NOS: 37 and 24, SEQ ID NOS: 38 and 25, SEQ ID NOS: 39 and 26, SEQ ID NOS: 40 and 27, SEQ ID NOS: 41 and 28 and SEQ ID NOS: 42 and 29.

The antibody or antibody fragments of the present disclosure can comprise a heavy chain variable region and a light chain variable region each region independently having at least 80%, 85%, 90%, 95%, 98% or 99% identity to a combination of amino acid sequences selected from one of SEQ ID NOS: 18-30 in combination with one of SEQ ID NOS: 31-43; with the proviso that the heavy and light chain variable regions cannot be SEQ ID NOS: 18 and 31 in combination; and said antibody or antibody fragments specifically bind to human Nectin-4 protein.

The antibody or antibody fragments of the present disclosure can comprise a heavy chain variable region and a light chain variable region each region independently having at least 80%, 85%, 90%, 95%, 98% or 99% identity to a pair of amino acid sequences selected from:: SEQ ID NOS: 32 and 19, SEQ ID NOS: 33 and 20, SEQ ID NOS: 34 and 21, SEQ ID NOS: 35 and 22, SEQ ID NOS: 36 and 23, SEQ ID NOS: 37 and 24, SEQ ID NOS: 38 and 25, SEQ ID NOS: 39 and 26, SEQ ID NOS: 40 and 27, SEQ ID NOS: 41 and 28 and SEQ ID NOS: 42 and 29, respectively; with the proviso that the heavy and light chain variable regions cannot be SEQ ID NOS: 18 and 31 in combination and said antibody or antibody fragments specifically bind to human Nectin-4 protein.

In another aspect, an antibody or antibody fragment of the present disclosure is multi-specific specifically bind to Nectin-4, or especially human Nectin-4 protein and CD3 and comprises a heavy chain variable region and a light chain variable region, wherein the heavy chain variable region includes three complementarity determining regions having sequences H1, H2, and H3, wherein:
the H1 sequence is selected from SEQ ID NO: 7 and SEQ ID NO: 8,
the H2 sequence is SEQ ID NO: 2, and
the H3 sequence is selected from SEQ ID NO: 9, SEQ ID NO: 10, and SEQ ID NO: 11; and
the light chain variable region includes three complementarity determining regions having sequences L1, L2, and L3, wherein:
the L1 sequence is X₄ASQGISGWX₅A (SEQ ID NO: 4);
the L2 sequence is AASTLOS (SEQ ID NO: 5); and
the L3 sequence is QQANSX₆PX₇T (SEQ ID NO: 6),
wherein X₄ is R or H; X₅ is L or E; X₆ is F or E; and X₇ is P or D; and with the proviso that X₁, X₂, X₃, X₄, X₅, X₆ and X₇ cannot simultaneously be, M, M, V, R, L, F and P, respectively, and
six anti-CD3 complementarity determining regions L4, L5, L6, L7, L8, and L9 wherein:
the L4 sequence is GFTFNTYAMN (SEQ ID NO: 44),
the L5 sequence is RIRSKYNNYATYYADSVKD (SEQ ID NO: 45),
the L6 sequence is HX₁₁NFX₁₂NSX₁₃VSWFX₁₄Y (SEQ ID NO: 46),
the L7 sequence is RSSTGAVTTSNYX₁₅N (SEQ ID NO: 47),
the L8 sequence is GTNKRAP (SEQ ID NO: 48), and
the L9 sequence is ALWYSNLWV (SEQ ID NO: 49),
wherein X₁₁ is G or S, X₁₂ is G or P, X₁₃ is Y or K, X₁₄ is A or Q and X₁₅ is A or D.

In another aspect of the multispecific antibody or antibody fragment of the present disclosure, the L6 sequence is any one of SEQ ID NOs: 50-53, and the L7 sequence is selected from SEQ ID NO: 54 and 55.

Exemplary Bi-Specific anti-Nectin-4×CD3 Antibodies

In certain embodiments the bi-specific anti-Nectin-4×CD3 antibodies and antibody fragments of the present disclosure include the combinations of H1, H2, H3, L1, L2, L3, L4, L5, L6, L7, L8 and L9 CDRs or the combinations of a heavy variable region (selected from SEQ ID NOS: 18, 25, 27, and 29) and a light chain variable region (selected from SEQ ID NOS: 56-60) set forth above for the isolated polypeptides with the proviso that the heavy and light chain variable regions cannot be SEQ ID NOS: 18 and 56 combination. Preferred anti-Nectin-4 antibodies and antibody fragments of the present disclosure are those that include the preferred combinations of these heavy and light chain variable regions set forth above for the isolated polypeptides.

In a preferred aspect, a multi-specific antibody or antibody fragment specifically binds to Nectin-4, or especially human Nectin-4 protein and to CD3 and comprises a heavy chain variable region and a light chain variable region that wherein the heavy chain variable region includes three complementarity determining regions, H1, H2, and H3, wherein: the H1 sequence is selected from SEQ ID NO: 7 and SEQ ID NO: 8,
the H2 sequence is selected from SEQ ID NO: 2, and
the H3 sequence is selected from SEQ ID NO: 9, SEQ ID NO: 10, and SEQ ID NO: 11, and
the light chain variable region includes three complementarity determining regions L1, L2, and L3, wherein:
the L1 sequence is selected from SEQ ID NO: 12, SEQ ID NO: 13, and SEQ ID NO: 14,
the L2 sequence is SEQ ID NO: 5,
the L3 sequence is selected from SEQ ID NO: 15, SEQ ID NO: 16, and SEQ ID NO: 17, and
six anti-CD3 complementarity determining regions L4, L5, L6, L7, L8, and L9 wherein:
the L4 sequence is GFTFNTYAMN (SEQ ID NO: 44),
the L5 sequence is RIRSKYNNYATYYADSVKD (SEQ ID NO: 45),
the L6 sequence is selected from HGNFGNSYVSWFAY (SEQ ID NO: 50),
HSNFGNSKVSWFAY (SEQ ID NO: 51), HGNFPNSKVSWFQY (SEQ ID NO: 52), and HSNFGNSKVSWFAY (SEQ ID NO: 53),
the L7 sequence is selected from RSSTGAVTTSNYAN (SEQ ID NO: 54) and RSSTGAVTTSNYDN (SEQ ID NO: 55),
the L8 sequence is GTNKRAP (SEQ ID NO: 48), and
the L9 sequence is ALWYSNLWV (SEQ ID NO: 49), with the proviso that the heavy and light chain variable regions cannot be SEQ ID NOS: 18 and 56 combination.

In another preferred aspect, a multi-specific antibody or antibody fragment of the present disclosure specifically binds to Nectin-4, or especially human Nectin-4 protein and to CD3 and comprises a heavy chain variable region and a light chain variable region, wherein the heavy chain variable region including three complementarity determining regions, H1, H2, and H3, wherein:
the H1 sequence is selected from SEQ ID NO: 7,
the H2 sequence is selected from SEQ ID NO: 2, and
the H3 sequence is selected from SEQ ID NO: 9, SEQ ID NO: 10, and SEQ ID NO: 11, and
the light chain variable region includes three complementarity determining regions L1, L2, and L3, wherein:
the L1 sequence is selected from SEQ ID NO: 12 and SEQ ID NO: 13,
the L2 sequence is SEQ ID NO: 5,
the L3 sequence is SEQ ID NO: 15, and six anti-CD3 complementarity determining regions L4, L5, L6, L7, L8, and L9 wherein:
the L4 sequence is GFTFNTYAMN (SEQ ID NO: 44),
the L5 sequence is RIRSKYNNYATYYADSVKD (SEQ ID NO: 45),
the L6 sequence is selected from HGNFGNSYVSWFAY (SEQ ID NO: 50), HSNFGNSKVSWFAY (SEQ ID NO: 51), HGNFPNSKVSWFQY (SEQ ID NO: 52), and HSNFGNSKVSWFAY (SEQ ID NO: 53),
the L7 sequence is selected from RSSTGAVTTSNYAN (SEQ ID NO: 54) and RSSTGAVTTSNYDN (SEQ ID NO: 55),
the L8 sequence is GTNKRAP (SEQ ID NO: 48), and
the L9 sequence is ALWYSNLWV (SEQ ID NO: 49), with the proviso that the heavy and light chain variable regions cannot be SEQ ID NOS: 18 and 56 combination.

In each of the previous embodiments, a multi-specific antibody or antibody fragment of the present disclosure may comprise a heavy chain variable region having a sequence selected from SEQ ID NOS: 18, 25, 27, and 29.

In each of the previous embodiments, a multi-specific antibody or antibody fragment of the present disclosure may comprise a light chain variable region having a sequence selected from SEQ ID NOS: 56-60.

In certain embodiments, a multi-specific antibody or antibody fragment of the present disclosure comprises a heavy chain variable region having a sequence of any one of SEQ ID NOs: 18, 25, 27, and 29, and a light chain variable region having a sequence of any one of SEQ ID NOs: 56-60 with the proviso that the heavy and light chain variable regions cannot be SEQ ID NOS: 18 and 56 combination.

In certain embodiments, a multi-specific antibody or antibody fragment of the present disclosure comprises a heavy chain variable region and a light chain variable region having any one pair of sequences selected from: SEQ ID NO: 25 and SEQ ID NO: 57, SEQ ID NO: 27 and SEQ ID NO: 58, SEQ ID NO: 29 and SEQ ID NO: 59, and SEQ ID NO: 29 and SEQ ID NO: 60.

In another embodiment, a multispecific antibody or antibody fragment of the present disclosure comprises a heavy chain variable region and a light chain variable region each region independently having at least 80%, 85%, 90%, 95%, 98% or 99% identity to a combination of amino acid sequences selected from one of SEQ ID NOS: 18, 25, 27, and 29 in combination with one of SEQ ID NOs: 56-60; with the proviso that the heavy and light chain variable regions cannot be SEQ ID NOS: 18 and 56 combination; and said antibody or antibody fragment specifically binds to human Nectin-4 protein.

In another embodiment, a multispecific antibody or antibody fragment of the present disclosure comprises a heavy chain variable region and a light chain variable region each region independently having at least 80%, 85%, 90%, 95%, 98% or 99% identity to a pair of amino acid sequences selected from: SEQ ID NOS: 25 and 57, SEQ ID NOS: 27 and 58, SEQ ID NOS: 29 and 59, SEQ ID NOS: 29 and 60; with the proviso that the heavy and light chain variable regions cannot be SEQ ID NOS: 18 and 56 combination and said antibody or antibody fragment specifically binds to human Nectin-4 protein.

In other embodiments, the amino acid sequence of the heavy and light chain variable regions outside of the complementarity determining regions may be mutated in accordance with the principles of substitution, insertion and deletion, as discussed in this application to provide these variants. In still further embodiments, the constant regions may be modified to provide these variants. In still further embodiments, both the amino acid sequence of the heavy and light chain variable regions outside of the complementarity determining regions and the constant regions may be modified to provide these variants.

In deriving these variants, one is guided by the process as described herein. The variants of the heavy chain and light chain variable regions may be prepared by introducing appropriate modifications into the nucleotide sequence encoding the heavy and light chain variable regions, or by peptide synthesis. Such modifications include, for example, deletions from, and/or insertions into and/or substitutions of residues within the amino acid sequences of the heavy and light chain variable regions. Any combination of deletion, insertion, and substitution can be made to arrive at the antibodies or antibody fragments of the present disclosure, provided that they possess the desired characteristics, e.g., antigen-binding to human Nectin-4 and/or conditional activity.

### Substitution, Insertion, and Deletion Variants

In certain embodiments, antibody or antibody fragment variants having one or more amino acid substitutions are provided. Sites of interest for substitutional mutagenesis include the CDRs and framework regions (FRs). Conservative substitutions are shown in Table 1 under the heading of “conservative substitutions.” More substantial changes are provided in Table 1 under the heading of “exemplary substitutions,” and as further described below in reference to amino acid side chain classes. Amino acid substitutions may be introduced into an antibody or antibody fragment of interest and the products screened for a desired activity, e.g., retained/improved antigen binding, or decreased immunogenicity.

TABLE 1
Amino acid substitutions

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; | Leu |
| | Phe; Norleucine | |
| Leu (L) | Norleucine; Ile; Val; | Ile |
| | Met; Ala; Phe | |
| Lys (K) | Arg; Gln; Asn | Arg |
| Met (M) | Leu; Phe; Ile | Leu |
| Phe (F) | Trp; Leu; Val; Ile; | Tyr |
| | Ala; Tyr | |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; | Leu |
| | Ala; Norleucine | |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more complementarity determining region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more CDR residues are mutated and the variant antibodies displayed on phage and screened for a particular biological activity (e.g. binding affinity).

Alterations (e.g., substitutions) may be made in CDRs, e.g., to improve antibody affinity. Such alterations may be made in CDR “hotspots,” i.e., residues encoded by codons that undergo mutation at high frequency during the somatic maturation process (see, e.g., Chowdhury, Methods Mol. Biol., vol. 207, pp. 179-196, 2008), and/or SDRs (a-CDRs), with the resulting variant V_{H} or V_{L} being tested for binding affinity. Affinity maturation by constructing and reselecting from secondary libraries has been described, e.g., in Hoogenboom et al. in Methods in Molecular Biology, vol. 178, pp. 1-37, 2001). In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more CDRs so long as such alterations do not substantially reduce the ability of the antibody or antibody fragment to bind antigen. For example, conservative alterations (e.g., conservative substitutions as provided herein) that do not substantially reduce binding affinity may be made in CDRs. Such alterations may be outside of CDR “hotspots” or SDRs. In certain embodiments of the variant V_{H} and V_{L} sequences provided above, each CDR either is unaltered, or contains no more than one, two or three amino acid substitutions.

A useful method for identification of residues or regions of an antibody that may be targeted for mutagenesis is called “alanine scanning mutagenesis” as described by Cunningham and Wells, Science, vol. 244, pp. 1081-1085, 1989. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody or antibody fragment with antigen is affected. Further substitutions may be introduced at the amino acid locations demonstrating functional sensitivity to the initial substitutions. Alternatively, or additionally, a crystal structure of an antigen-antibody complex to identify contact points between the antibody or antibody fragment and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody include the fusion to the N- or C-terminus of the antibody to an enzyme (e.g. for ADEPT) or a polypeptide which increases the serum half-life of the antibody.

Amino acid sequence modification(s) of the antibodies described herein are contemplated. For example, it may be desirable to improve the binding affinity and/or other biological properties of the antibody. It is known that when a humanized antibody is produced by simply grafting only CDRs in V_{H} and V_{L} of an antibody derived from a non-human animal in FRs of the V_{H} and V_{L} of a human antibody, the antigen binding activity is reduced in comparison with that of the original antibody derived from a non-human animal. It is considered that several amino acid residues of the V_{H} and V_{L} of the non-human antibody, not only in CDRs but also in FRs, are directly or indirectly associated with the antigen binding activity. Hence, substitution of these amino acid residues with different amino acid residues derived from FRs of the V_{H} and V_{L} of the human antibody would reduce of the binding activity. In order to resolve the problem, in antibodies grafted with human CDR, attempts have to be made to identify, among amino acid sequences of the FR of the V_{H} and V_{L} of human antibodies, an amino acid residue which is directly associated with binding to the antibody, or which interacts with an amino acid residue of CDR, or which maintains the three-dimensional structure of the antibody and which is directly associated with binding to the antigen. The reduced antigen binding activity could be increased by replacing the identified amino acids with amino acid residues of the original antibody derived from a non-human animal.

Modifications and changes may be made in the structure of the antibodies of the present disclosure, and in the DNA sequences encoding them, and still obtain a functional molecule that encodes an antibody with desirable characteristics.

In making the changes in the amino sequences, the hydropathic index of amino acids may be considered. The importance of the hydropathic amino acid index in conferring interactive biologic function on a protein is generally understood in the art. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of their hydrophobicity and charge characteristics these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophane (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

A further object of the present disclosure also encompasses function-conservative variants of the antibodies of the present disclosure.

Two amino acid sequences are “substantially homologous” or “substantially similar” when greater than 80%, preferably greater than 85%, preferably greater than 90% of the amino acids are identical, or greater than about 90%, preferably greater than 95%, are similar (functionally identical) over the whole length of the shorter sequence. Preferably, the similar or homologous sequences are identified by alignment using, for example, the GCG (Genetics Computer Group, Program Manual for the GCG Package, Version 7, Madison, Wis.) pileup program, or any of sequence comparison algorithms such as BLAST, FASTA, etc.

For example, certain amino acids may be substituted by other amino acids in a protein structure without appreciable loss of activity. Since the interactive capacity and nature of a protein define the protein's biological functional activity, certain amino acid substitutions can be made in a protein sequence, and, of course, in its DNA encoding sequence, while nevertheless obtaining a protein with like properties. It is thus contemplated that various changes may be made in the sequences of the antibodies or antibody fragments of the disclosure, or corresponding DNA sequences which encode said antibodies or antibody fragments, without appreciable loss of their biological activity.

It is known in the art that certain amino acids may be substituted by other amino acids having a similar hydropathic index or score and still result in a protein with similar biological activity, i.e. still obtain a biological functionally equivalent protein.

As outlined above, amino acid substitutions are generally therefore based on the relative similarity of the amino acid side-chain substituents, for example, their hydrophobicity, hydrophilicity, charge, size, and the like. Exemplary substitutions which take various of the foregoing characteristics into consideration are well known to those of skill in the art and include: arginine and lysine; glutamate and aspartate; serine and threonine; glutamine and asparagine; and valine, leucine and isoleucine.

### Glycosylation Variants

In certain embodiments, the anti-Nectin-4 antibodies or antibody fragments provided herein are altered to increase or decrease the extent to which the antibodies or antibody fragments are glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH, vol. 15, pp. 26-32, 1997. The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the disclosure may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol., vol. 336, pp. 1239-1249, 2004; Yamane-Ohnuki et al. Biotech. Bioeng., vol. 87, pp. 614-622, 2004. Examples of cell lines capable of producing defucosylated antibodies include Lec13 CHO cells deficient in protein fucosylation (Ripka et al. Arch. Biochem. Biophys., vol. 249, pp. 533-545, 1986; US Pat Appl No US 2003/0157108 A; and WO 2004/056312 A1, especially at Example 11), and knockout cell lines, such as alpha-1,6-fucosyltransferase gene, FUT8, knockout CHO cells (see, e.g., Yamane-Ohnuki et al. Biotech. Bioeng., vol. 87, pp. 614-622, 2004; Kanda, Y. et al., Biotechnol. Bioeng., vol. 94, pp. 680-688, 2006; and WO2003/085107).

Antibody variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878; U.S. Pat. No. 6,602,684; and US 2005/0123546. Antibody variants with at least one galactose residue in the oligosaccharide attached to the Fc region are also provided. Such antibody variants may have improved CDC function. Such antibody variants are described, e.g., in WO 1997/30087; WO 1998/58964; and WO 1999/22764.

### Fc Region Variants

In certain embodiments, one or more amino acid modifications may be introduced into the Fc region of the anti-Nectin-4 antibodies or antibody fragments provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In certain embodiments, the disclosure contemplates an antibody variant that possesses some but not all effector functions, which make it a desirable candidate for applications in which the half life of the antibody in vivo is important yet certain effector functions (such as ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol., vol. 9, pp. 457-492, 1991. Non-limiting examples of in vitro assays to assess ADCC activity of a molecule of interest is described in U.S. Pat. No. 5,500,362 (see also, e.g. Hellstrom et al. Proc. Nat'l Acad. Sci. USA, vol. 83, pp. 7059-7063, 1986) and Hellstrom, I et al., Proc. Nat'l Acad. Sci. USA, vol. 82, pp. 1499-1502, 1985; U.S. Pat. No. 5,821,337 (see also Bruggemann et al., J. Exp. Med., vol. 166, pp. 1351-1361, 1987). Alternatively, non-radioactive assays methods may be employed (see, for example, ACTI™ non-radioactive cytotoxicity assay for flow cytometry (CellTechnology, Inc. Mountain View, Calif.; and CytoTox 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. Proc. Nat'l Acad. Sci. USA, vol. 95, pp. 652-656, 1998. C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods, vol. 202, pp. 163-171, 1996; Cragg, M. S. et al., Blood, vol. 101, pp. 1045-1052, 2003; and Cragg, M. S, and M. J. Glennie, Blood, vol. 103, pp. 2738-2743, 2004). FcRn binding and in vivo clearance/half life determinations can also be performed using methods known in the art (see, e.g., Petkova, S. B. et al., Int'l. Immunol., vol. 18, pp. 1759-1769, 2006).

The variants of the antibodies or antibody fragments with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem., vol. 9, pp. 6591-6604, 2001).

In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol., vol. 164, pp. 4178-4184, 2000.

Antibodies with increased half-lives and improved binding to the neonatal Fc receptor (FcRn), which is responsible for the transfer of maternal IgGs to the fetus (Guyer et al., J. Immunol., vol. 117, pp. 587-593, 1976 and Kim et al., J. Immunol., vol. 24, p. 249, 1994), are described in US2005/0014934. Those antibodies comprise an Fc region with one or more substitutions therein which improve binding of the Fc region to FcRn. Such Fc variants include/e those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826). See also Duncan & Winter, Nature, vol. 322, pp. 738-740, 1988; U.S. Pat. Nos. 5,648,260; 5,624,821; and WO 94/29351 concerning other examples of Fc region variants.

### Cysteine Engineered Antibody Variants

In certain embodiments, it may be desirable to create cysteine engineered antibodies, e.g., “thioMAbs,” in which one or more residues of the anti-Nectin-4 antibodies or antibody fragments are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and 5400 (EU numbering) of the heavy chain Fc region. Cysteine engineered antibodies may be generated as described, e.g., in U.S. Pat. No. 7,521,541.

### Antibody Derivatives

In certain embodiments, the anti-Nectin-4 antibodies or antibody fragments provided herein may be further modified to contain additional nonproteinaceous moieties that are known in the art and readily available. The moieties suitable for derivatization of the antibody or antibody fragment include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone) polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight and may be branched or unbranched. The number of polymers attached to the antibody or antibody fragment may vary, and if more than one polymer is attached, they can be the same or different molecules. In general, the number and/or type of polymers used for derivatization can be determined based on considerations including, but not limited to, the particular properties or functions of the antibody or antibody fragment to be improved, whether the derivative will be used in a therapy under defined conditions, etc.

In another embodiment, conjugates of the antibodies or antibody fragments and nonproteinaceous moiety that may be selectively heated by exposure to radiation are provided. In one embodiment, the nonproteinaceous moiety is a carbon nanotube (Kam et al., Proc. Natl. Acad. Sci. USA, vol. 102, pp. 11600-11605, 2005). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

The anti-Nectin-4 antibodies or antibody fragments of the disclosure, or their variants, have a higher binding affinity to Nectin-4 under a condition in a tumor microenvironment than under a condition in a non-tumor microenvironment. In one embodiment, the condition in tumor microenvironment and the condition in a non-tumor microenvironment are both pH. The anti-Nectin-4 antibodies or antibody fragments of the disclosure thus can selectively bind to Nectin-4 at a pH about 5.0-6.8 but will have a lower binding affinity to Nectin-4 at a pH about 7.0-7.6 encountered in a normal, non-tumor microenvironment. As shown in the Examples to follow, the exemplary anti-Nectin-4 antibodies or antibody fragments of the present disclosure have higher binding affinity to Nectin-4 at pH 6.0 that at pH 7.4. In certain embodiments, the anti-Nectin-4 antibodies or antibody fragments of the present disclosure have a dissociation constant (Kd) with Nectin-4 under a condition in tumor microenvironment of about ≤1μ, ≤100 nM, ≤10 nM, ≤1 nM, ≤0.1 nM, ≤0.01 nM, or ≤0.001 nM (e.g. 10⁻⁸M or less, or from 10⁻⁸M to 10⁻¹³M, or from 10⁻⁹M to 10⁻¹³ M). In one embodiment, the ratio of the Kd of the antibody or antibody fragment with Nectin-4 at the condition in a non-tumor microenvironment to the Kd at the same condition in a tumor microenvironment is at least about 1.5:1, at least about 2:1, at least about 3:1, at least about 4:1, at least about 5:1, at least about 6:1, at least about 7:1, at least about 8:1, at least about 9:1, at least about 10:1, at least about 20:1, at least about 30:1, at least about 50:1, at least about 70:1, or at least about 100:1.

In another embodiment, the ratio of the binding activity of the antibody or antibody fragment with Nectin-4 at the condition in tumor microenvironment to the binding activity at the same condition in non-tumor microenvironment is at least about 1.5:1, at least about 2:1, at least about 3:1, at least about 4:1, at least about 5:1, at least about 6:1, at least about 7:1, at least about 8:1, at least about 9:1, at least about 10:1, at least about 20:1, at least about 30:1, at least about 50:1, at least about 70:1, or at least about 100:1.

In one embodiment, Kd is measured by a radiolabeled antigen binding assay (RIA) performed with the Fab version of an antibody of interest and its antigen using the following assay. Solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881 (1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in phosphate buffered saline (PBS) for two to five hours at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using surface plasmon resonance assays using a BIACORE®-2000 or a BIACORE®-3000 (BIAcore, Inc., Piscataway, N.J.) at 25° C. with immobilized antigen CM5 chips at about 10 response units (RU). Briefly, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (“0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (Kd) is calculated as the ratio k_{off}/kₒₙ. See, e.g., Chen et al., J. Mol. Biol. 293:865-881 (1999). If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

The anti-Nectin-4 antibodies of the disclosure may be a chimeric, humanized or human antibody. In one embodiment, an anti-Nectin-4 antibody fragment is employed, e.g., a Fv, Fab, Fab′, Fab′-SH, scFv, a diabody, a triabody, a tetrabody or an F(ab′)₂ fragment and multi-specific antibodies formed from antibody fragments. In another embodiment, the antibody is a full length antibody, e.g., an intact IgG antibody or other antibody class or isotype as defined herein. For a review of certain antibody fragments, see Hudson et al. Nat. Med., vol. 9, pp. 129-134, 2003. For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

The diabodies of the disclosure may be bivalent or bispecific. See, for example, EP 404,097; WO 1993/01161; Hudson et al., Nat. Med. 9:129-134 (2003); and Hollinger et al., Proc. Natl. Acad. Sci. USA, vol. 90, pp. 6444-6448, 1993 for examples of diabodies. Examples of triabodies and tetrabodies are also described in Hudson et al., Nat. Med., vol. 9, pp. 129-134, 2003.

In some embodiments, the disclosure comprises single-domain antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1).

Antibody fragments can be made by various techniques, including but not limited to proteolytic digestion of an intact antibody as well as production by recombinant host cells (e.g. E. coli or phage), as described herein.

In some embodiments, the anti-Nectin-4 antibodies of the disclosure may be chimeric antibodies. Certain chimeric antibodies are described, e.g., in U.S. Pat. No. 4,816,567; and Morrison et al., Proc. Natl. Acad. Sci. USA, vol. 81, pp. 6851-6855, 1984). In one example, the chimeric antibody comprises a non-human variable region (e.g., a variable region derived from a mouse, rat, hamster, rabbit, or non-human primate, such as a monkey) and a human constant region. In a further example, the chimeric antibody is a “class switched” antibody in which the class or subclass of the antibody has been changed relative to the class or subclass of the parent antibody. Chimeric antibodies include antigen-binding fragments thereof.

In certain embodiments, the chimeric antibody of the disclosure is a humanized antibody. Typically, such a non-human antibody is humanized to reduce immunogenicity to humans, while retaining the specificity and affinity of the parental non-human antibody. Generally, a humanized antibody comprises one or more variable domains in which CDRs (or portions thereof) are derived from a non-human antibody, and FRs (or portions thereof) are derived from human antibody sequences. A humanized antibody may optionally also comprise at least a portion of a human constant region. In some embodiments, some FR residues in a humanized antibody are substituted with corresponding residues from a non-human antibody (e.g., the antibody from which the CDR residues are derived), e.g., to restore or improve antibody specificity or affinity.

Humanized antibodies and methods of making them are reviewed, e.g., in Almagro and Fransson, Front. Biosci., vol. 13, pp. 1619-1633, 2008, and are further described, e.g., in Riechmann et al., Nature, vol. 332, pp. 323-329, 1988; Queen et al., Proc. Nat'l Acad. Sci. USA, vol. 86, pp. 10029-10033, 1989; U.S. Pat. Nos. 5,821,337, 7,527,791, 6,982,321, and 7,087,409; Kashmiri et al., Methods, vol. 36, pp. 25-34, 2005 (describing SDR (a-CDR) grafting); Padlan, Mol. Immunol., vol. 28, pp. 489-498, 1991 (describing “resurfacing”); Dall'Acqua et al., Methods, vol. 36, pp. 43-60, 2005 (describing “FR shuffling”); and Osbourn et al., Methods, vol. 36, pp. 61-68, 2005 and Klimka et al., Br. J. Cancer, vol. 83, pp. 252-260, 2000 (describing the “guided selection” approach to FR shuffling).

Human framework regions that may be used for humanization include but are not limited to: framework regions selected using the “best-fit” method (see, e.g., Sims et al. J. Immunol., vol. 151, p. 2296, 1993); framework regions derived from the consensus sequence of human antibodies of a particular subgroup of light or heavy chain variable regions (see, e.g., Carter et al. Proc. Natl. Acad. Sci. USA, vol. 89, p. 4285, 1992; and Presta et al. J. Immunol., vol. 151, p. 2623, 1993); human mature (somatically mutated) framework regions or human germline framework regions (see, e.g., Almagro and Fransson, Front. Biosci., vol. 13, pp. 1619-1633, 2008); and framework regions derived from screening FR libraries (see, e.g., Baca et al., J. Biol. Chem., vol. 272, pp. 10678-10684, 1997 and Rosok et al., J. Biol. Chem., vol. 271, pp. 22611-22618, 1996).

### H. Multi-Specific Antibodies and Antibody Fragments

The disclosure provides multi-specific anti-Nectin-4 antibodies, e.g. bispecific antibodies. Multi-specific antibodies are monoclonal antibodies that have binding specificities for at least two different sites. In certain embodiments, one of the binding specificities is for Nectin-4 and the other is for another antigen. In certain embodiments, bispecific conditionally active antibodies may bind to two different epitopes of Nectin-4. The multi-specific antibody binds to at least Nectin-4 and another antigen with a greater activity, affinity and/or avidity at a first physiological condition than at a second physiological condition. Bispecific antibodies may also be used to localize cytotoxic agents to cells which express Nectin-4. Bispecific antibodies can be prepared as full-length antibodies or antibody fragments.

In some embodiments, the first physiological condition is an aberrant condition and the second physiological condition is a normal physiological condition. For example, the aberrant condition may be a condition in a tumor microenvironment. The multi-specific antibody of the present disclosure may be referred to as a conditionally active multi-specific antibody.

In some embodiments, the conditionally active multispecific antibody is virtually inactive in binding to one or both of its target antigens or epitopes at a normal physiological condition but is active at an aberrant condition, optionally having a level of activity that is higher than the activity of the conditionally active multi-specific antibody at a normal physiological condition or the activity at a normal physiological condition of the parent antibody from which it is derived. In another embodiment, the conditionally active multispecific antibody is less active or virtually inactive at a pH of 7.0-7.6, but is active at a lower pH of 5.0-6.8. In some cases, the conditionally active multispecific antibody is reversibly or irreversibly inactivated at the normal physiological condition. In another example, the conditionally active multi-specific antibody may be more active in the lower pH environments found in the tumor microenvironment. The conditionally active multi-specific antibody may be used as a drug, therapeutic agent or diagnostic agent.

In some embodiments, the conditionally active multi-specific antibody or antibody fragment is less active or virtually inactive at a normal physiological condition (such as a non-tumor microenvironment) but is active at an aberrant condition (such as a tumor microenvironment), in comparison to the activity at the normal physiological condition of the parent or wild-type antibody or antibody fragment from which it is derived. As a result, the anti-Nectin-4 multi-specific antibodies or antibody fragments of the present disclosure may have a lower binding to Nectin-4 in normal physiological condition (such as a non-tumor microenvironment) in comparison to the parent or wild-type antibody or antibody fragment from which it is derived. For example, the conditionally active multi-specific antibody or antibody fragment is less active or virtually inactive at a pH of 7.0-7.6 in comparison to the parent or wild-type antibody or antibody fragment, but is active at a lower pH of 5.0-6.8 in comparison to the parent or wild-type antibody or antibody fragment. In some cases, the conditionally active multi-specific antibody or antibody fragment is reversibly or irreversibly inactivated at the normal physiological condition (such as a non-tumor microenvironment) in comparison to the parent or wild-type antibody or antibody fragment.

Techniques for making multispecific antibodies include, but are not limited to, recombinant co-expression of two immunoglobulin heavy chain-light chain pairs having different specificities (see Milstein and Cuello, Nature, vol. 305, pp. 537-540, 1983), WO 93/08829, and Traunecker et al., EMBO J. vol. 10, pp. 3655-3659, 1991), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, vol. 229, pp. 81-83, 1985); using leucine zippers to produce bi-specific antibodies (see, e.g., Kostelny et al., J. Immunol., vol. 148, pp. 1547-1553, 1992); using “diabody” technology for making bispecific antibody fragments (see, e.g., Hollinger et al., Proc. Natl. Acad. Sci. USA, vol. 90, pp. 6444-6448, 1993); and using single-chain Fv (scFv) dimers (see, e.g. Gruber et al., J. Immunol., vol. 152, pp. 5368-5374, 1994); and preparing trispecific antibodies as described, e.g., in Tutt et al. J. Immunol., vol. 147, pp. 60-69, 1991.

Engineered antibodies with three or more functional antigen binding sites, including “Octopus antibodies,” are also included herein (see, e.g. US 2006/0025576A1).

The anti-Nectin-4 antibodies or antibody fragments of the disclosure may be produced using recombinant methods and compositions, which are described in detail in US 2016/0017040.

The physical/chemical properties and/or biological activities of the anti-Nectin-4 antibodies or antibody fragments of the disclosure may be tested and measured by various assays known in the art. Some of these assays are described in U.S. Pat. No. 8,853,369.

### I. Immunoconjugates

In another aspect, the disclosure also provides immunoconjugates as described herein above, wherein the drug moiety is one or more cytotoxic agents, such as chemotherapeutic agents or drugs, growth inhibitory agents, toxins (e.g., protein toxins, enzymatically active toxins of bacterial, fungal, plant, or animal origin, or fragments thereof), and radioactive isotopes.

In one embodiment, the immunoconjugate as described herein above is an antibody-drug conjugate (ADC) conjugated to one or more drug moieties, including but not limited to a maytansinoid (see U.S. Pat. Nos. 5,208,020, 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res., vol. 53, pp. 3336-3342, 1993; and Lode et al., Cancer Res., vol. 58, pp. 2925-2928, 1998); an anthracycline such as daunomycin or doxorubicin (see Kratz et al., Current Med. Chem., vol. 13, pp. 477-523, 2006; Jeffrey et al., Bioorganic & Med. Chem. Letters, vol. 16, pp. 358-362, 2006; Torgov et al., Bioconj. Chem., vol. 16, pp. 717-721, 2005; Nagy et al., Proc. Natl. Acad. Sci. USA, vol. 97, pp. 829-834, 2000; Dubowchik et al., Bioorg. & Med. Chem. Letters, vol. 12, vol. 1529-1532, 2002; King et al., J. Med. Chem., vol. 45, pp. 4336-4343, 2002; and U.S. Pat. No. 6,630,579); methotrexate; vindesine; a taxane such as docetaxel, paclitaxel, larotaxel, tesetaxel, and ortataxel; a trichothecene; and CC1065.

In another embodiment, an immunoconjugate as described herein above, is conjugated to a drug moiety that is an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin,Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

In another embodiment, an as described herein above, is conjugated to a drug moiety that is a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², p³², Pb²¹² and radioactive isotopes of Lu. When the radioconjugate is used for detection, it may comprise a radioactive atom for scintigraphic studies, for example tc99m or I123, or a spin label for nuclear magnetic resonance (NMR) imaging (also known as magnetic resonance imaging, MRI), such as iodine-123, iodine-131, indium-111, fluorine-19, carbon-13, nitrogen-15, oxygen-17, gadolinium, manganese and iron.

In some embodiments, the immunoconjugate as described herein above, comprises a radioactive agent, which may be selected from an alpha emitter, a beta emitter and a gamma emitter. Examples of alpha emitters are ²¹¹At, ²¹⁰Bi, ²¹²Bi, ²¹¹Bi, ²²³Ra, ²²⁴Ra, ²³⁵Ac and ²²⁷Th Examples of beta-emitters are ⁶⁷Cu. ⁹⁰Y, ¹³¹I, ¹⁵³Sm, ¹⁶Ho and ¹³⁶Re. Examples of gamma emitters are ⁶⁰Co, ¹³⁷Ce, ⁵⁵Fe, ⁵⁴Mg, ²⁰³Hg, and ¹³³Ba.

Conjugates of an antibody/antibody fragment as described herein above, and cytotoxic agent may be made using a variety of bifunctional protein coupling agents such as N-succinimidyl-3-(2-pyridyldithio) propionate (SPDP), succinimidyl-4-(N-maleimidomethyl)cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science, vol. 238, pp. 1098-, 1987. Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res., vol. 52, pp. 127-131, 1992; U.S. Pat. No. 5,208,020) may be used.

The immunuoconjugates herein expressly contemplate, but are not limited to conjugates prepared with cross-linker reagents including, but not limited to, BMPS, EMCS, GMBS, HBVS, LC-SMCC, MBS, MPBH, SBAP, SIA, SLAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, Ill., U.S.A).

An exemplary embodiment of an ADC includes an antibody or antibody fragment (Ab) which targets a tumor cell, a drug moiety (D), and a linker moiety (L) that attaches Ab to D. In some embodiments, the antibody is attached to the linker moiety (L) through one or more amino acid residues, such as lysine and/or cysteine.

An exemplary ADC has Formula I as Ab-(L-D)ₚ, where p is 1 to about 20. In some embodiments, the number of drug moieties that can be conjugated to an antibody is limited by the number of free cysteine residues. In some embodiments, free cysteine residues are introduced into the antibody amino acid sequence by the methods described herein. Exemplary ADC's of Formula I include, but are not limited to, antibodies that have 1, 2, 3, or 4 engineered cysteine amino acids (Lyon et al., Methods in Enzym., vol. 502, pp. 123-138, 2012). In some embodiments, one or more free cysteine residues are already present in an antibody, without the use of engineering, in which case the existing free cysteine residues may be used to conjugate the antibody to a drug. In some embodiments, an antibody is exposed to reducing conditions prior to conjugation of the antibody in order to generate one or more free cysteine residues.

Linkers are used to conjugate a moiety to the antibody to form an immunoconjugate such as an ADC. Other suitable linkers are described in WO 2017/180842.

Some drug moieties that may be conjugated to the antibodies are include, but are not limited to, drug moieties described in WO 2017/180842.

Drug moieties may also include, but are not limited to, compounds with nucleolytic activity (e.g., a ribonuclease or a DNA endonuclease).

In certain embodiments, an immunoconjugate may comprise a highly radioactive atom. A variety of radioactive isotopes are available for the production of radioconjugated antibodies. Examples include At²¹¹, I¹³¹, I¹²⁵, Y⁹⁰, Re¹⁸⁶, Re¹⁸⁸, Sm¹⁵³, Bi²¹², p³², Pb²¹² and radioactive isotopes of Lu. In some embodiments, when an immunoconjugate is used for detection, it may comprise a radioactive atom for scintigraphic studies, for example Tc⁹⁹ or 1¹²³, or a spin label for nuclear magnetic resonance (NMR) imaging (also known as magnetic resonance imaging, MRI), such as zirconium-89, iodine-123, iodine-131, indium-111, fluorine-19, carbon-13, nitrogen-15, oxygen-17, gadolinium, manganese or iron. Zirconium-89 may be complexed to various metal chelating agents and conjugated to antibodies, e.g., for PET imaging (WO 2011/056983).

The radio- or other labels may be incorporated in the immunoconjugate in known ways. For example, a peptide may be biosynthesized or chemically synthesized using suitable amino acid precursors comprising, for example, one or more fluorine-19 atoms in place of one or more hydrogens. In some embodiments, labels such as Tc⁹⁹, I¹²³, Re¹⁸⁶, Re¹⁸⁸ and In¹¹¹ can be attached via a cysteine residue in the antibody. In some embodiments, yttrium-90 can be attached via a lysine residue of the antibody. In some embodiments, the IODOGEN method (Fraker et al., Biochem. Biophys. Res. Commun., vol. 80, pp. 49-57, 1978) can be used to incorporate iodine-123. “Monoclonal Antibodies in Immunoscintigraphy” (Chatal, CRC Press 1989) describes certain other methods.

In certain embodiments, an immunoconjugate as described herein above may comprise an antibody conjugated to a prodrug-activating enzyme. In some such embodiments, a prodrug-activating enzyme converts a prodrug (e.g., a peptidyl chemotherapeutic agent, see WO 81/01145) to an active drug, such as an anti-cancer drug. Such immunoconjugates are useful, in some embodiments, in antibody-dependent enzyme-mediated prodrug therapy (“ADEPT”). Enzymes that may be conjugated to an antibody include, but are not limited to, alkaline phosphatases, which are useful for converting phosphate-containing prodrugs into free drugs; arylsulfatases, which are useful for converting sulfate-containing prodrugs into free drugs; cytosine deaminase, which is useful for converting non-toxic 5-fluorocytosine into the anti-cancer drug, 5-fluorouracil; proteases, such as serratia protease, thermolysis, subtilisin, carboxypeptidases and cathepsins (such as cathepsins B and L), which are useful for converting peptide-containing prodrugs into free drugs; D-alanylcarboxypeptidases, which are useful for converting prodrugs that contain D-amino acid substituents; carbohydrate-cleaving enzymes such as β-galactosidase and neuraminidase, which are useful for converting glycosylated prodrugs into free drugs; β-lactamase, which is useful for converting drugs derivatized with β-lactams into free drugs; and penicillin amidases, such as penicillin V amidase and penicillin G amidase, which are useful for converting drugs derivatized at their amine nitrogens with phenoxyacetyl or phenylacetyl groups, respectively, into free drugs. In some embodiments, enzymes may be covalently bound to antibodies by recombinant DNA techniques well known in the art. See, e.g., Neuberger et al., Nature, vol. 312, pp. 604-608, 1984.

Drug loading in the conjugates is represented by m, the average number of drug moieties per antibody. Drug loading may range from 1 to 10 drug moieties per antibody, or from 1 to 5 drug moieties per antibody. The conjugates of the present disclosure may have a range of drug moieties, from 1 to 10 or from 1 to 5. The average number of drug moieties per antibody use in the preparation of the conjugates from conjugation reactions may be characterized by conventional means such as mass spectroscopy, ELISA assay, and HPLC.

For some antibody-drug conjugates (ADC), the drug loading may be limited by the number of attachment sites on the antibody. For example, where the attachment is a cysteine thiol, as in certain exemplary embodiments above, an antibody may have only one or several cysteine thiol groups, or may have only one or several sufficiently reactive thiol groups through which a linker may be attached. In certain embodiments, higher drug loading, e.g. n>5, may cause aggregation, insolubility, toxicity, or loss of cellular permeability of certain antibody-drug conjugates. In certain embodiments, the average drug loading for an ADC ranges from 1 to about 8; from about 2 to about 6; or from about 3 to about 5. Indeed, it has been shown that for certain ADCs, the optimal ratio of drug moieties per antibody may be less than 8, and may be about 2 to about 5 (U.S. Pat. No. 7,498,298).

In certain embodiments, fewer than the theoretical maximum of drug moieties are conjugated to an antibody during a conjugation reaction. An antibody may contain, for example, lysine residues that do not react with the drug-linker intermediate or linker reagent, as discussed below. Generally, antibodies do not contain many free and reactive cysteine thiol groups which may be linked to a drug moiety. Indeed, most cysteine thiol residues in antibodies exist as disulfide bridges. In certain embodiments, an antibody may be reduced with a reducing agent such as dithiothreitol (DTT) or tricarbonylethylphosphine (TCEP), under partial or total reducing conditions, to generate reactive cysteine thiol groups. In certain embodiments, an antibody is subjected to denaturing conditions to reveal reactive nucleophilic groups such as lysine or cysteine.

The loading (drug/antibody ratio or DAR) of an ADC or immunoconjugate of the present disclosure may be controlled in different ways, and for example, by: (i) limiting the molar excess of drug-linker intermediate or linker reagent relative to antibody, or (ii) limiting the conjugation reaction time or temperature, and/or (iii) partial or limiting reductive conditions for cysteine thiol modification.

### J. Methods and Compositions for Diagnostics and Detection

In certain embodiments, any of the isolated polypeptides or anti-Nectin-4 antibodies or antibody fragments as provided herein may be used for detecting the presence of Nectin-4 in a biological sample, either quantitatively or qualitatively. In certain embodiments, a biological sample comprises a cell or tissue, such as breast, pancreas, esophagus, lung and/or brain cells or tissue.

The present disclosure further provides a kit for diagnosis or detection, said kit comprising of a linker of any one of the embodiments of the linker described above, an immunoconjugate of any one of the embodiments of the immunoconjugate described above, or a pharmaceutical composition of any one of the embodiments of the pharmaceutical composition described herein below, and instructions for using the linker, the immunoconjugate and/or the pharmaceutical composition for diagnosis or detection of cancer.

A further aspect of the disclosure relates to an isolated polypeptide or an anti-Nectin-4 antibody or antibody fragment as described herein of the disclosure for diagnosing and/or monitoring a cancer or another disease in which Nectin-4 expression levels are increased or decreased from a normal physiological level at least one location in the body.

In a preferred embodiment, isolated polypeptides or antibodies or antibody fragments of the disclosure may be labelled with a detectable molecule or substance, such as a fluorescent molecule, a radioactive molecule or any other label known in the art as above described. For example, an antibody or antibody fragment of the disclosure may be labelled with a radioactive molecule. For example, suitable radioactive molecules include but are not limited to radioactive atoms used for scintigraphic studies such as ¹²³I, ¹²⁴I, ¹¹¹In, ¹⁸⁶Re, and ¹⁸⁸Re. Antibodies or antibody fragments of the disclosure may also be labelled with a spin label for nuclear magnetic resonance (NMR) imaging, such as iodine-123, iodine-131, indium-Ill, fluorine-19, carbon-13, nitrogen-15, oxygen-17, gadolinium, manganese or iron. Following administration of the antibody, the distribution of the radiolabeled antibody within the patient is detected. Any suitable known method can be used. Some non-limiting examples include, computed tomography (CT), position emission tomography (PET), magnetic resonance imaging (MRI), fluorescence, chemiluminescence and sonography.

Isolated polypeptides or antibodies or antibody fragments of the disclosure as described herein may be useful for diagnosing and staging of cancer and diseases associated with Nectin-4 overexpression. Cancers associated with Nectin-4 overexpression may include squamous cell cancer, small-cell lung cancer, non-small cell lung cancer, gastric cancer, pancreatic cancer, glial cell tumors such as glioblastoma and neurofibromatosis, cervical cancer, ovarian cancer, liver cancer, bladder cancer, hepatoma, breast cancer, colon cancer, melanoma, colorectal cancer, endometrial carcinoma, salivary gland carcinoma, kidney cancer, renal cancer, prostate cancer, vulval cancer, thyroid cancer, hepatic carcinoma, sarcomas, hematological cancers (leukemias), astrocytomas, and various types of head and neck cancer or other Nectin-4 expressing or overexpressing hyperproliferative diseases.

Isolated polypeptides or antibodies or antibody fragments of the disclosure as described herein may be useful for diagnosing diseases other than cancers for which Nectin-4 expression is increased or decreased. Both the (soluble or cellular Nectin-4 forms can be used for such diagnoses. Typically, such diagnostic methods involve use of a biological sample obtained from the patient. The biological sample encompasses a variety of sample types obtained from a subject that can be used in a diagnostic or monitoring assay. Biological samples include but are not limited to blood and other liquid samples of biological origin, solid tissue samples such as a biopsy specimen or a tissue culture or cells derived therefrom, and the progeny thereof. For example, biological samples include cells obtained from a tissue sample collected from an individual suspected of having a cancer associated with Nectin-4 overexpression, and in preferred embodiments from glioma, gastric, lung, pancreatic, breast, prostate, renal, hepatic and endometrial. Biological samples encompass clinical samples, cells in culture, cell supernatants, cell lysates, serum, plasma, biological fluid, and tissue samples.

In a particular embodiment, the disclosure is a method of diagnosing a cancer associated with Nectin-4 overexpression in a subject by detecting Nectin-4 on cells from the subject using the antibody of the disclosure. In particular, said method may include steps of:
1) contacting a biological sample of a subject with an antibody or antibody fragment according to the disclosure under conditions suitable for the antibody or antibody fragment to form complexes with cells in the biological sample that express Nectin-4; and
2) detecting and/or quantifying said complexes, whereby detection of said complexes is indicative of a cancer associated with Nectin-4 overexpression.

In order to monitor the progress of a cancer, the method according to the disclosure may be repeated at different times, in order to determine if antibody binding to the samples increases or decreases, wherefrom it can be determined if the cancer has progressed, regressed or stabilized.

In a particular embodiment, the invention is a method of diagnosing a disease associated with the expression or overexpression of Nectin-4. Examples of such diseases may include cancers, human immune disorders, thrombotic diseases (thrombosis and atherothrombosis), and cardiovascular diseases.

In one embodiment, an anti-Nectin-4 antibody or antibody fragment for use in a method of diagnosis or detection is provided. In a further aspect, a method of detecting the presence of Nectin-4 in a biological sample is provided. In a further aspect, a method of quantifying the amount of Nectin-4 in a biological sample is provided. In certain embodiments, the method comprises contacting the biological sample with an anti-Nectin-4 antibody or antibody fragment as described herein under conditions permissive for binding of the anti-Nectin-4 antibody or antibody fragment to Nectin-4 and detecting whether a complex is formed between the anti-Nectin-4 antibody or antibody fragment and Nectin-4. Such a method may be carried out in vitro or in vivo. In one embodiment, an anti-Nectin-4 antibody or antibody fragment is used to select subjects eligible for therapy. In some embodiments, the therapy will include administration of an anti-Nectin-4 antibody or antibody fragment to the subject.

In certain embodiments, labeled anti-Nectin-4 antibodies or antibody fragments are provided. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes ³²P, ¹⁴C, ¹²⁵I, ³H, and ¹³¹I, fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, β-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like.

### K. Pharmaceutical Formulations

The isolated polypeptides or anti-Nectin-4 antibodies or antibody fragments as described herein have cell killing activity. This cell killing activity extends to multiple different types of cell lines. Further, these isolated polypeptides or antibodies or antibody fragments of the present disclosure, once conjugated to a cytotoxic agent, can reduce tumor size and may exhibit reduced toxicity. Thus, the isolated polypeptides, anti-Nectin-4 antibodies, fragments or immunoconjugates thereof may be useful for treating proliferative diseases associated with Nectin-4 expression. The isolated polypeptides, antibodies, fragments or immunoconjugates may be used alone or in combination with any suitable agent or other conventional treatments.

The present disclosure provides a pharmaceutical composition comprising: a linker of any one of the embodiments of the linker described above, a conjugate of any one of the embodiments of the conjugate described above, or an immunoconjugate of any one of the embodiments of the immunoconjugate described above; and a pharmaceutically acceptable carrier.

In one embodiment of the above pharmaceutical composition, the present disclosure provides a single dose of the pharmaceutical composition comprising an amount of the immunoconjugate of about 135 mg, 235 mg, 335 mg, 435 mg, 535 mg, 635 mg, 735 mg, 835 mg, 935 mg, 1035 mg, 1135 mg, 1235 mg, or 1387 mg.

In any one of the embodiments of the above pharmaceutical composition, the present disclosure provides a single dose of the pharmaceutical composition comprising the immunoconjugate in a range of 135-235 mg, 235-335 mg, 335-435 mg, 435-535 mg, 535-635 mg, 635-735 mg, 735-835 mg, 835-935 mg, 935-1035 mg, 1035-1135 mg, 1135-1235 mg, or 1235-1387 mg.

In any one of the embodiments of the above pharmaceutical composition, the pharmaceutical composition further comprises an immune checkpoint inhibitor molecule.

In any one of the embodiments of the above pharmaceutical composition, in which the pharmaceutical composition further comprises an immune checkpoint inhibitor molecule, the immune checkpoint inhibitor molecule is an antibody or antibody fragment against an immune checkpoint.

In any one of the embodiments of the above pharmaceutical composition, the pharmaceutical composition further comprises an immune checkpoint inhibitor molecule, the immune checkpoint is selected from CTLA4, LAG3, TIM3, TIGIT, VISTA, BTLA, OX40, CD40, 4-1BB, PD-1, PD-L1, GITR, B7-H3, B7-H4, KIR, A2aR, CD27, CD70, DR3, and ICOS.

In any one of the embodiments of the above pharmaceutical composition, the pharmaceutical composition further comprises an immune checkpoint inhibitor molecule, the immune checkpoint is CTLA4, PD-1 or PD-L1.

In any one of the embodiments of the above pharmaceutical composition, the pharmaceutical composition further comprises an antibody or antibody fragment against an antigen selected from CTLA4, PD1, PD-L1, AXL, ROR2, CD3, HER2, B7-H3, ROR1, SFRP4 and a WNT protein.

The present disclosure provides a method of treating cancer comprising a step of administering a pharmaceutical composition of any one of the embodiments of the pharmaceutical composition described above, to a patient with cancer.

The present disclosure also provides use of a pharmaceutical composition of any one of the embodiments of the pharmaceutical composition described above, for the treatment of cancer.

The present disclosure further provides a kit for treatment, said kit comprising of a linker of any one of the embodiments of the linker described above, a conjugate of any one of the embodiments of the conjugate described above, or an immunoconjugate of any one of the embodiments of the immunoconjugate described above, or a pharmaceutical composition of any one of the embodiments of the pharmaceutical composition described above, and instructions for using the linker, the conjugate, or the immunoconjugate and/or the pharmaceutical composition for treatment of cancer.

The isolated polypeptides, anti-Nectin-4 antibodies or antibody fragments, immunconjugates, kits, or pharmaceutical compositions as described herein above, may be used to treat diseases associated with Nectin-4 expression, overexpression or activation. There are no particular limitations on the types of cancer or tissue that can be treated other than the requirement for Nectin-4 expression. Examples include squamous cell cancer, small-cell lung cancer, non-small cell lung cancer, gastric cancer, pancreatic cancer, glial cell tumors such as glioblastoma and neurofibromatosis, cervical cancer, ovarian cancer, liver cancer, bladder cancer, hepatoma, breast cancer, colon cancer, melanoma, colorectal cancer, endometrial carcinoma, salivary gland carcinoma, kidney cancer, renal cancer, prostate cancer, vulval cancer, thyroid cancer, hepatic carcinoma, sarcomas, hematological cancers (leukemias), astrocytomas, and various types of head and neck cancer. More preferable cancers are glioma, gastric, lung, pancreatic, breast, prostate, renal, hepatic and endometrial cancer.

The isolated polypeptides or anti-Nectin-4 antibodies or antibody fragments as described herein are potential activators of the innate immune response and thus may be used in the treatment of human immune disorders, such as sepsis. For example, an anti-Nectin-4 antibody or antibody fragment of the disclosure may also be used as adjuvants for immunization such as for vaccines and as anti-infection agents against, for example, bacteria, viruses and parasites.

The isolated polypeptides or anti-Nectin-4 antibody or antibody fragment may be used to protect against, prevent or treat thrombotic diseases such as venous and arterial thrombosis and atherothrombosis. For example, anti-Nectin-4 antibody or antibody fragment may also be used to protect against, prevent or treat cardiovascular diseases as well as to prevent or inhibit the entry of viruses such as Lassa and Ebola viruses and to treat viral infections.

In each of the embodiments of the treatment methods described herein, the isolated polypeptides, anti-Nectin-4 antibody, antibody fragment or anti-Nectin-4 antibody or antibody fragment immunoconjugate may be delivered in a manner consistent with conventional methodologies associated with management of the disease or disorder for which treatment is sought. In accordance with the disclosure herein, an effective amount of the antibody, antibody fragment or immunoconjugate is administered to a subject in need of such treatment for a time and under conditions sufficient to prevent or treat the disease or disorder. Thus, an aspect of the disclosure relates to a method for treating a disease associated with the expression of Nectin-4 comprising administering to a subject in need thereof with a therapeutically effective amount of an antibody, antibody fragment or immunoconjugate of the disclosure.

For administration, the anti-Nectin-4 antibody, antibody fragment or immunoconjugate may be formulated as a pharmaceutical composition. The pharmaceutical composition including an isolated polypeptide, anti-Nectin-4 antibody, antibody fragment or immunoconjugate of the present disclosure can be formulated according to known methods for preparing pharmaceutical compositions. In such methods, the therapeutic molecule is typically combined with a mixture, solution or composition containing a pharmaceutically acceptable carrier.

A pharmaceutically acceptable carrier is a material that can be tolerated by a recipient patient. Sterile phosphate-buffered saline is one example of a pharmaceutically acceptable carrier. Other suitable pharmaceutically acceptable carriers are well-known to those in the art. (See, e.g., Gennaro (ed.), Remington's Pharmaceutical Sciences (Mack Publishing Company, 19th ed. 1995)) Formulations may further include one or more excipients, preservatives, solubilizers, buffering agents, albumin to prevent protein loss on vial surfaces, etc.

The form of the pharmaceutical compositions, the route of administration, the dosage and the regimen naturally depend upon the condition to be treated, the severity of the illness, the age, weight, and sex of the patient, etc. These considerations can be taken into account by a skilled person to formulate suitable pharmaceutical compositions. The pharmaceutical compositions of the disclosure can be formulated for topical, oral, parenteral, intranasal, intravenous, intramuscular, subcutaneous or intraocular administration and the like.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition of, for example, sterilized water or physiological saline, permit the constitution of injectable solutions.

In some embodiments, tonicity agents, sometimes known as “stabilizers” are present to adjust or maintain the tonicity of a liquid in a composition. When used with large, charged biomolecules such as proteins and antibodies, they are often termed “stabilizers” because they can interact with the charged groups of the amino acid side chains, thereby lessening the potential for inter- and intra-molecular interactions. Tonicity agents can be present in any amount of from 0.1% to 25% by weight, preferably 1 to 5% of the pharmaceutical composition. Preferred tonicity agents include polyhydric sugar alcohols, preferably trihydric or higher sugar alcohols, such as glycerin, erythritol, arabitol, xylitol, sorbitol and mannitol.

Additional excipients include agents which can serve as one or more of the following: (1) bulking agents, (2) solubility enhancers, (3) stabilizers and (4) and agents preventing denaturation or adherence to the container wall. Such excipients may include: polyhydric sugar alcohols (enumerated above); amino acids such as alanine, glycine, glutamine, asparagine, histidine, arginine, lysine, ornithine, leucine, 2-phenylalanine, glutamic acid, threonine, etc.; organic sugars or sugar alcohols such as sucrose, lactose, lactitol, trehalose, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, α-monothioglycerol and sodium thio sulfate; low molecular weight proteins such as human serum albumin, bovine serum albumin, gelatin or other immunoglobulins; hydrophilic polymers such as polyvinylpyrrolidone; monosaccharides (e.g., xylose, mannose, fructose, glucose; disaccharides (e.g., lactose, maltose, sucrose); trisaccharides such as raffinose; and polysaccharides such as dextrin or dextran.

Non-ionic surfactants or detergents (also known as “wetting agents”) may be employed to help solubilize the therapeutic agent as well as to protect the therapeutic protein against agitation-induced aggregation, which also permits the formulation to be exposed to shear surface stress without causing denaturation of the active therapeutic protein or antibody. Non-ionic surfactants may be present in a concentration range of about 0.05 mg/ml to about 1.0 mg/ml, preferably about 0.07 mg/ml to about 0.2 mg/ml.

Suitable non-ionic surfactants include polysorbates (20, 40, 60, 65, 80, etc.), polyoxamers (184, 188, etc.), PLURONIC® polyols, TRITON®, polyoxyethylene sorbitan monoethers (TWEEN®-20, TWEEN®-80, etc.), lauromacrogol 400, polyoxyl 40 stearate, polyoxyethylene hydrogenated castor oil 10, 50 and 60, glycerol monostearate, sucrose fatty acid ester, methyl celluose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride

The doses used for the administration can be adapted as a function of various parameters, and in particular as a function of the mode of administration used, of the relevant pathology, or alternatively of the desired duration of treatment. To prepare pharmaceutical compositions, an effective amount of the antibody or antibody fragment may be dissolved or dispersed in a pharmaceutically acceptable carrier or aqueous medium.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions of the active compounds as free base or pharmacologically acceptable salts can be prepared in a water suitably mixed with a surfactant. Dispersions can also be prepared in glycerol, liquid polyethylene glycols, and mixtures thereof and in oils. Under ordinary conditions of storage and use, these preparations contain a preservative to prevent the growth of microorganisms.

The anti-Nectin-4 antibody or antibody fragment can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with one or more of the other ingredients enumerated above, as may be required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The preparation of more, or highly concentrated solutions for direct injection is also contemplated, where the use of dimethyl sulfoxide (DMSO) as solvent is envisioned to result in extremely rapid penetration, delivering high concentrations of the active agents to a small tumor area.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

The antibodies or antibody fragments may be formulated within a therapeutic mixture to deliver about 0.0001 to 10.0 milligrams, or about 0.001 to 5 milligrams, or about 0.001 to 1 milligram, or about 0.001 to 0.1 milligrams, or about 0.1 to 1.0 or even about 10 milligrams per dose. Multiple doses can also be administered at selected time intervals.

In addition to the compounds formulated for parenteral administration, such as intravenous or intramuscular injection, other pharmaceutically acceptable forms include, e.g. tablets or other solids for oral administration; time release capsules; and any other form currently used.

In certain embodiments, the use of liposomes and/or nanoparticles is contemplated for the introduction of antibodies or antibody fragments into host cells. The formation and use of liposomes and/or nanoparticles are known to those of skill in the art.

Nanocapsules can generally entrap compounds in a stable and reproducible way. To avoid side effects due to intracellular polymeric overloading, such ultrafine particles (sized around 0.1 μm) are generally designed using polymers able to degrade in vivo. Biodegradable polyalkyl-cyanoacrylate nanoparticles that meet these requirements are contemplated for use in the present disclosure, and such particles may be easily made.

Liposomes are formed from phospholipids that are dispersed in an aqueous medium and spontaneously form multilamellar concentric bilayer vesicles (also termed multilamellar vesicles (MLVs)). MLVs generally have diameters of from 25 nm to 4 μm. Sonication of MLVs results in the formation of small unilamellar vesicles (SUVs) with diameters in the range of 200 to 500 Å, containing an aqueous solution in the core. The physical characteristics of liposomes depend on pH, ionic strength and the presence of divalent cations

Pharmaceutical formulations containing an anti-Nectin-4 antibody or antibody fragment as described herein are prepared by mixing such antibody or antibody fragment having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain more than one active ingredient as necessary for the particular indication being treated. Preferably, ingredients with complementary activities that do not adversely affect each other may be combined into a single formulation. For example, it may be desirable to provide an EGFR antagonist (such as erlotinib), an anti-angiogenic agent (such as a VEGF antagonist which may be an anti-VEGF antibody) or a chemotherapeutic agent (such as a taxoid or a platinum agent) in addition to the anti-Nectin-4 antibody, antibody fragment or immunoconjugate of the present disclosure. Such active ingredients are suitably present in combination in amounts that are effective for the purpose intended.

In one embodiment, the anti-Nectin-4 antibody, antibody fragment or immunoconjugate of the present disclosure is combined in a formulation with another antibody or antibody fragment against an antigen selected from CTLA4, PD1, PD-L1, AXL, ROR2, CD3, HER2, B7-H3, ROR1, SFRP4 and a WNT protein including WNT1, WNT2, WNT2B, WNT3, WNT4, WNT5A, WNT5B, WNT6, WNT7A, WNT7B, WNT8A, WNT8B, WNT9A, WNT9B, WNT10A, WNT10B, WNT11, WNT16. The combination may be in the form of two separate molecules: the anti-Nectin-4 antibody, antibody fragment or immunoconjugate of the present disclosure, and another antibody or antibody fragment. Alternatively, the combination may also be the form of a single molecule with binding affinity to both Nectin-4 and the other antigen, thus forming a multispecific (e.g. bispecific) antibody.

Active ingredients may be encapsulated in microcapsules prepared, for example, by coacervation techniques or by interfacial polymerization. For example, hydroxymethylcellulose or gelatin-microcapsules and poly-(methylmethacrylate) microcapsules, respectively, in colloidal drug delivery systems (for example, liposomes, albumin microspheres, microemulsions, nanoparticles and nanocapsules) or in macroemulsions may be employed. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody or antibody fragment, which matrices may be in the form of shaped articles, e.g. films, or microcapsules.

The formulations to be used for in vivo administration are generally sterile. Sterility may be readily accomplished, e.g., by filtration through sterile filtration membranes.

### L. Therapeutic Methods and Compositions

The present disclosure provides a method of treating cancer comprising a step of administering an immunoconjugate of any one of the embodiments of the immunoconjugate described above or a pharmaceutical composition of any one of the embodiments of the pharmaceutical composition described above, to a patient with cancer.

The present disclosure also provides use of a linker of any one of the embodiments of the linker described above, an immunoconjugate of any one of the embodiments of the immunoconjugate described above, or a pharmaceutical composition of any one of the embodiments of the pharmaceutical composition described above, for the treatment of cancer.

Any of the isolated polypeptides, anti-Nectin-4 antibodies or antibody fragments, pharmaceutical compositions or immunoconjugates described herein above may be used in therapeutic methods as described below. In one aspect, an anti-Nectin-4 antibody or antibody fragment for use as a medicament is provided. In further aspects, an anti-Nectin-4 antibody or antibody fragment for use in treating cancer (e.g., breast cancer, non-small cell lung cancer, pancreatic cancer, brain cancer, cancer of pancreas, brain, kidney, ovary, stomach, leukemia, uterine endometrium, colon, prostate, thyroid, liver, osteosarcoma, and/or melanoma) is provided. In certain embodiments, an anti-Nectin-4 antibody or antibody fragment for use in a method of treatment is provided. In certain embodiments, the disclosure provides an anti-Nectin-4 antibody or antibody fragment for use in a method of treating an individual having cancer comprising administering to the individual an effective amount of the anti-Nectin-4 antibody or antibody fragment. In certain embodiments, the disclosure provides an anti-Nectin-4 antibody or antibody fragment for use in a method of treating an individual having an immune disorder (e.g., an autoimmune disorder), a cardiovascular disorder (e.g., atherosclerosis, hypertension, thrombosis), an infectious disease (e.g., Ebola virus, Marburg virus) or diabetes, comprising administering to the individual an effective amount of the anti-Nectin-4 antibody or antibody fragment. In one such embodiment, the method further comprises administering to the individual an effective amount of at least one additional therapeutic agent, e.g., as described below. In further embodiments, the disclosure provides an anti-Nectin-4 antibody or antibody fragment for use in inhibiting angiogenesis, inhibiting cell proliferation, inhibiting immune function, inhibiting inflammatory cytokine secretion (e.g., from tumor-associated macrophages), inhibiting tumor vasculature (e.g., intratumoral vasculature or tumor-associated vasculature), and/or inhibiting tumor stromal function.

In certain embodiments, the disclosure provides an anti-Nectin-4 antibody or antibody fragment for use in a method of inhibiting angiogenesis, inhibiting cell proliferation, inhibiting immune function, inhibiting inflammatory cytokine secretion (e.g., from tumor-associated macrophages), inhibiting tumor vasculature (e.g., intratumoral vasculature or tumor-associated vasculature), and/or inhibiting tumor stromal function in an individual comprising administering to the individual an effective of the anti-Nectin-4 antibody or antibody fragment to inhibit angiogenesis, inhibit cell proliferation, inhibit immune function, inhibit inflammatory cytokine secretion (e.g., from tumor-associated macrophages), inhibit tumor vasculature development (e.g., intratumoral vasculature or tumor-associated vasculature), and/or inhibit tumor stromal function. An “individual” according to any of the above embodiments is preferably a human.

In a further aspect, the disclosure provides for the use of an anti-Nectin-4 antibody or antibody fragment in the manufacture or preparation of a medicament. In one embodiment, the medicament is for treatment of cancer (in some embodiments, breast cancer, non-small cell lung cancer, pancreatic cancer, brain cancer, cancer of the pancreas, brain, kidney, ovary, stomach, leukemia, uterine endometrium, colon, prostate, thyroid, liver, osteosarcoma, and/or melanoma). In a further embodiment, the medicament is for use in a method of treating cancer comprising administering to an individual having cancer an effective amount of the medicament. In a further embodiment, the medicament is for use in a method of treating an immune disorder (e.g., an autoimmune disorder), a cardiovascular disorder (e.g., atherosclerosis, hypertension, thrombosis), an infectious disease (e.g., Ebola virus, Marburg virus) or diabetes, comprising administering to the individual an effective amount of the anti-Nectin-4 antibody or antibody fragment. In one such embodiment, the method further comprises administering to the individual an effective amount of at least one additional therapeutic agent, e.g., as described below. In a further embodiment, the medicament is for inhibiting angiogenesis, inhibiting cell proliferation, inhibiting immune function, inhibiting inflammatory cytokine secretion (e.g., from tumor-associated macrophages), inhibiting tumor vasculature (e.g., intratumoral vasculature or tumor-associated vasculature), and/or inhibiting tumor stromal function. In a further embodiment, the medicament is for use in a method of inhibiting angiogenesis, inhibiting cell proliferation, inhibiting immune function, inhibiting inflammatory cytokine secretion (e.g., from tumor-associated macrophages), inhibiting tumor vasculature (e.g., intratumoral vasculature or tumor-associated vasculature), and/or inhibiting tumor stromal function in an individual comprising administering to the individual an amount effective of the medicament to inhibit angiogenesis, inhibit cell proliferation, promote immune function, induce inflammatory cytokine section (e.g., from tumor-associated macrophages), inhibit tumor vasculature development (e.g., intratumoral vasculature or tumor-associated vasculature), and/or inhibit tumor stromal function. An “individual” according to any of the above embodiments may be a human.

In a further aspect, the disclosure provides a method for treating a cancer. In one embodiment, the method comprises administering to an individual having such cancer an effective amount of an anti-Nectin-4 antibody or antibody fragment. In one such embodiment, the method further comprises administering to the individual an effective amount of at least one additional therapeutic agent, as described below. An “individual” according to any of the above embodiments may be a human.

In a further aspect, the disclosure provides a method for treating an immune disorder (e.g., an autoimmune disorder), a cardiovascular disorder (e.g., atherosclerosis, hypertension, thrombosis), an infectious disease (e.g., Ebola virus, Marburg virus) or diabetes. In one such embodiment, the method further comprises administering to the individual an effective amount of at least one additional therapeutic agent, as described below. An “individual” according to any of the above embodiments may be a human.

In a further aspect, the disclosure provides a method for inhibiting angiogenesis, inhibiting cell proliferation, inhibiting immune function, inhibiting inflammatory cytokine secretion (e.g., from tumor-associated macrophages), inhibiting tumor vasculature (e.g., intratumoral vasculature or tumor-associated vasculature), and/or inhibiting tumor stromal function in an individual. In one embodiment, the method comprises administering to the individual an effective amount of an anti-Nectin-4 antibody or antibody fragment to inhibit angiogenesis, inhibit cell proliferation, promote immune function, induce inflammatory cytokine section (e.g., from tumor-associated macrophages), inhibit tumor vasculature development (e.g., intratumoral vasculature or tumor-associated vasculature), and/or inhibit tumor stromal function. In one embodiment, an “individual” is a human.

In a further aspect, the disclosure provides pharmaceutical formulations comprising any of the anti-Nectin-4 antibodies or antibody fragments provided herein, e.g., for use in any of the above therapeutic methods. In one embodiment, a pharmaceutical formulation comprises any of the anti-Nectin-4 antibodies or antibody fragments provided herein and a pharmaceutically acceptable carrier. In another embodiment, a pharmaceutical formulation comprises any of the anti-Nectin-4 antibodies or antibody fragments provided herein and at least one additional therapeutic agent, e.g., as described below.

In each and every treatment described above, the antibodies or antibody fragments of the disclosure can be used alone, as immunoconjugates or in combination with other agents in a therapy. For instance, an antibody of the disclosure may be co-administered with at least one additional therapeutic agent. In certain embodiments, an additional therapeutic agent is an anti-angiogenic agent. In certain embodiments, an additional therapeutic agent is a VEGF antagonist (in some embodiments, an anti-VEGF antibody, for example bevacizumab). In certain embodiments, an additional therapeutic agent is an EGFR antagonist (in some embodiments erlotinib). In certain embodiments, an additional therapeutic agent is a chemotherapeutic agent and/or a cytostatic agent. In certain embodiments, an additional therapeutic agent is a taxoid (e.g., paclitaxel) and/or a platinum agent (e.g., carboplatinum). In certain embodiments the additional therapeutic agent is an agent that enhances the patient's immunity or immune system.

Such combination therapies noted above encompass combined administration (where two or more therapeutic agents are included in the same or separate formulations), and separate administration, in which case, administration of the antibody or antibody fragment can occur prior to, simultaneously, and/or following, administration of the additional therapeutic agent and/or adjuvant. Antibodies or antibody fragments can also be used in combination with radiation therapy.

The anti-Nectin-4 antibodies or antibody fragments may be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody or antibody fragment need not be but is optionally formulated with one or more agents currently used to prevent or treat the disorder in question. The effective amount of such other agents depends on the amount of antibody or antibody fragment present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody or antibody fragment (when used alone or in combination with one or more other additional therapeutic agents) will depend on the type of disease to be treated, the type of antibody or antibody fragment, the severity and course of the disease, whether the antibody or antibody fragment is administered for preventive or therapeutic purposes, previous therapy, the patient's clinical history and response to the antibody or antibody fragment, and the discretion of the attending physician. The antibody or antibody fragment is suitably administered to the patient at one time or over a series of treatments. Depending on the type and severity of the disease, about 1 μg of antibody or antibody fragment/kg bodyweight of the patient to 40 mg of antibody or antibody fragment/kg bodyweight of the patient can be an initial candidate dosage for administration to the patient, whether, for example, by one or more separate administrations, or by continuous infusion. One typical daily dosage might range from about 1 μg of antibody or antibody fragment/kg bodyweight of the patient to 100 mg of antibody or antibody fragment/kg bodyweight of the patient or more, depending on the factors mentioned above. For repeated administrations over several days or longer, depending on the condition, the treatment would generally be sustained until a desired suppression of disease symptoms occurs. Such doses may be administered intermittently, e.g. every week or every three weeks (e.g. such that the patient receives from about two to about twenty, or e.g. about six doses of the antibody or antibody fragment). An initial higher loading dose, followed by one or more lower doses may be administered. However, other dosage regimens may be useful. The progress of this therapy is easily monitored by conventional techniques and assays.

Specific dosages of the anti-Nectin-4 antibody or antibody fragment of the present disclosure that may be administered for the prevention or treatment of a disease in a subject may be about 0.3, 0.6, 1.2, 1.8, 2.4, 3.0, 3.6, 4.2, 4.8, 5.4, 6.0, 6.6, 7.2, 7.8, 8.4, 9.0, 9.6 or 10.2 mg of antibody or antibody fragment/kg bodyweight of the patient. In certain embodiments, the dosage may be in a range of 0.3-2.4, 2.4-4.2, 4.2-6.0, 6.0-7.8, 7.8-10.2, 10.2-12, 12-14, 14-16, 16-18 or 18-20 mg of antibody or antibody fragment/kg bodyweight of the patient. The dosage of the antibody or antibody fragment will remain the same if administered in the form of a bispecific antibody, in combination with another immune checkpoint inhibitor or another antibody or antibody fragment or as an immunoconjugate. Further, a polypeptide having anti-Nectin-4 activity will be administered in the same amounts as the antibody or antibody fragment.

A single dose of pharmaceutical formulation of the present disclosure may contain an amount of the anti-Nectin-4 antibody or antibody fragment of the present disclosure of from about 45 μg of antibody or antibody fragment from about 13,600 mg, or from about 45 μg of antibody or antibody fragment from about 5440 mg. In some embodiments, a single dose of pharmaceutical formulation of the present disclosure may contain an amount of the anti-Nectin-4 antibody or antibody fragment of the present disclosure of from to 135 mg to 1,387 mg, or an amount such as 135, 235, 335, 435, 535, 635, 735, 835, 935, 1035, 1135, 1235, 1387 mg. In certain embodiments, the amount of the anti-Nectin-4 antibody or antibody fragment of the present disclosure in a single dose of the pharmaceutical formulation is in the range of 135-235, 235-335, 335-435, 435-535, 535-635, 635-735, 735-835, 835-935, 935-1035, 1035-1135, 1135-1235, 1235-1387 mg. The amount of the antibody or antibody fragment in the single dose of the pharmaceutical formulation will remain the same if administered in the form of a bispecific antibody, in combination with another immune checkpoint inhibitor or as an immunoconjugate, or in combination with another antibody or antibody fragment against another antigen as disclosed herein. Further, a polypeptide having anti-Nectin-4 activity will be included in the single dose of the pharmaceutical formulation in the same amounts as the antibody or antibody fragment.

In one example, the anti-Nectin-4 antibody or antibody fragment may be conjugated to an immune checkpoint inhibitor molecule or may form part of a bispecific antibody with an immune checkpoint inhibitor.

The combination can be the anti-Nectin-4 antibody or antibody fragment disclosed in this application and the immune checkpoint inhibitor molecule administered as separate molecules or as a bispecific antibody. Such a bispecific antibody has a binding activity to Nectin-4 and a second binding activity to the immune checkpoint.

The immune checkpoint may be selected from CTLA4, LAG3, TIM3, TIGIT, VISTA, BTLA, OX40, CD40, 4-1BB, PD-1, PD-L1, and GITR (Zahavi and Weiner, International Journal of Molecular Sciences, vol. 20, 158, 2019). Additional immune checkpoints include B7-H3, B7-H4, KIR, A2aR, CD27, CD70, DR3, and ICOS (Manni et al., Immune checkpoint blockade and its combination therapy with small-molecule inhibitors for cancer treatment, Bbacan, https://doi.org/10.1016/j.bbcan.2018.12.002, 2018).

The immune checkpoint is preferably CTLA4, PD-1 or PD-L1.

It is understood that any of the above formulations or therapeutic methods may be carried out using an antibody fragment or an immunoconjugate of the disclosure in place of or in addition to an anti-Nectin-4 antibody.

Enhancing the host's immune function to combat tumors is the subject of increasing interest. Conventional methods include (i) APC enhancement, such as (a) injection into the tumor of DNA encoding foreign MHC alloantigens, or (b) transfecting biopsied tumor cells with genes that increase the probability of immune antigen recognition (e.g., immune stimulatory cytokines, GM-CSF, co-stimulatory molecules B7.1, B7.2) of the tumor, (iii) adoptive cellular immunotherapy, or treatment with activated tumor-specific T-cells. Adoptive cellular immunotherapy includes isolating tumor-infiltrating host T-lymphocytes, expanding the population in vitro, such as through stimulation by IL-2 or tumor or both. Additionally, isolated T-cells that are dysfunctional may be also be activated by in vitro application of the anti-PD-L1 antibodies of the disclosure. T-cells that are so-activated may then be readministered to the host. One or more of these methods may be used in combination with administration of the antibody, antibody fragment or immunoconjugate of the present disclosure.

Traditional therapies for cancer include the following: (i) radiation therapy (e.g., radiotherapy, X-ray therapy, irradiation) or the use of ionizing radiation to kill cancer cells and shrink tumors. Radiation therapy can be administered either externally via external beam radiotherapy (EBRT) or internally via brachytherapy; (ii) chemotherapy, or the application of cytotoxic drug which generally affect rapidly dividing cells; (iii) targeted therapies, or agents which specifically affect the deregulated proteins of cancer cells (e.g., tyrosine kinase inhibitors imatinib, gefitinib; monoclonal antibodies, photodynamic therapy); (iv) immunotherapy, or enhancement of the host's immune response (e.g., vaccine); (v) hormonal therapy, or blockade of hormone (e.g., when tumor is hormone sensitive), (vi) angiogenesis inhibitor, or blockade of blood vessel formation and growth, and (vii) palliative care, or treatment directed to improving the quality of care to reduce pain, nausea, vomiting, diarrhea and hemorrhage. Pain medication such as morphine and oxycodone, anti-emetics such as ondansetron and aprepitant, can permit more aggressive treatment regimens.

In the treatment of cancer, any of the previously described conventional treatments for the treatment of cancer immunity may be conducted, prior, subsequent or simultaneous with the administration of the anti-Nectin-4 antibodies or antibody fragments. Additionally, the anti-Nectin-4 antibodies or antibody fragments may be administered prior, subsequent or simultaneous with conventional cancer treatments, such as the administration of tumor-binding antibodies (e.g., monoclonal antibodies, toxin-conjugated monoclonal antibodies) and/or the administration of chemotherapeutic agents.

### M. Articles of Manufacture and Kits

In another aspect of the disclosure, an article of manufacture containing an isolated polypeptide, an anti-Nectin-4 antibody or antibody fragment, or an immunoconjugate as described herein and other materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an antibody or antibody fragment of the disclosure. The label or package insert indicates that the composition is used for treating the condition of choice. Moreover, the article of manufacture may comprise (a) a first container with a composition contained therein, wherein the composition comprises an antibody or antibody fragment; and (b) a second container with a composition contained therein, wherein the composition comprises a further cytotoxic or otherwise therapeutic agent. The article of manufacture in this embodiment of the disclosure may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

It is understood that any of the above articles of manufacture may include an immunoconjugate of the disclosure in place of or in addition to an anti-Nectin-4 antibody or antibody fragment.

Finally, the disclosure also provides kits comprising at least one antibody or antibody fragment of the disclosure. Kits containing polypeptide, antibodies or antibody fragments, or antibody drug conjugate of the disclosure find use in detecting Nectin-4 expression (increase or decrease), or in therapeutic or diagnostic assays. Kits of the disclosure can contain an antibody coupled to a solid support, e.g., a tissue culture plate or beads (e.g., sepharose beads). Kits can be provided which contain antibodies for detection and quantification of Nectin-4 in vitro, e.g. in an ELISA or a Western blot. Such antibody useful for detection may be provided with a label such as a fluorescent or radiolabel.

The kits further contain instructions on the use thereof. In some embodiments, the instructions comprise instructions required by the U.S. Food and Drug Administration for in vitro diagnostic kits. In some embodiments, the kits further comprise instructions for diagnosing the presence or absence of cerebrospinal fluid in a sample based on the presence or absence of Nectin-4 in said sample. In some embodiments, the kits comprise one or more antibodies or antibody fragments. In other embodiments, the kits further comprise one or more enzymes, enzyme inhibitors or enzyme activators. In still other embodiments, the kits further comprise one or more chromatographic compounds. In yet other embodiments, the kits further comprise one or more compounds used to prepare the sample for spectroscopic assay. In further embodiments, the kits further comprise comparative reference material to interpret the presence or absence of Nectin-4 according to intensity, color spectrum, or other physical attributes of an indicator.

The following examples are illustrative, but not limiting, of the anti-Nectin-4 antibodies of the present disclosure. Other suitable modifications and adaptations of the variety of conditions and parameters normally encountered in the field, and which are obvious to those skilled in the art, are within the scope of the disclosure.

### EXAMPLES

TABLE ABBREVIATIONS FOR EXAMPLES
TABLE OF ABBREVIATIONS

Ac: acetyl
Ac₂O: acetic anhydride
Ag₂O: silver oxide
° C.: degree Celsius
cHy or CyH: cyclohexane
Cl: chloro
Cs₂CO₃: cesium carbonate
d: days/doublet (NMR)
DCM: dichloromethane
dd: doublet of doublets
ddd: doublet of doublet of doublets
dt: doublet of triplets
DIPEA: N,N-diisopropylethylamine
DMF: N,N-dimethylformamide
DMAP: 4-dimethylaminopyridine
DMSO: dimethyl sulfoxide
eq.: equivalents
Et₂O: diethyl ether
EtOAc: ethyl acetate
g: gram
¹H: hydrogen
h: hours
HCl: hydrogen chloride
H₂O: water
Hz: Hertz
J: scalar coupling constant
L: liter or Linker
M: molar
m: multiplet
MeCN: acetonitrile
MeOH: methanol
min: minute
mL: milli liter
MPLC: medium pressure liquid chromatography
MS: molecular sieves
N: normal
NaHCO₃: sodium hydrogencarbonate
Na₂SO₄: sodium sulfate
NEt₃: triethylamine
NMR: nuclear magnetic resonance
py: pyridine
R_{f}: retention factor (TLC)
rt: room temperature
t: triplet
THF: tetrahydrofuran
TLC: thin layer chromatography
pTs: paratoluenesulfonyl

### Example 1—Synthesis of SPC07

### Synthesis of (2S,3R,4S,5S,6S)-2-(4-formyl-2-nitrophenoxy)-6-(methoxycarbonyl)tetrahydro-2H-pyran-3,4,5-triyl triacetate (SYB-BA2-066)

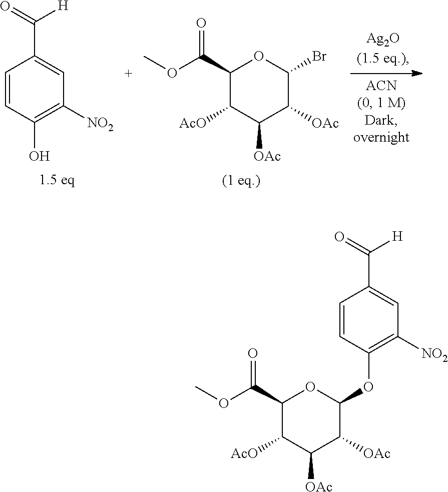 [SMILES: O=Cc1ccc(O)c([N+](=O)[O-])c1 ; COC(=O)[C@H]1O[C@H](Br)[C@H](OC(C)=O)[C@@H](OC(C)=O)[C@@H]1OC(C)=O ; COC(=O)[C@H]1O[C@@H](Oc2ccc(C=O)cc2[N+](=O)[O-])[C@H](OC(C)=O)[C@@H](OC(C)=O)[C@@H]1OC(C)=O]

To a solution of (2R,3R,4S,5S,6S)-2-bromo-6-(methoxycarbonyl)tetrahydro-2H-pyran-3,4,5-triyl triacetate (100 mg, 0.252 mmol, 1.0 eq.) in dry MeCN (2.5 mL, 0.1 M) was added 4-hydroxy-3-nitrobenzaldehyde (63 mg, 0.38 mmol, 1.5 eq.) and silver oxide (87 mg, 0.38 mmol, 1.5 eq.) at room temperature under a nitrogen atmosphere. The resulting reaction mixture was stirred in the dark at room temperature for 17 h. TLC showed full conversion of the donor (CyH/EtOAc 1:1, KMnO₄). The solution was filtered through Celite to remove solids and the filtrate was concentrated under reduced pressure. The residue was diluted with EtOAc (40 ml) and washed with saturated NaHCO₃ (6×20 ml), water and brine. The organic layer was dried over MgSO₄, filtered and concentrated under reduced pressure to afford (2S,3R,4S,5S,6S)-2-(4-formyl-2-nitrophenoxy)-6-(methoxycarbonyl)tetrahydro-2H-pyran-3,4,5-triyl triacetate (109 mg, 0.225 mmol, 89%) as a white solid.

¹H NMR (400 MHz, CDCl₃) δ 9.96 (s, 1H), 8.29 (d, J=2.0 Hz, 1H), 8.07 (dd, J=8.6, 2.0 Hz, 1H), 7.50 (d, J=8.6 Hz, 1H), 5.45-5.37 (m, 2H), 5.35-5.24 (m, 2H), 4.33 (d, J=8.4 Hz, 1H), 3.69 (s, 3H), 2.11 (s, 3H), 2.06 (s, 3H), 2.05 (s, 3H).

LC-MS (ESI): m/z=501 [M+H₂O]

### Synthesis of (2S,3R,4S,5S,6S)-2-(4-(1-hydroxybut-3-yn-1-yl)-2-nitrophenoxy)-6 (methoxycarbonyl)-tetrahydro-2H-pyran-3,4,5-triyl triacetate (SYB-BA2-060)

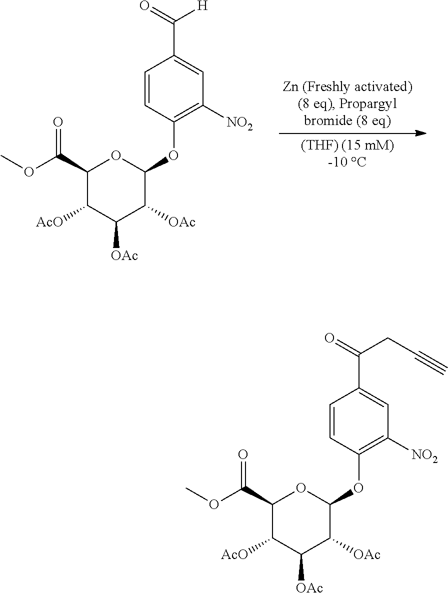 [SMILES: COC(=O)[C@H]1O[C@@H](Oc2ccc(C=O)cc2[N+](=O)[O-])[C@H](OC(C)=O)[C@@H](OC(C)=O)[C@@H]1OC(C)=O ; C#CCC(=O)c1ccc(O[C@@H]2O[C@H](C(=O)OC)[C@@H](OC(C)=O)[C@H](OC(C)=O)[C@H]2OC(C)=O)c([N+](=O)[O-])c1]

To a suspension of freshly activated zinc powder (HCl, H₂O, MeOH, Et₂O, high vacuum dry, 54 mg, 0.83 mmol, 8.0 eq.) in dry THF (2 mL, 0.4 M) was added allyl bromide (123 mg, 0.825 mmol, 8.0 eq.) at 0° C. under a nitrogen atmosphere. The reaction was warmed to room temperature and stirred for 1 h until all zinc was dissolved. The reaction solution was cooled to −15° C. and a solution of (2S,3R,4S,5S,6S)-2-(4-formyl-2-nitrophenoxy)-6-(methoxycarbonyl)tetrahydro-2H-pyran-3,4,5-triyl triacetate (50 mg, 0.103 mmol, 1.0 eq.) in THF (1 mL) was slowly added. The reaction mixture was stirred for 30 min at −15° C. and quenched by addition of a sat. aq. NH4Cl solution (20 mL). The aqueous solution was extracted with EtOAc (3*20 mL). The combined organic layers were washed with H₂O (30 mL), dried over Na₂SO₄, filtered and concentrated under reduced pressure. The crude product was purified by silica gel column chromatography (12 g, 20 CV, CyH:EtOAc: 95:5 to 20:80) to afford (2S,3R,4S,5S,6S)-2-(4-(1-hydroxybut-3-yn-1-yl)-2-nitrophenoxy)-6-(methoxycarbonyl)-tetrahydro-2H-pyran-3,4,5-triyl triacetate (14 mg, 1.16 mmol, 53%).

¹H NMR (400 MHz, CDCl₃) δ 7.87 (ddd, J=5.5, 2.2, 0.6 Hz, 1H), 7.58 (dddd, J=8.6, 6.3, 2.2, 0.6 Hz, 1H), 7.37 (d, J=8.6 Hz, 1H), 5.41-5.25 (m, 3H), 5.24-5.17 (m, 1H), 4.91 (t, J=6.2 Hz, 1H), 4.24-4.18 (m, 1H), 3.74 (s, 3H), 2.70-2.57 (m, 2H), 2.13 (s, 3H), 2.11 (t, J=2.6 Hz, 1H), 2.07 (s, 3H), 2.05 (s, 3H), 2.04 (d, J=3.8 Hz, 1H).

LC-MS (ESI): m/z=541 [M+H₂O]

### Synthesis of (2S,3S,4S,5R,6S)-2-(methoxycarbonyl)-6-(2-nitro-4-(1-(((4-nitrophenoxy) carbonyl)oxy) but-3-yn-1-yl) phenoxy)tetrahydro-2H-pyran-3,4,5-triyl triacetate (SYB-BA2-23)

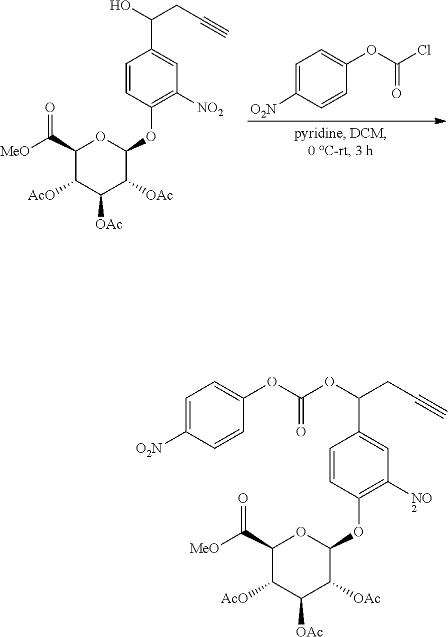 [SMILES: C#CCC(O)c1ccc(O[C@@H]2O[C@H](C(=O)OC)[C@@H](OC(C)=O)[C@H](OC(C)=O)[C@H]2OC(C)=O)c([N+](=O)[O-])c1 ; O=C(Cl)Oc1ccc([N+](=O)[O-])cc1 ; C#CCC(OC(=O)Oc1ccc([N+](=O)[O-])cc1)c1ccc(O[C@@H]2O[C@H](C(=O)OC)[C@@H](OC(C)=O)[C@H](OC(C)=O)[C@H]2OC(C)=O)c([N+](=O)[O-])c1]

A solution of (2S,3R,4S,5S,6S)-2-(4-(1-hydroxybut-3-yn-1-yl)-2-nitrophenoxy)-6-(methoxycarbonyl)tetrahydro-2H-pyran-3,4,5-triyl triacetate (68 mg, 0.13 mmol, 1 eq.), 4-nitrophenyl chloroformate (65 mg, 0.32 mmol, 2.5 eq.) in dichloromethane (2 mL) at 0° C. was added pyridine (100 μL, 1.3 mmol, 10 eq.), and the reaction was stirred for 3 hours at room temperature. After filtration over a pad of celite, the filtrate was washed with aq. sat. NaHCO₃ and extracted three times with EtOAc. The combined organic layers were dried, filtered and concentrated under reduced pressure. The residue was purified by FCC on silica (CyH/EtOAc 95:5 to 20:80, 20 g) to afford (2S,3S,4S,5R,6S)-2-(methoxycarbonyl)-6-(2-nitro-4-(1-(((4-nitrophenoxy) carbonyl)oxy) but-3-yn-1-yl) phenoxy)tetrahydro-2H-pyran-3,4,5-triyl triacetate (60 mg, 87 μmol, 67%).

R_{f}=0.30 product (KMnO₄)

¹H NMR (400 MHz, CDCl₃) δ 8.35-8.27 (m, 2H), 7.96 (d, J=2.2 Hz, 1H), 7.67 (dd, J=8.5, 2.3 Hz, 1H), 7.47-7.37 (m, 3H), 5.83 (t, J=6.5 Hz, 1H), 5.47-5.22 (m, 4H), 4.26 (d, J=8.8 Hz, 1H), 3.76 (s, 3H), 3.06-2.85 (m, 2H), 2.15 (s, 3H), 2.12 (t, J=2.6 Hz, 1H), 2.10 (s, 3H), 2.06 (s, 3H).

Synthesis of SPC07: 3 steps one purification optimization procedure.

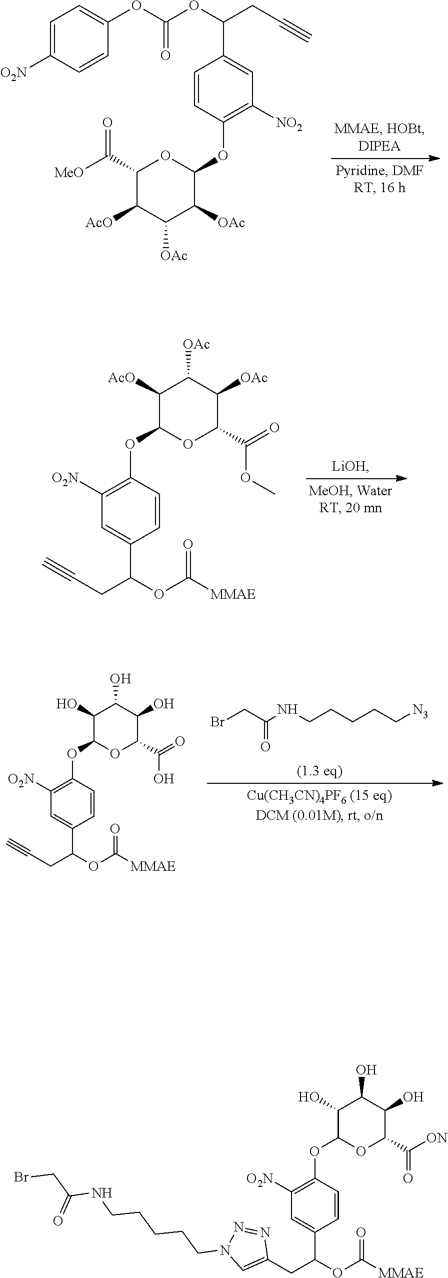

### (2S,3R,4S,5S,6S)-2-(4-((3R,4S,7S,10S)-4-((S)-sec-butyl)-3-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-7,10-diisopropyl-5,11-dimethyl-6,9,12-trioxo-2,13-dioxa-5,8,11-triazaheptadec-16-yn-14-yl)-2-nitrophenoxy)-6-(methoxycarbonyl)tetrahydro-2H-pyran-3,4,5-triyl triacetate (SYB-BA2-026)

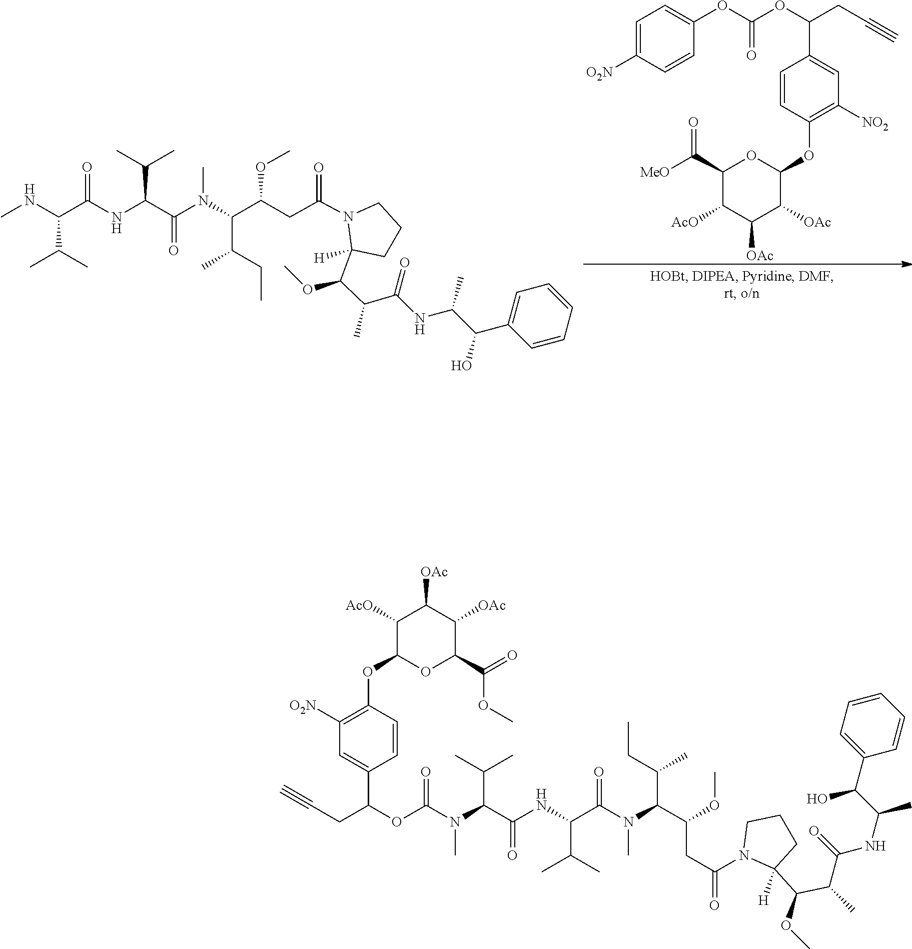

To a solution of (2S,3S,4S,5R,6S)-2-(methoxycarbonyl)-6-(2-nitro-4-(1-(((4-nitrophenoxy) carbonyl)oxy) but-3-yn-1-yl) phenoxy)tetrahydro-2H-pyran-3,4,5-triyl triacetate (60 mg, 0.87 mmol, 1 eq.), Monomethylauristatin E (63 mg, 0.87 mmol, 1 eq.) and DIPEA (23 μL, 0.13 mmol, 1.5 eq.) and pyridine (0.43 mL, 0.87 mmol, 1 eq. 0.2 M in DMF) in DMF (4 mL) was added HOBT (14 mg, 0.43 mmol, 1.05 eq.).

The reaction mixture was stirred overnight. LCMS showed formation of product plus some SM MMAE remaining.

No purification was attempted. The crude reaction was hydrolysed directly.

### (2S,3S,4S,5R,6S)-6-(4-((3R,4S,7S,10S)-4-((S)-sec-butyl)-3-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-7,10-diisopropyl-5,11-dimethyl-6,9,12-trioxo-2,13-dioxa-5,8,11-triazaheptadec-16-yn-14-yl)-2-nitrophenoxy)-3,4,5-trihydroxytetrahydro-2H-pyran-2-carboxylic acid (SYB-BA1-34)

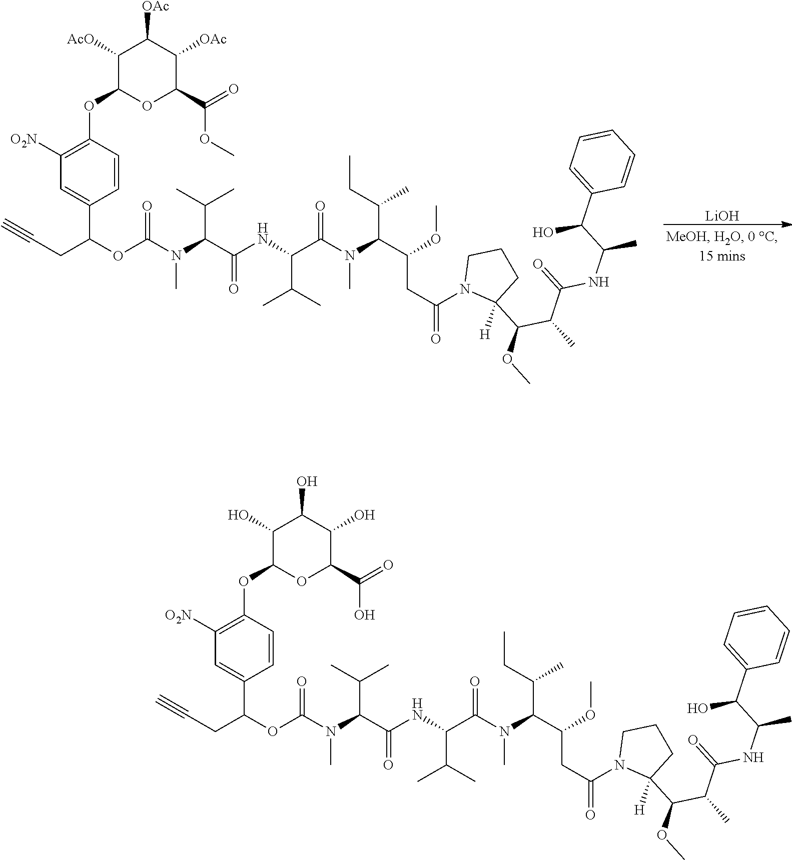

The reaction mixture (SYB-BA2-26) was diluted with MeOH (20 mL) and cooled to 0° C. To the resulting solution was added LiOH (8.8 eq., 1 M in H₂O, 0.76 mL, 0.76 mmol) and the reaction was followed by TLC (DCM/MeOH 9/1). The resulting solution was quenched with HCOOH (20 eq., 1 M in H₂O, 1.1 mL, 1.1 mmol) and concentrated. The reaction was diluted in water and extracted with DCM three times. LCMS of both organic and aqueous layers showed that the product stayed in the organic layer. The solvent was removed under reduced pressure. The residue in DMF was diluted with water and lyophilized overnight. The crude mixture was used for the click reaction. Purification at this stage is not necessary if the impurity is below 15%, this impurity will be removed during the reverse phase purification of the final click reaction.

### General Procedure for the Click Reaction

To a solution of alkyne derivative (1.0 eq.) in DCM (0.05 M) was added the corresponding azide (1.3 eq.) followed by the addition of tetrakis(acetonitrile) copper (i) hexafluoro phosphate (1.5 eq.). The solution was stirred at room temperature and the reaction was monitored by LCMS. After 4 hours (SM was consumed confirmed by LC-MS), the reaction mixture was concentrated under reduced pressure to remove DCM. Then the crude was diluted with DMF (0.05 M) and a solution ethylenediaminetetraacetic acid disodium salt dihydrate (EDTA·2Na·2H₂O) (4 eq.) in water (0.15 M) was added at 0° C. and stirring was continued at room temperature for 15 minutes. The reaction mixture was directly injected to the reverse phase column (25 μm) and the crude was purified using ACN in 0.1% FA in water. The pure fractions (confirmed by LCMS) were lyophilized to afford the desired compound as a white solid.

### (2S,3S,4S,5R,6S)-6-(4-((3R,4S,7S,10S)-15-(1-(5-(2-bromoacetamido)pentyl)-1H-1,2,3-triazol-4-yl)-4-((S)-sec-butyl)-3-(2-((S)-2-((1R,2R)-3-(((1S,2R)-1-hydroxy-1-phenylpropan-2-yl)amino)-1-methoxy-2-methyl-3-oxopropyl) pyrrolidin-1-yl)-2-oxoethyl)-7,10-diisopropyl-5,11-dimethyl-6,9,12-trioxo-2,13-dioxa-5,8,11-triazapentadecan-14-yl)-2-nitrophenoxy)-3,4,5-trihydroxytetrahydro-2H-pyran-2-carboxylic acid (SYB-BA2-35-SPC07 or SYB-BA1-36-SPC07)

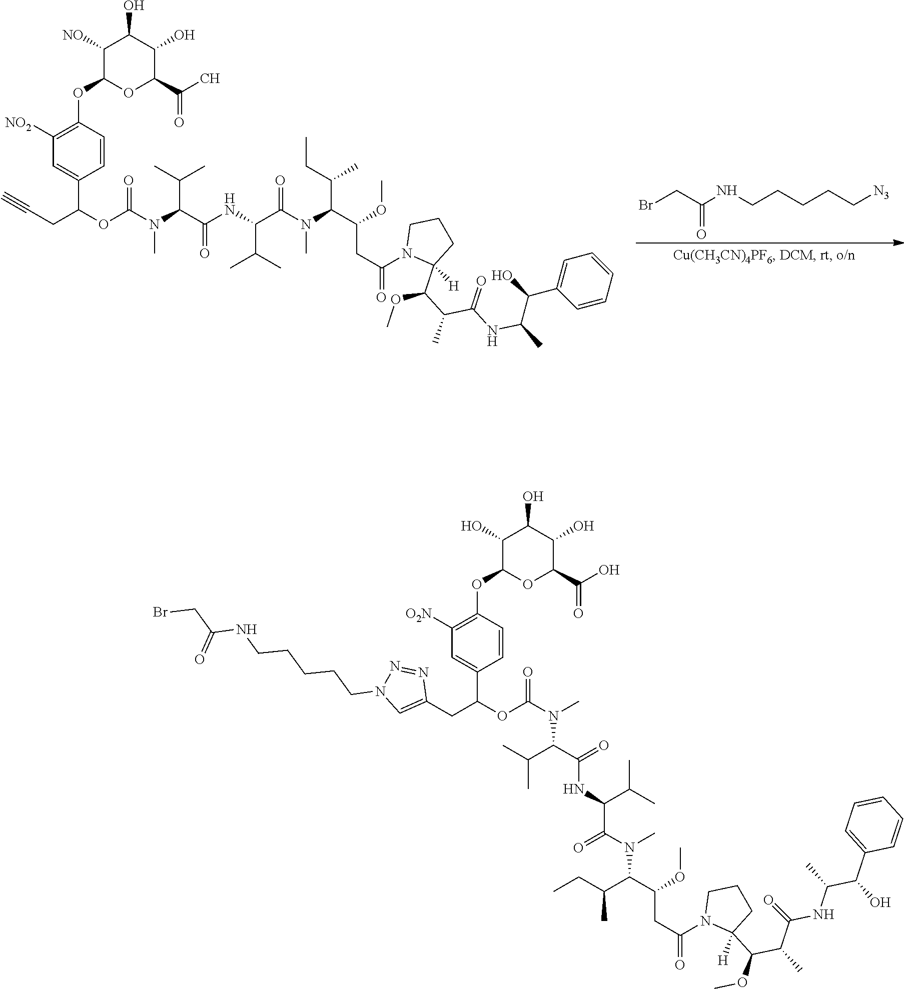

Following the general procedure for the Click reaction (2S,3S,4S,5R,6S)-6-(4-((3R,4S,7S,10S)-4-((S)-sec-butyl)-3-(2-((S)-2-((1R,2R)-3-(((1S,2R)-1-hydroxy-1-phenylpropan-2-yl)amino)-1-methoxy-2-methyl-3-oxopropyl) pyrrolidin-1-yl)-2-oxoethyl)-7,10-diisopropyl-5,11-dimethyl-6,9,12-trioxo-2,13-dioxa-5,8,11-triazaheptadec-16-yn-14-yl)-2-nitrophenoxy)-3,4,5-trihydroxytetrahydro-2H-pyran-2-carboxylic acid (44 mg, 39 μmol, 1.0 eq.) and N-(5-azidopentyl)-2-bromoacetamide (12 mg, 50 μmol, 1.3 eq.) gave the title product as a white solid (31 mg, 58%).

To a solution of (2S,3S,4S,5R,6S)-6-(4-((3R,4S,7S,10S)-4-((S)-sec-butyl)-3-(2-((S)-2-((1R,2R)-3-(((1S,2R)-1-hydroxy-1-phenylpropan-2-yl)amino)-1-methoxy-2-methyl-3-oxopropyl) pyrrolidin-1-yl)-2-oxoethyl)-7,10-diisopropyl-5,11-dimethyl-6,9,12-trioxo-2,13-dioxa-5,8,11-triazaheptadec-16-yn-14-yl)-2-nitrophenoxy)-3,4,5-trihydroxytetrahydro-2H-pyran-2-carboxylic acid crude from reaction (SYB-BA2-28 assumed 87 μmol, 1.0 eq.) and N-(5-azidopentyl)-2-bromoacetamide (28.2 mg, 113 μmol, 1.3 eq.) in anhydrous DCM (6 mL) was added tetrakis(acetonitrile) copper (I) hexafluorophosphate (48.7 mg, 131 μmol, 1.5 eq.). The reaction mixture was stirred overnight. DCM was concentrated under reduced pressure, the residue dissolved in DMF (1.5 mL) and treated with a solution of sodium 2,2′-(ethane-1,2-diylbis((carboxymethyl)azanediyl))diacetate dihydrate (128 mg, 0.350 mmol, 4 eq.) in H₂O (1.5 mL) at 0° C. The reaction was stirred for 15 min and the reaction mixture was directly injected to the reverse phase column (40 μm, 15 μm). The crude was purified using ACN (5/95-95/5, 20CV) in 0.1% FA with water. The pure fractions (confirmed by LCMS) were lyophilized to afford the desired compound as a white solid (42 mg, 31 μmmol, 35% over 3 steps).

¹H NMR (400 MHz, DMSO) δ 8.24 (m, 1H), 7.94-7.74 (m, 2H), 7.66-7.52 (m, 1H), 7.32-7.25 (m, 5H), 7.18 (m, 1H), 5.96-5.75 (m, 1H), 5.56-5.15 (m, 5H), 4.84-4.08 (m, 6H), 4.08-3.91 (m, 3H), 3.82 (m, 2H), 3.59 (s, 1H), 3.27-3.11 (m, 8H), 3.02 (m, 4H), 2.96-2.65 (m, 3H), 2.28 (s, 1H), 2.20-1.92 (m, 3H), 1.80-1.72 (m, 5H), 1.61-1.12 (m, 10H), 1.07-0.96 (m, 8H), 0.92-0.73 (m, 18H), 0.72-0.57 (m, 4H), 0.46 (d, J=6.5 Hz, 1H).

LCMS Purity was 99%.

Note: the sequence of MMAE coupling, Ac deprotection and click reaction can be done without reverse phase purification after MMAE coupling or acetyl deprotection. 40 mg of final product with purity >95% (44% over 3 steps) obtained from 60 mg activated benzylic alcohol.

LC-MS (ESI): m/z=1377 [M+H]⁺

### (2S,3S,4S,5R,6S)-6-(4-((3R,4S,7S,10S)-15-(1-(5-(2-bromoacetamido)pentyl)-1H-1,2,3-triazol-4-yl)-4-((S)-sec-butyl)-3-(2-((S)-2-((1R,2R)-3-(((1S,2R)-1-hydroxy-1-phenylpropan-2-yl)amino)-1-methoxy-2-methyl-3-oxopropyl) pyrrolidin-1-yl)-2-oxoethyl)-7,10-diisopropyl-5,11-dimethyl-6,9,12-trioxo-2,13-dioxa-5,8,11-triazapentadecan-14-yl)-2-nitrophenoxy)-3,4,5-trihydroxytetrahydro-2H-pyran-2-carboxylic acid (SYB-BA2-63-SPC07)

### (2S,3S,4S,5R,6S)-6-(4-((3R,4S,7S,10S)-15-(1-(5-(2-bromoacetamido)pentyl)-1H-1,2,3-triazol-4-yl)-4-((S)-sec-butyl)-3-(2-((S)-2-((1R,2R)-3-(((1S,2R)-1-hydroxy-1-phenylpropan-2-yl)amino)-1-methoxy-2-methyl-3-oxopropyl) pyrrolidin-1-yl)-2-oxoethyl)-7,10-diisopropyl-5,11-dimethyl-6,9,12-trioxo-2,13-dioxa-5,8,11-triazapentadecan-14-yl)-2-nitrophenoxy)-3,4,5-trihydroxytetrahydro-2H-pyran-2-carboxylic acid from reactions: (SYB-BA2-06, 31, 35, 37, 44, 53, 55) were combine in ACN/H₂O (1/1: 15/15 mL) and lyophilized for two days to afford (2S,3S,4S,5R,6S)-6-(4-((3R,4S,7S,10S)-15-(1-(5-(2-bromoacetamido) pentyl)-1H-1,2,3-triazol-4-yl)-4-((S)-sec-butyl)-3-(2-((S)-2-((1R,2R)-3-(((1S,2R)-1-hydroxy-1-phenylpropan-2-yl)amino)-1-methoxy-2-methyl-3-oxopropyl) pyrrolidin-1-yl)-2-oxoethyl)-7,10-diisopropyl-5,11-dimethyl-6,9,12-trioxo-2,13-dioxa-5,8,11-triazapentadecan-14-yl)-2-nitrophenoxy)-3,4,5-trihydroxytetrahydro-2H-pyran-2-carboxylic acid 3,4,5-trihydroxytetrahydro-2H-pyran-2-carboxylic acid (507 mg, 368 μmol)

### Example 2—Linker For SPC07

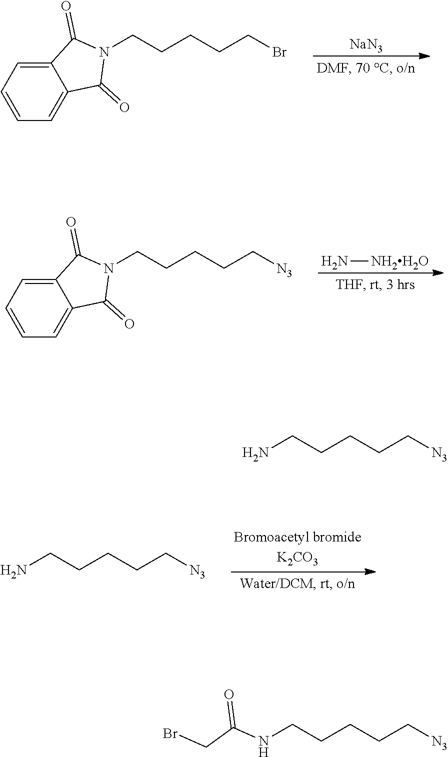

General procedure A for the azide substitution: To a solution of Br compound (1 eq.) in DMF (0.2 M) was added sodium azide (1.5 eq) and the reaction mixture was stirred at 70° C. for 16 h. It was then washed with sat. aq. NaHCO₃ solution and EtOAc. The combined organic layers were dried, filtered, and concentrated. The crude products were used without further purification.

General procedure B for Phthalimide deprotection: To a solution of phthalimide compound (1 eq.) in THF (0.15 M) was added Hydrazine monohydrate (40 eq.). The reaction mixture was stirred at room temperature for 4 hrs. THF was removed under reduced pressure and the residue was washed sat. aq. NaHCO₃ and EtOAc. The combined organic layers were dried, filtered, and concentrated. The crude products were used without further purification

Modified procedure B for Phthalimide deprotection: To a solution of phthalimide compound (1 eq.) in THF (0.2 M) was added Hydrazine monohydrate (3 eq.). The reaction mixture was stirred at room temperature overnight. The phthalic hydrazide was removed by filtration and washed with THF. The solvent was removed under reduced pressure to afford the desired amine. The crude products were used without further purification.

General procedure D for bromoacetylbromide substitution: To a solution of amine (1 eq.) in DCM (0.20 M) was added K₂CO₃ (1.5 eq.) in water (0.5 mL). Then 2-bromoacetylbromide (1.1 eq.) was added dropwise at 0° C. The reaction mixture was stirred at room temperature for 15 hrs. It was then washed with water and EtOAc. The combined organic layers were dried, filtered, and concentrated and purified by column chromatography.

2-(5-azidopentyl) isoindoline-1,3-dione (SYB-BA2-19) Following the general procedure A for azide substitution, A solution of 2-(5-bromopentyl) isoindoline-1,3-dione (3.00 g, 10.1 mmol, 1 eq.) and sodium azide (988 mg, 15.2 mmol, 1.5 eq.) in dimethyl formamide (20 mL, 0.5M) was stirred at 70° C. overnight. The reaction was cooled to room temperature, diluted with brine (100 mL) and extracted three times with ethyl acetate (3×50 mL). The combined organic layers were washed three times with water, dried over Na₂SO₄, filtered and concentrated. to afford 2-(5-azidopentyl) isoindoline-1,3-dione (2.58 g, 10.0 mmol, 98% yield).

1H NMR (400 MHz, CDCl₃) δ 7.91-7.78 (m, 2H), 7.76-7.65 (m, 2H), 3.78-3.62 (m, 2H), 3.27 (t, J=6.9 Hz, 2H), 1.78-1.60 (m, 4H), 1.50-1.37 (m, 2H).

5-azidopentan-1-amine (SYB-BA2-38) Following the general procedure B for phthalimide deprotection, To a solution of 2-(5-azidopentyl) isoindoline-1,3-dione (200 mg, 0.770 mmol, 1 eq.) in THF (3 mL) was added hydrazinehydrate (0.110 mL, 2.32 mmol, 3 eq.) and the reaction was stirred at room temperature overnight. The precipitate was filtered off, washed with THF (1×10 mL) and the filtrate was concentrated under reduced pressure to afford 5-azidopentan-1-amine (85 mg, 0.66 mmol, 86% yield)

1H NMR (400 MHz, CDCl₃) δ 3.27 (t, J=6.9 Hz, 2H), 2.70 (t, J=6.7 Hz, 2H), 1.62 (p, J=7.0 Hz, 2H), 1.53-1.35 (m, 4H).

N-(5-azidopentyl)-2-bromoacetamide (SYB-BA2-18), Following the general procedure D for bromoacetylbromide substitution, To a solution of 5-azidopentan-1-amine (500 mg, 3.9 mmol, 1 eq.) in DCM (20 mL) was added potassium carbonate (1.10 g, 7.8 mmol, 2 eq.), and 2-bromoacetylbromide (0.38 mL, 4.3 mmoL, 1.1 eq.) dropwise at 0° C. The reaction was stirred over night It was washed with water and EtOAc. The combined organic layers were dried over sodium sulfate, filtered and concentrated.
Purification of the residue by MPLC (SiO2) (20 g, 20 CV) (CyH:EtOAc: 100:0 to 40:60) afforded N-(5-azidopentyl)-2-bromoacetamide (639 mg, 2.57 mmol, 66%)

### Analytical Data

TLC [SiO2] (CyH:EtOAc: 50:50)
R_{f}=0.40 (UV, KMnO4)

¹H NMR (400 MHz, CDCl₃) δ 6.51 (s, 1H), 3.88 (s, 2H), 3.34-3.23 (m, 4H), 1.68-1.54 (m, 4H), 1.47-1.37 (m, 2H).

### Example 3—Synthesis Of SPC17

### (2S,3R,4S,5S,6S)-2-(4-formyl-2-nitrophenoxy)-6-(methoxycarbonyl)tetrahydro-2H-pyran-3,4,5-triyl triacetate SYB-BA2-066

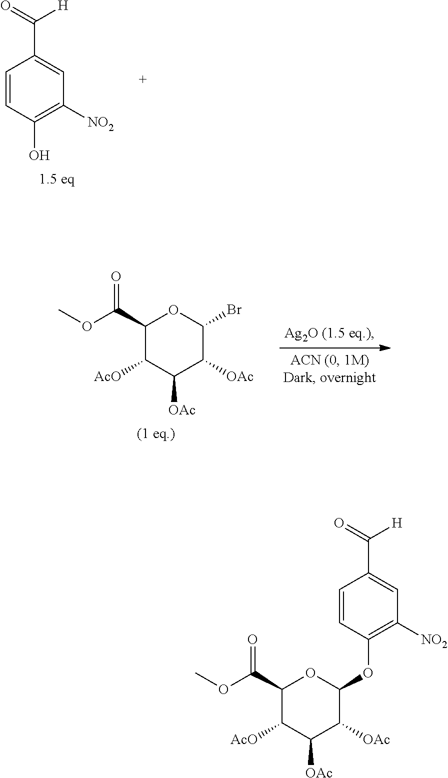 [SMILES: O=Cc1ccc(O)c([N+](=O)[O-])c1 ; COC(=O)[C@H]1O[C@H](Br)[C@H](OC(C)=O)[C@@H](OC(C)=O)[C@@H]1OC(C)=O ; COC(=O)[C@H]1O[C@@H](Oc2ccc(C=O)cc2[N+](=O)[O-])[C@H](OC(C)=O)[C@@H](OC(C)=O)[C@@H]1OC(C)=O]

To a solution of (2R,3R,4S,5S,6S)-2-bromo-6-(methoxycarbonyl)tetrahydro-2H-pyran-3,4,5-triyl triacetate (100 mg, 0.252 mmol, 1.0 eq.) in dry MeCN (2.5 mL, 0.1 M) was added 4-hydroxy-3-nitrobenzaldehyde (63 mg, 0.378 mmol, 1.5 eq.) and silver oxide (87 mg, 0.38 mmol, 1.5 eq.) at room temperature under a nitrogen atmosphere. The resulting reaction mixture was stirred in the dark at room temperature for 17 h. TLC showed full conversion of the donor (cHex/EtOAc 1:1, KMnO₄). The solution was filtered through Celite to remove solids and the filtrate was concentrated under reduced pressure. The residue was diluted with EtOAc (40 ml) and washed with saturated NaHCO₃ (6×20 ml), water and brine. The organic layer was dried over MgSO₄, filtered and concentrated under reduced pressure to afford (2S,3R,4S,5S,6S)-2-(4-formyl-2-nitrophenoxy)-6-(methoxycarbonyl)tetrahydro-2H-pyran-3,4,5-triyl triacetate (109 mg, 0.225 mmol, 89%) as a white solid.

¹H NMR (400 MHz, CDCl₃) δ 9.96 (s, 1H), 8.29 (d, J=2.0 Hz, 1H), 8.07 (dd, J=8.6, 2.0 Hz, 1H), 7.50 (d, J=8.6 Hz, 1H), 5.45-5.37 (m, 2H), 5.35-5.24 (m, 2H), 4.33 (d, J=8.4 Hz, 1H), 3.69 (s, 3H), 2.11 (s, 3H), 2.06 (s, 3H), 2.05 (s, 3H).

### (2S,3R,4S,5S,6S)-2-(4-(1-hydroxybut-3-yn-1-yl)-2-nitrophenoxy)-6-(methoxycarbonyl)-tetrahydro-2H-pyran-3,4,5-triyl triacetate SYB-BA1-060

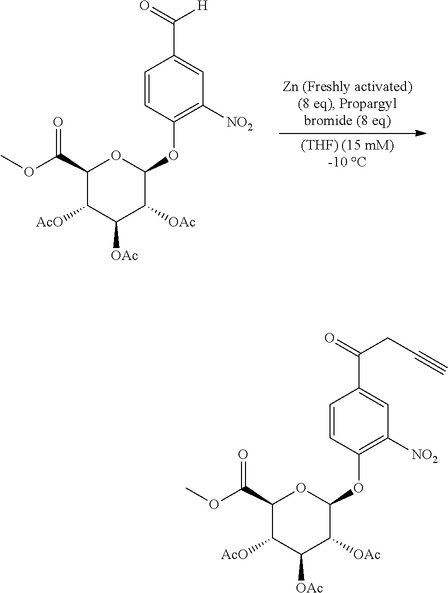 [SMILES: COC(=O)[C@H]1O[C@@H](Oc2ccc(C=O)cc2[N+](=O)[O-])[C@H](OC(C)=O)[C@@H](OC(C)=O)[C@@H]1OC(C)=O ; C#CCC(=O)c1ccc(O[C@@H]2O[C@H](C(=O)OC)[C@@H](OC(C)=O)[C@H](OC(C)=O)[C@H]2OC(C)=O)c([N+](=O)[O-])c1]

To a suspension of freshly activated zinc powder (HCl, H₂O, MeOH, Et₂O, high vacuum dry, 54 mg, 0.83 mmol, 8.0 eq.) in dry THF (2 mL, 0.4 M) was added allyl bromide (123 mg, 0.825 mmol, 8.0 eq.) at 0° C. under a nitrogen atmosphere. The reaction was warmed to room temperature and stirred for 1 h until all zinc was dissolved. The reaction solution was cooled to −15° C. and a solution of (2S,3R,4S,5S,6S)-2-(4-formyl-2-nitrophenoxy)-6-(methoxycarbonyl)tetrahydro-2H-pyran-3,4,5-triyl triacetate (50 mg, 0.103 mmol, 1.0 eq.) in THF (1 mL) was slowly added. The reaction mixture was stirred for 30 min at −15° C. and quenched by addition of a sat. aq. NH4Cl solution (20 mL). The aqueous solution was extracted with EtOAc (3*20 mL). The combined organic layers were washed with H₂O (30 mL), dried over Na₂SO₄, filtered and concentrated under reduced pressure. The crude product was purified by silica gel column chromatography (12 g, 20 CV, CyH:EtOAc: 95:0 to 20:80) to afford (2S,3R,4S,5S,6S)-2-(4-(1-hydroxybut-3-yn-1-yl)-2-nitrophenoxy)-6-(methoxycarbonyl)-tetrahydro-2H-pyran-3,4,5-triyl triacetate (14 mg, 1.16 mmol, 53%).

¹H NMR (400 MHz, CDCl₃) δ 7.87 (ddd, J=5.5, 2.2, 0.6 Hz, 1H), 7.58 (dddd, J=8.6, 6.3, 2.2, 0.6 Hz, 1H), 7.37 (d, J=8.6 Hz, 1H), 5.41-5.25 (m, 3H), 5.24-5.17 (m, 1H), 4.91 (t, J=6.2 Hz, 1H), 4.24-4.18 (m, 1H), 3.74 (s, 3H), 2.70-2.57 (m, 2H), 2.13 (s, 3H), 2.11 (t, J=2.6 Hz, 1H), 2.07 (s, 3H), 2.05 (s, 3H), 2.04 (d, J=3.8 Hz, 1H), (2S,3S,4S,5R,6S)-2-(methoxycarbonyl)-6-(2-nitro-4-(1-(((4-nitrophenoxy) carbonyl)oxy) but-3-yn-1-yl) phenoxy)tetrahydro-2H-pyran-3,4,5-triyl triacetate SYB-BA1-94 or SYB-BA2-23

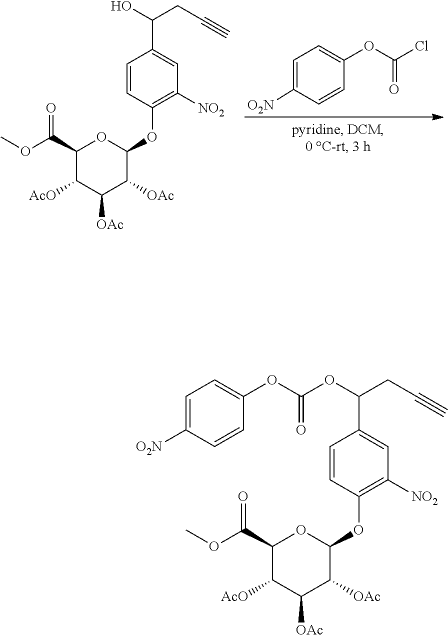 [SMILES: C#CCC(O)c1ccc(O[C@@H]2O[C@H](C(=O)OC)[C@@H](OC(C)=O)[C@H](OC(C)=O)[C@H]2OC(C)=O)c([N+](=O)[O-])c1 ; O=C(Cl)Oc1ccc([N+](=O)[O-])cc1 ; C#CCC(OC(=O)Oc1ccc([N+](=O)[O-])cc1)c1ccc(O[C@@H]2O[C@H](C(=O)OC)[C@@H](OC(C)=O)[C@H](OC(C)=O)[C@H]2OC(C)=O)c([N+](=O)[O-])c1]

A solution of (2S,3R,4S,5S,6S)-2-(4-(1-hydroxybut-3-yn-1-yl)-2-nitrophenoxy)-6-(methoxycarbonyl)-tetrahydro-2H-pyran-3,4,5-triyl triacetate SYB-BA1-060 (375 mg, 0.71 mmol, 1.0 eq.), 4-Nitrophenyl chloroformate (361 mg, 1.79 mmol, 2.5 eq.) in dichloromethane (8 mL, 0.1 M) at 0° C. was added pyridine (580 μL, 7.16 mmol, 10 eq.), and the reaction was stirred for 4 hours at room temperature. The reaction was diluted with EtOAc (100 mL) and washed with aq. sat. NaHCO₃. The aqueous layer was extracted three times with EtOAc. The combined organic layers were dried, filtered and concentrated under reduced pressure. The residue was purified by silica gel column chromatography (CyH/EtOAc 5 to 60%) to afford (2S,3S,4S,5R,6S)-2-(methoxycarbonyl)-6-(2-nitro-4-(1-(((4-nitrophenoxy) carbonyl)oxy) but-3-yn-1-yl) phenoxy)tetrahydro-2H-pyran-3,4,5-triyl triacetate (410 mg, 0.590 mmol, 83%).

R_{f}=0.30 product (KMnO₄)

¹H NMR (400 MHz, CDCl₃) δ 8.35-8.27 (m, 2H), 7.96 (d, J=2.2 Hz, 1H), 7.67 (dd, J=8.5, 2.3 Hz, 1H), 7.47-7.37 (m, 3H), 5.83 (t, J=6.5 Hz, 1H), 5.47-5.22 (m, 4H), 4.26 (d, J=8.8 Hz, 1H), 3.76 (s, 3H), 3.06-2.85 (m, 2H), 2.15 (s, 3H), 2.12 (t, J=2.6 Hz, 1H), 2.10 (s, 3H), 2.06 (s, 3H).

### (2S,3R,4S,5S,6S)-2-(4-((3R,4S,7S,10S)-4-((S)-sec-butyl)-3-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-7,10-diisopropyl-5,11-dimethyl-6,9,12-trioxo-2,13-dioxa-5,8,11-triazaheptadec-16-yn-14-yl)-2-nitrophenoxy)-6-(methoxycarbonyl)tetrahydro-2H-pyran-3,4,5-triyl triacetate SYB-BA1-029 or SYB-BA2-026

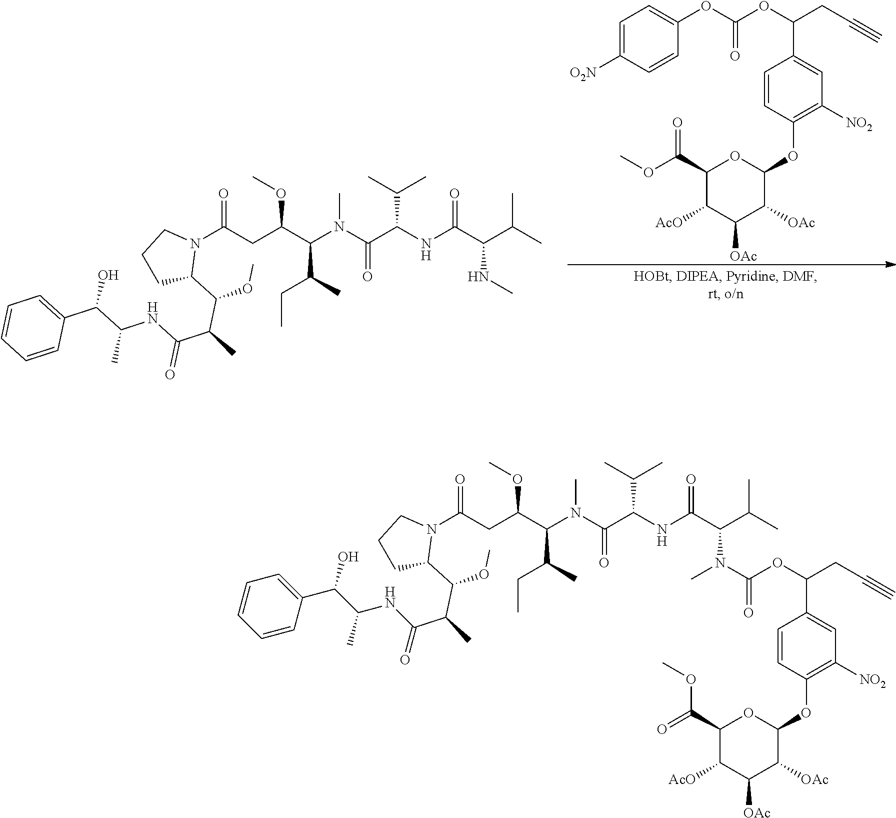

To a solution of (2S,3S,4S,5R,6S)-2-(methoxycarbonyl)-6-(2-nitro-4-(1-(((4-nitrophenoxy) carbonyl)oxy) but-3-yn-1-yl) phenoxy)tetrahydro-2H-pyran-3,4,5-triyl triacetate (110 mg, 0.16 mmol, 1 eq.), Monomethylauristatin E (115 mg, 0.160 mmol, 1.0 eq.) and DIPEA (61 μL, 0.35 mmol, 2.2 eq.) and pyridine (0.13 μL, 0.16 mmol, 1.0 eq.) in DMF (3.2 mL, 0.05 M) was added HOBT (26 mg, 0.17 mmol, 1.05 eq.).

The reaction mixture was directly injected to the reverse phase column and purified by reverse phase column chromatography using 5-70% methanol in 0.1% FA with water to afford (2S,3R,4S,5S,6S)-2-(4-((3R,4S,7S,10S)-4-((S)-sec-butyl)-3-(2-((S)-2-((1R,2R)-3-(((1S,2R)-1-hydroxy-1-phenylpropan-2-yl)amino)-1-methoxy-2-methyl-3-oxopropyl) pyrrolidin-1-yl)-2-oxoethyl)-7,10-diisopropyl-5,11-dimethyl-6,9,12-trioxo-2,13-dioxa-5,8,11-triazaheptadec-16-yn-14-yl)-2-nitrophenoxy)-6-(methoxycarbonyl)tetrahydro-2H-pyran-3,4,5-triyl triacetate (216 mg, 0.170 mmol, 60%) as a white solid.

¹H NMR (400 MHz, MeOD) δ 7.98-7.57 (m, 2H), 7.56-7.13 (m, 6H), 5.89-5.73 (m, 1H), 5.58-5.36 (m, 2H), 5.23 (q, J=8.6 Hz, 2H), 4.71 (d, J=8.7 Hz, 1H), 4.65-4.46 (m, 3H), 4.33-4.01 (m, 3H), 3.80-3.63 (m, 4H), 3.46-3.33 (m, 9H), 3.29-3.23 (m, 3H), 3.16-2.75 (m, 6H), 2.55-2.30 (m, 3H), 2.30-1.99 (m, 11H), 1.98-1.75 (m, 3H), 1.59 (s, 1H), 1.41 (s, 1H), 1.23-1.08 (m, 6H), 0.99-0.83 (m, 17H), 0.69-0.48 (m, 2H).

LC-MS (ESI): m/z=1268 [M+H]⁺

### (2S,3S,4S,5R,6S)-6-(4-((3R,4S,7S,10S)-4-((S)-sec-butyl)-3-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-7,10-diisopropyl-5,11-dimethyl-6,9,12-trioxo-2,13-dioxa-5,8,11-triazaheptadec-16-yn-14-yl)-2-nitrophenoxy)-3,4,5-trihydroxytetrahydro-2H-pyran-2-carboxylic acid SYB-BA1-34

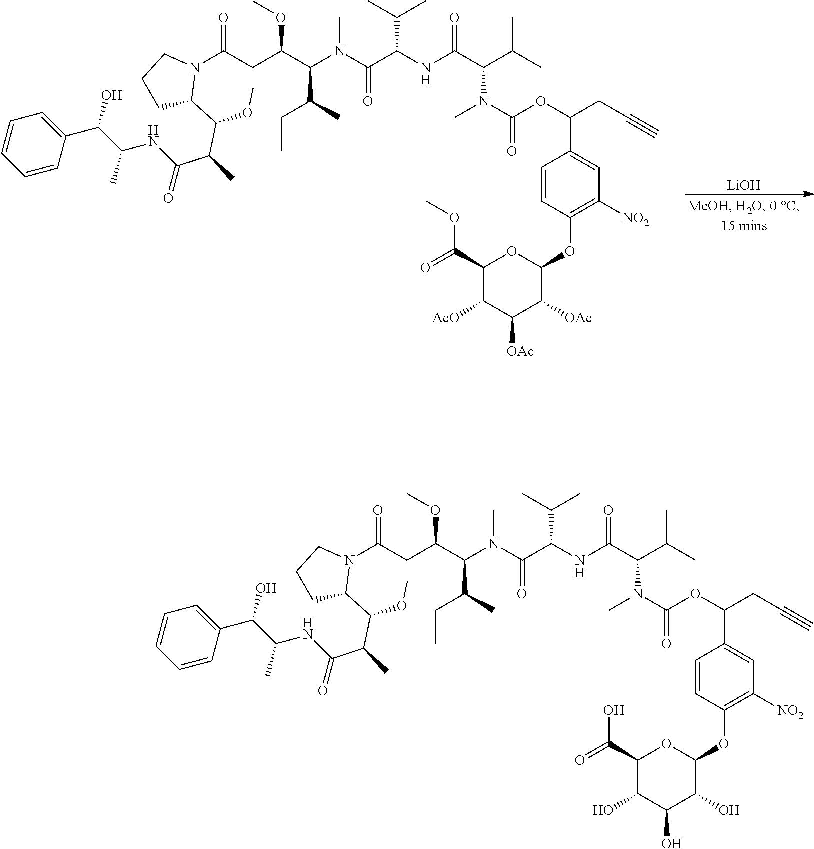 [SMILES: C#CCC(OC(=O)N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C)c1ccc(O[C@@H]2O[C@H](C(=O)OC)[C@@H](OC(C)=O)[C@H](OC(C)=O)[C@H]2OC(C)=O)c([N+](=O)[O-])c1 ; "LiOH" ; C#CCC(OC(=O)N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C)c1ccc(O[C@@H]2O[C@H](C(=O)O)[C@@H](O)[C@H](O)[C@H]2O)c([N+](=O)[O-])c1]

To a solution of (2S,3R,4S,5S,6S)-2-(4-((3R,4S,7S,10S)-4-((S)-sec-butyl)-3-(2-((S)-2-((1R,2R)-3-(((1S,2R)-1-hydroxy-1-phenylpropan-2-yl)amino)-1-methoxy-2-methyl-3-oxopropyl) pyrrolidin-1-yl)-2-oxoethyl)-7,10-diisopropyl-5,11-dimethyl-6,9,12-trioxo-2,13-dioxa-5,8,11-triazaheptadec-16-yn-14-yl)-2-nitrophenoxy)-6-(methoxycarbonyl)tetrahydro-2H-pyran-3,4,5-triyl triacetate SYB-BA1-029 or SYB-BA2-026 (200 mg, 158 μmmol, 1.0 eq.) in MeOH (6 mL, 0.03 M) cooled at 0° C., was added dropwise a cold solution (0° C.) of LiOH·H₂O (58 mg, 1.4 mmol, 8.8 eq.) in H₂O (6 mL). Stirring was continued for 15 minutes at 0° C. (followed by TLC DCM:MeOH 9:1) and the solution was neutralized with Amberlite Weakly acidic Cation exchanger hydrogen form during 15 minutes (pH followed on paper) and filtered. The aqueous layer was freeze dried overnight to afford (2S,3S,4S,5R,6S)-6-(4-((3R,4S,7S,10S)-4-((S)-sec-butyl)-3-(2-((S)-2-((1R,2R)-3-(((1S,2R)-1-hydroxy-1-phenylpropan-2-yl)amino)-1-methoxy-2-methyl-3-oxopropyl) pyrrolidin-1-yl)-2-oxoethyl)-7,10-diisopropyl-5,11-dimethyl-6,9,12-trioxo-2,13-dioxa-5,8,11-triazaheptadec-16-yn-14-yl)-2-nitrophenoxy)-3,4,5-trihydroxytetrahydro-2H-pyran-2-carboxylic acid which contained 8% impurity HPLC (Method ACN-high mass-2000: RT: 3.79 min, m/z=1109 [M+H]⁺). The impure product was purified reverse phase column chromatography (40 g, 15 μm) using MeOH in 0.1% FA with water to afford (2S,3S,4S,5R,6S)-6-(4-((3R,4S,7S,10S)-4-((S)-sec-butyl)-3-(2-((S)-2-((1R,2R)-3-(((1S,2R)-1-hydroxy-1-phenylpropan-2-yl)amino)-1-methoxy-2-methyl-3-oxopropyl) pyrrolidin-1-yl)-2-oxoethyl)-7,10-diisopropyl-5,11-dimethyl-6,9,12-trioxo-2,13-dioxa-5,8,11-triazaheptadec-16-yn-14-yl)-2-nitrophenoxy)-3,4,5-trihydroxytetrahydro-2H-pyran-2-carboxylic acid (89 mg, 79 μmmol, 50%, containing less than 3% impurity) and (56 mg, 50 μmmol, 31%, containing less than 10% impurity).

Purification at this stage is not necessary if the impurity is below 15%, this impurity will be removed during the reverse phase purification of the final click reaction.

¹H NMR (400 MHz, MeOD) δ 7.97-7.85 (m, 1H), 7.69 (m 1H), 7.50-7.17 (m, 6H), 5.80 (d, J=9.6 Hz, 1H), 5.28-5.07 (m, 1H), 4.75-4.42 (m, 2H), 4.39-3.43 (m, 10H), 3.35 (m, 6H), 3.30-3.22 (m, 5H), 3.14-2.69 (m, 6H), 2.62-2.07 (m, 5H), 2.05-1.51 (m, 5H), 1.41 (s, 2H), 1.31-0.26 (m, 26H).

LC-MS (ESI): m/z=1128 [M+H]⁺ LC-MS (ESI): m/z=1128 [M+H]⁺

### General Procedure for the Click Reaction

To a solution of alkyne derivative (1.0 eq.) in DCM (0.05 M) was added the corresponding azide (1.3 equiv.) followed by the addition of tetrakis(acetonitrile) copper (i) hexafluoro phosphate (1.5 equiv). The solution was stirred at room temperature and the reaction was monitored by LCMS the desired mass was found. After 4 hours (SM was consumed confirmed by LC-MS), the reaction mixture was concentrated under reduced pressure to remove the DCM. Then the crude was diluted with DMF and a solution ethylenediaminetetraacetic acid disodium salt dihydrate (EDTA·2Na·2·H₂O) (4 eq.) in water was added at 0° C. and stirring at room temperature was continued for 15 minutes. Then the reaction mixture was directly injected to the reverse phase column (25 μm) and the crude was purified using ACN in 0.1% FA in water. The pure fractions (confirmed by LCMS) was lyophilized to afford the desired compound as a white solid.

### (2S,3S,4S,5R,6S)-6-(4-((3R,4S,7S,10S)-15-(1-(1-bromo-2-oxo-6,9,12,15,18,21,24,27-octaoxa-3-azanonacosan-29-yl)-1H-1,2,3-triazol-4-yl)-4-((S)-sec-butyl)-3-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-7,10-diisopropyl-5,11-dimethyl-6,9,12-trioxo-2,13-dioxa-5,8,11-triazapentadecan-14-yl)-2-nitrophenoxy)-3,4,5-trihydroxytetrahydro-2H-pyran-2-carboxylic acid SYB-BA1-111 or SPC17

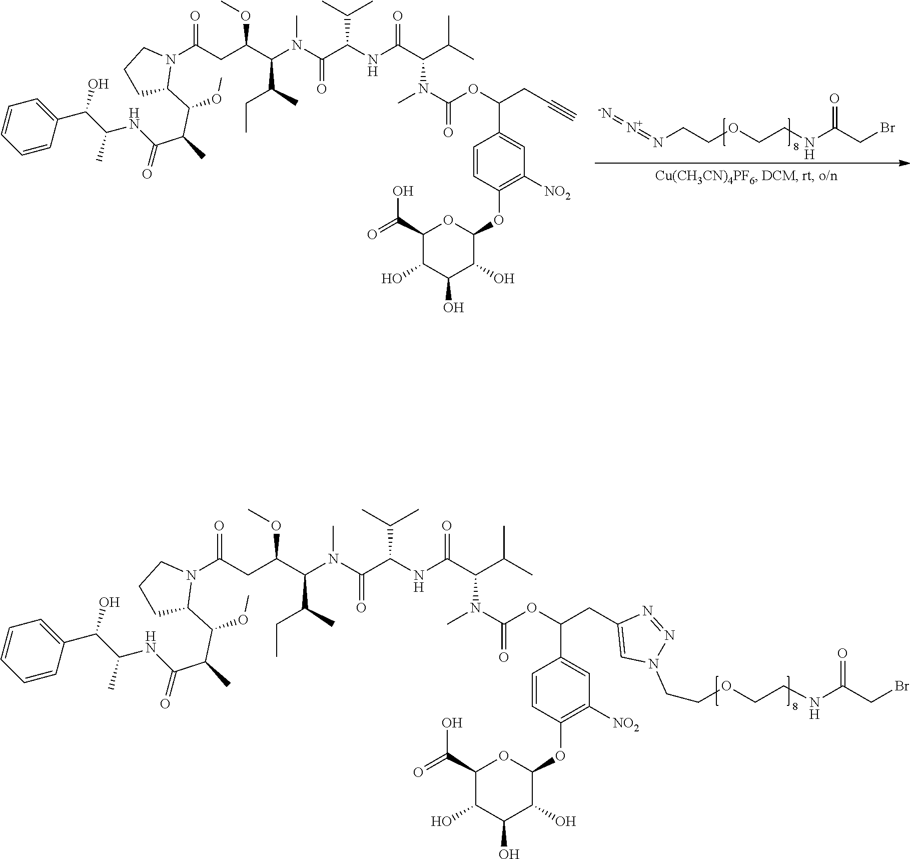

Following the general procedure for the Click reaction (2S,3S,4S,5R,6S)-6-(4-((3R,4S,7S,10S)-4-((S)-sec-butyl)-3-(2-((S)-2-((1R,2R)-3-(((1S,2R)-1-hydroxy-1-phenylpropan-2-yl)amino)-1-methoxy-2-methyl-3-oxopropyl) pyrrolidin-1-yl)-2-oxoethyl)-7,10-diisopropyl-5,11-dimethyl-6,9,12-trioxo-2,13-dioxa-5,8,11-triazaheptadec-16-yn-14-yl)-2-nitrophenoxy)-3,4,5-trihydroxytetrahydro-2H-pyran-2-carboxylic acid (40 mg, 35 μmol, 1 eq.) and N-(26-azido-3,6,9,12,15,18,21,24-octaoxahexacosyl)-2-bromoacetamide (25 mg, 46 μmol, 1.3 eq.) gave the title product as a white solid (9.7 mg, 5.8 μmmol, 16%).

¹H NMR (400 MHz, DMSO) δ 8.36-8.32 (m, 1H), 7.92-7.84 (m, 1H), 7.75-7.69 (m, 1H), 7.40-7.20 (m, 6H), 7.21-7.15 (m, 1H), 5.43-5.33 (m, 3H), 5.26-5.18 (m, 2H), 4.55-4.28 (m, 6H), 4.10-3.89 (m, 2H), 3.86 (s, 2H), 3.76 (s, 3H), 3.71-3.31 (m, 36H), 3.27-3.21 (m, 10H), 3.06-2.79 (m, 1H), 2.78-2.72 (m, 1H), 2.44-1.90 (m, 2H), 1.72-1.62 (m, 1H), 1.35-1.15 (m, 3H), 1.10-0.94 (m, 10H), 0.93-0.55 (m, 24H), 0.47 (d, J=6.6 Hz, 1H).

LCMS Purity was 96%.

LC-MS (ESI): m/z=1687 [M+H]⁺

### Example 4—Linker for SPC17

### N-(26-azido-3,6,9,12,15,18,21,24-octaoxahexacosyl)-2-bromoacetamide SSA-BA1-164

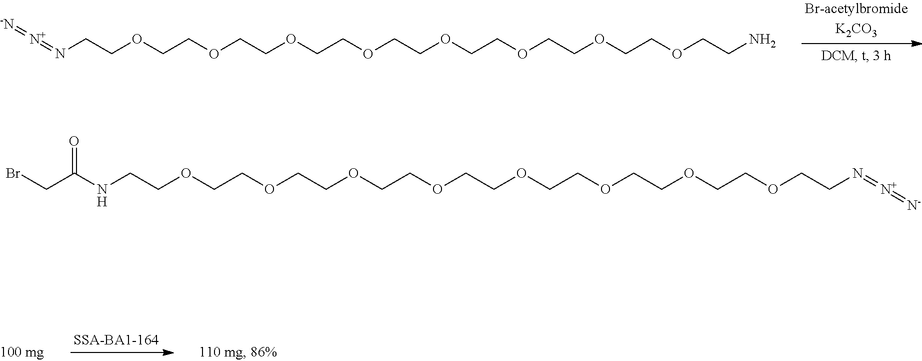

To a solution of 26-azido-3,6,9,12,15,18,21,24-octaoxahexacosan-1-amine (100 mg, 0.228 mmol, 1.0 eq.) in dry DCM (6 mL) were added K₂CO₃ (47 mg, 0.34 mmol, 1.5 eq.) followed by the addition of 2-bromoacetyl bromide (83 mg, 0.41 mmol, 1.8 eq.). The reaction mixture was stirred for 3 h at room temperature. After completion of the reaction, it was diluted with water and extracted twice with EtOAc. The combined organic layers were washed with water, filtered, and concentrated to afford the desired compound as pale-yellow liquid (110 mg, 86%). The crude product was used without further purification. 1H NMR (400 MHz, CDCl₃) δ 3.93 (s, 2H), 3.89 (s, 13H), 3.71-3.62 (m, 24H).

### Example 5—Synthesis Of SPC19

Synthesis steps for SPC19 up to the general procedure of the Click reaction are carried out as set forth above in Examples 1 and 3 for SPC07 and SPC17.

(2S,3S,4S,5R,6S)-6-(4-((3R,4S,7S,10S)-15-(1-(2-(2-bromoacetamido)ethyl)-1H-1,2,3-triazol-4-yl)-4-((S)-sec-butyl)-3-(2-((S)-2-((1R,2R)-3-(((1S,2R)-1-hydroxy-1-phenylpropan-2-yl)amino)-1-methoxy-2-methyl-3-oxopropyl) pyrrolidin-1-yl)-2-oxoethyl)-7,10-diisopropyl-5,11-dimethyl-6,9,12-trioxo-2,13-dioxa-5,8,11-triazapentadecan-14-yl)-2-nitrophenoxy)-3,4,5-trihydroxytetrahydro-2H-pyran-2-carboxylic acid SYB-BA1-132 SPC19

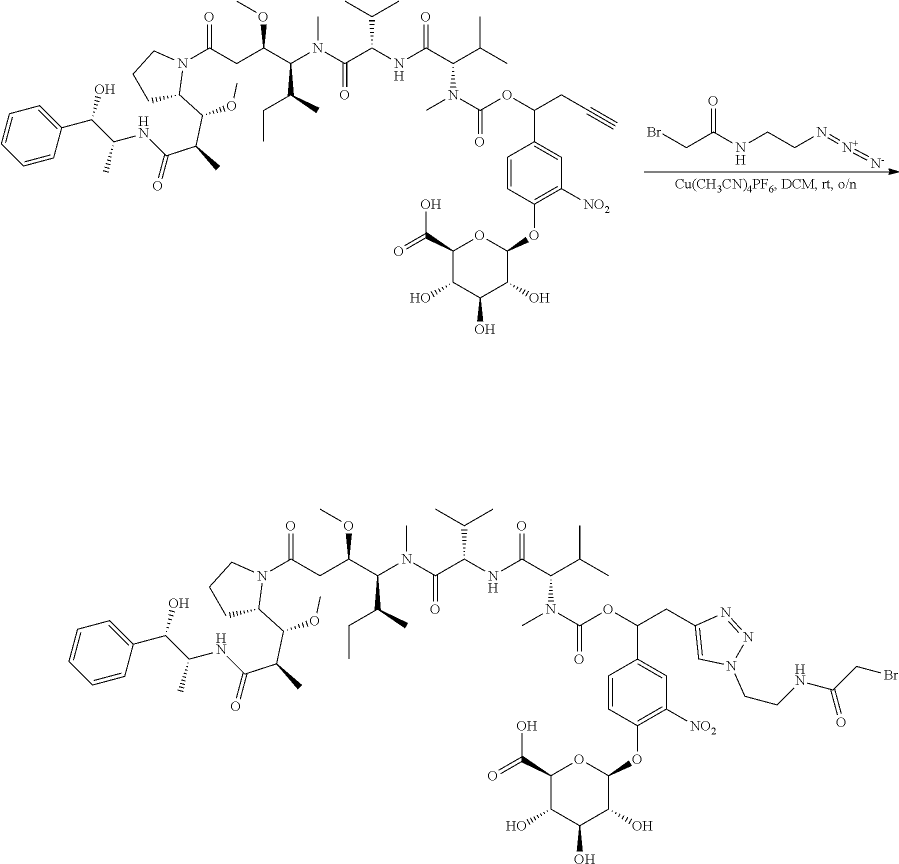

Following the general procedure for the Click reaction (2S,3S,4S,5R,6S)-6-(4-((3R,4S,7S,10S)-4-((S)-sec-butyl)-3-(2-((S)-2-((1R,2R)-3-(((1S,2R)-1-hydroxy-1-phenylpropan-2-yl)amino)-1-methoxy-2-methyl-3-oxopropyl) pyrrolidin-1-yl)-2-oxoethyl)-7,10-diisopropyl-5,11-dimethyl-6,9,12-trioxo-2,13-dioxa-5,8,11-triazaheptadec-16-yn-14-yl)-2-nitrophenoxy)-3,4,5-trihydroxytetrahydro-2H-pyran-2-carboxylic acid (33 mg, 29 μmol, 1.0 eq.) and N-(2-azidoethyl)-2-bromoacetamide (8 mg, 41 μmol, 1.4 eq.) gave the title product as a white solid (22 mg, 16 μmmol, 55%)

¹H NMR (400 MHz, DMSO) δ 8.48-8.42 (m, 1H), 8.18-8.12 (m, 1H), 7.91-7.87 (m, 1H), 7.83-7.69 (m, 1H), 7.42-7.23 (m, 6H), 7.23-7.13 (m, 1H), 6.12-5.68 (m, 1H), 5.50-5.31 (m, 2H), 5.29-5.15 (m, 2H), 4.55-4.27 (m, 5H), 4.24-3.68 (m, 7H), 3.54 (m, 7H), 3.30-3.11 (m, 12H), 3.10-2.63 (m, 6H), 2.46-1.90 (m, 4H), 1.87-1.41 (m, 4H), 1.11-0.95 (m, 8H), 0.94-0.56 (m, 19H), 0.48 (d, J=6.5 Hz, 1H).

LCMS Purity was 99%

LC-MS (ESI): m/z=1335 [M+H]⁺

### Example 6—Linker For SPC19

### 2-(2-azidoethyl) isoindoline-1,3-dione SSA-BA1-127

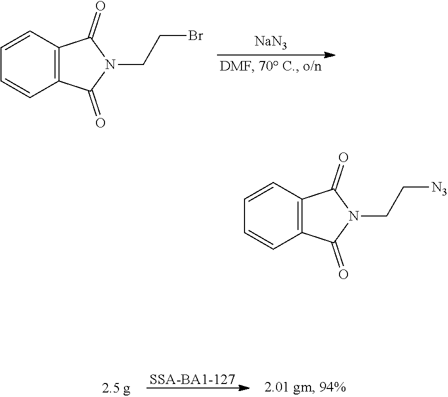 [SMILES: O=C1c2ccccc2C(=O)N1CCBr ; "DMF, 70° C., o/n" ; [N-]=[N+]=NCCN1C(=O)c2ccccc2C1=O ; "SSA-BA1-127" ; "NaN3"]

To a stirred solution of 2-(2-bromoethyl) isoindoline-1,3-dione (2.5 g, 9.8 mmol, 1.0 eq.) in DMF (49 mL, 0.2 M) was added sodium azide (0.96 g, 1.5 eq., 15 mmol) and the resultant reaction mixture was stirred at 70° C. overnight. After completion of the reaction, aq. 10% LiCl solution was added to it and extracted twice with EtOAc. The combined organic layers were washed with water, filtered, and concentrated to afford the desired compound as off-white solid (2.01 g, 94%). The crude product was used without further purification.

¹H NMR (500 MHz, CDCl₃) δ 7.90-7.83 (m, 2H), 7.76-7.71 (m, 2H), 3.90 (t, J=6.1 Hz, 2H), 3.59 (t, J=6.1 Hz, 2H).

### 2-azidoethan-1-amine SSA-BA1-131

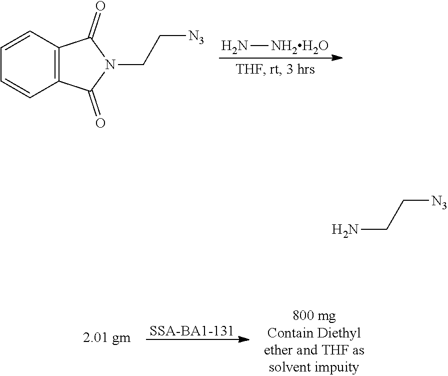

To a solution of 2-(2-azidoethyl) isoindoline-1,3-dione (2.01 g, 9.30 mmol, 1 eq.) in THF (46.5 mL, 0.2 molar) was added hydrazinehydrate (18.6 g, 18.1 mL, 372 mmol, 40 eq.) and the resultant reaction mixture was stirred at room temperature for 4 hours. After completion of the reaction, THF was removed (staying above 250 mbar) and the residue was washed 10% LiCl and extracted with di ethyl ether. The combined organic layers were dried, filtered, and concentrated (staying above 250 mbar) to afford the desired compound as colorless oil (800 mg, Crude, Contain THF and diethyl ether as solvent impurity).

Due to the volatile nature of the compound, it was difficult to remove the residual solvent and was used in the next step.

### N-(2-azidoethyl)-2-bromoacetamide SSA-BA1-134

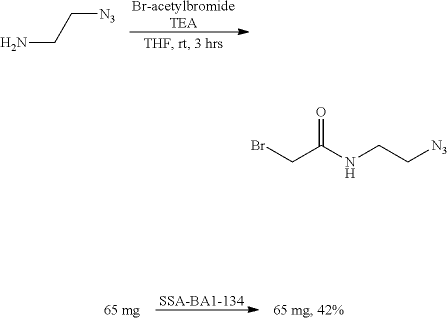 [SMILES: [N-]=[N+]=NCCN ; "Br-acetylbromideTEA" ; "THF, rt, 3 hrs" ; [N-]=[N+]=NCCNC(=O)CBr ; "SSA-BA1-134"]

A solution of 2-Bromoacetylbromide (0.15 mL, 1.5 mmol, 2.2 eq.) in dry THF (3 mL) was added dropwise to the mixture of of 2-azidoethan-1-amine (65 mg, 0.75 mmol, 1.0 eq.) and TEA (0.24 mL, 1.7 mmol, 2.5 eq.) in dry THF (4.5 mL, 0.2 M) at 0° C. The reaction mixture was allowed to stir for 1.3 h at room temperature. The excess of acid bromide was quenched by the addition of methanol (1 mL). The formed triethylammonium bromide salt was filtered off and the solvent was removed under reduced pressure. The crude product was dissolved in dichloromethane and washed three times with a saturated ammonium chloride solution and two times with distilled water. The combined organic layers were dried, filtered, and concentrated to afford a pale brown oil crude. The residue was purified by MPLC (SiO2, CyH/EtOAc 0 to 45%) to afford the desired compound as pale-yellow oil (65 mg, 42%).

¹H NMR (400 MHz, CDCl₃) δ 6.76 (s, 1H), 3.90 (s, 2H), 3.48 (d, J=4.1 Hz, 4H).

### Example 7—Large Scale Conjugation to Antibodies

Dilute TCEP to 5 mM: 5ul of 0.5M TCEP into 495 ul of SPC EDTA buffer (50 mM Tris pH8.5, 150 mM NaCl, 20 mM EDTA

Perform conjugation in 1.7 ml tube
1. Aliquot 97ul of SPC EDTA buffer
2. Aliquot 847ul of antibody at 5.9 mg/ml
3. Slowly mix in 22ul TCEP
4. Place tube in shaker at 25C, 300 rpm for 1 hour
5. Place on ice for 2 min
6. Slowly mix in 33.3ul linker payload (SPC004, SPC007, or SPC008)
7. Place tube in shaker at 25C, 300 rpm for 2 hours
8. Place on ice.

TABLE 2
GROUPS IN EXAMPLES 8 TO 18

| | | Heavy | Light |
|---|---|---|---|
| | | Chain | Chain |
| | | Variable | Variable |
| Groups | | Region | Region |
| Vehicle | control | | |
| BA-143-00-MC | Wildtype or parent | SEQ ID | SEQ ID |
| | Non-CAB conjugated | NO: 18 | NO: 31 |
| | to MC linker | | |
| BA-143-00-SPC04 | Wildtype or parent | SEQ ID | SEQ ID |
| | Non-CAB conjugated | NO: 18 | NO: 31 |
| | to SPC04 | | |
| BA-143-00-SPC07 | Wildtype or parent | SEQ ID | SEQ ID |
| | Non-CAB conjugated | NO: 18 | NO: 31 |
| | to linker compound | | |
| | SPC07 | | |
| BA-143-00-SPC08 | Wildtype or parent | SEQ ID | SEQ ID |
| | Non-CAB conjugated | NO: 18 | NO: 31 |
| | to SPC08 | | |
| BA-143-01-MC | CAB conjugated to | SEQ ID | SEQ ID |
| | control thalidomide | NO: 19 | NO: 32 |
| | linker | | |
| BA-143-01-SPC04 | CAB conjugated to | SEQ ID | SEQ ID |
| | SPC04 | NO: 19 | NO: 32 |
| BA-143-01-SPC07 | CAB conjugated to | SEQ ID | SEQ ID |
| | SPC07 | NO: 19 | NO: 32 |
| BA-143-01-SPC08 | CAB conjugated to | SEQ ID | SEQ ID |
| | SPC08 | NO: 19 | NO: 32 |

The sequences of the heavy chain and light chain variable regions are shown in FIGS. 2A-2B.

The structure of BA-143-00-MC (wildtype or parent non-CAB conjugated to MC):

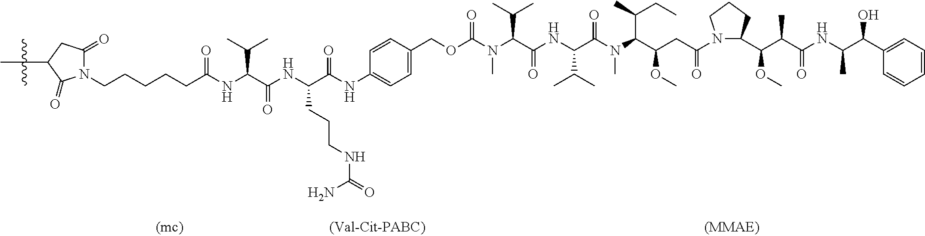 [SMILES: *C1CC(=O)N(CCCCCC(=O)N[C@H](C(=O)N[C@@H](CCCNC(N)=O)C(=O)Nc2ccc(COC(=O)N(C)[C@H](C(=O)N[C@H](C(=O)N(C)[C@@H]([C@@H](C)CC)[C@@H](CC(=O)N3CCC[C@H]3[C@H](OC)[C@@H](C)C(=O)N[C@H](C)[C@@H](O)c3ccccc3)OC)C(C)C)C(C)C)cc2)C(C)C)C1=O]

### Abbreviations For Examples 8 to 18

| | |
|---|---|
| ADC | antibody-drug conjugate |
| ° C. | degree Celsius |
| Af488 | Alexa Fluor 488 |
| C. | Celsius |
| BSA | bovine serum albumin |
| DAR | drug antibody ratio |
| DMEM | Dulbecco's Modified Eagle Medium |
| ECD | Extracellular Domain |
| ELISA | enzyme-linked immunosorbent assay |
| FACS | fluorescence-activated cell sorting |
| FBS | fetal bovine serum |
| g | gram |
| HCl | hydrochloric acid |
| hr | hour |
| HRP | horseradish peroxidase |
| huNectin 4 | human Nectin 4 extracellular domain |
| cynoNectin4 | cynomolgus Nectin 4 extracellular domain |
| ratNectin4 | rat Nectin4 extracellular domain |
| IC50 | 50% effective inhibitory concentration |
| Log | logarithm |
| MEM | minimum essential media |
| MFI | median fluorescence intensity |
| min | minute |
| mL | milliliter |
| MMAE | Monomethyl auristatin E |
| NA | not applicable |
| NaOH | sodium hydroxide |
| NEAA | non-essential amino acid |
| ng | nanogram |
| nm | nanometers |
| OD | optical density |
| PBS | phosphate buffered saline |
| PE | phycoerthrin |
| PK | Pharmacokinetics |
| RCF | relative centrifugal force |
| RLU | relative luminescence units |
| rpm | revolutions per minute |
| R.T. | room temperature |
| SD | standard deviation |
| TMB | 3,3′,5,5′ tetramethylbenzidine |
| μg | microgram |
| μL | microliter |
| T_{1/2} | the time required for concentration to decrease by 50% |
| Cₘₐₓ | maximum serum concentration after dosing |

### Example 8—PK Analysis Of ADCS With Affinity ELISA

### 1.1 Test Articles

Mouse serum samples were collected at 10 mins, 6 hrs, 24 hrs, 48 hrs, 96 hrs and 168 hrs and 96 hrs post the following antibody treatments (3 mg/kg) in in vivo PK study.
BA-143-00-MC (wildtype or parent non-CAB conjugated to MC)
BA-143-00-SPC04 (wildtype or parent non-CAB conjugated to SPC04)
BA-143-00-SPC07 (wildtype or parent non-CAB conjugated to SPC07)
BA-143-00-SPC08 (wildtype or parent non-CAB conjugated to SPC08)
BA-143-01-MC (CAB conjugated to MC)
BA-143-01-SPC04 (CAB conjugated to SPC04)
BA-143-01-SPC07 (CAB conjugated to SPC07)
BA-143-01-SPC08 (CAB conjugated to SPC08)

### 1.2 Formulations

Test articles were diluted in assay buffer (PBS with 1% BSA, pH6.0) by different dilution factors depending on the sample collecting time points.

The antibodies from the PK study were used as the standard controls for standard curves, first were diluted to 3 ug/mL using assay buffer with 1:200 normal mouse serum, and then 3-fold serially diluted for 10 concentration points.

### 1.3 pH Affinity ELISA Assay

1) Coat ELISA plates with 100 μL of 2 μg/mL recombinant human Nectin 4 antigen in carbonate-bicarbonate coating buffer.
2) Cover plates with sealing film and incubate overnight at 4° C.
3) Decant plates and tap out residual liquid on a stack of paper towels.
4) Add 200 μL Assay buffer. Shake at 200 RPM for 5 min at room temp
5) Empty plate and tap out residual liquid on paper towels
6) Repeat for total of three times
7) Add 200 μL of assay buffer to the wells. Cover with sealing film and place the plate onto a plate shaker set to 200 rpm for 60 minutes at room temperature.
8) Decant plates and tap out residual liquid on a stack of paper towels.
9) Dilute test articles and standard control antibodies and add 100 μL/well to the plates
10) Cover with sealing film and place the plates onto a plate shaker set to 200 rpm for 60 minutes at room temperature.
11) Decant plates and tap out residual liquid on a stack of paper towels.
12) Wash wells four times by dispensing 200 μL of wash buffer (PBS with 0.05% Tween20, pH6.0) to the wells and completely aspirate the contents.
13) Dilute the HRP secondary antibody at 1:2500 in assay buffer.
14) Add 100 μL HRP secondary antibody diluted in each well
15) Cover with sealing film and place the plates onto a plate shaker set to 200 rpm for 60 minutes at room temperature.
16) Decant plates and tap out residual liquid on a stack of paper towels.
17) Wash wells four times by dispensing 200 μL of wash buffer to the wells and completely aspirate the contents.

18) Dispense 50 μL per well of the TMB substrate solution (3, 3′, 5, 5′ tetramethylbenzidine, Life Technologies, Cat. #002023) into all wells of the plates. Incubate at room temperature for about 2 minutes 30 seconds.
19) Add 50 μL per well of 1N HCl into all wells of the plates.
20) Read plates at 450 nm using PerkinElmer, EnSpire 2300 Multilabel Reader.
21) Optical density values obtained with known concentration of antibodies were used to generate a non-linear regression curve with four parameters using Graph Pad Prism software version 9.0. The concentrations of antibody in the serum samples were determined by extrapolation from standard curve. Non-compartment pharmacokinetics analysis (T1/2 and Cmax) of serum concentrations using intravenous bolus input was conducted using PK Solver 2.0

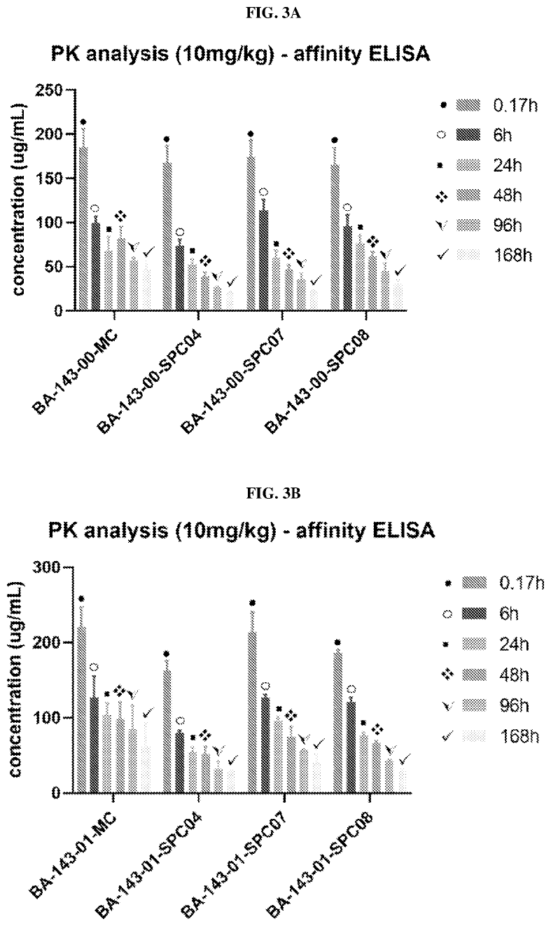

As shown in FIGS. 3A and 3B, no significant difference in the pharmacokinetics was observed between the control Non-CAB (BAP-143-00-MC) linker-conjugated antibody, the control CAB (BAP-143-01-MC) linker-conjugated antibody, the Non-CAB linker compound-conjugated antibodies (BA-143-00-SPC04, BA-143-00-SPC07, and BA-143-00-SPC08) and the CAB linker compound-conjugated antibodies (BA-143-01-SPC04, BA-143-01-SPC07, AND BA-143-01-SPC08) groups. The designations of “BA” and “BAP” are interchangeable.

The results are summarized in TABLE 3 (FIG. 3A) and TABLE 4 (FIG. 3B) below.

TABLE 3

| Test Sample | T½ (h) | C-Max (ug/mL) |
|---|---|---|
| BA-143-00-MC | 157.03 | 185.40 |
| BA-143-00-SPC04 | 116.13 | 167.79 |
| BA-143-00-SPC07 | 109.78 | 174.49 |
| BA-143-00-SPC08 | 126.24 | 165.50 |

TABLE 4

| Test Sample | T½ (h) | C-Max (ug/mL) |
|---|---|---|
| BA-143-01-MC | 175.6 | 220.3 |
| BA-143-01-SPC04 | 119.1 | 163.1 |
| BA-143-01-SPC07 | 131.4 | 214.9 |
| BA-143-01-SPC08 | 102.3 | 186.5 |

### Example 9—PK Analysis Of ADCS With MMAE ELISA

### 1.1 Test Articles

Mouse serum samples were collected at 10 mins, 6 hrs, 24 hrs, 48 hrs, 96 hrs and 168 hrs and 96 hrs post the following antibody treatments (3 mg/kg) in in vivo PK study.
BA-143-00-MC (wildtype or parent non-CAB conjugated to MC)
BA-143-00-SPC04 (wildtype or parent non-CAB conjugated to SPC04)
BA-143-00-SPC07 (wildtype or parent non-CAB conjugated to SPC07)
BA-143-00-SPC08 (wildtype or parent non-CAB conjugated to SPC08)
BA-143-01-MC (CAB conjugated to MC)
BA-143-01-SPC04 (CAB conjugated to SPC04)
BA-143-01-SPC07 (CAB conjugated to SPC07)
BA-143-01-SPC08 (CAB conjugated to SPC08)

### 1.2 Formulations

Test articles were diluted in zero calibrator buffer (Epitope Diagnostics, #KTR-782) by different dilution factors depending on the sample collecting time points.
The antibodies from the PK study were used as the standard controls for standard curves, first were diluted to 10 μg/mL using zero calibrator buffer with 1:200 normal mouse serum, and then 5-fold serially diluted for another 6 concentration points.

### 1.3 MMAE ELISA Assay

1) Add 25 uL of standards and serum samples into the wells (from Intact MMAE ADC ELISA kit, Epitope Diagnostics, #KTR-782).
2) Immediately add 100 uL of assay buffer (Epitope Diagnostics, #30799) 3) Seal the plate wells securely, cover with foil to protect from light, and rotate on plate shaker for 1 hour at 400 to 450 rpm.
4) Wash each well 5 times by dispensing 350 μL of working wash solution (Epitope Diagnostics, #KTR-782) into each well and then completely aspirating the contents.
5) Add 100 μL of diluted MMAE Tracer Antibody (Epitope Diagnostics, #30753) to each well. Tap the plate gently.
6) Seal the plate wells securely, cover with foil to protect from light, and rotate on plate shaker for 30 minutes at 400 to 450 rpm.
7) Wash each well 5 times by dispensing 350 μL of working wash solution into each well and then completely aspirating the contents.
8) Add 100 μL of ELISA HRP Substrate into each of the wells.
9) Cover the plate with aluminum foil to avoid exposure to light. Incubate plate static, at room temperature for 20 minutes.
10) Immediately add 100 μL of ELISA Stop Solution (Epitope Diagnostics, #10030) into each of the wells.
11) Read the absorbance at 450 nm with reference filter at 620 nm.

Optical density values obtained with known concentration of antibodies were used to generate a non-linear regression curve with four parameters using Graph Pad Prism software version 9.0. The concentrations of antibody in the serum samples were determine by extrapolation from standard curve. Non-compartment pharmacokinetics analysis (T1/2 and Cmax) of serum concentrations using intravenous bolus input was conducted using PK Solver 2.0.

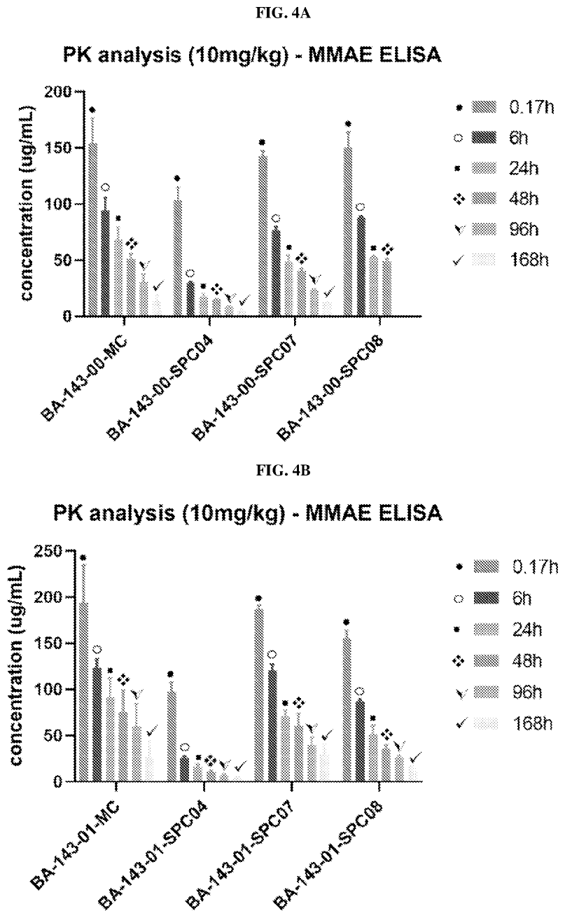

As shown in FIGS. 4A and 4B, no significant difference in the pharmacokinetics was observed between the control Non-CAB (BAP-143-00-MC) linker-conjugated antibody, the control CAB (BAP-143-01-MC) linker-conjugated antibody, the Non-CAB linker compound-conjugated antibodies (BA-143-00-SPC04, BA-143-00-SPC07, AND BA-143-00-SPC08) and the CAB linker compound-conjugated antibodies (BA-143-01-SPC04, BA-143-01-SPC07, AND BA-143-01-SPC08) groups.

The results are summarized in TABLE 5 (FIG. 4A) and TABLE 6 (FIG. 4B) below.

TABLE 5

| Test Sample | T½ (h) | C-Max (ug/mL) |
|---|---|---|
| BA-143-00-MC | 66.10 | 153.85 |
| BA-143-00-SPC04 | 81.74 | 103.70 |
| BA-143-00-SPC07 | 72.78 | 142.73 |
| BA-143-00-SPC08 | 33.04 | 150.48 |

TABLE 6

| Test Sample | T½ (h) | C-Max (ug/mL) |
|---|---|---|
| BA-143-01-MC | 79.07 | 193.98 |
| BA-143-01-SPC04 | 99.32 | 101.94 |
| BA-143-01-SPC07 | 114.34 | 187.30 |
| BA-143-01-SPC08 | 101.72 | 155.82 |

### Example 10—In Vivo Anti-Tumor Efficacy Experiments

### Example 10a. In Vivo Anti-Tumor Efficacy in the Subcutaneous xxt47D CDX Human Breast Cancer Xenograft Model in BABL/c Nude Mice

### Study Objective and Regulatory Compliance

The objective of this study was to evaluate the in vivo anti-tumor efficacy of test articles in the subcutaneous xxT47D CDX human breast cancer xenograft model in female BALB/c nude mice.

| Abbreviation | Definition |
|---|---|
| AAALAC | Association for Assessment and Accreditation |
| | of Laboratory Animal Care |
| IACUC | Institutional Animal Care and Use Committee |
| ATCC | American Type Culture Collection |
| Q4D | Once Every 4 Days |
| GLP | Good Laboratory Practice |
| IV | Intravenous |
| SEM | Standard Error of the Mean |
| TGI | Tumor Growth Inhibition |
| TV | Tumor Volume |
| RTV | Relative Tumor Volume |

### Experimental Design

TABLE 7-1
Description of experimental design

| | | | | Dose | | | |
|---|---|---|---|---|---|---|---|
| | | | Dose | volume | Conc. | | |
| Group | N^{a} | Treatment | (mg/kg) | (mL/kg ^{b}) | mg/mL | Route | Schedule |
| 1 | 8 | Vehicle | — | 10 | — | IV | Q4D × 4 |
| | | | | | | | doses |
| 2 | 8 | BA-143-00-MC | 3 | 10 | 0.3 | IV | Q4D × 4 |
| | | | | | | | doses |
| 3 | 8 | BA-143-00-SPC04 | 3 | 10 | 0.3 | IV | Q4D × 4 |
| | | | | | | | doses |
| 4 | 8 | BA-143-00-SPC07 | 3 | 10 | 0.3 | IV | Q4D × 4 |
| | | | | | | | doses |
| 5 | 8 | BA-143-00-SPC08 | 3 | 10 | 0.3 | IV | Q4D × 4 |
| | | | | | | | doses |
| 6 | 8 | BA-143-01-MC | 3 | 10 | 0.3 | IV | Q4D × 4 |
| | | | | | | | doses |
| 7 | 8 | BA-143-01-SPC04 | 3 | 10 | 0.3 | IV | Q4D × 4 |
| | | | | | | | doses |
| 8 | 8 | BA-143-01-SPC07 | 3 | 10 | 0.3 | IV | Q4D × 4 |
| | | | | | | | doses |
| 9 | 8 | BA-143-01-SPC08 | 3 | 10 | 0.3 | IV | Q4D × 4 |
| | | | | | | | doses |
| 10 | 8 | BA-B12-MC | 3 | 10 | 0.3 | IV | Q4D × 4 |
| | | | | | | | doses |
| 11 | 8 | BA-B12-SPC04 | 3 | 10 | 0.3 | IV | Q4D × 4 |
| | | | | | | | doses |
| 12 | 8 | BA-B12-SPC07 | 3 | 10 | 0.3 | IV | Q4D × 4 |
| | | | | | | | doses |
| 13 | 8 | BA-B12-SPC08 | 3 | 10 | 0.3 | IV | Q4D × 4 |
| | | | | | | | doses |

Note:
^{a}N: number of animals per group.
Dose volume: dosing volume was adjusted to 10 μL/g body weight.

### Materials

### Animals and Housing Conditions

### Animals

Species: Mus musculus
Strain: BALB/c nude
Age: 6-8 weeks
Sex: female
Body weight: 19.8-25.8 g Number of animals: 104 mice plus spare
Animal supplier: Beijing Vital River Laboratory Animal Technology Co., Ltd. No. of certificate of quality: 20170011008172

### Housing Condition

The mice were kept in individual ventilation cages at constant temperature and humidity with 5 animals in each cage.
Temperature: 20-26° C.
Humidity 40-70%.
Cages: Made of polycarbonate. The size is 300 mm×200 mm×180 mm. The bedding material is corn cob, which was changed twice per week.
Diet: Animals had free access to irradiation sterilized dry granule food during the entire study period.
Water: Animals had free access to sterile drinking water.

Cage identification: The identification labels for each cage contained the following information: number of animals, sex, strain, date received, treatment, study number, group number and the starting date of the treatment.

Animal Identification: Animals were Marked by Ear Tags.

### Test and Control Articles

BA-143-00-MC (wildtype or parent non-CAB conjugated to MC)
BA-143-00-SPC04 (wildtype or parent non-CAB conjugated to SPC04)
BA-143-00-SPC07 (wildtype or parent non-CAB conjugated to SPC07)
BA-143-00SPC08 (wildtype or parent non-CAB conjugated to SPC08)
BA-143-01-MC (CAB conjugated to MC)
BA-143-01-SPC04 (CAB conjugated to SPC04)
BA-143-01-SPC07 (CAB conjugated to SPC07)
BA-143-01-SPC08 (CAB conjugated to SPC08)
BA-B12-MC (isotype control antibody conjugated to MC)
BA-B12-SPC04 (isotype control antibody conjugated to SPC04)
BA-B12-SPC07 (isotype control antibody conjugated to SPC07)
BA-B12-SPC08 (isotype control antibody conjugated to SPC08)

### Experimental Methods and Procedures

### Cell Culture

xxT47D cells which were generated from the T47D tumor cells (ATCC, Manassas, VA, cat #ATCC® HTB-133™) by in vivo passaging for twice were used for this project. xxT47D cells were maintained in vitro as a monolayer culture in RPMI-1640 medium supplemented with 10% heat inactivated fetal bovine serum, 1% Antibiotic-Antimycotic at 37° C. with 5% CO₂ in air. The tumor cells were routinely subcultured twice per week by trypsin-EDTA treatment. The cells growing in an exponential growth phase were harvested and counted for tumor inoculation.

### Tumor Inoculation and Animal Grouping

Each mouse was inoculated with 0.18 mg 17-β-estradiol pellet 3 days before subcutaneously at the right flank with xxT47D CDX tumor cells (10×10⁶) in 0.2 mL of PBS supplemented with Matrigel (1:1) for tumor development. Treatments were started on day 9 after tumor inoculation when the average tumor size reached approximately 179 mm³. Animals were assigned into groups according to their tumor volume using an Excel-based stratified randomization program. Each group consisted of 8 tumor-bearing mice. The testing articles were administrated according to the experimental design shown in TABLE 10-1 above.

### Testing Article Preparation

TABLE 7-2
Description of test article preparation

| Compounds | Stock | Preparation | Concentration | Storage |
|---|---|---|---|---|
| Vehicle | — | PBS | — | 4° C. |
| BA-143-00-MC | 1.18 | Add 610 μL of BA-143-00-MC | 0.3 | 4° C. |
| | | (1.18 mg/mL) to 1.790 mL of PBS | | |
| | | and mix well. | | |
| BA-143-00-SPC04 | 1.52 | Add 474 μL of BA-143-00-SPC04 | 0.3 | 4° C. |
| | | (1.52 mg/mL) to 1.926 mL of PBS | | |
| | | and mix well. | | |
| BA-143-00-SPC07 | 1.72 | Add 419 μL of BA-143-00-SPC07 | 0.3 | 4° C. |
| | | (1.72 mg/mL) to 1.981 mL of PBS | | |
| | | and mix well. | | |
| BA-143-00-SPC08 | 1.71 | Add 421 μL of BA-143-00-SPC08 | 0.3 | 4° C. |
| | | (1.71 mg/mL) to 1.979 mL of PBS | | |
| | | and mix well. | | |
| BA-143-01-MC | 1.19 | Add 605 μL of BA-143-01-MC | 0.3 | 4° C. |
| | | (1.19 mg/mL) to 1.795 mL of PBS | | |
| | | and mix well. | | |
| BA-143-01-SPC04 | 1.62 | Add 444 pL of BA-143-01-SPC04 | 0.3 | 4° C. |
| | | (1.62 mg/mL) to 1.956 mL of PBS | | |
| | | and mix well. | | |
| BA-143-01-SPC07 | 1.85 | Add 389 μL of BA-143-01-SPC07 | 0.3 | 4° C. |
| | | (1.85 mg/mL) to2.011 mL of PBS | | |
| | | and mix well. | | |
| BA-143-01-SPC08 | 1.77 | Add 407 μL of BA-143-01-SPC08 | 0.3 | 4° C. |
| | | (1.77 mg/mL) to 1.993 mL of PBS | | |
| | | and mix well. | | |
| BA-B12-MC | 1.30 | Add 554 μL of BA-B12-MC (1.30 | 0.3 | 4° C. |
| | | mg/mL) to 1.846 mL of PBS and | | |
| | | mix well. | | |
| BA-B12-SPC04 | 1.56 | Add 462 μL of BA-B12-SPC04 | 0.3 | 4° C. |
| | | (1.56 mg/mL) to 1.938 mL of PBS | | |
| | | and mix well. | | |
| BA-B12-SPC07 | 1.64 | Add 439 μL of BA-B12-SPC07 | 0.3 | 4° C. |
| | | (1.64 mg/mL) to 1.961 mL of PBS | | |
| | | and mix well. | | |
| BA-B12-SPC08 | 1.71 | Add 421 μL of BA-B12-SPC08 | 0.3 | 4° C. |
| | | (1.71 mg/mL) to 1.979 mL of PBS | | |
| | | and mix well. | | |

Note:
The test article formulation was prepared before each dosing.

### Tumor Measurements and Endpoints

The major endpoint was to assess whether the tumor growth could be delayed. Tumor size was measured twice weekly in two dimensions using a caliper and was calculated using the formula: V=0.5 a×b² where a and b are the long and short diameters of the tumor, respectively. The tumor size was then used for calculations of T/C, TGI and RTV values.

The T/C value (in percent) is an indication of antitumor effectiveness; T and C are the mean volumes of the treated and control groups, respectively, on a given day.

TGI for each treatment group was calculated using the formula: TGI (%)=[1−(Ti−T0)/(Vi−V0)]×100; Ti is the average tumor volume of a treatment group on a given day, TO is the average tumor volume of the treatment group on day 0, Vi is the average tumor volume of the vehicle control group on the same day with Ti, and V0 is the average tumor volume of the vehicle group on day 0.

Individual RTV (relative tumor volume) was calculated by dividing the tumor volume on a specific day by its volume on day 0. The RTV value of each mouse was calculated individually which was then used for mean RTV calculation for a group.

### Sampling

As set forth in TABLE 10-3 below, ~50 μL serum was collected from 3 mice from groups 2 to 13 at 24 hours and 96 hours (right before the 2^{nd} dose) post the first dose, respectively.

TABLE 7-3

| | Time | Serum | Time | Serum |
|---|---|---|---|---|
| Group# | point | (μL) | point | (μL) |
| Group-2 | 24 hr post | 50 | 96 hr post | 50 |
| BA-143-00-MC, 3 mg/kg | 1st dosing | 50 | 1st dosing | 50 |
| IV, 10 μL/g, Q4D × 4 doses | | 50 | | 50 |
| Group-3 | 24 hr post | 50 | 96 hr post | 50 |
| BA-143-00-SPC04, 3 mg/kg | 1st dosing | 50 | 1st dosing | 50 |
| IV, 10 μL/g, Q4D × 4 doses | | 50 | | 50 |
| Group-4 | 24 hr post | 50 | 96 hr post | 50 |
| BA-143-00-SPC07, 3 mg/kg | 1st dosing | 50 | 1st dosing | 50 |
| IV, 10 μL/g, Q4D × 4 doses | | 50 | | 50 |
| Group-5 | 24 hr post | 50 | 96 hr post | 50 |
| BA-143-00-SPC08, 3 mg/kg | 1st dosing | 50 | 1st dosing | 50 |
| IV, 10 μL/g, Q4D × 4 doses | | 50 | | 50 |
| Group-6 | 24 hr post | 50 | 96 hr post | 50 |
| BA-143-01-MC, 3 mg/kg | 1st dosing | 50 | 1st dosing | 50 |
| IV, 10 μL/g, Q4D × 4 doses | | 50 | | 50 |
| Group-7 | 24 hr post | 50 | 96 hr post | 50 |
| BA-143-01-SPC04, 3 mg/kg | 1st dosing | 50 | 1st dosing | 50 |
| IV, 10 μL/g, Q4D × 4 doses | | 50 | | 50 |
| Group-8 | 24 hr post | 50 | 96 hr post | 50 |
| BA-143-01-SPC07, 3 mg/kg | 1st dosing | 50 | 1st dosing | 50 |
| IV, 10 μL/g, Q4D × 4 doses | | 50 | | 50 |
| Group-9 | 24 hr post | 50 | 96 hr post | 50 |
| BA-143-01-SPC08, 3 mg/kg | 1st dosing | 50 | 1st dosing | 50 |
| IV, 10 μL/g, Q4D × 4 doses | | 50 | | 50 |
| Group-10 | 24 hr post | 50 | 96 hr post | 50 |
| BA-B12-MC, 3 mg/kg | 1st dosing | 50 | 1st dosing | 50 |
| IV, 10 μL/g, Q4D × 4 doses | | 50 | | 50 |
| Group-11 | 24 hr post | 50 | 96 hr post | 50 |
| BA-B12-SPC04, 3 mg/kg | 1st dosing | 50 | 1st dosing | 50 |
| IV, 10 μL/g, Q4D × 4 doses | | 50 | | 50 |
| Group-12 | 24 hr post | 50 | 96 hr post | 50 |
| BA-B12-SPC07, 3 mg/kg | 1st dosing | 50 | 1st dosing | 50 |
| IV, 10 μL/g, Q4D × 4 doses | | 50 | | 50 |
| Group-13 | 24 hr post | 50 | 96 hr post | 50 |
| BA-B12-SPC08, 3 mg/kg | 1st dosing | 50 | 1st dosing | 50 |
| IV, 10 μL/g, Q4D × 4 doses | | 50 | | 50 |

### Statistical Analysis

The mean tumor volume of each group and SEM at different time points were calculated (TABLE 7-4). Statistical analysis of differences in the tumor volume among groups were conducted on the data obtained on Day 27 and Day 34 after the start of treatment at the end of the study.

One-way ANOVA was performed to compare the mean tumor volumes and RTVs among groups. Comparisons between groups were carried out with Games-Howell test, as a significant F-statistics was obtained.

All data were analyzed using IBM® SPSS Statistics® software (version 17.0.) p<0.05 was considered to be statistically significant.

The mean tumor volume of different groups is shown in TABLE 7-4 below.

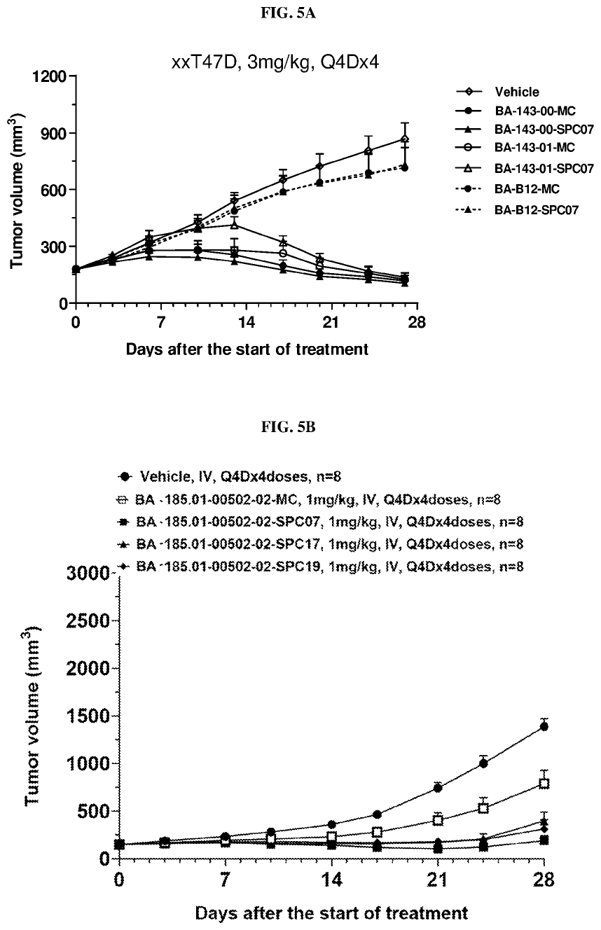

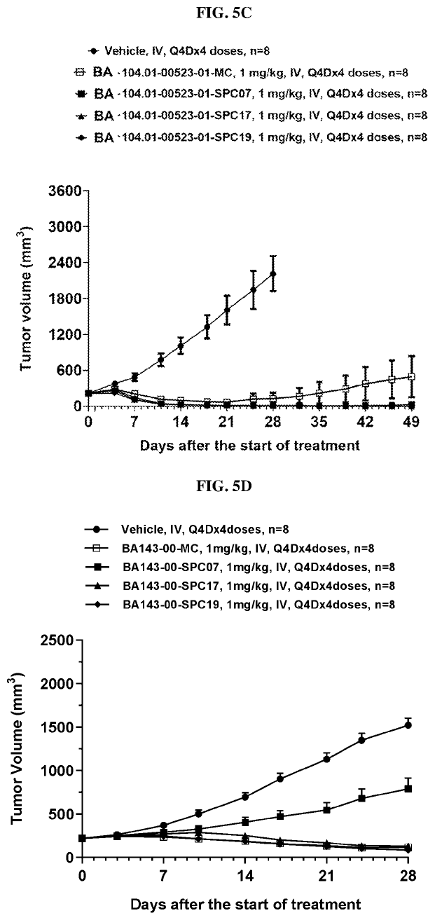

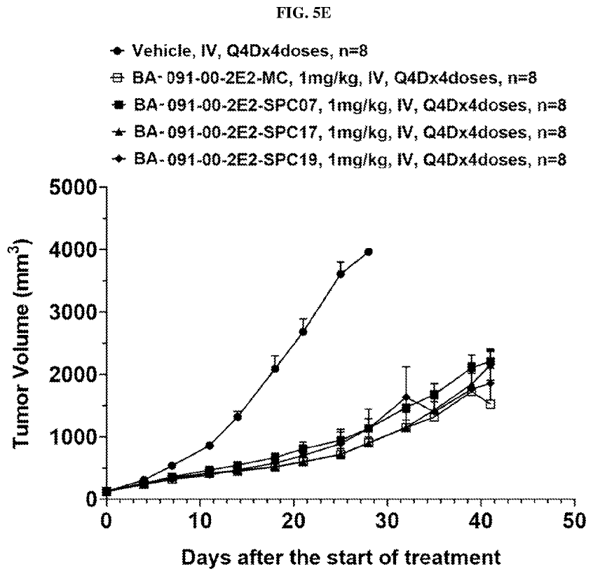

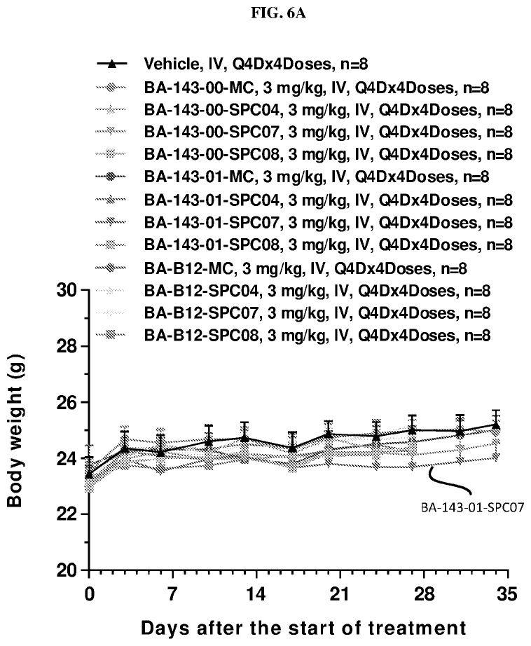

The mean tumor volume of Groups 1, 2, 4, 6, 8, 10, and 12 of TABLE 7-4 are shown in FIG. 5A.

TABLE 7-4
Tumor Volume

| | Tumor volume (mm³)^{a} | | | | | | | | | | | | |
|---|---|---|---|---|---|---|---|---|---|---|---|---|---|
| Days | G1^{b} | G2 | G3 | G4 | G5 | G6 | G7 | G8 | G9 | G10 | G11 | G12 | G13 |
| 0ª | 179 ± 14 | 179 ± 13 | 179 ± 14 | 179 ± 15 | 179 ± 14 | 179 ± 14 | 179 ± 15 | 179 ± 14 | 179 ± 14 | 179 ± 14 | 179 ± 15 | 179 ± 14 | 179 ± 14 |
| 3 | 236 ± 18 | 231 ± 16 | 235 ± 18 | 216 ± 13 | 224 ± 20 | 230 ± 18 | 212 ± 15 | 251 ± 19 | 227 ± 18 | 236 ± 21 | 215 ± 20 | 224 ± 16 | 228 ± 21 |
| 6 | 318 ± 36 | 278 ± 24 | 328 ± 17 | 245 ± 19 | 293 ± 41 | 277 ± 34 | 271 ± 18 | 349 ± 34 | 290 ± 29 | 313 ± 32 | 295 ± 38 | 295 ± 22 | 309 ± 33 |
| 10 | 426 ± 40 | 278 ± 33 | 429 ± 34 | 242 ± 26 | 373 ± 63 | 280 ± 50 | 376 ± 26 | 395 ± 42 | 388 ± 57 | 392 ± 45 | 423 ± 65 | 401 ± 46 | 398 ± 55 |
| 13 | 540 ± 43 | 256 ± 41 | 511 ± 47 | 220 ± 25 | 415 ± 66 | 279 ± 61 | 473 ± 43 | 413 ± 43 | 493 ± 92 | 486 ± 65 | 529 ± 92 | 502 ± 69 | 455 ± 67 |
| 17 | 650 ± 56 | 197 ± 31 | 576 ± 61 | 176 ± 22 | 436 ± 70 | 263 ± 63 | 579 ± 74 | 321 ± 34 | 546 ± 104 | 588 ± 82 | 621 ± 112 | 590 ± 84 | 490 ± 72 |
| 20 | 723 ± 65 | 157 ± 24 | 604 ± 79 | 142 ± 19 | 444 ± 77 | 196 ± 42 | 637 ± 87 | 236 ± 26 | 569 ± 113 | 639 ± 88 | 673 ± 114 | 633 ± 93 | 528 ± 81 |
| 24 | 806 ± 77 | 139 ± 27 | 601 ± 97 | 125 ± 16 | 427 ± 84 | 157 ± 38 | 681 ± 105 | 169 ± 21 | 561 ± 127 | 690 ± 103 | 719 ± 120 | 677 ± 108 | 555 ± 85 |
| 27 | 869 ± 85 | 119 ± 28 | 603 ± 118 | 105 ± 18 | 405 ± 93 | 126 ± 36 | 736 ± 118 | 136 ± 22 | 566 ± 146 | 714 ± 108 | 777 ± 130 | 732 ± 121 | 600 ± 92 |
| 31 | 916 ± 91 | 120 ± 34 | — | 94 ± 18 | — | 119 ± 35 | — | 114 ± 22 | — | — | — | — | — |
| 34 | 936 ± 95 | 124 ± 36 | — | 89 ± 18 | — | 106 ± 33 | — | 96 ± 22 | — | — | — | — | — |

Note:
^{a}Mean ± SEM
^{b}G1: Vehicle, G2: BA-143-00-MC (3 mg/kg), G3: BA-143-00-SPC04 (3 mg/kg), G4: BA-143-00-SPC07 (3 mg/kg), G5: BA-143-00-SPC08 (3 mg/kg), G6: BA-143-01-MC (3 mg/kg), G7: BA-143-01-SPC04 (3 mg/kg), G8: BA-143-01-SPC07 (3 mg/kg), G9: BA-143-01-SPC08 (3 mg/kg), G10: BA-B12-MC (3 mg/kg), G11: BA-B12-SPC04 (3 mg/kg), G12: BA-B12-SPC07 (3 mg/kg), G13: BA-B12-SPC08 (3 mg/kg).

### Results

### Mortality, Morbidity, and Body Weight Gain or Loss

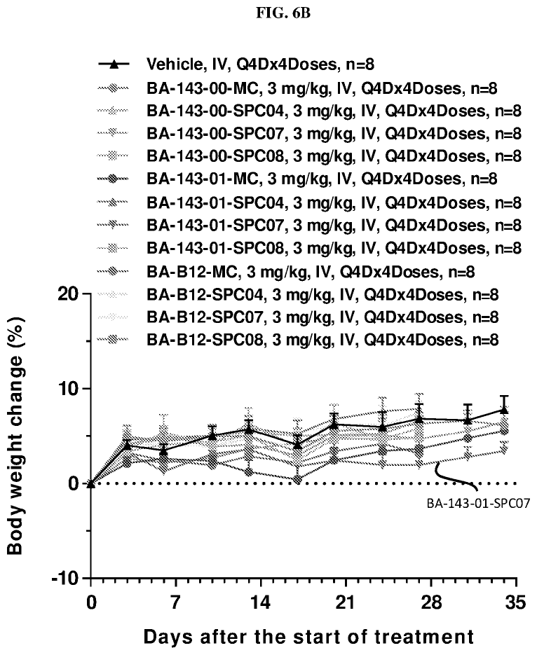

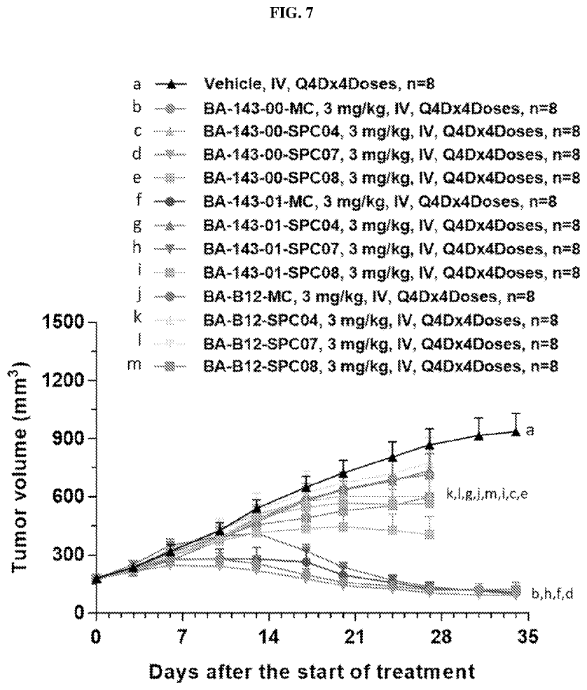

Animal body weight was monitored regularly as an indicator of toxicity. During this study, no group showed significant body weight loss (10% or above) (FIG. 6A). No death or morbidity was observed. Thus, no obvious toxicity was observed in association with the administration of the test articles to tumor-bearing BALB/c nude mice in the current dosing regimen.

FIG. 6A shows the mean body weight of different treatment groups. Data is presented as mean±SEM.

The relative body weight changes of different groups are shown in FIG. 6B.

FIG. 6B shows relative body weight changes (%) of different treatment groups. The percent change was calculated based on the animal weight on the first day of dosing. Data is presented as mean±SEM.

### Tumor Growth Inhibition Analysis

TABLE 7-5
Tumor growth inhibition when compared to vehicle group
(based on Day 27 data)

| | Tumor Size | T/C ^{b} | TGI | | | |
|---|---|---|---|---|---|---|
| Treatment | (mm³) ^{a} | (%) | (%) | RTV | p ^{c} | p ^{d} |
| Vehicle | 869 ± 85 | — | — | 4.95 ± 0.45 | — | — |
| BA-143-00-MC, 3 mg/kg | 119 ± 28 | 13.70 | 108.72 | 0.66 ± 0.14 | 0.001 | <0.001 |
| BA-143-00-SPC04, 3 mg/kg | 603 ± 118 | 69.46 | 38.45 | 3.73 ± 0.84 | 0.807 | 0.975 |
| BA-143-00-SPC07, 3 mg/kg | 105 ± 18 | 12.13 | 110.68 | 0.63 ± 0.15 | 0.001 | <0.001 |
| BA-143-00-SPC08, 3 mg/kg | 405 ± 93 | 46.65 | 67.22 | 2.13 ± 0.33 | 0.077 | 0.009 |
| BA-143-01-MC, 3 mg/kg | 126 ± 36 | 14.47 | 107.74 | 0.71 ± 0.19 | 0.001 | <0.001 |
| BA-143-01-SPC04, 3 mg/kg | 736 ± 118 | 84.72 | 19.25 | 4.26 ± 0.69 | 0.998 | 0.999 |
| BA-143-01-SPC07, 3 mg/kg | 136 ± 22 | 15.63 | 106.26 | 0.79 ± 0.15 | 0.001 | <0.001 |
| BA-143-01-SPC08, 3 mg/kg | 566 ± 146 | 65.12 | 43.94 | 3.14 ± 0.84 | 0.818 | 0.771 |
| BA-B12-MC, 3 mg/kg | 714 ± 108 | 82.25 | 22.33 | 4.03 ± 0.48 | 0.991 | 0.957 |
| BA-B12-SPC04, 3 mg/kg | 777 ± 130 | 89.41 | 13.34 | 4.53 ± 0.94 | 1.000 | 1.000 |
| BA-B12-SPC07, 3 mg/kg | 732 ± 121 | 84.30 | 19.76 | 4.08 ± 0.65 | 0.998 | 0.993 |
| BA-B12-SPC08, 3 mg/kg | 600 ± 92 | 69.09 | 38.90 | 3.4 ± 0.55 | 0.639 | 0.627 |

Notes:
^{a} Mean ± SEM.

b. Tumor Growth Inhibition was calculated by dividing the group average tumor volume of the treated group by the group average tumor volume of the vehicle control group (T/C).
c. p value calculated based on tumor size.
p value calculated based on RTV

TABLE 7-6
Tumor growth inhibition when compared to vehicle group
(based on Day 34 data)

| | Tumor | | | | | |
|---|---|---|---|---|---|---|
| | Size | T/C ^{b} | TGI | | | |
| Treatment | (mm³) ^{a} | (%) | (%) | RTV | p ^{c} | p ^{d} |
| Vehicle | 936 ± 95 | — | — | 5.35 ± 0.54 | — | — |
| BA-143-00-MC, | 124 ± 36 | 13.27 | 107.24 | 0.69 ± 0.19 | <0.001 | <0.001 |
| 3mg/kg | | | | | | |
| BA-143-00- | 89 ± 18 | 9.46 | 111.94 | 0.54 ± 0.15 | <0.001 | <0.001 |
| SPC07, 3mg/kg | | | | | | |
| BA-143-01-MC, | 106 ± 33 | 11.36 | 109.60 | 0.59 ± 0.18 | <0.001 | <0.001 |
| 3mg/kg | | | | | | |
| BA-143-01- | 96 ± 22 | 10.29 | 110.90 | 0.56 ± 0.14 | <0.001 | <0.001 |
| SPC07, 3mg/kg | | | | | | |

Note:
^{a} Mean ± SEM.
^{b} Tumor Growth Inhibition was calculated by dividing the group average tumor volume of the treated group by the group average tumor volume of the vehicle control group (T/C).
^{c} p value calculated based on tumor size.
^{d} p value calculated based on RTV.

### Summary and Discussion

In this study, the therapeutic efficacy of the treatment groups in TABLE 7-3 were evaluated using xxT47D CDX human breast cancer xenograft model. The tumor sizes of different groups at different time points after treatment are shown in TABLE 7-4, TABLE 7-5, TABLE 7-6 and FIG. 7.

The mean tumor size of the vehicle treated group reached 869 mm³ on Day 27 (RTV=4.95±0.45) and 936 mm³ on Day 34 (RTV=5.35±0.54) after the start of treatment, respectively. The treatment groups BA-143-00-MC (Group 2), BA-143-00-SPC07 (Group 4), BA-143-01-MC (Group 6) and BA-143-01-SPC07 (Group 8) at 3 mg/kg dose level all exhibited dramatic anti-tumor activities (TGI≥106.26%, p<0.001, compared to the vehicle group on Day 27 and Day 34). The treatment groups BA-143-00-SPC08 (Group 5) and BA-143-01-SPC08 (Group 9) at 3 mg/kg dose level exhibited medium anti-tumor activities (TGI=67.22% and 43.94%, p=0.077 and 0.818, compared to vehicle on Day 27). At the same time, other treatment groups (Group 3 and Group 7) and isotype control drugs produced nearly no antitumor activity (TGI<38.90%, p>0.05, compared to the vehicle group). (FIG. 7). No severe body weight loss or death/morbidity event was observed. Thus, no obvious toxicity was observed in association with the administration of the treatment groups.

### Example 10B. In Vivo Anti-Tumor Efficacy in the Subcutaneous LNCap FGC Human Prostate Cancer Xenograft Model in SCID Mice

The objective of this study was to evaluate the in vivo anti-tumor efficacy of BioAtla's test articles in the subcutaneous castrated LNCap-FGC human prostate cancer CDX model in male CB17.SCID mice.

Experiments similar to those described above in Example 10A were conducted with SPC and control linker conjugates to antibodies against prostate cancer cells. Male SCID mice (6-8 weeks) were inoculated subcutaneously in the right flank with 10×10⁶ human prostate cancer cells LNCap FGC (ECACC-89110211). Castration was performed when the average tumor volume reached about 114 mm³ (14 days after the cell inoculation). After 3 days of recovery, when the average tumor volume reached approximately 150 mm³, animals were randomized into groups of eight animals each and treated intravenously with vehicle or test conjugates at 1 mg/kg, once every four days, for a total of four doses (Q4D×4). Tumor sizes were monitored routinely. The efficacy was measured by % TGI (tumor growth inhibition) which indicates the difference in tumor volume change between vehicle-treated control group and the test article-treated cohort. The results are presented in FIG. 5B.

### Experimental Design

TABLE 7-7
Description of experimental design

| | | | Dose | | |
|---|---|---|---|---|---|
| Group | N^{a} | Treatment | (mg/kg) | Route | Schedule |
| 1 | 8 | Vehicle | — | IV | Q4D × 4 |
| | | | | | doses |
| 2 | 8 | BA-185.01-00502- | 1 | IV | Q4D × 4 |
| | | 02-MC | | | doses |
| 3 | 8 | BA-185.01-00502- | 1 | IV | Q4D × 4 |
| | | 02-SPC07 | | | doses |
| 4 | 8 | BA-185.01-00502- | 1 | IV | Q4D × 4 |
| | | 02-SPC17 | | | doses |
| 5 | 8 | BA-185.01-00502- | 1 | IV | Q4D × 4 |
| | | 02-SPC19 | | | doses |

### Example 10C. In Vivo Anti-Tumor Efficacy in the Subcutaneous NCI-H820 Human Lung Cancer Xenograft Model in BALB/c Nude Mice

Experiments similar to those described above in Example 10A were conducted with SPC and control linker conjugates to antibodies against lung cancer cells. Female BALB/c nude mice (6-8 weeks) were inoculated subcutaneously in the right flank with 10×10⁶ human lung cancer cells NCI-H820 (ATCC, Manassas, VA, cat #HTB-181). When the average tumor volume reached approximately 217 mm³, animals were randomized into groups of eight animals each and treated intravenously with vehicle or test conjugates at 1 mg/kg, once every four days, for a total of four doses (Q4D×4). Tumor sizes were monitored routinely. The efficacy was measured by % TGI (tumor growth inhibition) which indicates the difference in tumor volume change between vehicle-treated control group and the test article-treated cohort. The results are presented in FIG. 5C.

### Experimental Design

TABLE 7-8
Description of experimental design

| | | | Dose | | |
|---|---|---|---|---|---|
| Group | N^{a} | Treatment | (mg/kg) | Route | Schedule |
| 1 | 8 | Vehicle | — | IV | Q4D × 4 |
| | | | | | doses |
| 2 | 8 | BA-104.01-00523- | 1 | IV | Q4D × 4 |
| | | 01-MC | | | doses |
| 3 | 8 | BA-104.01-00523- | 1 | IV | Q4D × 4 |
| | | 01-SPC07 | | | doses |
| 4 | 8 | BA-104.01-00523- | 1 | IV | Q4D × 4 |
| | | 01-SPC17 | | | doses |
| 5 | 8 | BA-104.01-00523- | 1 | IV | Q4D × 4 |
| | | 01-SPC19 | | | doses |

### Example 10D. In Vivo Anti-Tumor Efficacy in the Subcutaneous xBT474 Human Breast Cancer Xenograft Model in BALB/c Nude Mice

Experiments similar to those described above in Example 10A were conducted with SPC and control linker conjugates to antibodies against breast cancer cells. Female BALB/c nude mice (6-8 weeks) were inoculated with 0.36 mg 17-β-estradiol pellet 3 days before subcutaneous cell inoculation in the right flank with 10×10⁶ human breast cancer cells xBT474 (generated from BT474 cells, ATCC® HTB-20™). When the average tumor volume reached approximately 220 mm³, animals were randomized into groups of eight animals each and treated intravenously with vehicle or test conjugates at 1 mg/kg, once every four days, for a total of four doses (Q4Dx4). Tumor sizes were monitored routinely. The efficacy was measured by % TGI (tumor growth inhibition) which indicates the difference in tumor volume change between vehicle-treated control group and the test article-treated cohort. The results are presented in FIG. 5D.

### Experimental Design

TABLE 7-9
Description of experimental design

| | | | Dose | | |
|---|---|---|---|---|---|
| Group | N^{a} | Treatment | (mg/kg) | Route | Schedule |
| 1 | 8 | Vehicle | — | IV | Q4D × 4 |
| | | | | | doses |
| 2 | 8 | BA-143-00-MC | 1 | IV | Q4D × 4 |
| | | | | | doses |
| 3 | 8 | BA-143-00-SPC07 | 1 | IV | Q4D × 4 |
| | | | | | doses |
| 4 | 8 | BA-143-00-SPC17 | 1 | IV | Q4D × 4 |
| | | | | | doses |
| 5 | 8 | BA-143-00-SPC19 | 1 | IV | Q4D × 4 |
| | | | | | doses |

TABLE 7-10

| | | | Heavy | Light |
|---|---|---|---|---|
| | | | Chain | Chain |
| | | | Variable | Variable |
| Groups | Treatment | | Region | Region |
| 1 | Vehicle | control | | |
| 2 | BA-143- | Wildtype or parent | SEQ ID | SEQ ID |
| | 00-MC | Non-CAB | NO: 19 | NO: 32 |
| | | conjugated to MC | | |
| | | linker | | |
| 3 | BA-143- | Wildtype or parent | SEQ ID | SEQ ID |
| | 00-SPC07 | Non-CAB | NO: 19 | NO: 32 |
| | | conjugated to linker | | |
| | | compound SPC07 | | |
| 4 | BA-143- | Wildtype or parent | SEQ ID | SEQ ID |
| | 00-SPC17 | Non-CAB | NO: 19 | NO: 32 |
| | | conjugated to linker | | |
| | | compound SPC17 | | |
| 5 | BA-143- | Wildtype or parent | SEQ ID | SEQ ID |
| | 00-SPC19 | non-CAB conjugated | NO: 19 | NO: 32 |
| | | to linker compound | | |
| | | SPC19 | | |

### Example 10E. In Vivo Anti-Tumor Efficacy in the Subcutaneous Mx-1 Human Breast Cancer Xenograft Model in BALB/c Nude Mice

Similar to Examples 10A and 10D above, experiments were conducted with SPC and control linker conjugates to antibodies against breast cancer cells. Female BALB/c nude mice (6-8 weeks) were inoculated subcutaneously in the right flank with 5×10⁶ human breast cancer cells Mx-1. When the average tumor volume reached approximately 124 mm³, animals were randomized into groups of eight animals each and treated intravenously with vehicle or test articles SPC conjugates at 1 mg/kg, once every four days, for a total of four doses (Q4Dx4). Tumor sizes were monitored routinely. The efficacy was measured by % TGI (tumor growth inhibition) which indicates the difference in tumor volume change between vehicle-treated control group and the test article-treated cohort. The results are presented in FIG. 5E.

### Experimental Design

TABLE 7-11
Description of experimental design

| | | | Dose | | |
|---|---|---|---|---|---|
| Group | N^{a} | Treatment | (mg/kg) | Route | Schedule |
| 1 | 8 | Vehicle | — | IV | Q4D × 4 |
| | | | | | doses |
| 2 | 8 | BA-091-00-2E2- | 1 | IV | Q4D × 4 |
| | | MC | | | doses |
| 3 | 8 | BA-091-00-2E2- | 1 | IV | Q4D × 4 |
| | | SPC07 | | | doses |
| 4 | 8 | BA-091-00-2E2- | 1 | IV | Q4D × 4 |
| | | SPC17 | | | doses |
| 5 | 8 | BA-091-00-2E2- | 1 | IV | Q4D × 4 |
| | | SPC19 | | | doses |

### Example 11—Human Serum Stability Assay

### 1.1 Test Articles

BA-143-00-MC (wildtype or parent non-CAB conjugated to MC)
BA-143-00-SPC04 (wildtype or parent non-CAB conjugated to SPC04)
BA-143-00-SPC07 (wildtype or parent non-CAB conjugated to SPC07)
BA-143-00-SPC08 (wildtype or parent non-CAB conjugated to SPC08)
BA-143-01-MC (CAB conjugated to MC)
BA-143-01-SPC04 (CAB conjugated to SPC04)
BA-143-01-SPC07 (CAB conjugated to SPC07)
BA-143-01-SPC08 (CAB conjugated to SPC08)

### Serum Incubation

Test articles were added to human serum at 0.2 mg/ml. The aliquoted test articles containing serum samples were incubated at 37° C. for 0, 7 and 14 days, and stored at −80° C. after collection for further analysis.

### Immuno-Capture Method

Suspended protein A beads were added to the test articles samples in serum and incubated for 30 min at room temperature with 1400 rpm shaking. The capture beads were collected through filter plate by centrifuging at 150 rcf for 1 min. Then the affinity captured test articles were eluted by 100 mM pH2.7 glycine-HCL buffer for 10 min at room temperature with 800 rpm shaking and neutralized by 2M pH 7.5 Tris buffer.

### RP-HPLC Method

After the samples were reduced by 150 mM DTT solution for 30 min at 37° C., reverse phase chromatography was performed to evaluate DAR profile. The changes in DAR (% DAR) were calculated as ((Day7 or Day14 DAR-Day0 DAR)/Day0 DAR)×100%.

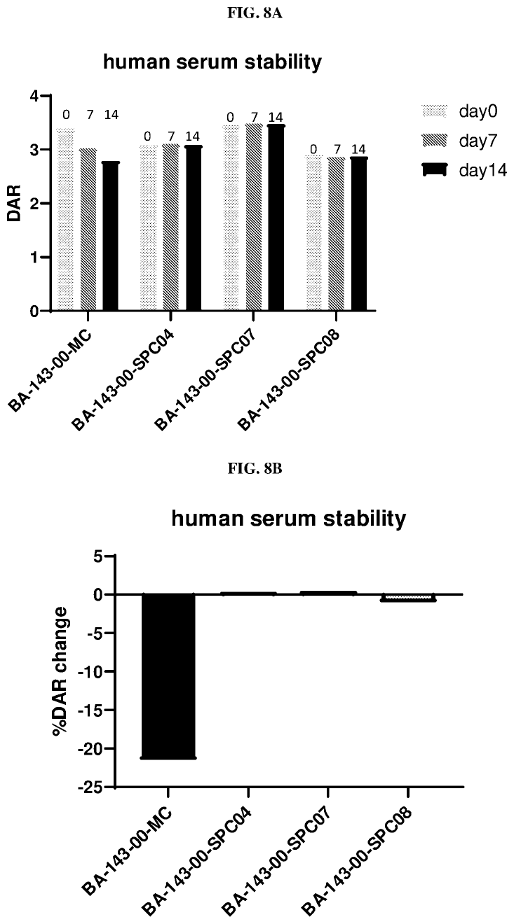

FIG. 8A shows there was the differences in the DAR in human serum of BA-143-00-MC, BA-143-00-SPC04, BA-143-00-SPC07, and BA-143-00-SPC08. FIG. 8B shows the differences in % DAR change between BA-143-00-MC and the groups BA-143-00-SPC04, BA-143-00-SPC07, and BA-143-00-SPC08. The inventive immunoconjugates were much more stable than the wildtype immunoconjugate (BA-143-00-MC); as such, the inventive delivery system would have excellent precision in delivering the drug to the intended target without unwanted drug release away from the intended target.

### Example 12—pH FACS Assay

### 1.1 Test Articles

BA-143-00-MC (wildtype or parent non-CAB conjugated to MC)
BA-143-00-SPC04 (wildtype or parent non-CAB conjugated to SPC04)
BA-143-00-SPC07 (wildtype or parent non-CAB conjugated to SPC07)
BA-143-00-SPC08 (wildtype or parent non-CAB conjugated to SPC08)
BA-143-01-MC (CAB conjugated to MC)
BA-143-01-SPC04 (CAB conjugated to SPC04)
BA-143-01-SPC07 (CAB conjugated to SPC07)
BA-143-01-SPC08 (CAB conjugated to SPC08)
Isotype control BA-B12-MC (isotype control antibody conjugated to MC)
Isotype control BA-B12-SPC04 (isotype control antibody conjugated to SPC04)
Isotype control BA-B12-SPC07 (isotype control antibody conjugated to SPC07)
Isotype control BA-B12-SPC08 (isotype control antibody conjugated to SPC08)

### 1.2 Formulations

Test articles were first diluted to 30 μg/mL in pH 6.0 or pH 7.4 FACS buffer, then 3-fold serially diluted in pH 6.0 or pH 7.4 FACS buffer.

### 1.3 Cell Culturing

293-human Nectin4 and 293-cynomolgus Nectin4 cells were maintained in stable cell line culture medium (MEM+10% FBS+1 mg/mL G418). T47D cells (ATCC, Cat #HTB133) were maintained in T47D culture medium (RPMI1640+10% FBS+0.2U/mL human insulin). BT474 cells (ATCC, Cat #HTB-20) were maintained in BT474 culture medium (Hybri-Care+10% FBS). The cells were routinely sub-cultured twice per week. The cells were harvested during exponential growth phase and counted for plating.

### 1.4 Cell Staining Using Test Antibodies

1) Seed 3×10⁶ cells to T-75 flasks and culture according to the instructions of vendors.
2) On the day of FACS analysis, remove and discard culture medium.
3) Briefly rinse the cell layer with PBS solution.
4) Add 1.5 mL of Detachin cell detachment solution (Genlantis Cat #100100) to each of the T-75 flasks. Wait until cell layer has dispersed.
5) Add 4.5 mL of culture media for the corresponding cell lines and resuspend cells by gently pipetting.
6) Pool the cells and transfer the cell suspension to a 50-mL conical tube.
7) Count the cells with trypan blue staining before centrifugation at 1500 rpm for 5 min at 4° C.
8) Wash the cells once with PBS
9) Resuspend the cells in pH 6.0 or pH 7.4 FACS buffer to 3.5×10⁶ cells/mL.
10) Aliquot 2×10⁵ cells in 100 μL pH 6.0 or pH 7.4 FACS buffer in 96-well U-bottom plates.
11) Spin down the cells and discard the buffer.
12) Serially dilute test articles in 3-fold dilutions starting at 30 μg/mL in pH 6.0 or pH 7.4 FACS buffer.
13) Add 100 μL/well of the diluted test articles to cells, gently mix well and incubate on ice with shaking (200 rpm) for one hour.
14) Centrifuge the cells at 2000 rpm for 3 min at 4° C. Wash the cells with 150 μL of pH 6.0 or pH 7.4 wash buffer twice.
15) Dilute the goat anti-human IgG Alexa Fluor 488 antibody 1:300 in pH 6.0 or pH 7.4 FACS buffers.
16) Add 100 μL of the diluted antibody from step above to the cells and incubate on ice with shaking (200 rpm) for 45 minutes, protected from light.
17) Pellet the cells and wash with 150 μL of pH 6.0 or pH 7.4 wash buffer three times.
18) Fix cells with 4% paraformaldehyde diluted in 1×PBS for 10 min at room temperature, then wash cells with 1×PBS.
19) Resuspend the cells in 100 μL of 1×PBS.
   Analyze the cells by NovoCyte Flow Cytometer using Ex488 nm/Em530 nm. Collect at least 3,000 singlet cells for each data point.

FACS buffer: add 1.5 ml of 30% BSA to 43.5 ml of wash buffer. Adjust pH to 6.0 or 7.4 using 6 N or 0.6 N HCl.

### 1.5 FACS Data Analysis

The MFI of AF488 in cell singlets was plotted using GraphPad Prism software version 7.03.

TABLE 8

| 293- | | | | BA-143- | | BA-143- | BA-143- | BA-143- |
|---|---|---|---|---|---|---|---|---|
| huNectin4, | BA-143- | BA-143- | BA-143- | 00- | BA-143- | 01- | 01- | 01- |
| EC50 (ng/ml) | 00-MC | 00-SPC04 | 00-SPC07 | SPC08 | 01-MC | SPC04 | SPC07 | SPC08 |
| pH6.0 | 56.43 | 112.9 | 116.4 | 125.3 | 102.8 | 143.8 | 147.7 | 124.3 |
| pH7.4 | 65.19 | 49.21 | 68.61 | 312.6 | 239.1 | 268.5 | 308.5 | 298.3 |
| ratio | 1.16 | 0.44 | 0.59 | 2.49 | 2.33 | 1.87 | 2.09 | 2.40 |
| (pH7.4/pH6.0) | | | | | | | | |

TABLE 9

| 293- | | | BA-143- | BA-143- | | BA-143- | BA-143- | BA-143- |
|---|---|---|---|---|---|---|---|---|
| cynoNectin4, | BA-143- | BA-143- | 00- | 00- | BA-143- | 01- | 01- | 01- |
| EC50 (ng/mL) | 00-MC | 00-SPC04 | SPC07 | SPC08 | 01-MC | SPC04 | SPC07 | SPC08 |
| pH6.0 | 50.56 | 61.22 | 73.64 | 68.36 | 105.4 | 105 | 110.3 | 155.6 |
| pH7.4 | 34.16 | 72.01 | 80.75 | 79.22 | 145.5 | 208.5 | 280.4 | 328.3 |
| ratio | 0.68 | 1.18 | 1.10 | 1.16 | 1.38 | 1.99 | 2.54 | 2.11 |
| (pH7.4/pH6.0) | | | | | | | | |

TABLE 10

| | | BA-143- | BA-143- | BA-143- | | BA-143- | BA-143- | BA-143- |
|---|---|---|---|---|---|---|---|---|
| BT474, EC50 | BA-143- | 00- | 00- | 00- | BA-143- | 01- | 01- | 01- |
| (ng/ml) | 00-MC | SPC04 | SPC07 | SPC08 | 01-MC | SPC04 | SPC07 | SPC08 |
| pH6.0 | 11.04 | 18.14 | 24.2 | 20.17 | 44.22 | 35.24 | 35.18 | 52.46 |
| pH7.4 | 8.842 | 32.72 | 20.38 | 15.67 | 220.9 | 286.5 | 332.4 | 381.2 |
| ratio | 0.80 | 1.80 | 0.84 | 0.78 | 5.00 | 8.13 | 9.45 | 7.27 |
| (pH7.4/pH6.0) | | | | | | | | |

TABLE 11

| | | BA-143- | BA-143- | BA-143- | | BA-143- | BA-143- | BA-143- |
|---|---|---|---|---|---|---|---|---|
| T47D, EC50 | BA-143- | 00- | 00- | 00- | BA-143- | 01- | 01- | 01- |
| (ng/mL) | 00-MC | SPC04 | SPC07 | SPC08 | 01-MC | SPC04 | SPC07 | SPC08 |
| pH6.0 | 8.612 | 21.27 | 17.38 | 19.68 | 61.06 | 64.8 | 72.39 | 62.45 |
| pH7.4 | 14.61 | 7.463 | 16.48 | 24.45 | 422.3 | 435.4 | 454.7 | 445.9 |
| ratio | 1.70 | 0.35 | 0.95 | 1.24 | 6.92 | 6.72 | 6.28 | 7.14 |
| (pH7.4/pH6.0) | | | | | | | | |

Tables 8-11 show that the inventive linker does not have an adverse effect on the conditionally active antibodies.

### Example 13—pH Titration ELISA Assay

### 1.1 Test Articles

BA-143-00-MC (wildtype or parent non-CAB conjugated to MC)
BA-143-00-SPC04 (wildtype or parent non-CAB conjugated to SPC04)
BA-143-00-SPC07 (wildtype or parent non-CAB conjugated to SPC07)
BA-143-00-SPC08 (wildtype or parent non-CAB conjugated to SPC08)
BA-143-01-MC (CAB conjugated to MC)
BA-143-01-SPC04 (CAB conjugated to SPC04)
BA-143-01-SPC07 (CAB conjugated to SPC07)
BA-143-01-SPC08 (CAB conjugated to SPC08)
BA-B12-MC (isotype control antibody conjugated to MC)
BA-B12-SPC04 (isotype control antibody conjugated to SPC04)
BA-B12-SPC07 (isotype control antibody conjugated to SPC07)
BA-B12-SPC08 (isotype control antibody conjugated to SPC08)

### 1.2 Formulations

Test articles were first diluted to 1000 ng/ml in pH 6.0 or pH 7.4 ELISA incubation buffer. Then 1000 ng/ml of test articles were 5-fold serially diluted in pH 6.0 or pH 7.4 ELISA incubation buffer.

### 1.3 pH Affinity ELISA Assay

1) Coat ELISA plates with 100 μL of 1 μg/mL recombinant human Nectin4 antigen or cynomolgus Nectin4 antigen in carbonate-bicarbonate coating buffer (Sigma Aldrich Chemical Co., Cat. #C3041).
2) Cover plates with sealing film and incubate overnight at 4° C.
3) Decant plates and tap out residual liquid on a stack of paper towels.
4) Wash wells twice by dispensing 200 μL of pH 6.0 or pH 7.4 ELISA incubation buffer to the wells and completely aspirate the contents.
5) Add 200 μL of pH 6.0 or pH 7.4 ELISA incubation buffer to the wells. Cover with sealing film and place the plate onto a plate shaker set to 50 rpm for 60 minutes at room temperature.
6) Decant plates and tap out residual liquid on a stack of paper towels.
7) Serially dilute test articles in 5-fold dilutions starting at 1000 ng/ml in pH 6.0 or pH 7.4 ELISA incubation buffer.
8) Add 100 μL/well of diluted test articles to the plates
9) Cover with sealing film and place the plates onto a plate shaker set to 50 rpm for 60 minutes at room temperature.
10) Decant plates and tap out residual liquid on a stack of paper towels.
11) Wash wells three times by dispensing 200 μL of pH 6.0 or pH 7.4 ELISA wash buffer to the wells and completely aspirate the contents.
12) Dilute the HRP secondary antibody at 1:2500 in pH 6.0 or pH 7.4 ELISA incubation buffer.
13) Add 100 μL HRP secondary antibody diluted in pH 6.0 or pH 7.4 ELISA incubation buffer to each well.
14) Cover with sealing film and place the plates onto a plate shaker set to 50 rpm for 60 minutes at room temperature.
15) Decant plates and tap out residual liquid on a stack of paper towels.
16) Wash wells three times by dispensing 200 μL of pH 6.0 or pH 7.4 ELISA wash buffer to the wells and completely aspirate the contents.
17) Dispense 50 μL per well of the TMB substrate solution (3, 3′, 5, 5′ tetramethylbenzidine, Life Technologies Cat. #002023) into all wells of the plates. Incubate at room temperature for about 3 minutes.
18) Add 50 μL per well of 1N HCl into all wells of the plates. Read plates at 450 nm using PerkinElmer, EnSpire 2300 Multilabel Reader.

ELISA incubation buffer: add sodium bicarbonate and BSA to 1×PBS buffer to have 2.5 g/L final sodium bicarbonate and 1% BSA, adjust pH to 6.0 or 7.4 using 1N HCl.

Carbonate-bicarbonate coating buffer: dissolve 1 Carbonate-Bicarbonate buffer capsule (Sigma Aldrich Chemical Co., Cat. #C3041-100CAP) in 100 mL water.

ELISA wash buffer: add sodium bicarbonate and Tween-20 to 1×PBS buffer to have 2.5 g/L final sodium bicarbonate and 0.1% Tween-20, adjust pH to 6.0 or 7.4 using 1N HCl.

TABLE 12
pH Titration ELISA - human Nectin4

| | | | | | | | BA-143- | |
|---|---|---|---|---|---|---|---|---|
| human Nectin4, | BA-143- | BA-143- | BA-143- | BA-143- | BA-143- | BA-143- | 01- | BA-143- |
| EC50 (ng/ml) | 00-MC | 00-SPC04 | 00-SPC07 | 00-SPC08 | 01-MC | 01-SPC04 | SPC07 | 01-SPC08 |
| pH6.0 | 22.8 | 25.19 | 20.2 | 19.25 | 55.12 | 49.22 | 66.55 | 163.3 |
| pH7.4 | 27.14 | 24.38 | 16.57 | 15.39 | 214.6 | 218.4 | 889 | 3825 |
| ratio | 1.19 | 0.97 | 0.82 | 0.80 | 3.89 | 4.44 | 13.36 | 23.42 |
| (pH7.4/pH6.0) | | | | | | | | |

TABLE 13
pH Titration ELISA - cyno Nectin4

| | | | | | | | BA-143- | |
|---|---|---|---|---|---|---|---|---|
| cyno Nectin4, | BA-143- | BA-143- | BA-143- | BA-143- | BA-143- | BA-143- | 01- | BA-143- |
| EC50 (ng/mL) | 00-MC | 00-SPC04 | 00-SPC07 | 00-SPC08 | 01-MC | 01-SPC04 | SPC07 | 01-SPC08 |
| pH6.0 | 46.1 | 44.65 | 48.56 | 36.93 | 165.2 | 133.8 | 191.3 | 369.5 |
| pH7.4 | 43.86 | 44.71 | 42.99 | 41.19 | 163.7 | 653.2 | 300556 | 1.36E ± 06 |
| ratio | 0.95 | 1.00 | 0.89 | 1.12 | 0.99 | 4.88 | N/A | N/A |
| (pH7.4/pH6.0) | | | | | | | | |

### Example 14—pH Range ELISA

### 1.1 Test Articles

BA-143-00-MC (wildtype or parent non-CAB conjugated to MC)
BA-143-00-SPC04 (wildtype or parent non-CAB conjugated to SPC04)
BA-143-00-SPC07 (wildtype or parent non-CAB conjugated to SPC07)
BA-143-00-SPC08 (wildtype or parent non-CAB conjugated to SPC08)
BA-143-01-MC (CAB conjugated to MC)
BA-143-01-SPC04 (CAB conjugated to SPC04)
BA-143-01-SPC07 (CAB conjugated to SPC07)
BA-143-01-SPC08 (CAB conjugated to SPC08)
BA-B12-MC (isotype control antibody conjugated to MC)
BA-B12-SPC04 (isotype control antibody conjugated to SPC04)
BA-B12-SPC07 (isotype control antibody conjugated to SPC07)
BA-B12-SPC08 (isotype control antibody conjugated to SPC08)

### 1.2 Formulations

Test articles were diluted to 50 or 100 ng/mL for wild type (WT) and 100 or 200 ng/ml for CABs in various pH ELISA incubation buffers ranging from pH 6.0 to pH 7.4.

### 1.3 pH Range ELISA Asssay

1) Coat ELISA plates with 100 μL of 1 μg/mL recombinant human Nectin4 antigen in carbonate-bicarbonate coating buffer.
2) Cover plates with sealing film and incubate overnight at 4° C.
3) Decant plates and tap out residual liquid on a stack of paper towels.
4) Wash wells twice by dispensing 200 μL of various pH incubation buffers to the wells and completely aspirate the contents.
5) Add 200 μL of various pH incubation buffers (pH6.0, 6.2, 6.5, 6.7, 7.0 and 7.4) to the wells. Cover with sealing film and place the plate onto a plate shaker (set to 200 rpm) for 60 minutes at room temperature.
6) Decant plates and tap out residual liquid on a stack of paper towels.
7) Serially dilute test articles in various pH incubation buffers (pH6.0, 6.2, 6.5, 6.7, 7.0 and 7.4) to 50 or 100 ng/mL for WT and 100 or 200 ng/ml for CABs.
8) Add 100 μL/well of diluted test articles to the plates.
9) Cover with sealing film and place the plates onto a plate shaker (set to 200 rpm) for 60 minutes at room temperature.
10) Decant plates and tap out residual liquid on a stack of paper towels.
11) Wash wells three times by dispensing 200 μL of various pH wash buffers (pH6.0, 6.2, 6.5, 6.7, 7.0 and 7.4) to the wells and completely aspirate the contents.
12) Dilute the HRP secondary antibody at 1:2500 in various pH incubation buffers (pH6.0, 6.2, 6.5, 6.7, 7.0 and 7.4).
13) Add 100 μL HRP secondary antibody diluted in various pH incubation buffers (pH6.0, 6.2, 6.5, 6.7, 7.0 and 7.4) to each well.
14) Cover with sealing film and place the plates onto a plate shaker (set to 200 rpm) for 60 minutes at room temperature.
15) Decant plates and tap out residual liquid on a stack of paper towels.
16) Wash wells three times by dispensing 200 μL of various pH wash buffers (pH6.0, 6.2, 6.5, 6.7, 7.0 and 7.4) to the wells and completely aspirate the contents.
17) Dispense 50 μL per well of the TMB substrate solution (Life Technologies Cat. #002023) into all wells of plates. Incubate at room temperature for 4 minutes.
18) Add 50 μL per well of 1N HCl into all wells of the plates. Read plates at 450 nm using PerkinElmer EnSpire 2300 Multilabel Reader.

ELISA incubation buffer or pH incubation buffer: add sodium bicarbonate and BSA to 1×PBS buffer to have 2.5 g/L final sodium bicarbonate and 1% BSA, adjust pH to 6.0 or 7.4 using 1N HCl.

Carbonate-bicarbonate coating buffer: dissolve 1 Carbonate-Bicarbonate buffer capsule (Sigma Aldrich Chemical Co., Cat. #C3041-100CAP) in 100 mL water.

pH wash buffer: add 10 mL 75 g/mL sodium bicarbonate solution to 290 mL of 1×PBS buffer. Adjust pH to 6.0 or 7.4 using 6N or 0.6N HCl.

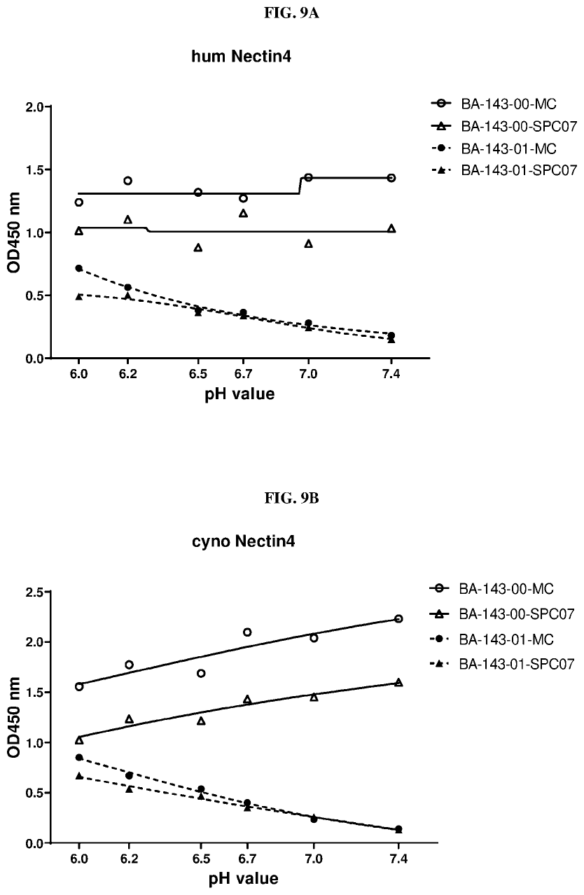

TABLE 14
pH Range ELISA - human Nectin4 (FIG. 9A)

| | BA-143- | BA-143- | BA-143- | BA-143- |
|---|---|---|---|---|
| antibodies | 00-MC | 00-SPC07 | 01-MC | 01-SPC07 |
| pH | N/A | N/A | 6.8 | 6.742 |
| inflection | | | | |
| point | | | | |

TABLE 15
pH Range ELISA - cyno Nectin4 (FIG. 9B)

| | BA-143- | BA-143- | BA-143- | BA-143- |
|---|---|---|---|---|
| antibodies | 00-MC | 00-SPC07 | 01-MC | 01-SPC07 |
| pH | 6.139 | 6.6 | 6.158 | 6.6 |
| inflection | | | | |
| point | | | | |

### Example 15—pH Affinity ELISA Assay

### 1.1 Test Articles

BA-143-00-MC (wildtype or parent non-CAB conjugated to MC)
BA-143-00-SPC04 (wildtype or parent non-CAB conjugated to SPC04)
BA-143-00-SPC07 (wildtype or parent non-CAB conjugated to SPC07)
BA-143-00-SPC08 (wildtype or parent non-CAB conjugated to SPC08)
BA-143-01-MC (CAB conjugated to MC)
BA-143-01-SPC04 (CAB conjugated to SPC04)
BA-143-01-SPC07 (CAB conjugated to SPC07)
BA-143-01-SPC08 (CAB conjugated to SPC08)
BA-B12-MC (isotype control antibody conjugated to MC)
BA-B12-SPC04 (isotype control antibody conjugated to SPC04)
BA-B12-SPC07 (isotype control antibody conjugated to SPC07)
BA-B12-SPC08 (isotype control antibody conjugated to SPC08)

### 1.2 Formulations

Test articles were first diluted to 10 μg/mL in pH 6.0 or pH 7.4 ELISA incubation buffer. Then 10 ug/mL of test articles were 10-fold serially diluted in pH 6.0 or pH 7.4 ELISA incubation buffer.

### 1.3 pH Affinity ELISA Assay

1) Coat ELISA plates with 100 μL of 1 μg/mL recombinant human Nectin4 antigen or cynomolgus Nectin4 antigen in carbonate-bicarbonate coating buffer.
2) Cover plates with sealing film and incubate overnight at 4° C.
3) Decant plates and tap out residual liquid on a stack of paper towels.
4) Wash wells twice by dispensing 200 μL of pH 6.0 or pH 7.4 ELISA incubation buffer to the wells and completely aspirate the contents.
5) Add 200 μL of pH 6.0 or pH 7.4 ELISA incubation buffer to the wells. Cover with sealing film and place the plate onto a plate shaker set to 50 rpm for 60 minutes at room temperature.
6) Decant plates and tap out residual liquid on a stack of paper towels.
7) Serially dilute test articles in 10-fold dilutions starting at 10 ug/mL in pH 6.0 or pH 7.4 ELISA incubation buffer.
8) Add 100 μL/well of diluted test articles to the plates
9) Cover with sealing film and place the plates onto a plate shaker set to 50 rpm for 60 minutes at room temperature.
10) Decant plates and tap out residual liquid on a stack of paper towels.
11) Wash wells three times by dispensing 200 μL of pH 6.0 or pH 7.4 ELISA wash buffer to the wells and completely aspirate the contents.
12) Dilute the HRP secondary antibody at 1:2500 in pH 6.0 or pH 7.4 ELISA incubation buffer.
13) Add 100 μL HRP secondary antibody diluted in pH 6.0 or pH 7.4 ELISA incubation buffer to each well
14) Cover with sealing film and place the plates onto a plate shaker set to 50 rpm for 60 minutes at room temperature.
15) Decant plates and tap out residual liquid on a stack of paper towels.
16) Wash wells three times by dispensing 200 μL of pH 6.0 or pH 7.4 ELISA wash buffer to the wells and completely aspirate the contents.
17) Dispense 50 μL per well of the TMB substrate solution (3, 3′, 5, 5′ tetramethylbenzidine, Life Technologies Cat #002023) into all wells of the plates. Incubate at room temperature for about 3 minutes.
18) Add 50 μL per well of 1N HCl into all wells of the plates. Read plates at 450 nm using PerkinElmer, EnSpire 2300 Multilabel Reader.

ELISA incubation buffer: add sodium bicarbonate and BSA to 1×PBS buffer to have 2.5 g/L final sodium bicarbonate and 1% BSA, adjust pH to 6.0 or 7.4 using 1N HCl.

ELISA wash buffer: add sodium bicarbonate and Tween-20 to 1×PBS buffer to have 2.5 g/L final sodium bicarbonate and 0.1% Tween-20, adjust pH to 6.0 or 7.4 using 1N HCl.

Carbonate-bicarbonate coating buffer: dissolve 1 Carbonate-Bicarbonate buffer capsule (Sigma Aldrich Chemical Co., Cat. #C3041-100CAP) in 100 mL water.

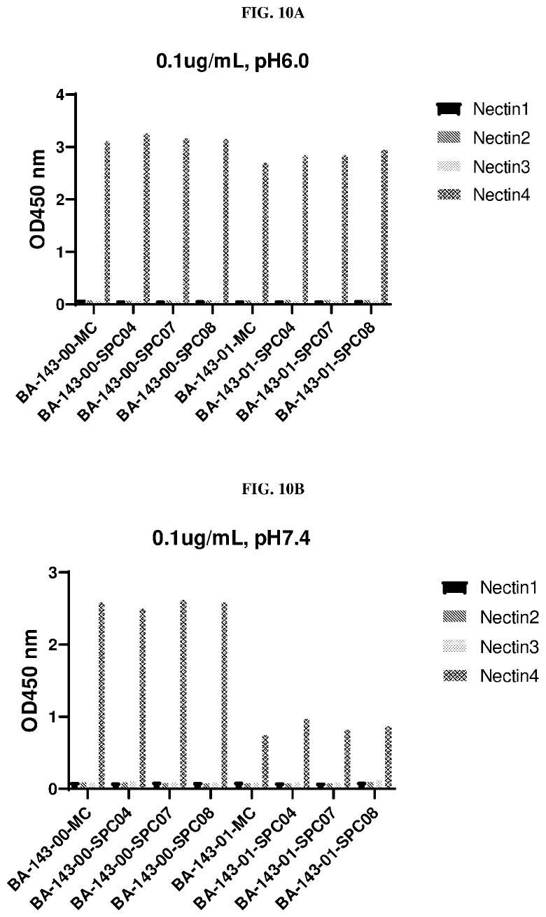

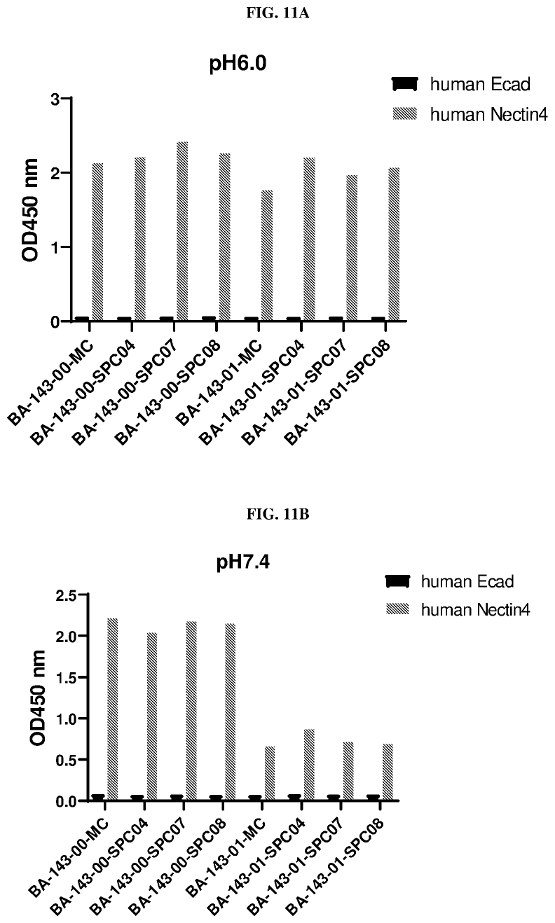

FIGS. 10A and 10B show BA-143-00-MC, BA-143-00-SPC04, BA-143-00-SPC07, BA-143-00-SPC08, BA-143-01-MC, BA-143-01-SPC04, BA-143-01-SPC07, and BA-143-01-SPC08 specifically bind to Nectin4, but that BA-143-01-MC, BA-143-01-SPC04, BA-143-01-SPC07, and BA-143-01-SPC08 exhibit reduced binding to Nectin4 at pH 7.4 in comparison to BA-143-00-MC, BA-143-00-SPC04, BA-143-00-SPC07, and BA-143-00-SPC08.

### Example 16—pH Affinity ELISA Assay Non-Related Antigen

### 1.1 Test Articles

BA-143-00-MC (wildtype or parent non-CAB conjugated to MC)
BA-143-00-SPC04 (wildtype or parent non-CAB conjugated to SPC04)
BA-143-00-SPC07 (wildtype or parent non-CAB conjugated to SPC07)
BA-143-00-SPC08 (wildtype or parent non-CAB conjugated to SPC08)
BA-143-01-MC (CAB conjugated to MC)
BA-143-01-SPC04 (CAB conjugated to SPC04)
BA-143-01-SPC07 (CAB conjugated to SPC07)
BA-143-01-SPC08 (CAB conjugated to SPC08)
BA-B12-MC (isotype control antibody conjugated to MC)
BA-B12-SPC04 (isotype control antibody conjugated to SPC04)
BA-B12-SPC07 (isotype control antibody conjugated to SPC07)
BA-B12-SPC08 (isotype control antibody conjugated to SPC08)

### 1.2 Formulations

Test articles were diluted to 50 ng/mL for wild type samples and 100 ng/ml for CAB samples in pH 6.0 or pH 7.4 ELISA incubation buffer.

1.3 pH Affinity ELISA Assay
1) Coat ELISA plates with 100 μL of 1 μg/mL recombinant human Nectin4 antigen or human E-cadherin antigen in carbonate-bicarbonate coating buffer (Sigma Aldrich Chemical Co., Cat #C3041).
2) Cover plates with sealing film and incubate overnight at 4° C.
3) Decant plates and tap out residual liquid on a stack of paper towels.
4) Wash wells twice by dispensing 200 μL of pH 6.0 or pH 7.4 ELISA incubation buffer to the wells and completely aspirate the contents.
5) Add 200 μL of pH 6.0 or pH 7.4 ELISA incubation buffer to the wells. Cover with sealing film and place the plate onto a plate shaker set to 50 rpm for 60 minutes at room temperature.
6) Decant plates and tap out residual liquid on a stack of paper towels.
7) Add 100 μL/well of diluted test articles to the plates
8) Cover with sealing film and place the plates onto a plate shaker set to 50 rpm for 60 minutes at room temperature.
9) Decant plates and tap out residual liquid on a stack of paper towels.
10) Wash wells three times by dispensing 200 μL of pH 6.0 or pH 7.4 ELISA wash buffer to the wells and completely aspirate the contents.
11) Dilute the HRP secondary antibody at 1:2500 in pH 6.0 or pH 7.4 ELISA incubation buffer.
12) Add 100 μL HRP secondary antibody diluted in pH 6.0 or pH 7.4 ELISA incubation buffer to each well.
13) Cover with sealing film and place the plates onto a plate shaker set to 50 rpm for 60 minutes at room temperature.
14) Decant plates and tap out residual liquid on a stack of paper towels.
15) Wash wells three times by dispensing 200 μL of pH 6.0 or pH 7.4 ELISA wash buffer to the wells and completely aspirate the contents.
16) Dispense 50 μL per well of the TMB substrate solution into all wells of the plates. Incubate at room temperature for about 3 minutes.
17) Add 50 μL per well of 1N HCl into all wells of the plates. Read plates at 450 nm using PerkinElmer, EnSpire 2300 Multilabel Reader.

ELISA incubation buffer: add sodium bicarbonate and BSA to 1×PBS buffer to have 2.5 g/L final sodium bicarbonate and 1% BSA, adjust pH to 6.0 or 7.4 using 1N HCl.

Carbonate-bicarbonate coating buffer: dissolve 1 Carbonate-Bicarbonate buffer capsule (Sigma Aldrich Chemical Co., Cat. #C3041-100CAP) in 100 mL water.

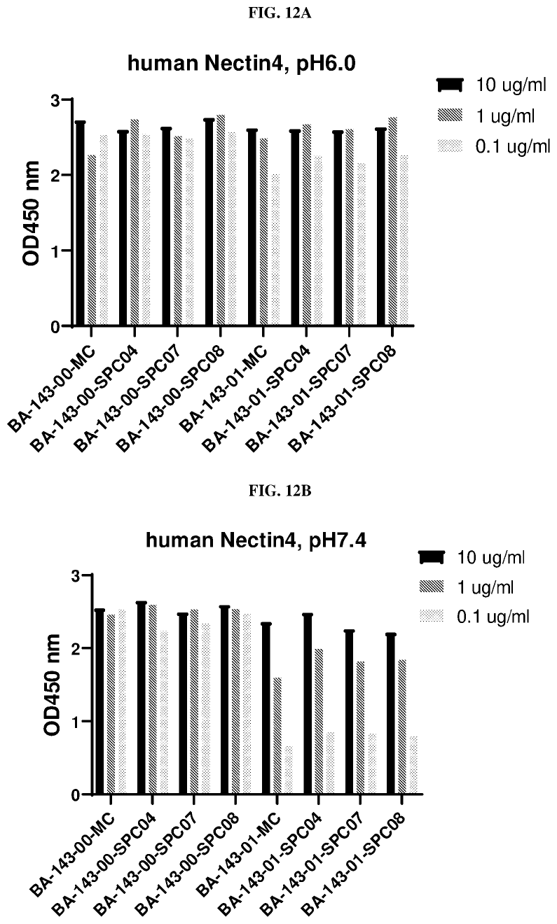

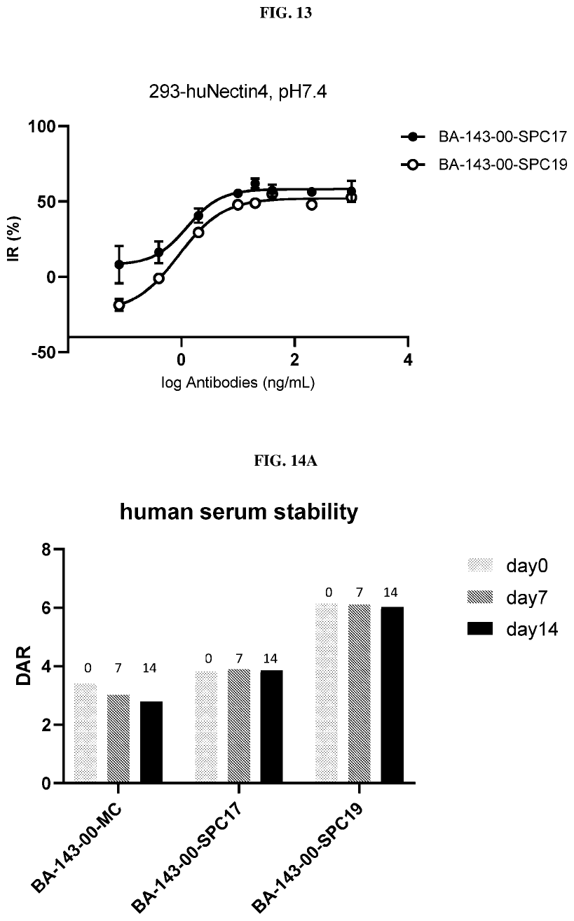

FIGS. 11A and 11B show BA-143-00-MC, BA-143-00-SPC04, BA-143-00-SPC07, BA-143-00-SPC08, BA-143-01-MC, BA-143-01-SPC04, BA-143-01-SPC07, and BA-143-01-SPC08 specifically bind to human Nectin4, but that BA-143-01-MC, BA-143-01-SPC04, BA-143-01-SPC07, and BA-143-01-SPC08 exhibit reduced binding to human Nectin4 at pH 7.4 in comparison to BA-143-00-MC, BA-143-00-SPC04, BA-143-00-SPC07, and BA-143-00-SPC08.

### Example 17—pH Affinity ELISA Assay Cross Species

### 1.1 Test Articles

BA-143-00-MC (wildtype or parent non-CAB conjugated to MC)
BA-143-00-SPC04 (wildtype or parent non-CAB conjugated to SPC04)
BA-143-00-SPC07 (wildtype or parent non-CAB conjugated to SPC07)
BA-143-00-SPC08 (wildtype or parent non-CAB conjugated to SPC08)
BA-143-01-MC (CAB conjugated to MC)
BA-143-01-SPC04 (CAB conjugated to SPC04)
BA-143-01-SPC07 (CAB conjugated to SPC07)
BA-143-01-SPC08 (CAB conjugated to SPC08)
BA-B12-MC (isotype control antibody conjugated to MC)
BA-B12-SPC04 (isotype control antibody conjugated to SPC04)
BA-B12-SPC07 (isotype control antibody conjugated to SPC07)
BA-B12-SPC08 (isotype control antibody conjugated to SPC08)

### 1.2 Formulations

Test articles were first diluted to 10 μg/mL in pH 6.0 or pH 7.4 ELISA incubation buffer. Then 10 ug/mL of test articles were 10-fold serially diluted in pH 6.0 or pH 7.4 ELISA incubation buffer.

### 1.3 pH Affinity ELISA Assay

1) Coat ELISA plates with 100 μL of 1 μg/mL recombinant human Nectin4 antigen or cynomolgus in carbonate-bicarbonate coating buffer (Sigma Aldrich Chemical Co. Cat. #C3041).
2) Cover plates with sealing film and incubate overnight at 4° C.
3) Decant plates and tap out residual liquid on a stack of paper towels.
4) Wash wells twice by dispensing 200 μL of pH 6.0 or pH 7.4 ELISA incubation buffer to the wells and completely aspirate the contents.
5) Add 200 μL of pH 6.0 or pH 7.4 ELISA incubation buffer to the wells. Cover with sealing film and place the plate onto a plate shaker set to 50 rpm for 60 minutes at room temperature.
6) Decant plates and tap out residual liquid on a stack of paper towels.
7) Serially dilute test articles in 10-fold dilutions starting at 10 μg/mL in pH 6.0 or pH 7.4 ELISA incubation buffer.
8) Add 100 μL/well of diluted test articles to the plates
9) Cover with sealing film and place the plates onto a plate shaker set to 50 rpm for 60 minutes at room temperature.
10) Decant plates and tap out residual liquid on a stack of paper towels.
11) Wash wells three times by dispensing 200 μL of pH 6.0 or pH 7.4 ELISA wash buffer to the wells and completely aspirate the contents.
12) Dilute the HRP secondary antibody at 1:2500 in pH 6.0 or pH 7.4 ELISA incubation buffer.
13) Add 100 μL HRP secondary antibody diluted in pH 6.0 or pH 7.4 ELISA incubation buffer to each well.
14) Cover with sealing film and place the plates onto a plate shaker set to 50 rpm for 60 minutes at room temperature.
15) Decant plates and tap out residual liquid on a stack of paper towels.
16) Wash wells three times by dispensing 200 μL of pH 6.0 or pH 7.4 ELISA wash buffer to the wells and completely aspirate the contents.
17) Dispense 50 μL per well of the TMB substrate solution (3, 3′, 5, 5′ tetramethylbenzidine Life Technologies Cat. #002023) into all wells of the plates. Incubate at room temperature for about 3 minutes.
18) Add 50 μL per well of 1N HCl into all wells of the plates. Read plates at 450 nm using PerkinElmer, EnSpire 2300 Multilabel Reader.

ELISA incubation buffer: add sodium bicarbonate and BSA to 1×PBS buffer to have 2.5 g/L final sodium bicarbonate and 1% BSA, adjust pH to 6.0 or 7.4 using 1N HCl.

Carbonate-bicarbonate coating buffer: dissolve 1 Carbonate-Bicarbonate buffer capsule (Sigma Aldrich Chemical Co., Cat. #C3041-100CAP) in 100 mL water.

FIGS. 12A and 12B show BA-143-00-MC, BA-143-00-SPC04, BA-143-00-SPC07, BA-143-00-SPC08, BA-143-01-MC, BA-143-01-SPC04, BA-143-01-SPC07, and BA-143-01-SPC08 specifically bind to human Nectin4 at 0.1 μl/ml, 1 μl/ml, and 10 μl/ml. However, 0.1 μl/ml of each of BA-143-01-MC, BA-143-01-SPC04, BA-143-01-SPC07, and BA-143-01-SPC08 exhibited reduced binding to human Nectin4 at pH 7.4 in comparison to BA-143-00-MC, BA-143-00-SPC04, BA-143-00-SPC07, and BA-143-00-SPC08.

TABLES 16-18 below show BA-143-00-MC, BA-143-00-SPC04, BA-143-00-SPC07, BA-143-00-SPC08, BA-143-01-MC, BA-143-01-SPC04, BA-143-01-SPC07, and BA-143-01-SPC08 binding to human Nectin4 (TABLE 16), to cyno Nectin4 (TABLE 17), and to rat Nectin4 (TABLE 18).

TABLE 16

| human Nectin4, | BA-143 | BA-143- | BA-143- | BA-143- | BA-143- | BA-143-01- | BA-143- | BA-143- |
|---|---|---|---|---|---|---|---|---|
| EC50 (ng/mL) | 00-MC | 00-SPC04 | 00-SPC07 | 00-SPC08 | 01-MC | SPC04 | 01-SPC07 | 01-SPC08 |
| pH6.0 | 22.8 | 25.19 | 20.2 | 19.25 | 55.12 | 49.22 | 66.55 | 163.3 |
| pH7.4 | 27.14 | 24.38 | 16.57 | 15.39 | 214.6 | 218.4 | 889 | 3825 |
| ratio (pH7.4/pH6.0) | 1.19 | 0.97 | 0.82 | 0.80 | 3.89 | 4.44 | 13.36 | 23.42 |

TABLE 17

| cyno Nectin4, EC50 | BA-143- | BA-143- | BA-143- | BA-143- | BA-143- | BA-143-01- | BA-143- | BA-143- |
|---|---|---|---|---|---|---|---|---|
| (ng/ml) | 00-MC | 00-SPC04 | 00-SPC07 | 00-SPC08 | 01-MC | SPC04 | 01-SPC07 | 01-SPC08 |
| pH6.0 | 46.1 | 44.65 | 48.56 | 36.93 | 165.2 | 133.8 | 191.3 | 369.5 |
| pH7.4 | 43.86 | 44.71 | 42.99 | 41.19 | 163.7 | 653.2 | 300556 | 1.36E ± 06 |
| ratio (pH7.4/pH6.0) | 0.95 | 1.00 | 0.89 | 1.12 | 0.99 | 4.88 | N/A | N/A |

TABLE 18

| rat Nectin4, EC50 | BA-143- | BA-143- | BA-143- | BA-143- | BA-143- | BA-143-01- | BA-143- | BA-143- |
|---|---|---|---|---|---|---|---|---|
| (ng/mL) | 00-MC | 00-SPC04 | 00-SPC07 | 00-SPC08 | 01-MC | SPC04 | 01-SPC07 | 01-SPC08 |
| pH6.0 | 30.93 | 26.34 | 23.64 | 21.83 | 57.46 | 52.16 | 107.5 | 138.6 |
| pH7.4 | 21.81 | 21.2 | 21.98 | 15.89 | 346.2 | 360.2 | 487.2 | 3.46E ± 02 |
| ratio | 0.71 | 0.80 | 0.93 | 0.73 | 6.03 | 6.91 | 4.53 | N/A |
| (pH7.4/pH6.0) | | | | | | | | |

### Example 18—pH Potency Assay (293 Human Nectin4 Cells

### 1.1 Test Articles

BA-143-00-SPC17 (wildtype or parent non-CAB conjugated to SPC17)
BA-143-00-SPC19 (wildtype or parent non-CAB conjugated to SPC19)

### 1.2 Cell Culturing

293-huNectin4, 293-cynoNectin4 and 293-ratNectin4 were maintained in stable clone culture medium (MEM+10% FBS+1 mg/mL G418). The cells were routinely sub-cultured twice per week. The cells were harvested during exponential growth phase and counted for plating.

### 1.3 Formulations

1) Seed the 4000 cells per well in 50 μL of pH 7.4 assay media and culture overnight
2) Make serial dilution of test article ADC stocks in pH 7.4 assay media in duplicates starting at 2 ug/mL
3) Add 50 mL of serially diluted 2×ADC stocks to the wells (Final starting concentration is 1 mg/mL)
   Incubate the treated cells at 37° C. in a 5% CO₂ incubator for 72 hours

### 1.4 Celltiter-Glo Luminescent Cell Viability Assay

1) Thaw the CellTiter-Glo buffer and equilibrate to room temperature prior to use
2) Equilibrate the lyophilized CellTiter-Glo substrate to room temperature prior to use
3) Transfer the entire liquid volume of CellTiter-Glo buffer into the amber bottle containing CellTiter-Glo substrate to reconstitute the lyophilized enzyme/substrate mixture. This forms the CellTiter-Glo reagent
4) Mix by gently vortex to obtain a homogeneous solution
5) Equilibrate the plate and its contents to room temperature
6) Add 70 mL CellTiter-Glo reagent in each well. Mix contents for 2 minutes on an orbital shaker at 100 rpm to induce cell lysis
7) Allow the plate to incubate at room temperature for 10 minutes to stabilize luminescent signal
8) Record luminescence on the SpectraMax i3X plate reader

### 1.5 Data Analysis

The inhibitions of different dose of test antibodies were plotted in concentration-response luminescence signals as shown in FIG. 13 and IC50 was calculated as shown in Table 19. The data were interpreted by GraphPad Prism software.

TABLE 19

| | BA-143-00- | BA-143-00- |
|---|---|---|
| clone | SPC17 | SPC19 |
| IC50 (ng/mL), pH 7.4 | 1.266 | 0.8975 |

### Example 19—Human Serum Stability Assay

1.1 Test Articles ADC
BA-143-00-SPC17 (wildtype or parent non-CAB conjugated to SPC17)
BA-143-00-SPC19 (wildtype or parent non-CAB conjugated to SPC19)

### 1.2 Serum Incubation

Test article ADCs were added to human serum at 0.2 mg/ml. The aliquoted ADC containing serum samples were incubated at 37° C. for 0, 7 and 14 days, and stored at −80° C. after collection for further analysis.

### 1.3 Immuno-Capture Method

Suspended protein A beads were added to the ADC samples in serum and incubated for 30 min at R.T. with 1400 rpm shaking. The capture beads were collected through filter plate by centrifuging at 150rcf for 1 min. Then the affinity captured ADCs were eluted by 100 mM pH2.7 glycine-HCL buffer for 10 min R.T. with 800 rpm shaking and neutralized by 2M pH7.5 Tris buffer.

### 1.4 RP-HPCL Method

After the samples were reduced by 150 mM DTT solution for 30 min at 37° C., reverse phase chromatography was performed to evaluate DAR profile. The changes in DAR (% DAR) were calculated as ((Day7 or Day14 DAR-Day0 DAR)/Day0 DAR)×100%.

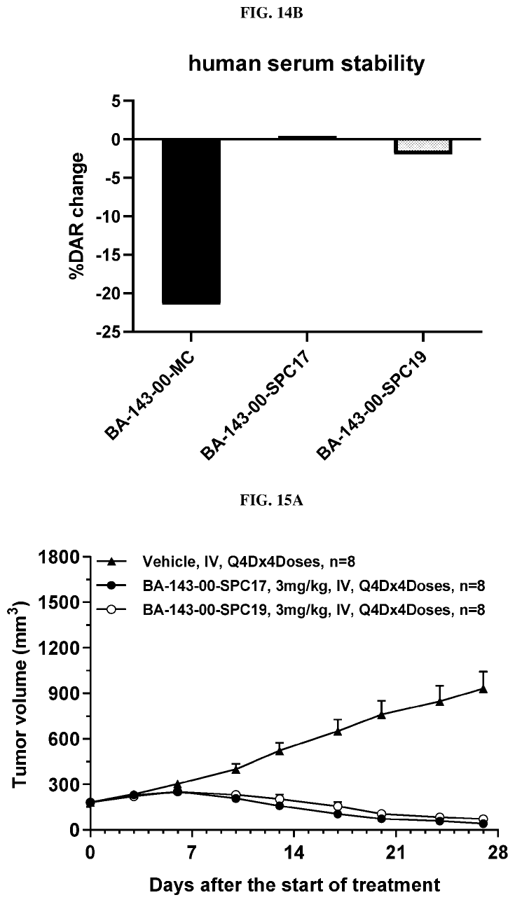

FIG. 14A shows was no difference in the DAR in human serum between Day 0, Day 7, and Day 14, for each of BA-143-00-SPC17, and BA-143-00-SPC19. However, there was a difference in % DAR change between the groups BA-143-00-SP17, and BA-143-00-SPC19 as shown in FIG. 14B.

### Example 20—In Vivo Test of Efficacy in Subcutaneous Xxt47D CDX Human Breast Cancer Xenograft Model In BABL/c Nude Mice

### Study Objective and Regulatory Compliance

The objective of this study was to evaluate the in vivo anti-tumor efficacy of the test articles in the subcutaneous xxT47D CDX human breast cancer xenograft model in female BALB/c nude mice.
This project was performed in compliance with the internal operating standards. The data generated by the project may not satisfy GLP standards.

### Abbreviations

| Abbreviation | Definition |
|---|---|
| AAALAC | Association for Assessment and Accreditation |
| | of Laboratory Animal Care |
| IACUC | Institutional Animal Care and Use Committee |
| ATCC | American Type Culture Collection |
| Q4D | Once every 4 days |
| GLP | Good Laboratory Practice |
| IV | Intravenous |
| SEM | Standard error of the mean |
| TGI | Tumor growth inhibition |
| TV | Tumor volume |
| RTV | Relative tumor volume |

### Experimental Design

TABLE 20-1
Description of experimental design

| | | | Dose | Dose volume | Conc. | | |
|---|---|---|---|---|---|---|---|
| Group | N^{a} | Treatment | (mg/kg) | (mL/kg ^{b}) | mg/mL | Route | Schedule |
| 1 | 8 | Vehicle | — | 10 | — | IV | Q4Dx4 doses |
| 2 | 8 | BA-143-00-SPC07 | 3 | 10 | 0.3 | IV | Q4Dx4 doses |
| 3 | 8 | BA-143-00-SPC14 | 3 | 10 | 0.3 | IV | Q4Dx4 doses |
| 4 | 8 | BA-143-00-SPC15 | 3 | 10 | 0.3 | IV | Q4Dx4 doses |
| 5 | 8 | BA-143-00-SPC16 | 3 | 10 | 0.3 | IV | Q4Dx4 doses |
| 6 | 8 | BA-143-00-SPC17 | 3 | 10 | 0.3 | IV | Q4Dx4 doses |
| 7 | 8 | BA-143-00-SPC18 | 3 | 10 | 0.3 | IV | Q4Dx4 doses |
| 8 | 8 | BA-143-00-SPC19 | 3 | 10 | 0.3 | IV | Q4Dx4 doses |
| 9 | 8 | BA-143-00-SPC20 | 3 | 10 | 0.3 | IV | Q4Dx4 doses |

Note:
^{a}N: number of animals per group.
^{b} Dose volume: dosing volume was adjusted to 10 μL/g body weight.

### Materials

### Animals and Housing Condition

### Animals

Species: Mus musculus
Strain: BALB/c nude
Age: 6-8 weeks
Sex: female
Body weight: 18.0-22.5 g
Number of animals: 72 mice plus spare
Animal supplier: Zhejiang Vital River Laboratory Animal Technology Co., Ltd. No. of certificate of quality: 20220718Abzz0619000528

### Housing Condition

The mice were kept in individual ventilation cages at constant temperature and humidity with 3/5 animals in each cage.
Temperature: 20-26° C.
Humidity 40-70%.
   Cages: Made of polycarbonate. The size is 300 mm×200 mm×180 mm. The bedding material is corn cob, which was changed twice per week.
   Diet: Animals had free access to irradiation sterilized dry granule food during the entire study period.
   Water: Animals had free access to sterile drinking water.
   Cage identification: The identification labels for each cage contained the following information: number of animals, sex, strain, date received, treatment, study number, group number and the starting date of the treatment.
   Animal identification: Animals were marked by ear tags.

### Test and Control Articles

BA-143-00-SPC07 (wildtype or parent non-CAB conjugated to SPC07)
BA-143-00-SPC14 (wildtype or parent non-CAB conjugated to SPC14)
BA-143-00-SPC15 (wildtype or parent non-CAB conjugated to SPC15)
BA-143-00-SPC16 (wildtype or parent non-CAB conjugated to SPC16)
BA-143-00-SPC17 (wildtype or parent non-CAB conjugated to SPC17)
BA-143-00-SPC18 (wildtype or parent non-CAB conjugated to SPC18)
BA-143-00-SPC19 (wildtype or parent non-CAB conjugated to SPC19)
BA-143-00-SPC20 (wildtype or parent non-CAB conjugated to SPC20)

### Experimental Instruments and Reagents

### Instruments

Instrument name: Carbon dioxide incubator
Supplier: Thermo Fisher
Equipment type: Heracell 240i
Instrument name: Centrifuge
Supplier: Eppendorf
Equipment type: 5424R
Instrument name: Analytical balance
Supplier: Sartorius
Equipment type: SECURA225D-1CN
Instrument name: Balance
Supplier: Changzhou tianzhiping
Equipment type: EL-2KJ
Instrument name: Caliper
Supplier: Mitutoyo
Equipment type: 0~150 mm

### Reagents

Product identification: PBS
Manufacturer: Hyclone
Cat number: SH30256.01
Lot number: AH29787896
Product identification: FBS
Manufacturer: DongLing
Cat number: FBSV500
Lot number: A22JV
Product identification: RPMI 1640
Manufacturer: Gibco
Cat number: 22400089
Lot number: 2462009
Product identification: Antibiotic-Antimycotic
Manufacturer: Meilunbio
Cat number: MA0110
Lot number: MA0110-Apr-15H
Product identification: Tryspin-EDTA
Manufacturer: Gibco
Cat number: 25200072
Lot number: 2488393
Product identification: Matrigel
Manufacturer: Corning
Cat number: 354234
Lot number: 2012002

### Experimental Methods and Procedures Methods

### Cell Culture

xxT47D cell which was generated from the T47D tumor cells (ATCC, Manassas, VA, cat #ATCC® HTB-133™) by in vivo passaging for twice will be used for this project. xxT47D cells will be maintained in vitro as a monolayer culture in RPMI-1640 medium supplemented with 0.2 Units/mL bovine insulin, 10% heat inactivated fetal bovine serum, 100 U/mL penicillin and 100 μg/mL streptomycin at 37° C. with 5% CO₂ in air. The tumor cells will be routinely subcultured twice per week by trypsin-EDTA treatment. The cells growing in an exponential growth phase will be harvested and counted for tumor inoculation.

### Tumor Inoculation and Animal Group Testing

Each mouse was inoculated with 0.18 mg 17-β-estradiol pellet 3 days before subcutaneously at the right flank with xxT47D CDX tumor cells (10×10⁶) in 0.2 mL of PBS supplemented with Matrigel (1:1) for tumor development. Treatments were started on day 8 after tumor inoculation when the average tumor size reached approximately 181 mm³. Animals were assigned into groups according to their tumor volume using an Excel-based stratified randomization program. Each group consisted of 8 tumor-bearing mice. The testing articles were administrated according to the experimental design shown in TABLE 17-1.

### Testing Article Preparation

TABLE 20-2
Description of test article preparation

| | Stock | | Concentration | |
|---|---|---|---|---|
| Compounds | (mg/mL) | Preparation | (mg/mL) | Storage |
| Vehicle | — | PBS | — | 4° C. |
| BA-143-00-SPC07 | 1.630 | Add 442 μL of BA-143-00-SPC07 (1.630 | 0.3 | 4° C. |
| | mg/mL | mg/mL) to 1.958 mL of PBS and mix well. | | |
| BA-143-00-SPC14 | 1.670 | Add 431 μL of BA-143-00-SPC07 (1.670 | 0.3 | 4° C. |
| | mg/mL | mg/mL) to 1.969 mL of PBS and mix well. | | |
| BA-143-00-SPC15 | 1.660 | Add 434 μL of BA-143-00-SPC07 (1.660 | 0.3 | 4° C. |
| | mg/mL | mg/mL) to 1.966 mL of PBS and mix well. | | |
| BA-143-00-SPC16 | 1.700 | Add 424 μL of BA-143-00-SPC07 (1.700 | 0.3 | 4° C. |
| | mg/mL | mg/mL) to 1.976 mL of PBS and mix well. | | |
| BA-143-00-SPC17 | 1.700 | Add 424 μL of BA-143-00-SPC07 (1.700 | 0.3 | 4° C. |
| | mg/mL | mg/mL) to 1.976 mL of PBS and mix well. | | |
| BA-143-00-SPC18 | 1.690 | Add 426 μL of BA-143-00-SPC07 (1.690 | 0.3 | 4° C. |
| | mg/mL | mg/mL) to 1.974 mL of PBS and mix well. | | |
| BA-143-00-SPC19 | 1.610 | Add 447 μL of BA-143-00-SPC07 (1.610 | 0.3 | 4° C. |
| | mg/mL | mg/mL) to 1.953 mL of PBS and mix well. | | |
| BA-143-00-SPC20 | 1.650 | Add 436 μL of BA-143-00-SPC07 (1.650 | 0.3 | 4° C. |
| | mg/mL | mg/mL) to 1.964 mL of PBS and mix well. | | |

Note:
The test article formulation was prepared before each dosing.

### Observations

All the procedures related to animal handling, care and the treatment in the study were performed according to the guidelines approved by the Institutional Animal Care and Use Committee (IACUC) of WuXi AppTec following the guidance of the Association for Assessment and Accreditation of Laboratory Animal Care (AAALAC). At the time of routine monitoring, the animals were daily checked for any effects of tumor growth and treatments on normal behavior such as mobility, food and water consumption (by looking only), body weight gain/loss (body weights were measured twice weekly), eye/hair matting and any other abnormal effect as stated in the protocol. Death and observed clinical signs were recorded for each animal in each group.

### Tumor Measurements and Endpoints

The major endpoint was to assess whether the tumor growth could be delayed. Tumor size was measured twice weekly in two dimensions using a caliper and was calculated using the formula: V=0.5 a×b² where a and b are the long and short diameters of the tumor, respectively. The tumor size was then used for calculations of T/C, TGI and RTV values.

The T/C value (in percent) is an indication of antitumor effectiveness; T and C are the mean volumes of the treated and control groups, respectively, on a given day.

TGI for each treatment group was calculated using the formula: TGI (%)=[1−(Ti−T0)/(Vi−V0)]×100; Ti is the average tumor volume of a treatment group on a given day, TO is the average tumor volume of the treatment group on day 0, V.

i is the average tumor volume of the vehicle control group on the same day with Ti, and V0 is the average tumor volume of the vehicle group on day 0.

Individual RTV (relative tumor volume) was calculated by dividing the tumor volume on a specific day by its volume on day 0. The RTV value of each mouse was calculated individually which was then used for mean RTV calculation for a group.

### Sampling

~50 μL serum was collected from 3 mice from group 2 to 9 at 24 hours and 96 hours (right before the 2^{nd} dose) post the first dose, respectively. The detailed sample list is in TABLE 20-3.

TABLE 20-3

| | Animal | Time | Serum | Time | Serum |
|---|---|---|---|---|---|
| Group# | No. | point | (μL) | point | (μL) |
| Group-2 | 499203 | 24 hr post | 50 | 96 hr post | 50 |
| BA-143-00-SPC07, 3 mg/kg | 499212 | 1st dosing | 50 | 1st dosing | 50 |
| IV, 10 μL/g, Q4D × 4 doses | 499217 | | 50 | | 50 |
| Group-3 | 499226 | 24 hr post | 50 | 96 hr post | 50 |
| BA-143-00-SPC14, 3 mg/kg | 499259 | 1st dosing | 50 | 1st dosing | 50 |
| IV, 10 μL/g, Q4D × 4 doses | 499261 | | 50 | | 50 |
| Group-4 | 499209 | 24 hr post | 50 | 96 hr post | 50 |
| BA-143-00-SPC15, 3 mg/kg | 499231 | 1st dosing | 50 | 1st dosing | 50 |
| IV, 10 μL/g, Q4D × 4 doses | 499239 | | 50 | | 50 |
| Group-5 | 499206 | 24 hr post | 50 | 96 hr post | 50 |
| BA-143-00-SPC16, 3 mg/kg | 499213 | 1st dosing | 50 | 1st dosing | 50 |
| IV, 10 μL/g, Q4D × 4 doses | 499218 | | 50 | | 50 |
| Group-6 | 499210 | 24 hr post | 50 | 96 hr post | 50 |
| BA-143-00-SPC17, 3 mg/kg | 499228 | 1st dosing | 50 | 1st dosing | 50 |
| IV, 10 μL/g, Q4D × 4 doses | 499233 | | 50 | | 50 |
| Group-7 | 499229 | 24 hr post | 50 | 96 hr post | 50 |
| BA-143-00-SPC18, 3 mg/kg | 499230 | 1st dosing | 50 | 1st dosing | 50 |
| IV, 10 μL/g, Q4D × 4 doses | 499247 | | 50 | | 50 |
| Group-8 | 499214 | 24 hr post | 50 | 96 hr post | 50 |
| BA-143-00-SPC19, 3 mg/kg | 499222 | 1st dosing | 50 | 1st dosing | 50 |
| IV, 10 μL/g, Q4D × 4 doses | 499224 | | 50 | | 50 |
| Group-9 | 499202 | 24 hr post | 50 | 96 hr post | 50 |
| BA-143-00-SPC20, 3 mg/kg | 499204 | 1st dosing | 50 | 1st dosing | 50 |
| IV, 10 μL/g, Q4D × 4 doses | 499219 | | 50 | | 50 |

### Statistical Analysis

The mean tumor volume of each group and SEM at different time points were calculated (TABLE 20-4). Statistical analysis of differences in the tumor volume among groups were conducted on the data obtained on Day 27 after the start of treatment at the end of the study. One-way ANOVA was performed to compare the mean tumor volumes and RTVs among groups. Comparisons between groups were carried out with Games-Howell test, as a significant F-statistics was obtained. All data were analyzed using Prism 8.0. p<0.05 was considered to be statistically significant.

### Results

### Mortality, Morbidity, and Body Weight Gain or Loss

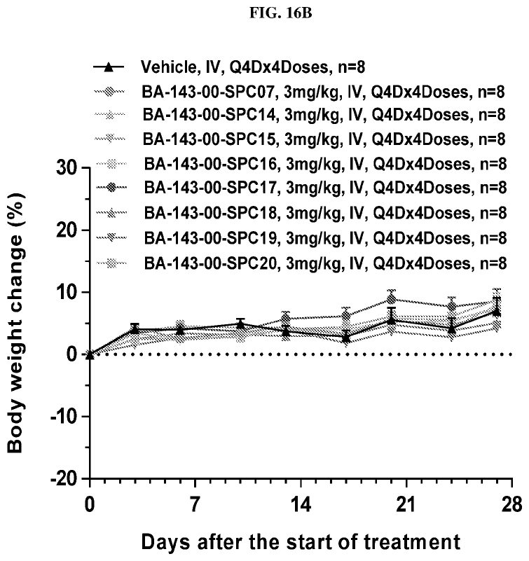

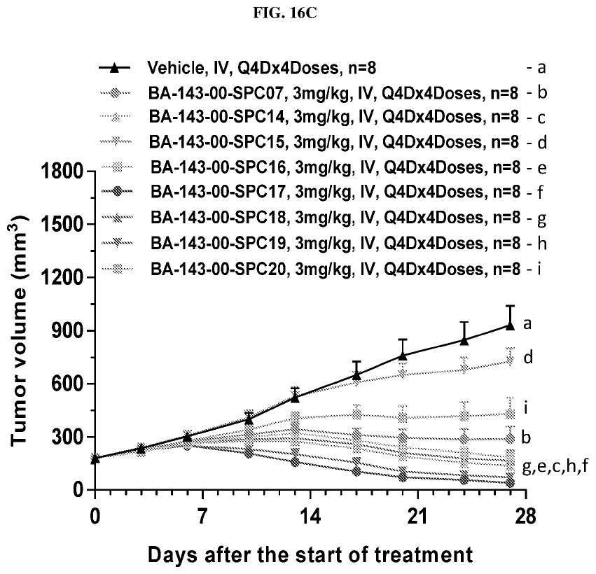

Animal body weight was monitored regularly as an indicator of toxicity. During this study, no group showed significant body weight loss (10% or above) (FIG. 16A). No death or morbidity was observed. Thus, no obvious toxicity was observed in association with the administration of the test articles to tumor-bearing BALB/c nude mice in the current dosing regimen.

Body weights and relative body weight changes of different groups are shown in FIG. 16A and FIG. 16B, respectively.

FIG. 16A shows the mean body weight of different treatment groups is shown. Data is presented as mean±SEM.

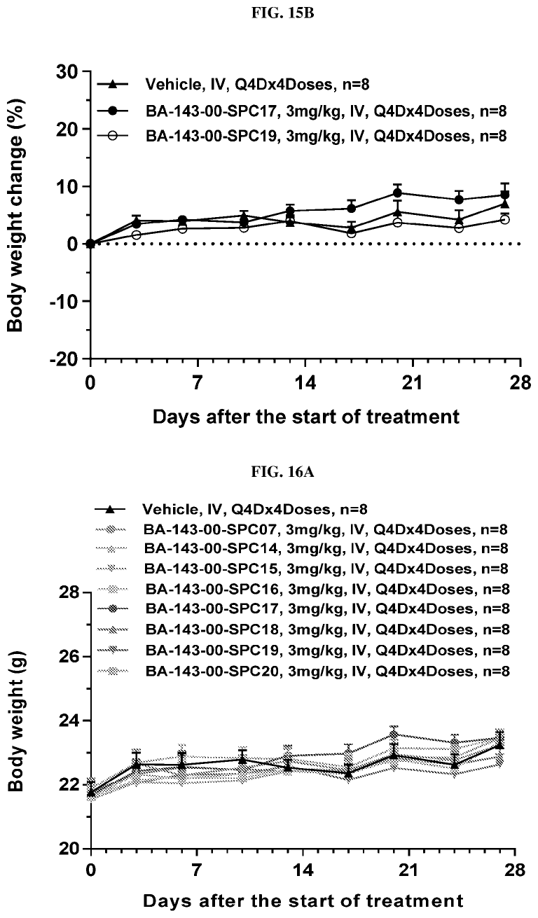

FIG. 16B shows the relative body weight changes (%) of different treatment groups is shown. In particular, the relative body weight changes of vehicle group (G1), BA-143-00-SPC17 (G6) and BA-143-00-SPC19 (G8) are shown in FIG. 15B. The percent change was calculated based on the animal weight on the first day of dosing. Data is presented as mean±SEM.

### Tumor Volume

The mean tumor volume of different groups is shown in TABLE 20-4 and in FIG. 16C. In particular, the mean tumor volume of vehicle group (G1), BA-143-00-SPC17 (G6) and BA-143-00-SPC19 (G8) are shown in FIG. 15A.

TABLE 20-4

| | Tumor volume (mm³) ^{a} | | | | | | | | |
|---|---|---|---|---|---|---|---|---|---|
| Days | G1 | G2 | G3 | G4 | G5 | G6 | G7 | G8 | G9 |
| 0 | 181 ± 12 | 181 ± 12 | 181 ± 12 | 181 ± 14 | 181 ± 14 | 181 ± 13 | 181 ± 12 | 181 ± 12 | 181 ± 13 |
| 3 | 235 ± 17 | 231 ± 14 | 215 ± 12 | 239 ± 16 | 222 ± 13 | 229 ± 16 | 219 ± 16 | 221 ± 14 | 227 ± 15 |
| 6 | 302 ± 23 | 276 ± 11 | 261 ± 14 | 306 ± 28 | 268 ± 15 | 251 ± 18 | 271 ± 19 | 251 ± 17 | 277 ± 14 |
| 10 | 399 ± 37 | 313 ± 25 | 277 ± 17 | 411 ± 40 | 299 ± 16 | 207 ± 21 | 286 ± 20 | 231 ± 24 | 341 ± 21 |
| 13 | 522 ± 52 | 344 ± 28 | 275 ± 19 | 529 ± 52 | 320 ± 17 | 158 ± 17 | 292 ± 29 | 203 ± 30 | 406 ± 33 |
| 17 | 651 ± 75 | 311 ± 37 | 233 ± 28 | 607 ± 60 | 282 ± 30 | 104 ± 12 | 259 ± 43 | 155 ± 28 | 427 ± 53 |
| 20 | 759 ± 90 | 295 ± 48 | 189 ± 23 | 648 ± 65 | 241 ± 35 | 72 ± 10 | 210 ± 52 | 105 ± 22 | 408 ± 68 |
| 24 | 847 ± 102 | 286 ± 60 | 155 ± 23 | 679 ± 68 | 212 ± 33 | 57 ± 8 | 178 ± 54 | 83 ± 21 | 417 ± 79 |
| 27 | 931 ± 110 | 288 ± 70 | 137 ± 29 | 726 ± 73 | 183 ± 33 | 41 ± 7 | 165 ± 58 | 70 ± 21 | 431 ± 90 |

Note:
^{a} Mean ± SEM
b. G1: Vehicle, G2: BA-143-00-SPC07 (3 mg/kg), G3: BA-143-00-SPC14 (3 mg/kg), G4: BA-143-00-SPC15 (3 mg/kg), G5: BA-143-00-SPC16 (3 mg/kg), G6: BA-143-00-SPC17 (3 mg/kg), G7: BA-143-00-SPC18 (3 mg/kg), G8: BA-143-00-SPC19 (3 mg/kg), G9: BA-143-00-SPC20 (3 mg/kg).

### Tumor Growth Inhibition Analysis

TABLE 20-5 shows tumor growth inhibition when compared to vehicle group (based on Day 27 data).

TABLE 20-5

| | Tumor Size | T/C ^{b} | TGI | | | |
|---|---|---|---|---|---|---|
| Treatment | (mm³) ^{ª} | (%) | (%) | RTV | p ^{c} | p ^{d} |
| Vehicle | 931 ± 110 | — | — | 5.19 ± 0.48 | — | — |
| BA-143-00-SPC07, 3 mg/kg | 288 ± 70 | 30.98 | 85.66 | 1.79 ± 0.47 | <0.0001 | <0.0001 |
| BA-143-00-SPC14, 3 mg/kg | 137 ± 29 | 14.72 | 105.88 | 0.81 ± 0.21 | <0.0001 | <0.0001 |
| BA-143-00-SPC15, 3 mg/kg | 726 ± 73 | 77.97 | 27.32 | 4.10 ± 0.45 | 0.2066 | 0.2199 |
| BA-143-00-SPC16, 3 mg/kg | 183 ± 33 | 19.65 | 99.71 | 0.99 ± 0.16 | <0.0001 | <0.0001 |
| BA-143-00-SPC17, 3 mg/kg | 41 ± 7 | 4.37 | 118.71 | 0.22 ± 0.05 | <0.0001 | <0.0001 |
| BA-143-00-SPC18, 3 mg/kg | 165 ± 58 | 17.74 | 102.11 | 0.84 ± 0.26 | <0.0001 | <0.0001 |
| BA-143-00-SPC19, 3 mg/kg | 70 ± 21 | 7.54 | 114.79 | 0.41 ± 0.14 | <0.0001 | <0.0001 |
| BA-143-00-SPC20, 3 mg/kg | 431 ± 90 | 46.28 | 66.69 | 2.43 ± 0.49 | <0.0001 | <0.0001 |

Note:
^{a} Mean ± SEM.
^{b} The tumor growth inhibition value was calculated by dividing the group average tumor volume of the treated group by the group average tumor volume of the vehicle control group (T/C).
^{c} p value calculated based on tumor size.
^{d} p value calculated based on RTV.

### Tumor Growth Curve

Tumor growth curve is shown in FIG. 16C. The tumor growth curves of different treatment groups are shown. Data is presented as mean±SEM.

### Summary and Discussion

This study evaluated the therapeutic efficacy of the test articles using the xxT47D CDX human breast cancer xenograft model. The tumor sizes of different groups at different time points after treatment are shown in

TABLE 20-4, TABLE 20-5 and FIG. 16C.

The mean tumor size of the vehicle-treated group reached 931 mm³ on Day 27 (RTV=5.19±0.48) after the start of treatment. The test articles BA-143-00-SPC15 at 3 mg/kg dose level produced nearly no antitumor activity (TGI=27.32%, p=0.2066, compared to vehicle). BA-143-00-SPC20 at 3 mg/kg dose level produced good antitumor activity (TGI=66.69%, p<0.0001, compared to vehicle) (TABLE 20-5).

The test articles BA-143-00-SPC14, BA-143-00-SPC16, BA-143-00-SPC17, BA-143-00-SPC18 and BA-143-00-SPC19 at 3 mg/kg dose level exhibited great anti-tumor activities (TGI=105.88%, 99.71%, 118.71%, 102.11% and 114.79%, all p values were less than 0.0001, compared to vehicle). (TABLE 20-5).

No severe body weight loss or death/morbidity event was observed. Thus, no obvious toxicity was observed in association with the administration of the test articles in this study.

The following anti-Nectin-4 antibodies were used in Examples 21-35 of the present disclosure as conjugates with a linker payload:

| Antibody Clones | Light chain variable region | Heavy chain variable region |
|---|---|---|
| BAP-143-wildtype | BA-143-VK-wildtype (SEQ ID NO 31) | BA-143-VH-wildtype (SEQ ID NO: 18) |
| or BAP-143-00-00 | | |
| or benchmark (BM) | | |
| BAP-143-00-01 | BA-143-00-01-VK (SEQ ID NO: 32) | BA-143-00-01-VH (SEQ ID NO: 19) |
| BAP-143-00-02 | BA-143-00-02-VK (SEQ ID NO: 33) | BA-143-00-02-VH (SEQ ID NO: 20) |
| BAP-143-00-03 | BA-143-00-03-VK (SEQ ID NO: 34) | BA-143-00-03-VH (SEQ ID NO: 21) |
| BAP-143-00-04 | BA-143-00-04-VK (SEQ ID NO: 35) | BA-143-00-04-VH (SEQ ID NO: 22) |
| BAP-143-00-05 | BA-143-00-05-VK (SEQ ID NO: 36) | BA-143-00-05-VH (SEQ ID NO: 23) |
| BAP-143-00-06 | BA-143-00-06-VK (SEQ ID NO: 37) | BA-143-00-06-VH (SEQ ID NO: 24) |
| BAP-143-06-24-16 | BA-143-06-24-16-VK (SEQ ID NO: 38) | BA-143-06-24-16-VH (SEQ ID NO: 25) |
| BAP-143-06-33-03 | BA-143-06-33-03-VK (SEQ ID NO: 39) | BA-143-06-33-03-VH (SEQ ID NO: 26) |
| BAP-143-06-33-16 | BA-143-06-33-16-VK (SEQ ID NO: 40) | BA-143-06-33-16-VH (SEQ ID NO: 27) |
| BAP-143-06-96-09 | BA-143-06-96-09-VK (SEQ ID NO: 41) | BA-143-06-96-09-VH (SEQ ID NO: 28) |
| BAP-143-07-03-12 | BAP-143-07-03-12-VK (SEQ ID NO: 42) | BAP-143-07-03-12-VH (SEQ ID NO: 29) |
| BAP-143-07-11-06 | BAP-143-07-11-06-VK (SEQ ID NO: 43) | BAP-143-07-11-06-VH (SEQ ID NO: 30) |

VK - refers to the light chain variable region
VH - refers to the heavy chain variable region

### Example 21—Binding Activity Of The Anti-Nectin-4 CAB ADCS and the WT ADC to Human Nectin-4

The binding activities of anti-Nectin-4 CAB ADCs and the WT ADC to human Nectin-4 were measured by ELISA in phosphate buffered saline supplemented with sodium bicarbonate (PSB), using a BM (benchmark) antibody as a control. The EC50 values of the anti-Nectin-4 CAB ADCs and the WT ADC for binding to human Nectin-4 at pH 6.0 and pH 7.4 are summarized in TABLE 21.

TABLE 21

| | Human Nectin4, EC50 (ng/mL) | | |
|---|---|---|---|
| hit name | PSB, pH 6.0 | PSB, pH 7.4 | pH 7.4/pH 6.0 |
| Benchmark (BM) | 31.55 | 20.2 | 0.64 |
| or BAP-143-00-00 | | | |
| BAP-143-00-01 | 31.26 | n/a | n/a |
| BAP-143-00-02 | 87.57 | n/a | n/a |
| BAP-143-00-03 | 34.41 | 245.2 | 7.13 |
| BAP-143-00-04 | 25.78 | 60.99 | 2.37 |
| BAP-143-00-05 | 44.11 | 141.1 | 3.20 |
| BAP-143-00-06 | 24.7 | 69.78 | 2.83 |

### Example 22: Binding Activity Of Anti-Nectin-4 CAB ADCS and the WT ADC To Human Nectin-4

The binding activities of more conditionally active anti-Nectin-4 antibodies to human Nectin-4 were similarly measured by ELISA. The EC50 values of the anti-Nectin-4 CAB ADCs and the WT ADC for binding to human Nectin-4 at pH 6.0 and pH 7.4 are summarized in TABLE 22.

TABLE 22

human Nectin-4, EC50 [ng/ml]

| hit name | PSB, pH 6.0 | PSB, pH 7.4 | pH 7.4/6.0 |
|---|---|---|---|
| BM (benchmark) | 21.65 | 21.59 | 1.00 |
| BAP-143-06-33-03 | 57.55 | n/a | n/a |
| BAP-143-06-24-16 | 36.8 | 133.4 | 3.63 |
| BAP-143-06-33-16 | 66.92 | n/a | n/a |
| BAP-143-07-03-12 | 53.32 | n/a | n/a |

### Example 23: Binding Activity of Anti-Nectin-4 CAB ADCs and the WT ADC to Cyno Nectin-4

The binding activities of anti-Nectin-4 CAB ADCs and the WT ADC to cynomolgus (cyno) Nectin-4 were similarly measured by ELISA. The EC50 values for binding to the cyno Nectin-4 at pH 6.0 and pH 7.4 for the anti-Nectin-4 CAB ADCs and the WT ADC are summarized in TABLE 23.

TABLE 23

cyno Nectin-4, EC50 [ng/ml]

| hit name | PSB, pH 6.0 | PSB, pH 7.4 | pH 7.4/6.0 |
|---|---|---|---|
| BM (benchmark) | 14.57 | 12.78 | 0.88 |
| BAP-143-06-33-03 | 39.51 | 72.29 | 1.83 |
| BAP-143-06-24-16 | 26.11 | 28.24 | 1.08 |
| BAP-143-06-33-16 | 38.43 | n/a | n/a |
| BAP-143-07-03-12 | 23.92 | 86.04 | 3.60 |

### Example 24: Binding Activity Of Anti-Nectin-4 CAB ADCS and the WT ADC To Human Nectin-4

The binding activities of anti-Nectin-4 CAB ADCs and the WT ADC to human Nectin-4 with pH range titration were similarly measured by ELISA. The pH inflection points of the anti-Nectin-4 CAB ADCs and the WT ADC to human Nectin-4 are summarized in TABLE 24.

TABLE 24

| | | BAP- | BAP- | BAP- | BAP- | BAP- | BAP- |
|---|---|---|---|---|---|---|---|
| | Benchmark | 143- | 143- | 143- | 143- | 143- | 143- |
| hit name | (BM) | 00-01 | 00-02 | 00-03 | 00-04 | 00-05 | 00-06 |
| pH | n/a | 6.59 | 6.70 | 6.71 | 6.81 | 6.99 | 6.77 |
| inflection | | | | | | | |
| point | | | | | | | |

### Example 25: Binding Activity of Anti-Nectin-4 CAB ADCS and the WT ADC to Human Nectin-4

The binding activities of anti-Nectin-4 CAB ADCs and the WT ADC to human Nectin-4 with pH range titration were similarly measured by ELISA. The pH inflection points of the anti-Nectin-4 CAB ADCs and the WT ADC to human Nectin-4 are summarized in TABLE 25.

TABLE 25

| | BM | BAP143- | BAP143- | BAP143- | BAP143- | BAP143- |
|---|---|---|---|---|---|---|
| hit name | (benchmark) | 00-01 | 06-33-03 | 06-24-16 | 06-33-16 | 07-03-12 |
| pH inflection | N/A | 6.58 | 6.85 | N/A | 6.6 | 6.51 |
| point | | | | | | |

### Example 26: Binding Activity Of Anti-Nectin-4 CAB ADCS and the WT ADC Measured by FACS

FACS analysis was carried out using HEK293 cells expressing human Nectin-4. The anti-Nectin-4 CAB ADCs and the WT ADC also consistently showed higher binding activity to the HEK293 cells expressing human Nectin-4 at pH 6.0 than at pH 7.4. The EC50 values for binding to the HEK293 cells expressing human Nectin-4 by the exemplary anti-Nectin-4 CAB ADCs and the WT ADC are summarized in TABLE 26.

TABLE 26

| | BM | BAP143- | BAP143- | BAP143- | BAP143- | BAP143- | BAP143- |
|---|---|---|---|---|---|---|---|
| hit name | (benchmark) | 00-01 | 00-02 | 00-03 | 00-04 | 00-05 | 00-06 |
| EC50 pH 6.0 | 123.7 | 148.5 | 253.6 | 184 | 159 | 206.7 | 195.7 |
| EC50 pH 7.4 | 148.1 | 489.6 | 410.9 | 372.1 | 336.4 | 352.4 | 239.1 |
| Ratio (pH | 1.2 | 3.3 | 1.6 | 2.0 | 2.1 | 1.7 | 1.2 |
| 7.4/6.0) | | | | | | | |

### Example 27: Binding Activity of Anti-Nectin-4 CAB ADCS and the WT ADC Measured by FACS

Binding activities of anti-Nectin-4 CAB ADCs and the WT ADC to HEK293 cells expressing cyno Nectin-4 cells were measured by FACS at pH 6.0 and pH 7.4. The conditionally active antibodies consistently showed higher binding activities to the cyno Nectin-4 cells at pH 6.0 than at pH 7.4. The EC50 values for binding to the cyno Nectin-4 cells expressing cyno Nectin-4 by the anti-Nectin-4 CAB ADCs and the WT ADC are summarized in TABLE 27.

TABLE 27

| | BM | BAP143- | BAP143- | BAP143- | BAP143- | BAP143- | BAP143- |
|---|---|---|---|---|---|---|---|
| hit name | (benchmark) | 00-01 | 00-02 | 00-03 | 00-04 | 00-05 | 00-06 |
| EC50 pH 6.0 | 64.5 | 107.2 | 159.6 | 107.1 | 97.1 | 114.8 | 87.9 |
| EC50 pH 7.4 | 112 | 439.2 | 367.5 | 389.6 | 204.3 | 290.3 | 280.9 |
| Ratio (pH | 1.7 | 4.1 | 2.3 | 3.6 | 2.1 | 2.5 | 3.2 |
| 7.4/6.0) | | | | | | | |

### Example 28: Binding Activity of Anti-Nectin-4 CAB ADCS and the WT ADC Measured by FACS

Binding activities of anti-Nectin-4 CAB ADCs and the WT ADC to T47D cells that express human Nectin-4 were measured by FACS at pH 6.0 and pH 7.4. The conditionally active antibodies consistently showed higher binding activities to the T47D cells at pH 6.0 than at pH 7.4. The EC50 values for binding to the T47D cells by the anti-Nectin-4 CAB ADCs and the WT ADC are summarized in TABLE 28.

TABLE 28

| | BM | BAP143- | BAP143- | BAP143- | BAP143- | BAP143- | BAP143- |
|---|---|---|---|---|---|---|---|
| hit name | (benchmark) | 00-01 | 00-02 | 00-03 | 00-04 | 00-05 | 00-06 |
| EC50 pH 6.0 | 14.06 | 28.27 | 57.8 | 30.82 | 17.99 | 34.86 | 19.54 |
| EC50 pH 7.4 | 12.71 | 189.2 | 165.2 | 111.3 | 37.43 | 83.96 | 50.38 |
| Ratio (pH | 0.9 | 6.7 | 2.9 | 3.6 | 2.1 | 2.4 | 2.6 |
| 7.4/6.0) | | | | | | | |

### Example 29: Binding Activity of Anti-NECTIN-4 CAB ADCS and the WT ADC Measured by FACS

FACS analysis was carried out using HEK293 cells expressing human Nectin-4. The anti-Nectin-4 CAB ADCs and the WT ADC also consistently showed higher binding activity to the HEK293 cells expressing human Nectin-4 at pH 6.0 than at pH 7.4. The EC50 values for binding to the HEK293 cells expressing human Nectin-4 by the exemplary anti-Nectin-4 CAB ADCs and the WT ADC are summarized in TABLE 29.

TABLE 29

| | BM | BAP143- | BAP143- | BAP143- | BAP143- | BAP143- |
|---|---|---|---|---|---|---|
| hit name | (benchmark) | 00-01 | 06-33-03 | 06-33-16 | 06-24-16 | 07-03-12 |
| EC50 pH 6.0 | 254.2 | 251.8 | 362 | 271.4 | 325.5 | 333 |
| EC50 pH 7.4 | 283.8 | 560.7 | 1005 | 406.2 | 471.5 | 675.1 |
| Ratio (pH | 1.1 | 2.2 | 2.8 | 1.5 | 1.4 | 2.0 |
| 7.4/6.0) | | | | | | |

### Example 30: Binding Activity of Anti-Nectin-4 CAB ADCS and the WT ADC Measured by FACS

Binding activities of further exemplary anti-Nectin-4 CAB ADCs and the WT ADC to HEK293 cells expressing cyno Nectin-4 were measured by FACS at pH 6.0 and pH 7.4. The conditionally active antibodies consistently showed higher binding activities to the cyno Nectin-4 cells at pH 6.0 than at pH 7.4. The EC50 values for binding to the cyno Nectin-4 cells expressing cyno Nectin-4 by the anti-Nectin-4 CAB ADCs and the WT ADC are summarized in TABLE 30.

TABLE 30

| | BM | BAP-143- | BAP-143- | BAP-143- | BAP-143- | BAP-143- |
|---|---|---|---|---|---|---|
| hit name | (benchmark) | 00-01 | 06-33-03 | 06-24-16 | 06-33-16 | 07-03-12 |
| EC50 pH 6.0 | 161.1 | 176.1 | 213.8 | 207.4 | 174 | 194.2 |
| EC50 pH 7.4 | 181.8 | 351.4 | 574 | 287.4 | 257.3 | 352 |
| Ratio (pH | 1.1 | 2.0 | 2.7 | 1.4 | 1.5 | 1.8 |
| 7.4/6.0) | | | | | | |

### Example 31: Binding Activity of Anti-Nectin-4 CAB ADCS and the WT ADC Measured by FACS

Binding activities of further exemplary anti-Nectin-4 CAB ADCs and the WT ADC to T47D cells that express human Nectin-4 were measured by FACS at pH 6.0 and pH 7.4. The conditionally active anti-bodies consistently showed higher binding activities to the T47D cells at pH 6.0 than at pH 7.4. The EC50 values for binding to the T47D cells by the anti-Nectin-4 CAB ADCs and the WT ADC are summarized in TABLE 31.

TABLE 31

| | BM | BAP-143- | BAP-143- | BAP-143- | BAP-143- | BAP-143- |
|---|---|---|---|---|---|---|
| hit name | (benchmark) | 00-01 | 06-33-03 | 06-24-16 | 06-33-16 | 07-03-12 |
| EC50 pH 6.0 | 28.81 | 41.24 | 30.18 | 25.29 | 20.37 | 47.06 |
| EC50 pH 7.4 | 34.95 | 255.9 | 447.9 | 73.17 | 68.39 | 212.3 |
| Ratio (pH | 1.2 | 6.2 | 14.8 | 2.9 | 3.4 | 4.5 |
| 7.4/6.0) | | | | | | |

### Example 32: In Vitro Cell Killing of HEK293 Cells Expressing Human Nectin-4

In vitro cell killing of HEK293 cells expressing human Nectin-4 was similarly analyzed using HEK293 cells expressing human Nectin-4 at pH values of 6.0 and 7.4. in vitro The EC50 values for the cell killing of HEK293 cells by the anti-Nectin-4 CAB ADCs and the WT ADC are shown in TABLE 32.

TABLE 32

| | BA-143-00- | BA-143-00- | BA-143-00- | BA-143-00- | BA-143-01- | BA-143- | BA-143- | BA-143- |
|---|---|---|---|---|---|---|---|---|
| IC50 (ng/mL) | MC | SPC04 | SPC07 | SPC08 | MC | 01-SPC04 | 01-SPC07 | 01-SPC08 |
| pH6.0 | 21.53 | 17.48 | 10.52 | 14.91 | 20.48 | 15.18 | 10.92 | 12.42 |
| pH7.4 | 20.08 | 13.22 | 8.771 | 11.67 | 23.41 | 15.54 | 10.96 | 12.05 |
| pH7.4/pH6.0 | 0.93 | 0.76 | 0.83 | 0.78 | 1.14 | 1.02 | 1.00 | 0.97 |

### Example 33: In Vivo Test of the Efficacy of Representative Anti-Nectin-4 CAB ADCs and the WT ADC in the Subcutaneous T47D CDX Model

### 1. Study Objective and Regulatory Compliance

The objective of this project is to evaluate the in vivo anti-tumor efficacy of representative anti-Nectin-4 CAB ADCs and the WT ADC in the treatment of the subcutaneous T47D breast cancer CDX model in BALB/c nude mice. LP1 is a proprietary linker payload.

### 1. Abbreviations

| Abbreviation | Definition |
|---|---|
| AAALAC | Association for Assessment and Accreditation |
| | of Laboratory Animal Care |
| IACUC | Institutional Animal Care and Use Committee |
| Q4D | Once every 4 days |
| GLP | Good Laboratory Practice |
| IV | Intravenous |
| SEM | Standard error of the mean |
| TGI | Tumor growth inhibition |
| TV | Tumor volume |
| RTV | Relative tumor volume |

### 2, Experimental Design

TABLE 33-1
Description of experimental design

| | | | Dose | Dose | | |
|---|---|---|---|---|---|---|
| | | | (mg/ | Volume | | |
| Group | N | Treatment | kg) | (mL/Kg) | Route | Schedule |
| 1 | 7 | Vehicle | — | 10 | IV | Q4Dx4 |
| | | | | | | doses |
| 2 | 7 | B12-LP1 | 3 | 10 | IV | Q4Dx4 |
| | | | | | | doses |
| 3 | 7 | Benchmark | 3 | 10 | IV | Q4Dx4 |
| | | (BM) or BAP- | | | | doses |
| | | 143-00-00-LP1 | | | | |
| 4 | 7 | BAP-143-00-01-LP1 | 3 | 10 | IV | Q4Dx4 |
| | | | | | | doses |
| 5 | 7 | BAP-143-00-02-LP1 | 3 | 10 | IV | Q4Dx4 |
| | | | | | | doses |
| 6 | 7 | BAP-143-00-03-LP1 | 3 | 10 | IV | Q4Dx4 |
| | | | | | | doses |
| 7 | 7 | BAP-143-00-04-LP1 | 3 | 10 | IV | Q4Dx4 |
| | | | | | | doses |
| 8 | 7 | BAP-143-00-05-LP1 | 3 | 10 | IV | Q4Dx4 |
| | | | | | | doses |
| 9 | 7 | BAP-143-00-06-LP1 | 3 | 10 | IV | Q4Dx4 |
| | | | | | | doses |

Note:
a. N: number of animals per group.
b. Dose volume: dosing volume was adjusted to 10 μL/g body weight.
c. B12 is isotype control

### Materials

### Animals and Housing Condition

### Animals

Species: Mus musculus
Strain: BALB/c nude
Age: 6-8 weeks
Sex: female
Body weight: 18-22 g
Number of animals: 63 mice plus spare
Animal supplier: Shanghai Lingchang Biological Technology Co., LTD.
No. of certificate of quality: 20180003002490

### Housing Condition

The mice were kept in individual ventilation cages at constant temperature and humidity with 4 or 3 animals in each cage.
Temperature: 20-26° C.
Humidity 40-70%.
Cages: Made of polycarbonate. The size is 300 mm×200 mm×180 mm. The bedding material is corn cob, which was changed twice per week.
Diet: Animals had free access to irradiation sterilized dry granule food during the entire study period.
Water: Animals had free access to sterile drinking water.
Cage identification: The identification labels for each cage contained the following information: number of animals, sex, strain, received date, treatment, study number, group number and the starting date of the treatment.
Animal identification: Animals were marked by ear tags.

### Experimental Methods and Procedures

### Cell Culture

T47D cells which were generated from T47D tumor cells (ATCC, Manassas, VA, cat #ATCC® HTB-133™) by in vivo passaging twice will be used for this project. T47D cells were maintained in vitro as a monolayer culture in RPMI-1640 medium supplemented with 0.2 Units/ml bovine insulin, 10% heat inactivated fetal bovine serum, 100 U/ml penicillin and 100 μg/ml streptomycin at 37° C. with 5% CO₂ in air. The tumor cells were routinely subcultured twice per week by trypsin-EDTA treatment. The cells growing in an exponential growth phase were harvested and counted for tumor inoculation.

### Tumor Inoculation and Animal Grouping

Each mouse was inoculated with 0.18 mg 17-β-estradiol pellet 3 days before subcutaneously cell inoculation on the right flank with xxT47D tumor cells (10×10⁶ with Matrigel) in 0.2 ml of PBS for tumor development. Treatments were started on day 6 after tumor inoculation when the average tumor size reached approximately 152 mm³. Animals were assigned into groups according to their tumor volume using an Excel-based program for stratification and randomization. Each group consisted of 7 tumor-bearing mice. The testing articles were administered according to the experimental design shown in TABLE 30-1.

### Testing Article Preparation

TABLE 33-2
Description of test article preparation

| Compounds | Stock | Preparation | Conc. |
|---|---|---|---|
| B12-LP1 | 1.12 | 0.56 mL stock plus | 0.3 |
| | mg/mL | 1.54 mL PBS. | mg/mL |
| BAP-143- | 1.46 | 0.43 mL stock plus | 0.3 |
| 00-00-LP1 | mg/mL | 1.67 mL PBS. | mg/mL |
| BAP-143- | 1.55 | 0.41 mL stock plus | 0.3 |
| 00-01-LP1 | mg/mL | 1.69 mLPBS. | mg/mL |
| BAP-143- | 1.2 | 0.53 mL stock plus | 0.3 |
| 00-02-LP1 | mg/mL | 1.57 mL PBS. | mg/mL |
| BAP-143- | 1.54 | 0.41 mL stock plus | 0.3 |
| 00-03-LP1 | mg/mL | 1.69 mL PBS. | mg/mL |
| BAP-143- | 1.1 | 0.57 mL stock plus | 0.3 |
| 00-04-LP1 | mg/mL | 1.53 mL PBS. | mg/mL |
| BAP-143- | 1.36 | 0.46 mL stock plus | 0.3 |
| 00-05-LP1 | mg/mL | 1.64 mL PBS. | mg/mL |
| BAP-143- | 1.56 | 0.40 mL stock plus | 0.3 |
| 00-06-LP1 | mg/mL | 1.70 mL PBS. | mg/mL |

Note:
The test article formulation was prepared before each dosing.

### Observation

All the procedures related to animal handling, care and the treatment in the study were performed according to the guidelines approved by the Institutional Animal Care and Use Committee (IACUC) of WuXi AppTec following the guidance of the Association for Assessment and Accreditation of Laboratory Animal Care (AAALAC). During routine monitoring, the animals were checked daily for any treatment effects on tumor growth and normal behavior such as mobility, food and water consumption (by looking only), body weight gain/loss (measured twice weekly), eye/hair matting and any other abnormal effects as stated in the protocol. Morbidity/mortality and other adverse observed events were recorded.

### Tumor Measurements and Endpoints

The major endpoint was to assess whether the tumor growth could be delayed. Tumor size was measured twice weekly in two dimensions using a caliper and was calculated using the formula: V=0.5 a×b² where a and b are the long and short diameters of the tumor, respectively. The tumor size was then used for calculations of T/C values. The T/C value (in percent) is an indication of antitumor effectiveness; T and C are the mean volumes of the treated and control groups, respectively, on a given day.

TGI for each treatment group was calculated using the formula: TGI (%)=[1−(Ti−T0)/(Vi−V0)]×100; Ti is the average tumor volume of a treatment group on a given day, TO is the average tumor volume of the treatment group on day 0, Vi is the average tumor volume of the vehicle control group on the same day with Ti, and V0 is the average tumor volume of the vehicle group on day 0.

Individual RTV (relative tumor volume) was calculated by dividing the tumor volume on a specific day by its volume on day 0. The RTV value of each mouse was calculated individually which was then used for mean RTV calculation for a group.

### Sampling

~50 μL serum was collected from 3 mice per group at 24 hours and 96 hours (right before the 2nd dose) post the first dose, respectively.

### Statistical Analysis

The mean tumor volume and SEM of each group at different time points were calculated (Table 3-1). Statistical analysis of difference in the tumor volume among groups were conducted on the data obtained on Day 28 after the start of treatment.
One-way ANOVA was performed to compare the mean tumor volumes and RTVs among groups. A significant F-statistics was obtained and comparisons between groups were carried out with Games-Howell test. All data were analyzed using IBM® SPSS Statistics® software (version 17.0.). p<0.05 was considered to be statistically significant.

### Results

### Tumor Volume

The mean tumor volume of different groups is shown in TABLE 33-3.

TABLE 33-3
Tumor volume

| | Tumor volume (mm³) ^{a} | | | | | | | | |
|---|---|---|---|---|---|---|---|---|---|
| Days | G1^{b} | G2 | G3 | G4 | G5 | G6 | G7 | G8 | G9 |
| 0 | 153 ± 13 | 153 ± 15 | 152 ± 18 | 152 ± 16 | 152 ± 15 | 152 ± 14 | 152 ± 14 | 152 ± 15 | 152 ± 17 |
| 4 | 220 ± 20 | 178 ± 24 | 148 ± 19 | 173 ± 20 | 174 ± 16 | 143 ± 16 | 167 ± 16 | 171 ± 23 | 157 ± 25 |
| 7 | 316 ± 27 | 263 ± 41 | 72 ± 17 | 93 ± 13 | 100 ± 15 | 83 ± 10 | 131 ± 15 | 122 ± 24 | 88 ± 15 |
| 11 | 451 ± 28 | 361 ± 66 | 36 ± 8 | 52 ± 4 | 57 ± 7 | 50 ± 4 | 145 ± 24 | 90 ± 20 | 31 ± 8 |
| 14 | 574 ± 36 | 460 ± 89 | 19 ± 5 | 36 ± 4 | 48 ± 6 | 31 ± 4 | 176 ± 32 | 75 ± 20 | 19 ± 5 |
| 18 | 733 ± 50 | 634 ± 163 | 7 ± 4 | 25 ± 2 | 26 ± 4 | 13 ± 2 | 123 ± 19 | 49 ± 10 | 12 ± 4 |
| 21 | 828 ± 51 | 632 ± 161 | 6 ± 3 | 15 ± 3 | 19 ± 2 | 9 ± 3 | 116 ± 23 | 41 ± 10 | 6 ± 3 |
| 25 | 969 ± 69 | 660 ± 145 | 4 ± 2 | 7 ± 2 | 16 ± 4 | 4 ± 1 | 160 ± 43 | 39 ± 13 | 5 ± 3 |
| 28 | 1059 ± 84 | 760 ± 175 | 3 ± 2 | 6 ± 2 | 11 ± 3 | 2 ± 1 | 164 ± 37 | 39 ± 12 | 4 ± 2 |

Note:
^{a.} Mean ± SEM
^{b}G1: Vehicle, G2: B12-LP1 (3 mg/kg), G3: benchmark (BM) or BAP-143-00-00-LP1 (3 mg/kg), G4: BAP-143-00-01-LP1 (3 mg/kg), G5: BAP-143-00-02-LP1 (3 mg/kg), G6: BAP-143-00-03-LP1 (3 mg/kg), G7: BAP-143-00-04-LP1 (3 mg/kg), G8: BAP-143-00-05-LP1 (3 mg/kg), G9: BAP-143-00-06-LP1 (3 mg/kg).

### Tumor Growth Inhibition Analysis

TABLE 33-4
Tumor growth inhibition (based on Day 28 data)

| | Tumor | | | | | |
|---|---|---|---|---|---|---|
| | Size | T/C ^{b} | TGI | | | |
| Treatment | (mm³) ^{a} | (%) | (%) | p ^{c} | RTV ^{a} | p ^{d} |
| Vehicle | 1059 ± 84 | — | — | — | 7.2 ± 0.7 | 0.345 |
| B12-LP1 (3 mg/kg) | 760 ± 175 | 71.72 | 33.00 | 0.813 | 4.7 ± 0.7 | 0.001 |
| BAP-143-00-00-LP1 (3 mg/kg) | 3 ± 2 | 0.25 | 116.52 | 0.000 | 0 ± 0 | 0.001 |
| BAP-143-00-01-LP1 (3 mg/kg) | 6 ± 2 | 0.53 | 116.21 | 0.000 | 0 ± 0 | 0.001 |
| BAP-143-00-02-LP1 (3 mg/kg) | 11 ± 3 | 1.05 | 115.52 | 0.000 | 0.1 ± 0 | 0.001 |
| BAP-143-00-03-LP1 (3 mg/kg) | 2 ± 1 | 0.18 | 116.61 | 0.000 | 0 ± 0 | 0.001 |
| BAP-143-00-04-LP1 (3 mg/kg) | 164 ± 37 | 15.50 | 98.71 | 0.000 | 1.2 ± 0.3 | 0.001 |
| BAP-143-00-05-LP1 (3 mg/kg) | 39 ± 12 | 3.71 | 112.43 | 0.000 | 0.2 ± 0.1 | 0.001 |
| BAP-143-00-06-LP1 (3 mg/kg) | 4 ± 2 | 0.35 | 116.42 | 0.000 | 0 ± 0 | 0.001 |

Note:
^{a.} Mean + SEM.
^{b.} p value calculated based on tumor size.
^{c.} p value calculated based on RTV.

### 6.3 Tumor Growth Curve

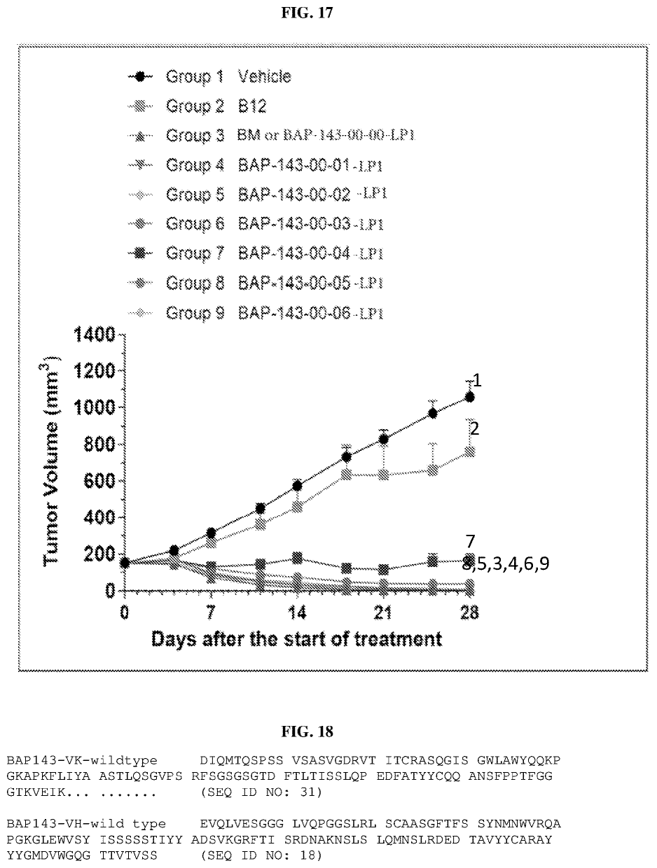

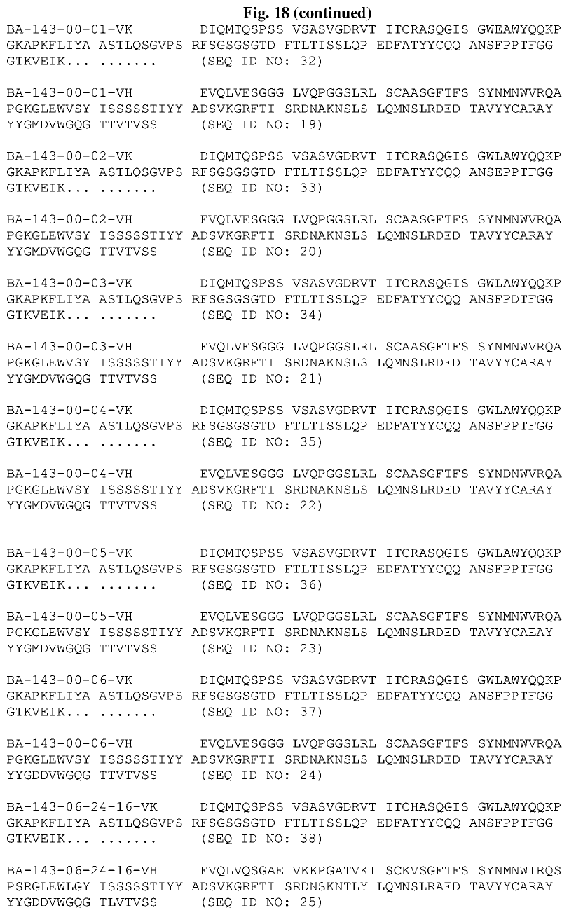

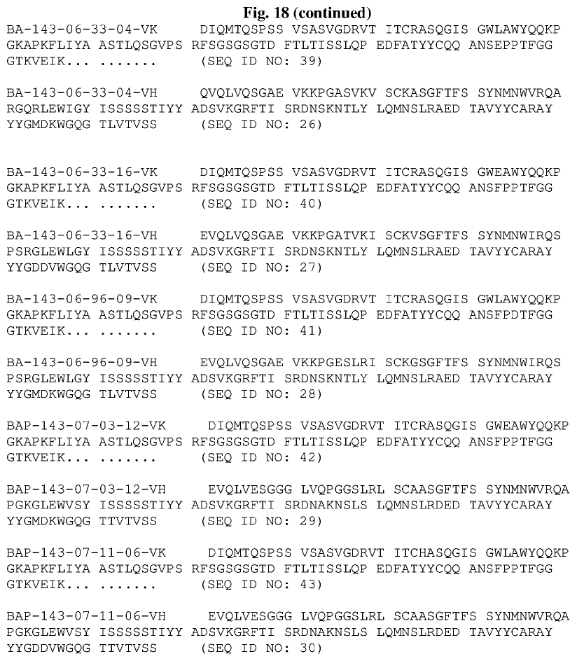

Tumor growth curve is shown in FIG. 17. Data presented are mean±SEM.

### Summary and Discussion

In this study, the therapeutic efficacy of representative anti-Nectin-4 CAB ADCs and the WT ADC of the present disclosure were evaluated using xxT47D human breast xenograft model. The tumor volume of different groups after treatment are shown in TABLE 33-3, TABLE 33-4 and FIG. 17. The mean tumor volume of the vehicle group reached 1,059 mm³ on Day 28 (RTV=7.2±0.7) after treatment initiation.

The test articles BAP-143-00-00-LP1 (or benchmark (BM)), BAP-143-00-01-LP1, BAP-143-00-02-LP1, BAP-143-00-03-LP1, BAP-143-00-04-LP1, BAP-143-00-05-LP1 and BAP-143-00-06-LP1 at 3 mg/kg dose exhibited excellent anti-tumor activities with all TGI % are bigger than 98% (p value=0). And all these treatments significantly delayed tumor growth (RTV from 0.0 to 1.2 on Day 28, p value=0.001). Among them, BAP-143-00-04 showed relative lower efficacy (TGI=98.71%, RTV=1.2±0.3)

B12-LP1 didn't significantly affect tumor growth under the current regimen (TGI=33%, p value=0.813). During this study, Mouse #18964 in the 3 mg/kg BAP-143-00-04-LP1 group died on day 18 and mouse #18946 in the 3 mg/kg BAP-143-00-06-LP1 group died on day 4 for unknown reasons. The other mice did not appear to be overtly sick. Thus, no obvious toxicity was observed in association with the administration of exemplary anti-Nectin-4 CAB ADCs and the WT ADC of the present disclosure.

### Example 34: Binding Activity Of Conditionally Active Anti-Nectin4 Antibodies Measured By SPR Analysis

Binding kinetics of anti-Nectin4 antibodies were measured by surface plasmon resonance using a SPR2/4 instrument (Sierra Sensors, Hamburg, Germany) and flat amine sensor chips. The SPR sensor contained four flow cells (FC1-FC4), each of which was addressed individually or in groups. huNectin4-His was immobilized in FC2, and cynoNectin4-mFc in FC4. No protein was immobilized in FC1 and FC3 which were used as control surfaces for FC2 and FC4, respectively.

All injections were done at a flow rate of 25 μL/min at 25° C. The sensor surface was activated with 1-ethyl-3-(3-dimethylaminopropyl)-carbodiimide (EDC) and N-hydroxysuccinimide (NHS) (200 mM/50 mM) for 480 seconds. HuNectin4-His (2 ug/mL in 10 mM NaAc, pH4.5) was injected for 480s and the surface was inactivated by injecting 1M ethanolamine-HCl for 480s. CynoNectin4 was immobilized using the same conditions as described for huNectin4-His, except that the cynoNectin4 was diluted into 10 mM NaAc buffer having pH 5.0. The control surface was activated and deactivated using the same conditions, but without injecting protein. PBST buffer (PBS pH 7.4 with 0.05% TWEEN20™) was used as running buffer for the surface preparation. The running solution was switched to PBST with 30 mM sodium bicarbonate with the pH adjusted as indicated in the figures before the analyte injections. The instrument was equilibrated with the running solution for one hour before the first analyte injection.

100 μL analyte diluted in the corresponding running solution (34.25 nM, 13.70 nM, 6.85 nM, 3.42 nM, 1.37 nM, and 0.0 nM) was injected over flow cells 1 and 2 or 3 and 4. The off-rate was measured for 360s. The chip surface was regenerated after each cycle of interaction analysis by injecting 6 μL of 10 mM glycine (pH 2.0). Flow cells 1 and 3 without immobilized protein were used as control surfaces for reference subtraction.

In addition, data with buffer only as analyte (0 nM analyte) was subtracted from each run. Double subtracted data was fitted with the provided analysis software Analyzer R2 (Sierra Sensors) using a 1:1 binding model. A molecular weight of 150 kDa was used to calculate the molar concentrations of the analytes. RatNectin4-His was immobilized in FC3 using the same conditions as described for cynoNectin4.

Binding activities at pH 6.0, pH. 6.5 and pH 7.4 of conditionally active anti-Nectin4 antibodies to human Nectin4, cynoNectin4, and ratNectin4, and of conditionally active anti-Nectin4 antibodies to human Nectin4 and cyanoNectin4, were measured by SPR analysis. The results are shown in TABLES 34-1 to 34-3 and TABLES 35-1 to 35-2, respectively, below.

TABLE 34-1

| | pH7.4 | | | pH6.5 | | |
|---|---|---|---|---|---|---|
| humanNectin4 | Ka [M · s] | Kd[s⁻¹] | KD[M] | Ka [M · s] | Kd[s⁻¹] | KD[M] |
| BM | 3.03E+06 | 3.85E−04 | 1.27E−10 | 3.53E+06 | 3.60E−04 | 1.02E−10 |
| BAP-143-00-01 | 2.85E+05 | 5.17E−04 | 1.81E−09 | 6.05E+05 | 4.74E−04 | 7.85E−10 |
| BAP-143-06-33-04 | 1.50E+06 | 8.80E−05 | 5.88E−11 | 2.31E+06 | 3.99E−04 | 1.73E−10 |
| BAP-143-06-24-16 | 7.78E+05 | 5.21E−06 | 6.70E−12 | 2.04E+06 | 9.92E−06 | 4.87E−12 |
| BAP-143-06-33-16 | 1.19E+05 | 2.01E−05 | 1.70E−10 | 2.11E+06 | 5.94E−05 | 2.82E−11 |
| BAP-143-07-03-12 | 4.34E+05 | 4.01E−04 | 9.25E−10 | 7.54E+05 | 4.83E−04 | 6.41E−10 |

| | pH6.0 | | |
|---|---|---|---|
| humanNectin4 | Ka [M · s] | Kd[s⁻¹] | KD[M] |
| BM | 4.01E+06 | 5.96E−04 | 1.49E−10 |
| BAP-143-00-01 | 1.04E+06 | 7.71E−04 | 7.39E−10 |
| BAP-143-06-33-04 | 3.82E+06 | 7.46E−04 | 1.95E−10 |
| BAP-143-06-24-16 | 3.50E+06 | 3.30E−04 | 9.44E−11 |
| BAP-143-06-33-16 | 1.77E+06 | 1.95E−04 | 1.10E−10 |
| BAP-143-07-03-12 | 1.26E+06 | 8.69E−04 | 6.90E−10 |

TABLE 34-2

| | pH7.4 | | | pH6.5 | | |
|---|---|---|---|---|---|---|
| cynoNectin4 | Ka [M · s] | Kd[s⁻¹] | KD[M] | Ka [M · s] | Kd[s⁻¹] | KD[M] |
| BM | 2.89E+06 | 7.04E−04 | 2.44E−10 | 3.27E+06 | 6.82E−04 | 2.09E−10 |
| BAP-143-00-01 | 2.40E+05 | 7.81E−04 | 3.25E−09 | 4.57E+05 | 8.59E−04 | 1.88E−09 |
| BAP-143-06-33-04 | 1.24E+06 | 2.39E−04 | 1.94E−10 | 1.97E+06 | 6.43E−04 | 3.26E−10 |
| BAP-143-06-24-16 | 6.55E+05 | 8.26E−05 | 1.26E−10 | 1.68E+06 | 8.99E−05 | 5.34E−11 |
| BAP-143-06-33-16 | 7.39E+04 | 1.75E−04 | 2.37E−09 | 1.60E+06 | 2.60E−04 | 1.625E−10 |
| BAP-143-07-03-12 | 3.52E+05 | 6.51E−04 | 1.85E−09 | 5.49E+05 | 8.76E−04 | 1.60E−09 |

| | pH6.0 | | |
|---|---|---|---|
| cynoNectin4 | Ka [M · s] | Kd[s⁻¹] | KD[M] |
| BM | 3.71E+06 | 1.15E−03 | 3.11E−10 |
| BAP-143-00-01 | 7.69E+05 | 1.42E−03 | 1.85E−09 |
| BAP-143-06-33-04 | 3.21E+06 | 1.49E−03 | 4.63E−10 |
| BAP-143-06-24-16 | 2.80E+06 | 5.79E−04 | 2.07E−10 |
| BAP-143-06-33-16 | 1.46E+06 | 2.73E−04 | 1.87E−10 |
| BAP-143-07-03-12 | 9.83E+05 | 1.13E−03 | 1.15E−09 |

TABLE 34-3

| | pH7.4 | | | pH6.5 | | |
|---|---|---|---|---|---|---|
| ratNectin4 | Ka [M · s] | Kd[s⁻¹] | KD[M] | Ka [M · s] | Kd[s⁻¹] | KD[M] |
| BM | 5.31E+06 | 7.19E−04 | 1.35E−10 | 6.19E+06 | 6.67E−04 | 1.08E−10 |
| BAP-143-00-01 | 3.61E+05 | 8.75E−04 | 2.42E−09 | 7.00E+05 | 9.53E−04 | 1.36E−09 |
| BAP-143-06-33-04 | 2.27E+06 | 3.85E−04 | 1.69E−10 | 4.07E+06 | 6.10E−04 | 1.50E−10 |
| BAP-143-06-24-16 | 1.04E+06 | 1.78E−04 | 1.71E−10 | 3.39E+06 | 1.28E−04 | 3.78E−11 |
| BAP-143-06-33-16 | 1.35E+05 | 2.61E−04 | 1.94E−09 | 3.01E+06 | 3.04E−04 | 1.01E−10 |
| BAP-143-07-03-12 | 5.57E+05 | 7.31E−04 | 1.31E−09 | 7.87E+05 | 8.68E−04 | 1.10E−09 |

| | pH6.0 | | |
|---|---|---|---|
| ratNectin4 | Ka [M · s] | Kd[s⁻¹] | KD[M] |
| BM | 6.54E+06 | 1.15E−03 | 1.75E−10 |
| BAP-143-00-01 | 1.36E+06 | 1.59E−03 | 1.17E−09 |
| BAP-143-06-33-04 | 6.63E+06 | 1.51E−03 | 2.27E−10 |
| BAP-143-06-24-16 | 6.10E+06 | 6.35E−04 | 1.04E−10 |
| BAP-143-06-33-16 | 2.57E+06 | 3.94E−04 | 1.54E−10 |
| BAP-143-07-03-12 | 1.62E+06 | 1.20E−03 | 7.38E−10 |

TABLE 35-1

| | pH7.4 | | | pH6.5 | | |
|---|---|---|---|---|---|---|
| humanNectin4 | Ka [M · s] | Kd[s⁻¹] | KD[M] | Ka [M · s] | Kd[s⁻¹] | KD[M] |
| BM | 3.87E+06 | 3.65E−03 | 9.43E−10 | 4.56E+06 | 3.12E−03 | 6.85E−10 |
| BAP-143-00-01 | 6.86E+05 | 2.67E−03 | 3.89E−09 | 4.76E+05 | 2.45E−03 | 5.15E−09 |
| BAP-143-00-02 | 4.90E+05 | 4.78E−03 | 9.76E−09 | 6.17E+05 | 5.74E−03 | 9.31E−09 |
| BAP-143-00-03 | 1.31E+06 | 3.20E−03 | 2.45E−09 | 7.71E+05 | 2.29E−03 | 2.97E−09 |
| BAP-143-00-04 | 3.71E+06 | 5.25E−03 | 1.42E−09 | 2.94E+06 | 4.39E−03 | 1.50E−09 |
| BAP-143-00-05 | 1.22E+06 | 6.20E−03 | 5.09E−09 | 7.26E+05 | 4.53E−03 | 6.23E−09 |
| BAP-143-00-06 | 3.52E+06 | 1.43E−03 | 4.06E−10 | 3.30E+06 | 7.26E−04 | 2.20E−10 |

| | pH6.0 | | |
|---|---|---|---|
| humanNectin4 | Ka [M · s] | Kd[s⁻¹] | KD[M] |
| BM | 2.15E+06 | 3.22E−03 | 1.49E−09 |
| BAP-143-00-01 | 2.98E+05 | 3.67E−03 | 1.23E−08 |
| BAP-143-00-02 | no binding | | |
| BAP-143-00-03 | 2.03E+05 | 1.22E−03 | 6.02E−09 |
| BAP-143-00-04 | 6.02E+05 | 1.55E−03 | 2.58E−09 |
| BAP-143-00-05 | 3.80E+05 | 2.42E−03 | 6.37E−09 |
| BAP-143-00-06 | 7.07E+05 | 1.14E−03 | 1.61E−09 |

TABLE 35-2

| | pH7.4 | | | pH6.5 | | |
|---|---|---|---|---|---|---|
| cynoNectin4 | Ka [M · s] | Kd[s⁻¹] | KD[M] | Ka [M · s] | Kd[s⁻¹] | KD[M] |
| BM | 3.07E+06 | 1.02E−03 | 3.33E−10 | 3.36E+06 | 1.75E−03 | 5.20E−10 |
| BAP-143-00-01 | 1.46E+05 | 1.62E−03 | 1.11E−08 | 4.57E+05 | 1.34E−03 | 2.94E−09 |
| BAP-143-00-02 | 3.34E+05 | 1.95E−03 | 5.86E−09 | 3.80E+05 | 1.79E−03 | 4.71E−09 |
| BAP-143-00-03 | 2.15E+05 | 8.26E−04 | 3.85E−09 | 6.68E+05 | 1.24E−03 | 1.86E−09 |
| BAP-143-00-04 | 8.40E+05 | 1.12E−03 | 1.33E−09 | 1.60E+06 | 1.80E−03 | 1.12E−09 |
| BAP-143-00-05 | 3.22E+05 | 1.57E−03 | 4.89E−09 | 5.99E+05 | 2.37E−03 | 3.96E−09 |
| BAP-143-00-06 | 8.39E+05 | 3.35E−04 | 4.00E−10 | 1.93E+06 | 7.69E−04 | 3.98E−10 |

| | pH6.0 | | |
|---|---|---|---|
| cynoNectin4 | Ka [M · s] | Kd[s⁻¹] | KD[M] |
| BM | 3.29E+06 | 3.49E−03 | 1.06E−09 |
| BAP-143-00-01 | 8.17E+05 | 2.48E−03 | 3.04E−09 |
| BAP-143-00-02 | 4.43E+05 | 2.42E−03 | 5.46E−09 |
| BAP-143-00-03 | 1.01E+06 | 2.35E−03 | 2.33E−09 |
| BAP-143-00-04 | 1.82E+06 | 3.77E−03 | 2.08E−09 |
| BAP-143-00-05 | 6.39E+05 | 4.49E−03 | 7.02E−09 |
| BAP-143-00-06 | 2.52E+06 | 1.41E−03 | 5.60E−10 |

### METHODS USED IN THE EXAMPLES

The ELISA assay for EXAMPLES 21-23 was performed using the following protocol:
1) Coat ELISA plates with 100 μL of 1 μg/mL recombinant human Nectin4 antigen or cynoNectin4 antigen in carbonate-bicarbonate coating buffer.
2) Cover plates with sealing film and incubate overnight at 4° C.
3) Decant plates and tap out residual liquid on a stack of paper towels.
4) Wash wells twice by dispensing 200 μL of pH 6.0 or pH 7.4 ELISA incubation buffer to the wells and completely aspirate the contents.
5) Add 200 μL of pH 6.0 or pH 7.4 ELISA incubation buffer to the wells. Cover with sealing film and place the plate onto a plate shaker set to 50 rpm for 60 minutes at room temperature.
6) Decant plates and tap out residual liquid on a stack of paper towels.
7) Serially dilute test articles in 3-fold dilutions starting at 3000 ng/ml in pH 6.0 or pH 7.4 ELISA incubation buffer.
8) Add 100 μL/well of diluted test articles to the plates
9) Cover with sealing film and place the plates onto a plate shaker set to 50 rpm for 60 minutes at room temperature.
10) Decant plates and tap out residual liquid on a stack of paper towels.
11) Wash wells three times by dispensing 200 μL of pH 6.0 or pH 7.4 ELISA wash buffer to the wells and completely aspirate the contents.
12) Dilute the HRP secondary antibody at 1:2500 in pH 6.0 or pH 7.4 ELISA incubation buffer.
13) Add 100 μL HRP secondary antibody diluted in pH 6.0 or pH 7.4 ELISA incubation buffer to each well
14) Cover with sealing film and place the plates onto a plate shaker set to 50 rpm for 60 minutes at room temperature.
15) Decant plates and tap out residual liquid on a stack of paper towels.
16) Wash wells three times by dispensing 200 μL of pH 6.0 or pH 7.4 ELISA wash buffer to the wells and completely aspirate the contents.
17) Dispense 50 μL per well of the TMB substrate solution into all wells of the plates. Incubate at room temperature for about 2 minutes 15 seconds or 2 minutes.
18) Add 50 μL per well of 1N HCl into all wells of the plates. Read plates at 450 nm using PerkinElmer, EnSpire 2300 Multilabel Reader.

The ELISA assay for EXAMPLES 24-25 was performed using the following protocol:
1) Coat ELISA plates with 100 μL of 1 μg/mL recombinant human Nectin4 antigen in carbonate-bicarbonate coating buffer
2) Cover plates with sealing film and incubate overnight at 4° C.
3) Decant plates and tap out residual liquid on a stack of paper towels
4) Wash wells twice by dispensing 200 μL of various pH incubation buffer to the wells and completely aspirate the contents
5) Add 200 μL of various pH incubation buffers (pH5.5, 6.0, 6.2, 6.5, 6.7, 7.0 and 7.4) to the wells. Cover with sealing film and place the plate onto a plate shaker (set to 200 rpm) for 60 minutes at room temperature
6) Decant plates and tap out residual liquid on a stack of paper towels
7) Serially dilute test substances in various pH incubation buffers (pH5.5, 6.0, 6.2, 6.5, 6.7, 7.0 and 7.4) to 30 ng/ml
8) Add 100 μL/well of diluted test substances to the plates
9) Cover with sealing film and place the plates onto a plate shaker (set to 200 rpm) for 60 minutes at room temperature.
10) Decant plates and tap out residual liquid on a stack of paper towels.
11) Wash wells three times by dispensing 200 μL of various pH wash buffers (pH5.5, 6.0, 6.2, 6.5, 6.7, 7.0 and 7.4) to the wells and completely aspirate the contents
12) Dilute the HRP secondary antibody at 1:2500 in various pH incubation buffers (pH5.5, 6.0, 6.2, 6.5, 6.7, 7.0 and 7.4)
13) Add 100 μL HRP secondary antibody diluted in various pH incubation buffers (pH5.5, 6.0, 6.2, 6.5, 6.7, 7.0 and 7.4) to each well.
14) Cover with sealing film and place the plates onto a plate shaker (set to 200 rpm) for 60 minutes at room temperature.
15) Decant plates and tap out residual liquid on a stack of paper towels.
16) Wash wells three times by dispensing 200 μL of various pH wash buffer (pH5.5, 6.0, 6.2, 6.5, 6.7, 7.0 and 7.4) to the wells and completely aspirating the contents
17) Dispense 50 μL per well of the TMB substrate solution into all wells of plates. Incubate at room temperature for 3 minutes.
18) Add 50 μL per well of 1N HCl into all wells of the plates. Read plates at 450 nm. Plot the average OD values (from 2 replicates) at the different pH values against the pH of the buffer using Softmax Pro software (Molecular Devices). Curve fitting was done using the 4-parameter model built into the software. The inflection point of the pH curve (50% binding activity) equals parameter C of the fitting equation. Binding activity at pH 6.0 was set to 100%.

The fluorescence-activated cell sorting (FACS) assay for EXAMPLES 26-31 was performed using the following protocol.
1) Seed 3×10⁶ cells to T-75 flasks and culture according to the instructions of vendors.
2) On the day of FACS analysis, remove and discard culture medium.
3) Briefly rinse the cell layer with PBS solution.
4) Add 1.5 mL of Detachin solution to each of the T-75 flasks. Wait until cell layer has dispersed.
5) Add 4.5 mL of culture media for the corresponding cell lines and resuspend cells by gently pipetting.
6) Pool the cells and transfer the cell suspension to a 50-mL conical tube.
7) Count the cells with trypan blue staining before centrifugation at 1500 rpm for 5 min at 4° C.
8) Wash the cells once with PBS
9) Resuspend the cells in pH 6.0 or pH 7.4 FACS buffer to 3.5×10⁶ cells/mL.
10) Aliquot 3.5×10⁵ cells in 100 μL pH 6.0 or pH 7.4 FACS buffer in 96-well U-bottom plates.
11) Spin down the cells and discard the buffer.
12) Serially dilute test articles in 3-fold dilutions starting at 30 μg/mL in pH 6.0 or pH 7.4 FACS buffer.
13) Add 100 μL/well of the diluted test articles to cells, gently mix well and incubate on ice with shaking (200 rpm) for one hour.
14) Centrifuge the cells at 1500 rpm for 5 min at 4° C. Wash the cells with 150 μL of pH 6.0 or pH 7.4 wash buffer twice.
15) Dilute the goat anti-human IgG AF488 antibody 1:300 in pH 6.0 or pH 7.4 FACS buffers.
16) Add 100 μL of the diluted antibody from step above to the cells and incubate on ice with shaking (200 rpm) for 45 minutes, protected from light.
17) Pellet the cells and wash with 150 μL of pH 6.0 or pH 7.4 wash buffer three times.
18) Fix cells with 4% PFA diluted in 1×PBS for 10 min at R.T., then wash cells with 1×PBS.
19) Resuspend the cells in 100 μL of 1×PBS.
20) Analyze the cells by NovoCyte Flow Cytometer using Ex488 nm/Em530 nm. Collect at least 5,000 singlet cells for each data point.
21) The MFI of AF488 in cell singlets was plotted using GraphPad Prism software version 7.03.

### Protocol Used for Examples 32-33

### 1.1 Cell Culturing

HEK293-huNectin4 were maintained in stable cell line culture medium (MEM+10% Fetal Bovine Serum (FBS)+1 mg/mL G418). The cells were routinely sub-cultured twice per week. The cells were harvested during exponential growth phase and counted for plating.

### 1.2 Formulations

1) Make 5-fold serial dilution of 10×test article ADC or B12 isotype ADC stocks in pH 6.0 or pH 7.4 assay media in duplicates starting at 50 μg/mL
2) Centrifuge the plates and gently remove the culture medium then add 90 μL of pH assay media before adding ADCs
3) Add 10 μL of serially diluted 10×ADCs or B12 sample stocks to the wells containing 3000 cells (Final starting concentration is 5 μg/mL)
4) Incubate the treated cells at 37° C. in a 5% CO₂ incubator for 72 hours

### 1.3 Cell Titer-Glo Luminescent Cell Viability Assay

1) Thaw the CellTiter-Glo buffer and equilibrate to room temperature prior to use
2) Equilibrate the lyophilized CellTiter-Glo substrate to room temperature prior to use
3) Transfer the entire liquid volume of CellTiter-Glo buffer into the amber bottle containing CellTiter-
Glo substrate to reconstitute the lyophilized enzyme/substrate mixture. This forms the CellTiter-Glo reagent
4) Mix by gently vortex to obtain a homogeneous solution
5) Equilibrate the plate and its contents to room temperature
6) Add 70 μL CellTiter-Glo reagent in each well. Mix contents for 2 minutes on an orbital shaker at 100 rpm to induce cell lysis
5) Allow the plate to incubate at room temperature for 10 minutes to stabilize luminescent signal
6) Record luminescence on the SpectraMax i3X plate reader

### 1.4 Data Analysis

The inhibitions of different dose of test antibodies were plotted in concentration-response luminescence signals and IC50 was calculated. The data were interpreted by GraphPad Prism software.

### Example 34—Conditionally Active Bispecific Antibodies Targeting Nectin-4

Nectin-4 is a predictive marker for cancer diagnosis and can be a target for development of targeted therapeutics. It may play a mechanistic role in cancer metastasis and angiogenesis of serval types of primary tumors. Nectin-4 is a target for adenocarcinomas in general. Nectin-4 expression has a significant correlation with tumor grade and stages associated to tumor progression.

Bispecific antibodies were generated which have little to no binding to CD3 and the target antigen in healthy tissue (normal alkaline microenvironment). However, in acid conditions that mirror the tumor microenvironment (high glycolysis) the binding of the antibodies to their target molecule was strong. These molecules demonstrated binding to both recombinant TAA/CD3 and TAA/CD3 expressing cells under acidic conditions that were present in the tumor microenvironment, but not in normal tissues.

A dual-CAB (CAB TAA×CAB CD3) bispecific antibody was developed targeting the well-established tumor associated antigen Nectin-4. These bispecific antibodies were active against target positive human tumor xenografts. Importantly, complete tumor regression was observed upon treatment with these CAB bispecific antibodies. Reversible CAB bispecifics yielded a superior therapeutic index relative to other formats, including prodrugs.

### Example 35—Conditionally Active Bispecific Antibodies Targeting Nectin-4 (CAB Nectin-4×CAB CD3)

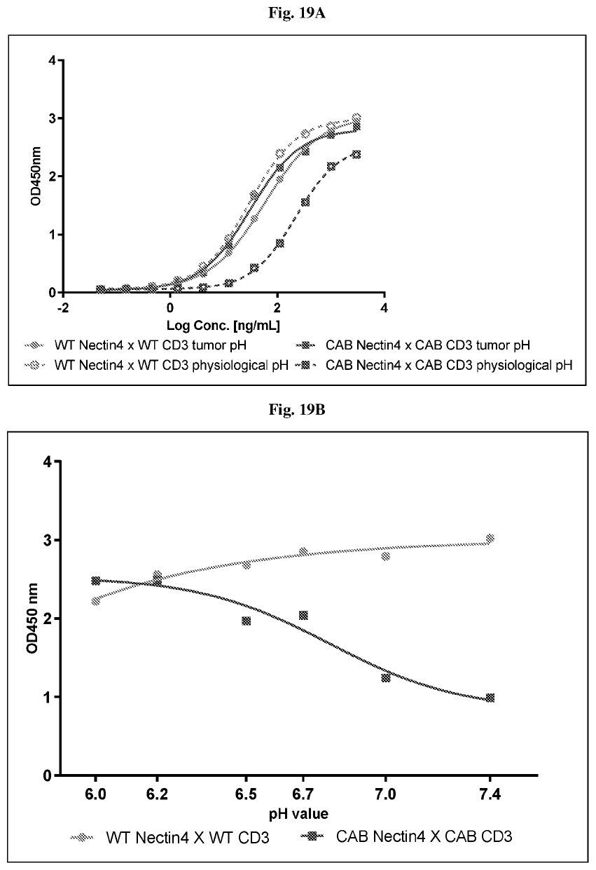

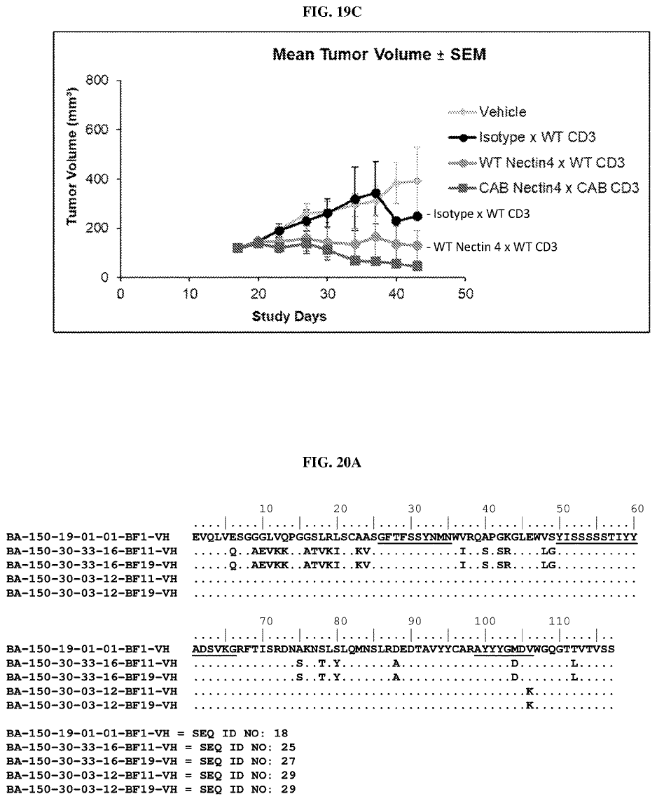

CAB Nectin-4×CAB CD3 bispecific antibody showed high affinity to recombinant human Nectin-4 ECD and CD3 epsilon/delta heterodimer protein like wild type (WT) Nectin-4×WT CD3 in a tumor microenvironment pH but showed lower affinity in the physiological pH (FIG. 19A). pH affinity ELISA applied human CD3 as capture antigen, human Nectin-4-mFc as detection followed by anti-mouse IgG HRP conjugated antibody. CAB Nectin-4×CAB CD3 showed higher affinity in tumor microenvironment pH, but lower binding under the physiological pH.

CAB Nectin-4×CAB CD3 pH profile showed that the affinity to human CD3 and human B7H3 were higher in an acidic tumor microenvironment pH of 6.0-6.5 and lower in a physiological pH (7.4) (FIG. 19B). CAB Nectin-4×CAB CD3 demonstrated a differential binding with human CD3 as capture antigen, human Nectin-4-mFc as detection following with anti-mouse IgG HRP conjugated antibody within the pH range 6.0-7.4. The affinity binding of WT Nectin-4×WT CD3 remained at a similar level.

CAB Nectin-4×CAB CD3 dosed at 2.5 mg/kg BIW×4 led to similar tumor regression as WT Nectin-4×WT CD3 in NCI-H358 MiXeno model at the same dose (FIG. 19C). The in vivo efficacy study showed that CAB Nectin-4×CAB CD3 dosed at 2.5 mg/kg BIW×4 demonstrated a comparable tumor regression in NCI-H358 MiXeno model as WT Nectin-4×WT CD3.

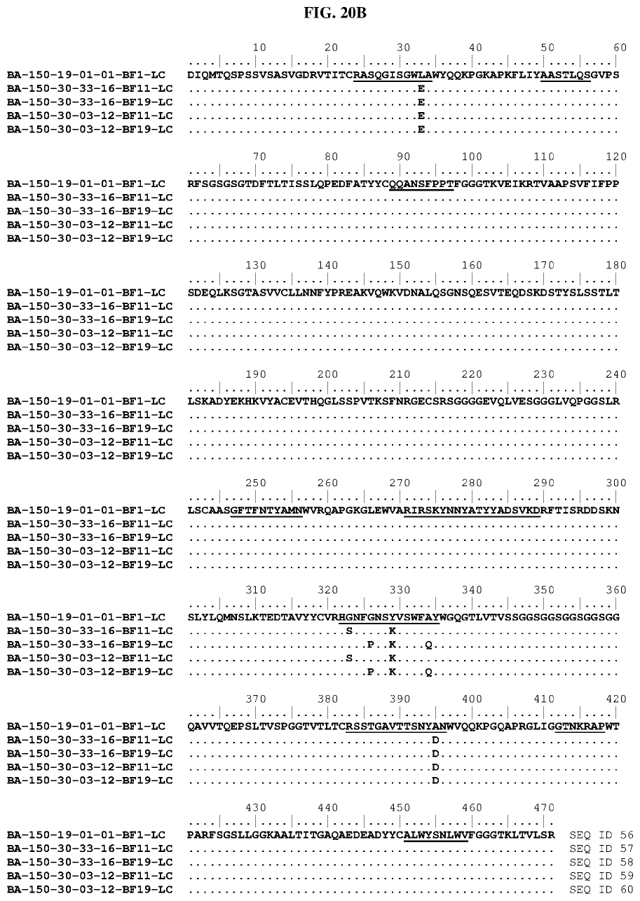

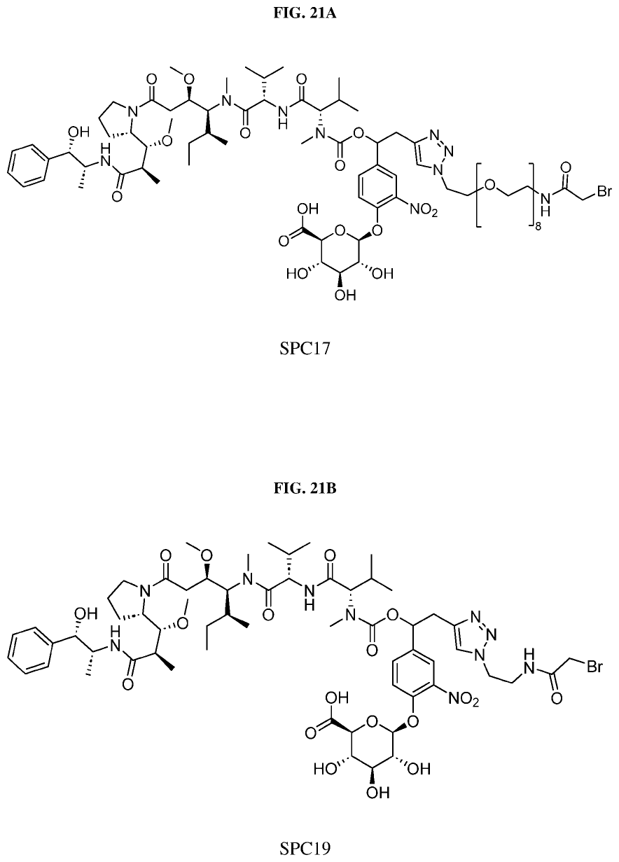

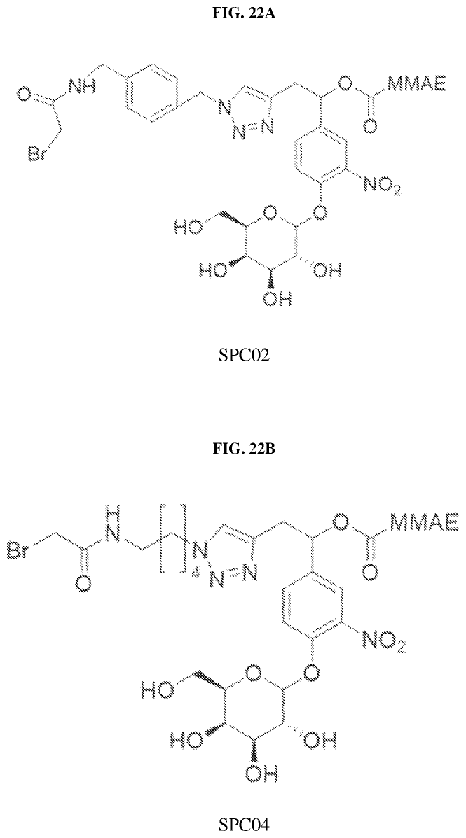

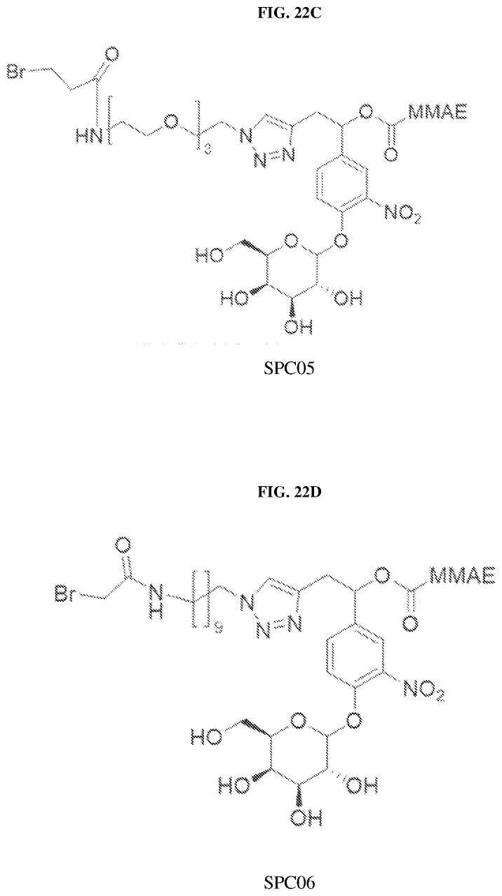

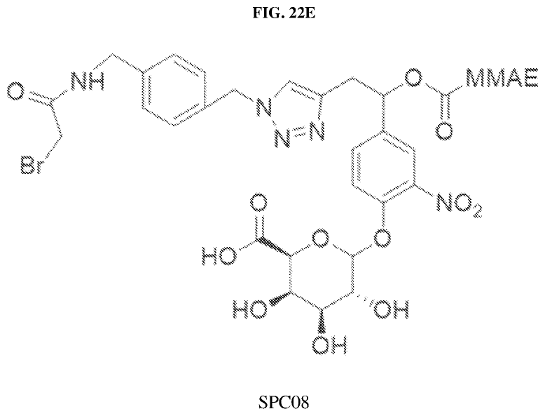

Bispecific antibodies that bind to Nectin-4 and CD3 (FIGS. 20A-20B) were constructed including the heavy and light chains as shown in the Table below.

| Antibody Clone | light chain | heavy chain |
|---|---|---|
| WT Nectin4 x WT CD3 | BA-150-19-01-01-BF1-LC | BA-150-19-01-01-BF1-VH |
| | (SEQ ID NO: 56) | (SEQ ID NO: 18) |
| CAB Nectin4 x CAB CD3 | BA-150-30-03-12-BF19-LC | BA-150-30-03-12-BF19-VH |
| | (SEQ ID NO: 60) | (SEQ ID NO: 29) |
| Isotype x WT CD3 | BA-150-HEL-BF1, isotype | BA-150-19-01-01-BF1-VH |
| (negative control - | | (SEQ ID NO: 18) |
| B12 x WT CD3) | | |

The heavy and light chains of the antibodies are: BA-150-19-01-01-BF1-V_{H} (SEQ ID NO: 18), BA-150-30-33-16-BF11-V_{H} (SEQ ID NO: 25), BA-150-30-33-16-BF19-V_{H} (SEQ ID NO: 27), BA-150-30-03-12-BF11-V_{H} (SEQ ID NO: 29) and BA-150-30-03-12-BF19-V_{H} (SEQ ID NO: 29). BA-150-19-01-01-BF1-LC (SEQ ID NO: 56), BA-150-30-33-16-BF11-LC (SEQ ID NO: 57), BA-150-30-33-16-BF19-LC (SEQ ID NO: 58), BA-150-30-03-12-BF11-LC (SEQ ID NO: 59), and BA-150-30-03-12-BF19-LC (SEQ ID NO: 60).

### CONCLUSIONS

CAB B7H3×CAB CD3 and CAB Nectin-4×CAB CD3 bispecific antibodies have increased binding under tumor conditions compared to normal conditions. The pH profile ELISA confirmed the differential affinity within the pH ranges from 6.0 to 7.4.

CAB B7H3×CAB CD3 and CAB Nectin-4×CAB CD3 bispecific antibodies have similar efficacy in cancer cell line derived MiXeno models in vivo compared to the non-CAB benchmark antibodies.

The present disclosure transformed bispecific solid tumor therapies through the widening of the therapeutic index.

Protocol for Differential Affinity Binding of CAB Nectin-4×CAB CD3 bispecific antibody Measured by ELISA

### 1.1 Test Articles

WT Nectin-4×WT CD3 (BA-150-19-01-01-BF1, benchmark) CAB Nectin-4×CAB CD3 (BA-150-30-03-12-BF19)

### 1.2 Formulations

Test articles were first diluted to 3000 ng/mL in pH 6.0 or pH 7.4 ELISA incubation buffer.
Then 3000 ng/ml of test articles were 3-fold serially diluted in pH 6.0 or pH 7.4 ELISA incubation buffer.

### 1.3 pH Affinity ELISA Assay

1) Coat ELISA plates with 100 μL of 0.5 μg/mL recombinant human CD3 antigen in carbonate-bicarbonate coating buffer.
2) Cover plates with sealing film and incubate overnight at 4° C.
3) Decant plates and tap out residual liquid on a stack of paper towels.
4) Wash wells twice by dispensing 200 μL of pH 6.0 or pH 7.4 ELISA incubation buffer to the wells and completely aspirate the contents.
5) Add 200 μL of pH 6.0 or pH 7.4 ELISA incubation buffer to the wells. Cover with sealing film and place the plate onto a plate shaker set to 50 rpm for 60 minutes at room temperature.
6) Decant plates and tap out residual liquid on a stack of paper towels.
7) Serially dilute test articles in 3-fold dilutions starting at 3000 ng/mL in pH 6.0 or pH 7.4 ELISA incubation buffer.
8) Add 100 μL/well of diluted test articles to the plates
9) Cover with sealing film and place the plates onto a plate shaker set to 50 rpm for 60 minutes at room temperature.
10) Decant plates and tap out residual liquid on a stack of paper towels.
11) Wash wells three times by dispensing 200 μL of pH 6.0 or pH 7.4 ELISA wash buffer to the wells and completely aspirate the contents.
12) Dilute the HRP secondary antibody at 1:2500 in pH 6.0 or pH 7.4 ELISA incubation buffer.
13) Add 100 μL HRP secondary antibody diluted in pH 6.0 or pH 7.4 ELISA incubation buffer to each well
14) Cover with sealing film and place the plates onto a plate shaker set to 50 rpm for 60 minutes at room temperature.
15) Decant plates and tap out residual liquid on a stack of paper towels.
16) Wash wells three times by dispensing 200 μL of pH 6.0 or pH 7.4 ELISA wash buffer to the wells and completely aspirate the contents.
17) Dispense 50 μL per well of the TMB substrate solution into all wells of the plates. Incubate at room temperature for about 5 minutes.
18) Add 50 μL per well of 1N HCl into all wells of the plates. Read plates at 450 nm using PerkinElmer, EnSpire 2300 Multilabel Reader.

Protocol for Measuring the pH range Binding Affinity of CAB Nectin-4×CAB CD3

### 2.1 Test Articles

WT Nectin-4×WT CD3 (BA-150-19-01-01-BF1, benchmark)

### CAB Nectin-4×CAB CD3 (BA-150-30-03-12-BF19)

### 2.2 Formulations

Test articles were diluted to 100 ng/mL in various pH ELISA incubation buffer range from pH 6.0 to pH 7.4.

### 2.3 pH Affinity ELISA Assay

1) Coat ELISA plates with 100 μL of 0.5 μg/mL recombinant human CD3 antigen in carbonate-bicarbonate coating buffer.
2) Cover plates with sealing film and incubate overnight at 4° C.
3) Decant plates and tap out residual liquid on a stack of paper towels.
4) Wash wells twice by dispensing 200 μL of various pH incubation buffer to the wells and completely aspirate the contents.
5) Add 200 μL of various pH incubation buffers (pH6.0, 6.2, 6.5, 6.7, 7.0 and 7.4) to the wells. Cover with sealing film and place the plate onto a plate shaker (set to 200 rpm) for 60 minutes at room temperature.
6) Decant plates and tap out residual liquid on a stack of paper towels.
7) Serially dilute test substances in various pH incubation buffers (pH6.0, 6.2, 6.5, 6.7, 7.0 and 7.4) to 100 ng/mL.
8) Add 100 μL/well of diluted test articles to the plates.
9) Cover with sealing film and place the plates onto a plate shaker (set to 200 rpm) for 60 minutes at room temperature.
10) Decant plates and tap out residual liquid on a stack of paper towels.
11) Wash wells three times by dispensing 200 μL of various pH wash buffers (pH6.0, 6.2, 6.5, 6.7, 7.0 and 7.4) to the wells and completely aspirate the contents.
12) Dilute the HRP secondary antibody at 1:2500 in various pH incubation buffers (pH6.0, 6.2, 6.5, 6.7, 7.0 and 7.4).
13) Add 100 μL HRP secondary antibody diluted in various pH incubation buffers (pH6.0, 6.2, 6.5, 6.7, 7.0 and 7.4) to each well.
14) Cover with sealing film and place the plates onto a plate shaker (set to 200 rpm) for 60 minutes at room temperature.
15) Decant plates and tap out residual liquid on a stack of paper towels.
16) Wash wells three times by dispensing 200 μL of various pH wash buffer (pH6.0, 6.2, 6.5, 6.7, 7.0 and 7.4) to the wells and completely aspirating the contents.
17) Dispense 50 μL per well of the TMB substrate solution into all wells of plates. Incubate at room temperature for about 4 minutes.
18) Add 50 μL per well of 1N HCl into all wells of the plates. Read plates at 450 nm using PerkinElmer EnSpire 2300 Multilabel Reader.

In vivo Efficacy Protocol for CAB Nectin4×CAB CD3

### 3.1 Test Articles

Vehicle (PBS buffer)
B12×WT CD3 (BA-150-HEL-BF1, isotype)
WT Nectin-4×WT CD3 (BA-150-19-01-01-BF1, benchmark)
CAB Nectin-4×CAB CD3 (BA-150-30-03-12-BF19)
   3.2 In vivo Efficacy Study
   Each mouse was inoculated subcutaneously in the right front flank region with NCI-H358 tumor cells (2×10⁶), and next day followed with i.v. human PBMC (10×10⁶) inoculation. Mice were randomly allocated to the different study groups (8 mice/group) when the mean tumor size reaches 120 mm³. Test articles are dosed at 2.5 mg/kg BIW×4 weeks, and tumor size as well as body weight are monitored twice per week.

It is to be understood, however, that even though numerous characteristics and advantages of the present disclosure have been set forth in the foregoing description, together with details of the structure and function of the disclosure, the disclosure is illustrative only, and changes may be made in detail, especially in matters of shape, size and arrangement of parts within the principles of the disclosure to the full extent indicated by the broad general meanings of the terms in which the appended claims are expressed.

All documents mentioned herein are hereby incorporated by reference in their entirety or alternatively to provide the disclosure for which they were specifically relied upon. The applicant(s) do not intend to dedicate any disclosed embodiments to the public, and to the extent any disclosed modifications or alterations may not literally fall within the scope of the claims, they are considered to be part hereof under the doctrine of equivalents.

1. A conjugate of Formula I, 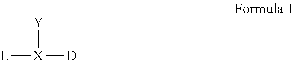
wherein:
L is a linker represented by: 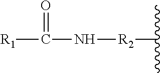
where R₁ is a nucleophilic functional group capable of coupling to an antibody, said nucleophilic functional group being selected from the group consisting of a C₁-C₁₀ alkyl halide, a C₁-C₁₀ alkyl alcohol, a phenol, a C₁-C₁₀ alkyl thiol, and a C₁-C₁₀ alkyl azide, and R₂ is a substituted or unsubstituted C₁-C₂₀ alkylene, or a substituted or unsubstituted (PEG)ₙ moiety represented by 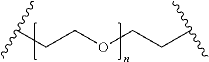
where n is 1 to 20;
X is a substituted nitrophenyl represented by 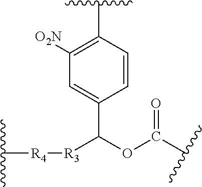
where R₃ is a C₁-C₂₀ alkylene, and R₄ is a triazole ring having a C═C double bond in the ring;
Y is a glycoside or glycoside derivative; and
D is a drug; and
Y is bonded to X at the site of a wavy line covalent attachment on the nitrophenyl ring,
D is bonded to X at the site of a wavy line covalent attachment on the carbonyl group, and
L is bonded to X via bonding of a wavy line covalent attachment between R₂ and R₄.

2. The conjugate of claim 1, wherein Riis a C₁-C₆ alkyl halide with a halide selected from the group consisting of Br, I, Cl, and F, a C₁-C₂ alkyl alcohol, a 4-phenol, or a C₁-C₂ alkyl thiol.

3. The conjugate of claim 2, wherein R₁ is —CH₂—Br.

4. The conjugate of claim 1, wherein R₂ is an unsubstituted C₁-C₂₀ alkylene.

5. The conjugate of claim 4, wherein R₂ is an unsubstituted C₂-C₅ alkylene.

6. The conjugate of claim 1, wherein R₂ is an unsubstituted (PEG)ₙ where n is 8.

7. The conjugate of claim 1, wherein R₃ is —CH₂—.

8. The conjugate of claim 1, wherein the glycoside or glycoside derivative Y is selected from the following structures:

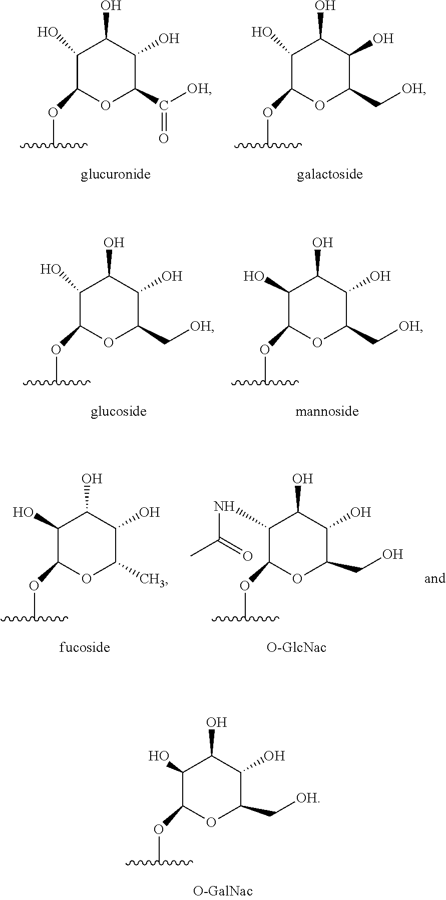

9. The conjugate of claim 8, wherein the glycoside or glycoside derivative Y is a glucuronide.

10. The conjugate of claim 1, wherein the triazole ring is formed with a linker-intermediate (L^{int}) having a formula represented by: 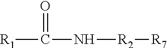
where R₁, R₂ and n are as defined in claim 1; and
R₇ is an azide group or an acetylene group, wherein the nitrogen atoms of the azide group or the carbons of the acetylene group become ring members in the triazole ring.

11. The conjugate of claim 10, wherein the triazole ring is formed with the azide group or the acetylene group, shown below, bonded to L: 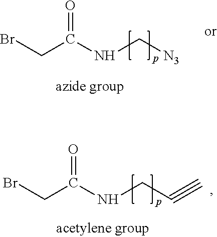
where p is 2 to 5.

12. The conjugate of claim 1, wherein the triazole ring is formed with an azide group or an acetylene group, shown below, bonded to L: 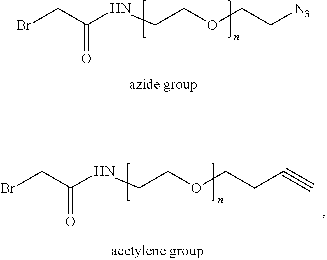
where n is 8.

13. The conjugate of claim 1, wherein the conjugate has a structure represented by SPC07:

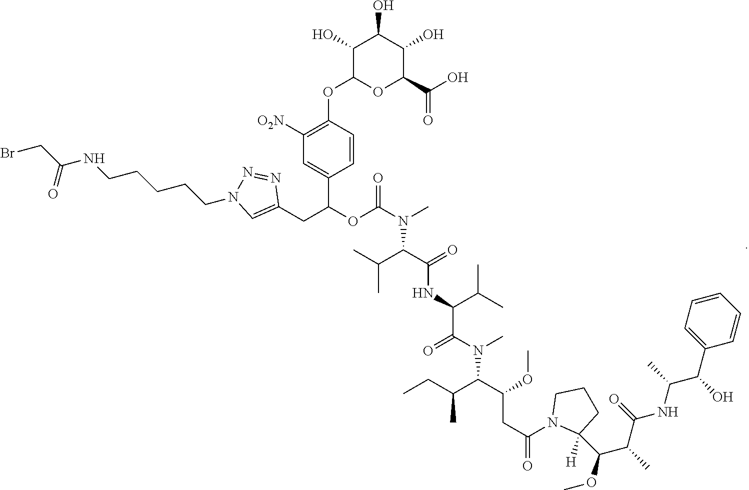 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(Cc1cn(CCCCCNC(=O)CBr)nn1)c1ccc(OC2O[C@H](C(=O)O)[C@@H](O)[C@H](O)[C@H]2O)c([N+](=O)[O-])c1)C(C)C]

14. The conjugate of claim 1, wherein the conjugate has a structure represented by SPC19

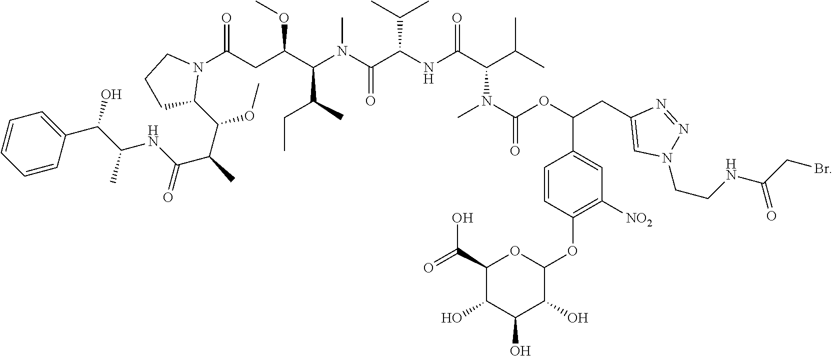 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(Cc1cn(CCNC(=O)CBr)nn1)c1ccc(OC2O[C@H](C(=O)O)[C@@H](O)[C@H](O)[C@H]2O)c([N+](=O)[O-])c1)C(C)C]

15. The conjugate of claim 11, wherein Y is not a galactoside when R₂ is a substituted or unsubstituted (PEG)ₙ.

16. The conjugate of claim 1, wherein the conjugate has a structure represented by SPC17

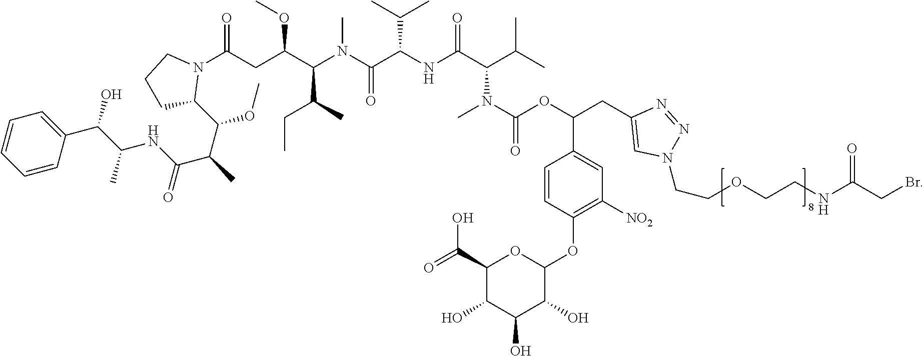

17. The conjugate of claim 1, wherein the conjugate has a structure represented by SPC02 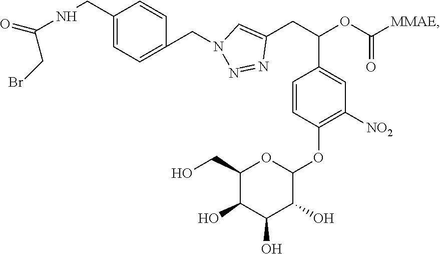
wherein MMAE is monomethyl auristatin E.

18. The conjugate of claim 1, wherein the conjugate has a structure represented by SPC04 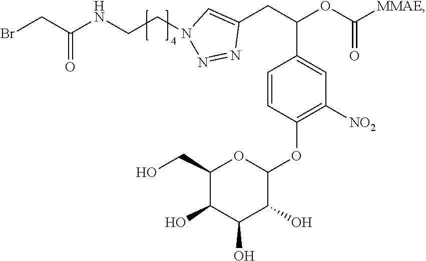
wherein MMAE is monomethyl auristatin E.

19. The conjugate of claim 1, wherein the conjugate has a structure represented by SPC05 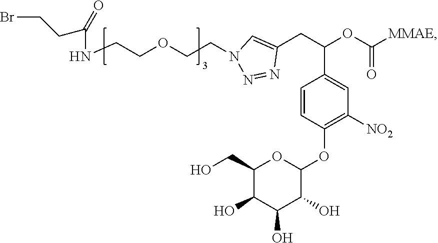
wherein MMAE is monomethyl auristatin E.

20. The conjugate of claim 1, wherein the conjugate has a structure represented by SPC06 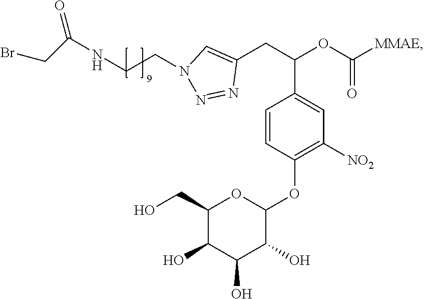
wherein MMAE is monomethyl auristatin E.

21. The conjugate of claim 1, wherein the conjugate has a structure represented by SPC08 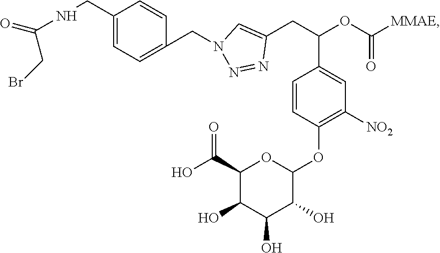
wherein MMAE is monomethyl auristatin E.

22. The conjugate of claim 1, wherein the drug D is selected from the group consisting of an auristatin, a dolastatin, a maytansinoid, a calicheamicin, a pyrrolobenzodiazepine, an anthracycline, a ribonuclease, and a DNA endonuclease.

23. The conjugate of claim 22, wherein the drug D is an auristatin selected from the group consisting of monomethyl auristatin E (MMAE) and monomethyl auristatin F (MMAF).

24-34. (canceled)

35. An immunoconjugate of Formula II, 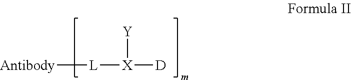
wherein:
L is represented by: 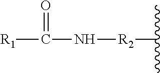
where R₁ is nucleophilic functional group capable of coupling to an antibody selected from the group consisting of a C₁-C₁₀ alkyl halide, a C₁-C₁₀ alkyl alcohol, a phenol, a C₁-C₁₀ alkyl thiol, and a C₁-C₁₀ alkyl azide,
R₂ is a substituted or unsubstituted C₁-C₂₀ alkylene, or a substituted or unsubstituted (PEG)ₙ moiety represented by 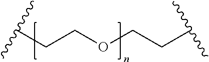
where n is 1 to 20, and
X is a substituted nitrophenyl represented by 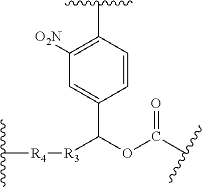
where R₃ is a C₁-C₂₀ alkylene; R₄ is a triazole ring having a C═C double bond in the ring;
Y is a glycoside or glycoside derivative;
D is a drug;
the wavy lines indicate a site of covalent attachment; and
m is 1 to 10,
wherein the term Antibody in Formula II represents an antibody or antibody fragment.

36-84. (canceled)

85. A pharmaceutical composition comprising:
the immunoconjugate of claim 35; and
a pharmaceutically acceptable carrier.

86-92. (canceled)

93. A method of treating cancer comprising a step of administering the immunoconjugate of claim 30 to a patient with cancer.

94. Use of the conjugate of the immunoconjugate of claim 35, for the treatment of cancer.

95-97. (canceled)


SEQUENCE LISTING (60 sequences)

SEQ ID NO: 1 - protein, 10 aa, synthetic construct
  REGION 1..10: note = Synthetic sequence HC CDR1
  VARIANT 9
GFTFSSYNXN

SEQ ID NO: 2 - protein, 17 aa, synthetic construct
  REGION 1..17: note = Synthetic sequence HC CDR2
YISSSSSTIYYADSVKG

SEQ ID NO: 3 - protein, 8 aa, synthetic construct
  REGION 1..8: note = Synthetic sequence HC CDR3
  VARIANT 6
  VARIANT 8
AYYYGXDX

SEQ ID NO: 4 - protein, 11 aa, synthetic construct
  REGION 1..11: note = Synthetic sequence LC CDR1
  VARIANT 1
  VARIANT 10
XASQGISGWXA

SEQ ID NO: 5 - protein, 7 aa, synthetic construct
  REGION 1..7: note = Synthetic Sequence LC CDR2
AASTLQS

SEQ ID NO: 6 - protein, 9 aa, synthetic construct
  REGION 1..9: note = Synthetic sequence LC CDR3
  VARIANT 6
  VARIANT 8
QQANSXPXT

SEQ ID NO: 7 - protein, 10 aa, synthetic construct
  REGION 1..10: note = Synthetic sequence HC CDR1
GFTFSSYNMN

SEQ ID NO: 8 - protein, 10 aa, synthetic construct
  REGION 1..10: note = Synthetic sequence HC CDR1
GFTFSSYNDN

SEQ ID NO: 9 - protein, 8 aa, synthetic construct
  REGION 1..8: note = Synthetic sequence HC CDR3
AYYYGMDV

SEQ ID NO: 10 - protein, 8 aa, synthetic construct
  REGION 1..8: note = Synthetic sequence HC CDR3
AYYYGDDV

SEQ ID NO: 11 - protein, 8 aa, synthetic construct
  REGION 1..8: note = Synthetic sequence HC CDR3
AYYYGMDK

SEQ ID NO: 12 - protein, 11 aa, synthetic construct
  REGION 1..11: note = Synthetic sequence LC CDR1
RASQGISGWLA

SEQ ID NO: 13 - protein, 11 aa, synthetic construct
  REGION 1..11: note = Synthetic sequence LC CDR1
RASQGISGWEA

SEQ ID NO: 14 - protein, 11 aa, synthetic construct
  REGION 1..11: note = Synthetic sequence LC CDR1
HASQGISGWLA

SEQ ID NO: 15 - protein, 9 aa, synthetic construct
  REGION 1..9: note = Synthetic sequence LC CDR3
QQANSFPPT

SEQ ID NO: 16 - protein, 9 aa, synthetic construct
  REGION 1..9: note = Synthetic sequence LC CDR3
QQANSEPPT

SEQ ID NO: 17 - protein, 9 aa, synthetic construct
  REGION 1..9: note = Synthetic sequence LC CDR3
QQANSFPDT

SEQ ID NO: 18 - protein, 117 aa, synthetic construct
  REGION 1..117: note = Synthetic sequence HC variable region
EVQLVESGGGLVQPGGSLRLSCAASGFTFSSYNMNWVRQAPGKGLEWVSYISSSSSTIYYADSVKGRFTISRDNAKNSLSLQMNSLRDEDTAVYYCARAYYYGMDVWGQGTTVTVSS

SEQ ID NO: 19 - protein, 117 aa, synthetic construct; identical to SEQ ID NO: 18
  REGION 1..117: note = Synthetic sequence HC variable region

SEQ ID NO: 20 - protein, 117 aa, synthetic construct; identical to SEQ ID NO: 18
  REGION 1..117: note = Synthetic sequence HC variable region

SEQ ID NO: 21 - protein, 117 aa, synthetic construct; identical to SEQ ID NO: 18
  REGION 1..117: note = Synthetic sequence HC variable region

SEQ ID NO: 22 - protein, 117 aa, synthetic construct
  REGION 1..117: note = Synthetic sequence HC variable region
EVQLVESGGGLVQPGGSLRLSCAASGFTFSSYNDNWVRQAPGKGLEWVSYISSSSSTIYYADSVKGRFTISRDNAKNSLSLQMNSLRDEDTAVYYCARAYYYGMDVWGQGTTVTVSS

SEQ ID NO: 23 - protein, 117 aa, synthetic construct
  REGION 1..117: note = Synthetic sequence HC variable region
EVQLVESGGGLVQPGGSLRLSCAASGFTFSSYNMNWVRQAPGKGLEWVSYISSSSSTIYYADSVKGRFTISRDNAKNSLSLQMNSLRDEDTAVYYCAEAYYYGMDVWGQGTTVTVSS

SEQ ID NO: 24 - protein, 117 aa, synthetic construct
  REGION 1..117: note = Synthetic sequence HC variable region
EVQLVESGGGLVQPGGSLRLSCAASGFTFSSYNMNWVRQAPGKGLEWVSYISSSSSTIYYADSVKGRFTISRDNAKNSLSLQMNSLRDEDTAVYYCARAYYYGDDVWGQGTTVTVSS

SEQ ID NO: 25 - protein, 117 aa, synthetic construct
  REGION 1..117: note = Synthetic sequence HC variable region
EVQLVQSGAEVKKPGATVKISCKVSGFTFSSYNMNWIRQSPSRGLEWLGYISSSSSTIYYADSVKGRFTISRDNSKNTLYLQMNSLRAEDTAVYYCARAYYYGDDVWGQGTLVTVSS

SEQ ID NO: 26 - protein, 117 aa, synthetic construct
  REGION 1..117: note = Synthetic sequence HC variable region
QVQLVQSGAEVKKPGASVKVSCKASGFTFSSYNMNWVRQARGQRLEWIGYISSSSSTIYYADSVKGRFTISRDNSKNTLYLQMNSLRAEDTAVYYCARAYYYGMDKWGQGTLVTVSS

SEQ ID NO: 27 - protein, 117 aa, synthetic construct; identical to SEQ ID NO: 25
  REGION 1..117: note = Synthetic sequence HC variable region

SEQ ID NO: 28 - protein, 117 aa, synthetic construct
  REGION 1..117: note = Synthetic sequence HC variable region
EVQLVQSGAEVKKPGESLRISCKGSGFTFSSYNMNWIRQSPSRGLEWLGYISSSSSTIYYADSVKGRFTISRDNSKNTLYLQMNSLRAEDTAVYYCARAYYYGMDKWGQGTLVTVSS

SEQ ID NO: 29 - protein, 117 aa, synthetic construct
  REGION 1..117: note = Synthetic sequence HC variable region
EVQLVESGGGLVQPGGSLRLSCAASGFTFSSYNMNWVRQAPGKGLEWVSYISSSSSTIYYADSVKGRFTISRDNAKNSLSLQMNSLRDEDTAVYYCARAYYYGMDKWGQGTTVTVSS

SEQ ID NO: 30 - protein, 117 aa, synthetic construct; identical to SEQ ID NO: 24
  REGION 1..117: note = Synthetic sequence HC variable region

SEQ ID NO: 31 - protein, 107 aa, synthetic construct
  REGION 1..107: note = Synthetic sequence LC variable region
DIQMTQSPSSVSASVGDRVTITCRASQGISGWLAWYQQKPGKAPKFLIYAASTLQSGVPSRFSGSGSGTDFTLTISSLQPEDFATYYCQQANSFPPTFGGGTKVEIK

SEQ ID NO: 32 - protein, 107 aa, synthetic construct
  REGION 1..107: note = Synthetic sequence LC variable region
DIQMTQSPSSVSASVGDRVTITCRASQGISGWEAWYQQKPGKAPKFLIYAASTLQSGVPSRFSGSGSGTDFTLTISSLQPEDFATYYCQQANSFPPTFGGGTKVEIK

SEQ ID NO: 33 - protein, 107 aa, synthetic construct
  REGION 1..107: note = Synthetic sequence LC variable region
DIQMTQSPSSVSASVGDRVTITCRASQGISGWLAWYQQKPGKAPKFLIYAASTLQSGVPSRFSGSGSGTDFTLTISSLQPEDFATYYCQQANSEPPTFGGGTKVEIK

SEQ ID NO: 34 - protein, 107 aa, synthetic construct
  REGION 1..107: note = Synthetic sequence LC variable region
DIQMTQSPSSVSASVGDRVTITCRASQGISGWLAWYQQKPGKAPKFLIYAASTLQSGVPSRFSGSGSGTDFTLTISSLQPEDFATYYCQQANSFPDTFGGGTKVEIK

SEQ ID NO: 35 - protein, 107 aa, synthetic construct; identical to SEQ ID NO: 31
  REGION 1..107: note = Synthetic sequence LC variable region

SEQ ID NO: 36 - protein, 107 aa, synthetic construct; identical to SEQ ID NO: 31
  REGION 1..107: note = Synthetic sequence LC variable region

SEQ ID NO: 37 - protein, 107 aa, synthetic construct; identical to SEQ ID NO: 31
  REGION 1..107: note = Synthetic sequence LC variable region

SEQ ID NO: 38 - protein, 107 aa, synthetic construct
  REGION 1..107: note = Synthetic sequence LC variable region
DIQMTQSPSSVSASVGDRVTITCHASQGISGWLAWYQQKPGKAPKFLIYAASTLQSGVPSRFSGSGSGTDFTLTISSLQPEDFATYYCQQANSFPPTFGGGTKVEIK

SEQ ID NO: 39 - protein, 107 aa, synthetic construct; identical to SEQ ID NO: 33
  REGION 1..107: note = Synthetic sequence LC variable region

SEQ ID NO: 40 - protein, 107 aa, synthetic construct; identical to SEQ ID NO: 32
  REGION 1..107: note = Synthetic sequence LC variable region

SEQ ID NO: 41 - protein, 107 aa, synthetic construct; identical to SEQ ID NO: 34
  REGION 1..107: note = Synthetic sequence LC variable region

SEQ ID NO: 42 - protein, 107 aa, synthetic construct; identical to SEQ ID NO: 32
  REGION 1..107: note = Synthetic sequence LC variable region

SEQ ID NO: 43 - protein, 107 aa, synthetic construct; identical to SEQ ID NO: 38
  REGION 1..107: note = Synthetic sequence LC variable region

SEQ ID NO: 44 - protein, 10 aa, synthetic construct
  REGION 1..10: note = Synthetic Sequence
GFTFNTYAMN

SEQ ID NO: 45 - protein, 19 aa, synthetic construct
  REGION 1..19: note = Synthetic sequence
RIRSKYNNYATYYADSVKD

SEQ ID NO: 46 - protein, 14 aa, synthetic construct
  REGION 1..14: note = Synthetic sequence
  VARIANT 2
  VARIANT 5
  VARIANT 8
  VARIANT 13
HXNFXNSXVSWFXY

SEQ ID NO: 47 - protein, 14 aa, synthetic construct
  REGION 1..14: note = Synthetic sequence
  VARIANT 13
RSSTGAVTTSNYXN

SEQ ID NO: 48 - protein, 7 aa, synthetic construct
  REGION 1..7: note = Synthetic sequence
GTNKRAP

SEQ ID NO: 49 - protein, 9 aa, synthetic construct
  REGION 1..9: note = Synthetic sequence
ALWYSNLWV

SEQ ID NO: 50 - protein, 14 aa, synthetic construct
  REGION 1..14: note = Synthetic sequence
HGNFGNSYVSWFAY

SEQ ID NO: 51 - protein, 14 aa, synthetic construct
  REGION 1..14: note = Synthetic sequence
HSNFGNSKVSWFAY

SEQ ID NO: 52 - protein, 14 aa, synthetic construct
  REGION 1..14: note = Synthetic sequence
HGNFPNSKVSWFQY

SEQ ID NO: 53 - protein, 14 aa, synthetic construct
  REGION 1..14: note = Synthetic sequence
HSNFGNSKVSWFAY

SEQ ID NO: 54 - protein, 14 aa, synthetic construct
  REGION 1..14: note = Synthetic sequence
RSSTGAVTTSNYAN

SEQ ID NO: 55 - protein, 14 aa, synthetic construct
  REGION 1..14: note = Synthetic sequence
RSSTGAVTTSNYDN

SEQ ID NO: 56 - protein, 471 aa, synthetic construct
  REGION 1..471: note = Synthetic sequence
DIQMTQSPSSVSASVGDRVTITCRASQGISGWLAWYQQKPGKAPKFLIYAASTLQSGVPSRFSGSGSGTDFTLTISSLQPEDFATYYCQQAQSFPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSRSGGGGEVQLVESGGGLVQPGGSLRLSCAASGFTFNTYAMNWVRQAPGKGLEWVARIRSKYNNYATYYADSVKDRFTISRDDSKNSLYLQMNSLKTEDTAVYYCVRHGNFGNSYVSWFAYWGQGTLVTVSSGGSGGSGGSGGSGGQAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLSR

SEQ ID NO: 57 - protein, 471 aa, synthetic construct
  REGION 1..471: note = Synthetic sequence
DIQMTQSPSSVSASVGDRVTITCRASQGISGWEAWYQQKPGKAPKFLIYAASTLQSGVPSRFSGSGSGTDFTLTISSLQPEDFATYYCQQAQSFPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSRSGGGGEVQLVESGGGLVQPGGSLRLSCAASGFTFNTYAMNWVRQAPGKGLEWVARIRSKYNNYATYYADSVKDRFTISRDDSKNSLYLQMNSLKTEDTAVYYCVRHSNFGNSKVSWFAYWGQGTLVTVSSGGSGGSGGSGGSGGQAVVTQEPSLTVSPGGTVTLTCRSSTGAVTTSNYDNWVQQKPGQAPRGLIGGTNKRAPWTPARFSGSLLGGKAALTITGAQAEDEADYYCALWYSNLWVFGGGTKLTVLSR

SEQ ID NO: 58 - protein, 471 aa, synthetic construct
  REGION 1..471: note = Synthetic sequence
DIQMTQSPSSVSASVGDRVTITCRASQGISGWEAWYQQKPGKAPKFLIYAASTLQSGVPSRFSGSGSGTDFTLTISSLQPEDFATYYCQQAQSFPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSRSGGGGEVQLVESGGGLVQPGGSLRLSCAASGFTFNTYAMNWVRQAPGKGLEWVARIRSKYNNYATYYADSVKDRFTISRDDSKNSLYLQMNSLKTEDTAVYYCVRHGNFPNSKVSWFQYWGQGTLVTVSSGGSGGSGGSGGSGGQAVVTQEPSLTVSPGGTVTLTCRSSTGAVTTSNYDNWVQQKPGQAPRGLIGGTNKRAPWTPARFSGSLLGGKAALTITGAQAEDEADYYCALWYSNLWVFGGGTKLTVLSR

SEQ ID NO: 59 - protein, 471 aa, synthetic construct
  REGION 1..471: note = Synthetic sequence
DIQMTQSPSSVSASVGDRVTITCRASQGISGWEAWYQQKPGKAPKFLIYAASTLQSGVPSRFSGSGSGTDFTLTISSLQPEDFATYYCQQAQSFPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSRSGGGGEVQLVESGGGLVQPGGSLRLSCAASGFTFNTYAMNWVRQAPGKGLEWVARIRSKYNNYATYYADSVKDRFTISRDDSKNSLYLQMNSLKTEDTAVYYCVRHSNFPNSKVSWFAYWGQGTLVTVSSGGSGGSGGSGGSGGQAVVTQEPSLTVSPGGTVTLTCRSSTGAVTTSNYDNWVQQKPGQAPRGLIGGTNKRAPWTPARFSGSLLGGKAALTITGAQAEDEADYYCALWYSNLWVFGGGTKLTVLSR

SEQ ID NO: 60 - protein, 471 aa, synthetic construct; identical to SEQ ID NO: 58
  REGION 1..471: note = Synthetic sequence